(19) United States
(12) Patent Application Publication
(10) Pub. No.: US 2025/0144067 A1
(43) Pub. Date: May 8, 2025

(54) PERSONALIZED STARVATION THERAPY FOR CANCER

(71) Applicant: Faeth Therapeutics, Inc., Austin, TX (US)

(72) Inventors: Oliver D.K. MADDOCKS, Glasgow (GB); Swati MISHRA, Cambridge (GB)

(21) Appl. No.: 18/676,700

(22) Filed: May 29, 2024

Related U.S. Application Data
(63) Continuation of application No. PCT/US2022/081307, filed on Dec. 9, 2022.
(60) Provisional application No. 63/288,062, filed on Dec. 10, 2021.

(51) Int. Cl. A61K 31/351; A61K 45/06; A61P 35/00

(52) U.S. Cl. CPC A61K 31/351; A61K 45/06; A61P 35/00

(57) ABSTRACT

Disclosed herein are methods of administering an inhibitor of the mevalonate pathway with a diet low in cholesterol or lipids. The methods of the present invention can be used to treat conditions such as cancer.

### CROSS-REFERENCE

This application is a continuation of International Application No. PCT/US2022/081307, filed Dec. 9, 2022, which claims the benefit of U.S. Provisional Application No. 63/288,062, filed on Dec. 10, 2021, which is incorporated herein by reference in its entirety.

### BACKGROUND

The mevalonate pathway, also referred to as the isoprenoid pathway or HMG-CoA reductase pathway, is a metabolic pathway that can play a role in multiple cellular processes. Cholesterol is an energy-rich, waxy hydrophobic compound synthesized by animals through the mevalonate pathway. Accumulations of excessive levels of serum cholesterol from dietary consumption or biosynthesis over long periods of time can be correlated with cardiovascular diseases.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Compared to normal cells, many cancer cells have evolved differential metabolic and nutritional demands for survival, proliferation, and metastases. Current therapies for the treatment for cancer or other pathologies can be ineffective due to patient-specific factors. Personalized methods and formulations can be developed for therapy of various diseases, including cancer.

In view of the foregoing, provided herein, in some embodiments, is a method of treating a condition in a subject in need thereof, the method comprising: a) administering to the subject a therapeutically effective amount of an inhibitor of a mevalonate pathway; and b) administering to the subject a diet, wherein the diet comprises less than about 40 g/day of lipids. In some embodiments, disclosed herein is a method of treating a condition in a subject in need thereof, the method comprising: a) administering to the subject a therapeutically effective amount of an inhibitor of a mevalonate pathway; and b) administering to the subject a diet, wherein the diet comprises less than about 200 mg/day of cholesterol.

In some embodiments, provided herein is a method of treating a condition in a subject in need thereof, the method comprising: a) administering to the subject a therapeutically effective amount of an HMG-CoA reductase inhibitor; and b) administering to the subject a diet, wherein the diet comprises less than about 40 g/day of lipids. In some embodiments, disclosed herein is a method of treating a condition in a subject in need thereof, the method comprising: a) administering to the subject a therapeutically effective amount of an HMG-CoA reductase inhibitor; and b) administering to the subject a diet, wherein the diet comprises less than about 200 mg/day of cholesterol.

In some embodiments, provided herein is a method of treating a condition in a subject in need thereof, the method comprising: a) administering to the subject a therapeutically effective amount of a cholesterol lowering agent; and b) administering to the subject a diet, wherein the diet is formulated to reduce a level of fat in the subject. In some embodiments, disclosed herein is a method of treating a condition in a subject in need thereof, the method comprising: a) administering to the subject a therapeutically effective amount of a cholesterol lowering agent; and b) administering to the subject a diet, wherein the diet is formulated to reduce a level of cholesterol in the subject.

In some embodiments, provided herein is a composition comprising: a) a cholesterol lowering agent; and b) a dietary product, wherein the dietary product is formulated to reduce a level of fat in a subject. In some embodiments, provided herein is a composition comprising: a) a cholesterol lowering agent; and b) a dietary product, wherein the dietary product is formulated to reduce a level of cholesterol in a subject.

In some embodiments, provided herein is a pharmaceutical composition comprising: a) a therapeutically effective amount of a cholesterol lowering agent; and b) a therapeutically effective amount of a dietary product, wherein the dietary product is formulated to reduce a level of fat in a subject. In some embodiments, provided herein is a pharmaceutical composition comprising: a) a therapeutically effective amount of a cholesterol lowering agent; and b) a therapeutically effective amount of a dietary product, wherein the dietary product is formulated to reduce a level of cholesterol in a subject.

In some embodiments, provided herein is a kit comprising: a) an effective amount of a cholesterol lowering agent; and b) an effective amount of a dietary product, wherein the dietary product is formulated to reduce a level of fat in a subject. In some embodiments, provided herein is a kit comprising: a) an effective amount of a cholesterol lowering agent; and b) an effective amount of a dietary product, wherein the dietary product is formulated to reduce a level of cholesterol in a subject.

### BRIEF DESCRIPTION OF THE DRAWINGS

FIG. 1 illustrates the experimental layout of the statin and complete/cholesterol-free media study.

FIG. 2A-2M show growth of cancer cell lines in complete and cholesterol-free media subjected to increasing doses of simvastatin.

FIG. 3A-3J show growth of pancreas and lung cancer cell lines in complete and cholesterol-free media subjected to increasing doses of simvastatin.

FIG. 4A-4I show growth of colorectal, breast, head and neck, blood, and kidney cancer cell lines in complete and cholesterol-free media subjected to increasing doses of simvastatin.

FIG. 5A-5M show growth of lung, pancreas, head and neck, and colorectal cancer cell Ines in complete and cholesterol-free media subjected to increasing doses of fluvastatin sodium.

FIG. 6 shows the plate layout of the drug validation study in H292 cells.

FIG. 7A-7C show growth of cancer cell lines in the three media conditions subjected to increasing doses of several drugs from the FDA screen under validation.

FIG. 8 shows the plate layout of the drug validation studies incorporating four media conditions.

FIG. 9A-9C show the growth of cancer cell lines in the four media conditions subjected to increasing doses of neticonazole.

FIG. 10A-10D show the growth of cancer cell lines in the four media conditions subjected to increasing doses of cepharanthine.

FIG. 11A-11B show the growth of cancer cell lines in the four media conditions subjected to increasing doses of alverine citrate.

FIG. 12A-12B show the growth of cancer cell lines in the four media conditions subjected to increasing doses of berbamine.

FIG. 13A-13C show the growth of cancer cell lines in the four media conditions subjected to increasing doses of pitolisant.

FIG. 14A-14G show the growth of cancer cell lines in the four media conditions subjected to increasing dose of avapritinib.

FIG. 15A-15D show the growth of cancer cell lines in the four media conditions subjected to increasing doses of toremifene.

FIG. 16A-16C show the growth of cancer cell lines in the four media conditions subjected to increasing doses of isavuconazole.

FIG. 17 shows the growth of H292 lung cancer tumors in vivo in mice receiving a normal cholesterol diet, low cholesterol diet, or low cholesterol diet and atorvastatin.

FIG. 18 shows the growth of SW480 colorectal tumors in vivo in mice receiving a normal cholesterol diet, a low cholesterol diet, a low cholesterol diet and atorvastatin, or a low cholesterol diet and toremifene.

FIG. 19 shows the growth of PC9 lung cancer tumors in vivo in mice receiving a low cholesterol diet; a low cholesterol diet and simvastatin; or a low cholesterol diet, simvastatin, and toremifene.

### DETAILED DESCRIPTION

The mevalonate pathway, also referred to as the isoprenoid pathway or HMG-CoA reductase pathway, is a metabolic pathway that plays a key role in multiple cellular processes. The mevalonate pathway begins with acetyl-CoA and converts mevalonate into sterol isoprenoids, such as cholesterol, the indispensable precursor of bile acids, lipoproteins, and steroid hormones; and into nonsterol isoprenoids, such as dolichol, heme-A, isopentenyl-tRNA, and ubiquinone. The mevalonate-isoprenoid pathway involves first the synthesis of 3-hydroxy-3-methylglutaryl-CoA (HMG)-CoA from acetyl-CoA through acetoacetyl-CoA. HMG-CoA reductase (HMGR), a highly regulated enzyme, catalyzes the conversion of HMG-CoA to mevalonic acid. HMGR is the rate-limiting enzyme of the mevalonate pathway. Mevalonate kinase (MK) is the second essential enzyme of the isoprenoid/cholesterol biosynthesis pathway, after HMGR, catalyzing the phosphorylation of mevalonic acid into phosphomevalonate. MK activity is regulated via feedback inhibition by intermediates in the isoprenoid/cholesterol pathway, including geranylpyrophosphate, farnesylpyrophosphate, and geranylgeranylpyrophosphate. The intermediates of the mevalonate biosynthetic pathway play important roles in the post-translational modification of a multitude of proteins involved in intracellular signaling and are essential in cell growth and differentiation, gene expression, protein glycosylation, and cytoskeletal assembly.

Cholesterol is an energy-rich, waxy hydrophobic compound synthesized by animals through the mevalonate pathway. Cholesterol functions as a source of energy, a precursor of steroid hormones and vitamin D, a structural component of cells, and is involved in multiple signaling pathways. Because the human body is able to synthesize cholesterol, cholesterol is classified as a non-essential nutrient. Accumulations of excessive levels of serum cholesterol from dietary consumption or biosynthesis over long periods of time can be positively correlated with cardiovascular diseases. Cholesterol synthesis as well as cholesterol uptake is shown to be upregulated in many cancer cells. Fast dividing cancer cells may depend on cholesterol as a source of high energy to sustain rapid proliferation.

Tumors comprise both cancer cells and non-cancer cells. Non-cancer cells can be recruited into tumors and can form up to 90% of a tumor volume. The non-cancerous cells, or tumor-stroma (“stromal”) cells, can develop with tumor growth and are considered cancer-associated cells. A main type of stromal cell population is fibroblasts, or cancer-associated fibroblasts (CAFs). The stroma can also comprise immune cells and non-cellular components like collagen. Tumors can comprise cancer cells, stromal cells, and other stromal components. Effective cancer therapies of the disclosure can affect one or more of the cell populations in a tumor. The methods disclosed herein can influence at least one group of cells selected from: 1) cancer cells; 2) stromal cells, such as CAFs and tumor infiltrating immune cells; and (3) other stromal components, such as collagen.

In some embodiments, disclosed herein is a method of treating a condition, the method comprising: a) administering to a subject in need thereof a therapeutically effective amount of an inhibitor of a mevalonate pathway; and b) administering to the subject a diet, wherein the diet comprises less than about 40 g/day of lipids. In some embodiments, disclosed herein is a method of treating a condition, the method comprising: a) administering to a subject in need thereof a therapeutically effective amount of an inhibitor of a mevalonate pathway; and b) administering to the subject a diet, wherein the diet comprises less than about 200 mg/day of cholesterol.

In some embodiments, disclosed herein is a method of treating a condition, the method comprising: a) administering to a subject in need thereof a therapeutically effective amount of an HMG-CoA reductase inhibitor; and b) administering to the subject a diet, wherein the diet composes less than about 40 g/day of lipids. In some embodiments, disclosed herein is a method of treating a condition, the method comprising: a) administering to a subject in need thereof a therapeutically effective amount of an HMG-CoA reductase inhibitor; and b) administering to the subject a diet, wherein the diet comprises less than about 200 mg/day of cholesterol.

In some embodiments, provided herein is a method of treating a condition in a subject in need thereof, the method comprising: a) administering to the subject a therapeutically effective amount of a cholesterol lowering agent; and b) administering to the subject a diet, wherein the diet is formulated to reduce a level of fat in the subject. In some embodiments, disclosed herein is a method of treating a condition in a subject in need thereof, the method comprising: a) administering to the subject a therapeutically effective amount of a cholesterol lowering agent; and b) administering to the subject a diet, wherein the diet is formulated to reduce a level of cholesterol in the subject.

### Therapeutic Agents

Disclosed herein are methods of treating a condition comprising administering a therapeutic agent. In some embodiments, the therapeutic agent is an inhibitor of a mevalonate pathway. In some embodiments, the therapeutic agent is an HMG-CoA reductase inhibitor. In some embodiments, the therapeutic agent is a statin.

Statins are inhibitors of HMG-CoA reductase (HMGR), the rate-limiting enzyme of the mevalonate pathway. Statins block the conversion of HMG-CoA to mevalonic acid by occupying the catalytic portion of HMGR, specifically the binding site of HMG-CoA, thus blocking access of the HMG-CoA substrate to the active site. By interrupting cholesterol synthesis in the liver, statins can activate the production of microsomal HMGR and cell surface low-density lipoprotein (LDL) receptors. This process can result in increased clearance of LDL from the blood stream and decreased blood LDL cholesterol levels.

HMGR inhibitors can be presecribed to patients with hyperlipidemia to reduce serum cholesterol by decreasing cholesterol biosynthesis. Further reduction in cholesterol levels can be achieved through reduced dietary intake of cholesterol, e.g., by following a plant-based diet. In a similar manner, cholesterol-dependent cancer cells can be therapeutically targeted by using statins to inhibit cholesterol biosynthesis along with a diet lacking in cholesterol.

In some embodiments, the therapeutic agent is a lipid lowering agent, a cholesterol lowering agent, or a triglyceride lowering agent. In some embodiments, the methods disclosed herein comprise administering a lipid lowering agent, a cholesterol lowering agent, or a triglyceride lowering agent. A cholesterol lowering agent can reduce serum cholesterol levels by inhibiting cholesterol biosynthesis, reducing cholesterol reabsorption, or increasing cholesterol metabolism. In some embodiments, the methods disclosed herein comprise administering two or more lipid lowering agents or cholesterol lowering agents.

In some embodiments, the methods disclosed herein comprise administering a statin. In some embodiments, the statin is atorvastatin or a pharmaceutically acceptable salt thereof. In some embodiments, the statin is atorvastatin calcium. In some embodiments, the statin is fluvastatin or a pharmaceutically acceptable salt thereof. In some embodiments, the statin is fluvastatin sodium. In some embodiments, the statin is pravastatin or a pharmaceutically acceptable salt thereof. In some embodiments, the statin is pravastatin sodium. In some embodiments, the statin is rosuvastatin or a pharmaceutically acceptable salt thereof. In some embodiments, the statin is rosuvastatin calcium. In some embodiments, the statin is simvastatin or a pharmaceutically acceptable salt thereof. In some embodiments, the statin is simvastatin sodium. In some embodiments, the statin is lovastatin or a pharmaceutically acceptable salt thereof. In some embodiments, the statin is lovastatin sodium. In some embodiments, the statin is pitavastatin or a pharmaceutically acceptable salt thereof. In some embodiments, the statin is pitavastatin calcium. In some embodiments, the statin is pitavastatin magnesium.

In some embodiments, a therapeutic agent of the disclosure can be a cholesteryl ester transfer protein (CETP) inhibitor. In some embodiments, the CETP inhibitor is torcetrapib, dalcetrapib, evacetrapib, obicetrapib, or anacetrapib. In some embodiments, the CETP inhibitor is anacetrapib. In some embodiments, a therapeutic agent of the disclosure can be a lecithin-cholesterol acyltransferase (LCAT) inhibitor.

In some embodiments, a therapeutic agent of the disclosure can be a bile acid-binding resin or a bile acid sequestrant. The agents bind to bile acids and prevent their reabsorption in the gastrointestinal system, promoting bile acid excretion. Disruption of bile acid reabsorption can have the effect of reducing cholesterol levels, e.g., low density lipoprotein (LDL) cholesterol levels.

In some embodiments, a therapeutic agent of the disclosure can be a cholesterol absorption inhibitor. In some embodiments, the cholesterol absorption inhibitor is ezetimibe. In some embodiments, a therapeutic agent of the disclosure can be a fixed combination of a statin and a cholesterol absorption inhibitor. In some embodiments, the combination cholesterol absorption inhibitor and statin is ezetimibe/simvastatin, ezetimibe/atorvastatin, or ezetimibe/rosuvastatin. In some embodiments, the combination cholesterol absorption inhibitor and statin is ezetimibe/simvastatin. In some embodiments, the combination cholesterol absorption inhibitor and statin is ezetimibe/atorvastatin. In some embodiments, the combination cholesterol absorption inhibitor and statin is ezetimibe/rosuvastatin.

### Pharmaceutically-Acceptable Salts

Provided herein is the use of pharmaceutically-acceptable salts of any therapeutic agent or compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound provided herein. The inorganic base comprises a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound provided herein. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of provided herein. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Pharmaceutical Compositions

A pharmaceutical composition of the disclosure can be used or administered, for example, before, during, or after treatment of a subject with, for example, another pharmaceutical agent.

In some embodiments, a subject is a human. Subjects can be, for example, elderly adults, adults, adolescents, pre-adolescents, children, toddlers, infants, neonates, and non-human animals. In some embodiments, a subject is a patient.

A pharmaceutical composition provided herein can be a combination of any pharmaceutical compounds or therapeutic agents described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically effective amounts as pharmaceutical compositions by various forms and routes including, for example, intravenous, subcutaneous, intramuscular, oral, parenteral, ophthalmic, subcutaneous, transdermal, nasal, vaginal, and topical administration. In some embodiments, a compound or pharmaceutical composition of the disclosure is administered orally. In some embodiments, a compound or pharmaceutical composition of the disclosure is administered intravenously.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino) propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use as described herein include liquid, powder, gel, nanosuspension, nanoparticle, microgel, aqueous or oily suspensions, emulsion, and any combination thereof.

Non-limiting examples of pharmaceutically-acceptable excipients suitable for use herein include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 h.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16, or about 24 h.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of pharmaceutically active agents suitable for combination with compositions of the disclosure include anti-infectives, i.e., aminoglycosides, antiviral agents, antimicrobials, anticholinergics/antispasmotics, antidiabetic agents, antihypertensive agents, antineoplastics, cardiovascular agents, central nervous system agents, coagulation modifiers, hormones, immunologic agents, immunosuppressive agents, and ophthalmic preparations.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

In some embodiments, provided herein dietary product is formulated to reduce a level of lipid, fat, or cholesterol in a subject. Dietary products described herein can be formulated as a powder, a gel, a solution, a suspension, a paste, a solid, a liquid, a liquid concentrate, a powder which may be reconstituted, a shake, a concentrate, a pill, a bar, a tablet, a capsule or a ready-to-use product. The dietary product can be formulated for oral, parenteral, intravenous, or enteral administration. A dietary product can also be a pharmaceutical composition when the supplement is in the form of a tablet, pill, capsule, liquid, aerosol, injectable solution, or other pharmaceutically acceptable formulation. In some embodiments, the dietary product is a beverage. In some embodiments, the dietary product is administered once, twice, three, four, five, six, seven eight, nine, or ten times a day.

In some embodiments, provided herein is a composition comprising: a) a cholesterol lowering agent; and b) a dietary product, wherein the dietary product is formulated to reduce a level of fat, a level of cholesterol, or both in a subject. In some embodiments, provided herein is a composition comprising: a) a cholesterol lowering agent; and b) a dietary product, wherein the dietary product is formulated to reduce a level of fat in a subject. In some embodiments, provided herein is a composition comprising: a) a cholesterol lowering agent; and b) a dietary product, wherein the dietary product is formulated to reduce a level of cholesterol in a subject.

In some embodiments, provided herein is a pharmaceutical composition comprising: a) a therapeutically effective amount of a cholesterol lowering agent; and b) a therapeutically effective amount of a dietary product, wherein the dietary product is formulated to reduce a level of fat, a level of cholesterol, or both in a subject. In some embodiments, provided herein is a pharmaceutical composition comprising: a) a therapeutically effective amount of a cholesterol lowering agent; and b) a therapeutically effective amount of a dietary product, wherein the dietary product is formulated to reduce a level of fat in a subject. In some embodiments, provided herein is a pharmaceutical composition comprising: a) a therapeutically effective amount of a cholesterol lowering agent; and b) a therapeutically effective amount of a dietary product, wherein the dietary product is formulated to reduce a level of cholesterol in a subject.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

In some embodiments, provided herein is a kit comprising: a) an effective amount of a cholesterol lowering agent; b) an effective amount of a dietary product, wherein the dietary product is formulated to reduce a level of fat in the subject; and optionally, c) written instructions for use of the kit. In some embodiments, provided herein is a kit comprising: a) an effective amount of a cholesterol lowering agent; b) an effective amount of a dietary product, wherein the dietary product is formulated to reduce a level of cholesterol in the subject; and optionally, c) written instructions for use of the kit.

### Methods of Administration

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 h of the onset of the symptoms, within the first 24 h of the onset of the symptoms, within the first 6 h of the onset of the symptoms, or within 3 h of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Multiple therapeutic agents can be administered in any order or simultaneously. In some embodiments, a compound of the invention is administered in combination with, before, or after treatment with another therapeutic agent. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

In some embodiments, a method of the disclosure can comprise administering an additional therapeutic agent. In some embodiments, the additional therapeutic agent is a chemotherapy. In some embodiments, the additional therapeutic agent is a radiotherapy. In some embodiments, the additional therapeutic agent is an immunotherapy.

In some embodiments, the additional therapeutic agent is a chemotherapy. In some embodiments, the chemotherapy comprises administering an antimetabolite. In some embodiments, the antimetabolite is an antifolate, pyrimidine antagonist, purine antagonist, or a ribonucleotide reductase inhibitor. In some embodiments, the chemotherapy is an alkylating agent. In some embodiments, the alkylating agent is an oxazaphosphorine, nitrogen mustard, imidazotetrazine, nitrosourea, alkyl sulfonate, hydrazine, or platinum-based agent. In some embodiments, the chemotherapy is a topoisomerase inhibitor. In some embodiments, the topoisomerase inhibitor is a topoisomerase I inhibitor or a topoisomerase II inhibitor. In some embodiments, the chemotherapy is a mitotic inhibitor. In some embodiments, the mitotic inhibitor is a vinca alkaloid, taxane, or nontaxane microtubule inhibitor. In some embodiments, the chemotherapy is an antitumor antibiotic. In some embodiments, the antitumor antibiotic is bleomycin, actinomycin D, an anthracycline, or mitomycin. In some embodiments, the chemotherapy is a protein kinase inhibitor. In some embodiments, the protein kinase inhibitor is a BCR-ABL and c-KIT tyrosine kinase inhibitor; EGFR tyrosine kinase inhibitor, ALK tyrosine kinase inhibitor, V600E mutated-BRAF oncogene inhibitor, MEK inhibitor, Bruton kinase inhibitor, Janus kinase inhibitor, or CDK inhibitor. In some embodiments, the chemotherapy is a selective estrogen receptor modulator (SERM).

In some embodiments, the chemotherapy is one or more of drugs listed in TABLE 1 or TABLE 2.

In some embodiments, the chemotherapy can include one or more of the following categories of anti-cancer agents:
(i) antiproliferative/antineoplastic drugs and combinations thereof, such as alkylating agents (e.g., cisplatin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin, and dacarbazine); a ntimetabolites (e.g., gemcitabine, antifolates, such as fluoropyrimidines (5-FU and tegafur), raltitrexed, methotrexate, pemetrexed, cytosine arabinoside, floxuridine, cytarabine, 6-mercaptopurine, 6-thioguanine, fludarabine phosphate, pentostatine, and hydroxyurea); antibiotics (e.g., anthracyclines such as adriamycin, bleomycin, doxorubicin, daunomycin, epirubicin, idarubicin, mitomycin-C, dactinomycin, and mithramycin); antimitotic agents (e.g., vinca alkaloids such as vincristine, vinblastine, vindesine, and vinorelbine; taxanes/taxoids such as paclitaxel, nab-paclitaxel, docetaxel, and cabazitaxel; and polokinase inhibitors); proteasome inhibitors, e.g., carfilzomib and bortezomib; interferon therapy; and topoisomerase inhibitors (e.g., epipodophyllotoxins such as etoposide and teniposide, amsacrine, topotecan, irinotecan, mitoxantrone, and camptothecin); bleomcin, dactinomycin, daunorubicin, doxorubicin, epirubicin, idarubicin, ara-C, paclitaxel (Taxol™), nab-paclitaxel, docetaxel, mithramycin, deoxycoformycin, mitomycin-C, L-asparaginase, interferons (e.g., IFN-alpha), etoposide, teniposide, DNA-demethylating agents, (e.g., azacitidine or decitabine); and histone de-acetylase (HDAC) inhibitors (e.g., vorinostat, MS-275, panobinostat, romidepsin, valproic acid, mocetinostat (MGCD0103), and pracinostat SB939);
(ii) cytostatic agents such as antiestrogens (e.g., tamoxifen, fulvestrant, toremifene, raloxifene, droloxifene and iodoxyfene), antiandrogens (e.g., bicalutamide, flutamide, nilutamide and cyproterone acetate), LHRH antagonists or LHRH agonists (e.g., goserelin, leuprorelin, and buserelin), progestogens (e.g., megestrol acetate), aromatase inhibitors (e.g., as anastrozole, letrozole, vorazole and exemestane) and inhibitors of 5Î±-reductase such as finasteride; and navelbene, CPT-II, anastrazole, letrazole, capecitabine, reloxafme, cyclophosphamide, ifosamide, and droloxafine;
(iii) anti-invasion agents, e.g., dasatinib and bosutinib (SKI-606), and metalloproteinase inhibitors, inhibitors of urokinase plasminogen activator receptor function or antibodies to heparanase;
(iv) inhibitors of growth factor function: e.g., such inhibitors include growth factor antibodies and growth factor receptor antibodies, e.g., the anti-erbB2 antibody trastuzumab, the anti-EGFR antibody panitumumab, the anti-erbB1 antibody cetuximab, tyrosine kinase inhibitors, e.g., inhibitors of the epidermal growth factor family (e.g., EGFR family tyrosine kinase inhibitors such as gefitinib, erlotinib, canertinib (CI-1033), afatinib, vandetanib, osimertinib, rociletinib, and lapatinib), and antibodies to costimulatory molecules such as CTLA-4, 4-IBB and PD-I, or antibodies to cytokines (IL-10, TGF-beta); inhibitors of the hepatocyte growth factor family; inhibitors of the insulin growth factor family; modulators of protein regulators of cell apoptosis (e.g., Bcl-2 inhibitors); inhibitors of the platelet-derived growth factor family such as imatinib and/or nilotinib (AMN107); inhibitors of serine/threonine kinases (e.g., Ras/Raf signaling inhibitors such as farnesyl transferase inhibitors, sorafenib, tipifarnib, and lonafarnib), inhibitors of cell signaling through MEK and/or AKT kinases, c-kit inhibitors, Abl kinase inhibitors, PI3K inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors; CCR2, CCR4, or CCR6 antagonists; and RAF kinase inhibitors.
(v) antiangiogenic agents such as those that inhibit the effects of VEGF, e.g., the anti-VEGF antibody bevacizumab; thalidomide; lenalidomide; and e.g., a VEGF receptor tyrosine kinase inhibitor such as vandetanib, vatalanib, sunitinib, axitinib, and pazopanib;
(vi) gene therapy approaches, including, e.g., approaches to replace aberrant genes such as aberrant p53 or aberrant BRCA1 or BRCA2;
(vii) immunotherapy approaches, including, e.g., antibody therapy such as alemtuzumab, rituximab, ibritumomab tiuxetan, and ofatumumab; interferons such as interferon α; interleukins such as IL-2 (aldesleukin); interleukin inhibitors e.g., IRAK4 inhibitors; cancer vaccines including prophylactic and treatment vaccines such as HPV vaccines, e.g., Gardasil®, Cervarix®, Oncophage®, and sipuleucel-T (Provenge®); gp100; dendritic cell-based vaccines (such as Ad.p53 DC); toll-like receptor modulators e.g., TLR-7 or TLR-9 agonists; PD-1, PD-L1, PD-L2, and CTL4-A modulators (e.g., nivolumab), antibodies and vaccines; indoleamine 2,3-dioxygenase 1 (IDO1) inhibitors (such as indoximod); anti-PD-1 monoclonal antibodies (such as MK-3475 and nivolumab); anti-PDL1 monoclonal antibodies (such as MEDI-4736 and RG-7446); anti-PDL2 monoclonal antibodies; and anti-CTLA-4 antibodies (such as ipilumumab);
(viii) cytotoxic agents, e.g., fludaribine (fludara), cladribine, and pentostatin;
(ix) targeted therapies, e.g., PI3K inhibitors, e.g., idelalisib and perifosine; SMAC (second mitochondria-derived activator of caspases) mimetics, also known as inhibitor of apoptosis proteins (IAP) antagonists. These agents act to suppress IAPs, e.g., XIAP, cIAP1, and cIAP2, and thereby re-establish cellular apoptotic pathways. Particular SMAC mimetics include birinapant (TL32711), LCL161 (Novartis), AEG 40730, SM-164, LBW242, ML101, AT-406, GDC-0917, AEG 35156, and HGS1029; and agents which target ubiquitin proteasome system (UPS), e.g., bortezomib, carfilzomib, marizomib (NPI-0052), and MLN9708; and
(x) chimeric antigen receptors, anticancer vaccines, and arginase inhibitors.

The therapeutic agents used in the methods of the disclosure can be a single agent or a combination of agents. In some embodiments, such combinations include agents that have different mechanisms of action. Such combinations can be administered simultaneously, separately, or sequentially. In some embodiments, a combination therapy provided herein is simultaneously administered. In some embodiments, a combination therapy provided herein is separately administered. In some embodiments, a combination therapy provided herein is sequentially administered.

In some embodiments, the additional therapeutic agent is an immunotherapy. In some embodiments, the immunotherapy is a cancer treatment vaccine. In some embodiments, the immunotherapy is a checkpoint inhibitor. In some embodiments, the checkpoint inhibitor blocks CTLA-4, PD-1, or PD-L1. In some embodiments, the checkpoint inhibitor is ipilimumab, nivolumab, or pembrolizumab. In some embodiments, the immunotherapy is an immune system modulator. In some embodiments, the immunotherapy is a monoclonal antibody. In some embodiments, the immunotherapy is a T-cell transfer therapy.

In some embodiments, disclosed herein is a method of treating a condition by 1) administering a therapeutically effective amount of an inhibitor of a mevalonate pathway; b) administering to the subject a diet, wherein the diet comprises less than about 40 g/day of lipids, wherein the subject consumes a 2000 kcal/day diet; and c) an anticancer agent. In some embodiments, disclosed herein is a method of treating a condition by 1) administering a therapeutically effective amount of an inhibitor of a mevalonate pathway; b) administering to the subject a diet, wherein the diet comprises less than about 200 mg/day of cholesterol, wherein the subject consumes a 2000 kcal/day; and c) an anticancer agent. In some embodiments, the anticancer agent is a SERM inhibitor.

In some embodiments, disclosed herein is a method of treating a condition by 1) administering a therapeutically effective amount of an inhibitor of a mevalonate pathway; b) administering to the subject a diet, wherein the diet comprises less than about 40 g/day of lipids, wherein the subject consumes a 2000 kcal/day diet; and c) an anticancer agent. In some embodiments, disclosed herein is a method of treating a condition by 1) administering a therapeutically effective amount of an inhibitor of a mevalonate pathway; b) administering to the subject a diet, wherein the diet comprises less than about 200 mg/day of cholesterol, wherein the subject consumes a 2000 kcal/day diet; and c) an anticancer agent. In some embodiments, the anticancer agent is a SERM inhibitor.

In some embodiments, disclosed herein is a method of treating a condition by 1) administering a therapeutically effective amount of a first inhibitor of a mevalonate pathway; b) a therapeutically effective amount of a second inhibitor of the mevalonate pathway; and c) administering to the subject a diet, wherein the diet comprises less than about 40 g/day of lipids, wherein the subject consumes a 2000 kcal/day diet. In some embodiments, disclosed herein is a method of treating a condition by 1) administering a therapeutically effective amount of a first inhibitor of a mevalonate pathway; b) a therapeutically effective amount of a second inhibitor of the mevalonate pathway; and c) administering to the subject a diet, wherein the diet comprises less than about 200 mg/day of cholesterol, wherein the subject consumes a 2000 kcal/day diet.

### Dosing

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein can be administered or present in a composition in a range of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be administered or present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a dose can be expressed in terms of an amount of the drug divided by the mass of the subject, for example, milligrams of drug per kilograms of subject body mass. In some embodiments, a compound is administered in an amount ranging from about 5 mg/kg to about 50 mg/kg, about 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg.

In some embodiments, an inhibitor of a mevalonate pathway, a HMG-CoA reductase inhibitor, or a statin of the disclosure can be administered in an amount of from about 5 mg to about 10 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, or about 90 mg to about 100 mg.

In some embodiments, the inhibitor of a mevalonate pathway is an HMG-CoA reductase inhibitor. In some embodiments, an HMG-CoA reductase inhibitor used in the methods disclosed herein is a statin.

In some embodiments, the statin is simvastatin or a pharmaceutically acceptable salt thereof. In some embodiments, the statin is simvastatin sodium. In some embodiments, a method of the disclosure comprises administering simvastatin or a pharmaceutically acceptable salt thereof in an amount of from about 5 mg/day to about 45 mg/day. In some embodiments, a method of the disclosure comprises administering simvastatin or a pharmaceutically acceptable salt thereof in an amount of from about 5 mg/day to about 10 mg/day, from about 10 mg/day to about 15 mg/day, from about 15 mg/day to about 20 mg/day, from about 20 mg/day to about 25 mg/day, from about 25 mg/day to about 30 mg/day, from about 30 mg/day to about 35 mg/day, from about 35 mg/day to about 40 mg/day, or from about 40 mg/day to about 45 mg/day. In some embodiments, a method of the disclosure comprises administering simvastatin or a pharmaceutically acceptable salt thereof in an amount of from about 10 mg/day to about 20 mg/day. In some embodiments, a method of the disclosure comprises administering simvastatin or a pharmaceutically acceptable salt thereof in an amount of about 5 mg/day, about 10 mg/day, about 15 mg/day, about 20 mg/day, about 25 mg/day, about 30 mg/day, about 35 mg/day, about 40 mg/day, or about 45 mg/day. In some embodiments, a method of the disclosure comprises administering simvastatin or a pharmaceutically acceptable salt thereof in an amount of about 10 mg/day. In some embodiments, a method of the disclosure comprises administering simvastatin or a pharmaceutically acceptable salt thereof in an amount of about 15 mg/day. In some embodiments, a method of the disclosure comprises administering simvastatin or a pharmaceutically acceptable salt thereof in an amount of about 20 mg/day.

In some embodiments, the statin is fluvastatin or a pharmaceutically acceptable salt thereof. In some embodiments, the statin is fluvastatin sodium. In some embodiments, a method of the disclosure comprises administering fluvastatin or a pharmaceutically acceptable salt thereof in an amount of from about 5 mg/day to about 80 mg/day. In some embodiments, a method of the disclosure comprises administering fluvastatin or a pharmaceutically acceptable salt thereof in an amount of from about 5 mg/day to about 10 mg/day, from about 10 mg/day to about 15 mg/day, from about 15 mg/day to about 20 mg/day, from about 20 mg/day to about 25 mg/day, from about 25 mg/day to about 30 mg/day, from about 30 mg/day to about 35 mg/day, from about 35 mg/day to about 40 mg/day, from about 40 mg/day to about 45 mg/day, from about 45 mg/day to about 50 mg/day, from about 50 mg/day to about 55 mg/day, from about 55 mg/day to about 60 mg/day, from about 60 mg/day to about 65 mg/day, from about 65 mg/day to about 70 mg/day, from about 70 mg/day to about 75 mg/day, or from about 75 mg/day to about 80 mg/day. In some embodiments, a method of the disclosure comprises administering fluvastatin or a pharmaceutically acceptable salt thereof in an amount of from about 10 mg/day to about 20 mg/day. In some embodiments, a method of the disclosure comprises administering fluvastatin or a pharmaceutically acceptable salt thereof in an amount of from about 20 mg/day to about 30 mg/day. In some embodiments, a method of the disclosure comprises administering fluvastatin or a pharmaceutically acceptable salt thereof in an amount of from about 30 mg/day to about 40 mg/day. In some embodiments, a method of the disclosure comprises administering fluvastatin or a pharmaceutically acceptable salt thereof in an amount of about 5 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, or about 80 mg/day. In some embodiments, a method of the disclosure comprises administering fluvastatin or a pharmaceutically acceptable salt thereof in an amount of about 10 mg/day. In some embodiments, a method of the disclosure comprises administering fluvastatin or a pharmaceutically acceptable salt thereof in an amount of about 15 mg/day. In some embodiments, a method of the disclosure comprises administering fluvastatin or a pharmaceutically acceptable salt thereof in an amount of about 20 mg/day. In some embodiments, fluvastatin or a pharmaceutically acceptable salt thereof can be administered once or twice a day.

In some embodiments, the statin is atorvastatin or a pharmaceutically acceptable salt thereof. In some embodiments, the statin is atorvastatin calcium. In some embodiments, a method of the disclosure comprises administering atorvastatin or a pharmaceutically acceptable salt thereof in an amount of from about 5 mg/day to about 80 mg/day. In some embodiments, a method of the disclosure comprises administering atorvastatin or a pharmaceutically acceptable salt thereof in an amount of from about 5 mg/day to about 10 mg/day, from about 10 mg/day to about 15 mg/day, from about 15 mg/day to about 20 mg/day, from about 20 mg/day to about 25 mg/day, from about 25 mg/day to about 30 mg/day, from about 30 mg/day to about 35 mg/day, from about 35 mg/day to about 40 mg/day, from about 40 mg/day to about 45 mg/day, from about 45 mg/day to about 50 mg/day, from about 50 mg/day to about 55 mg/day, from about 55 mg/day to about 60 mg/day, from about 60 mg/day to about 65 mg/day, from about 65 mg/day to about 70 mg/day, from about 70 mg/day to about 75 mg/day, or from about 75 mg/day to about 80 mg/day. In some embodiments, a method of the disclosure comprises administering atorvastatin or a pharmaceutically acceptable salt thereof in an amount of from about 10 mg/day to about 20 mg/day. In some embodiments, a method of the disclosure comprises administering atorvastatin or a pharmaceutically acceptable salt thereof in an amount of from about 20 mg/day to about 30 mg/day. In some embodiments, a method of the disclosure comprises administering atorvastatin or a pharmaceutically acceptable salt thereof in an amount of from about 30 mg/day to about 40 mg/day. In some embodiments, a method of the disclosure comprises administering atorvastatin or a pharmaceutically acceptable salt thereof in an amount of about 5 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, or about 80 mg/day. In some embodiments, a method of the disclosure comprises administering atorvastatin or a pharmaceutically acceptable salt thereof in an amount of about 10 mg/day. In some embodiments, a method of the disclosure comprises administering atorvastatin or a pharmaceutically acceptable salt thereof in an amount of about 15 mg/day. In some embodiments, a method of the disclosure comprises administering atorvastatin or a pharmaceutically acceptable salt thereof in an amount of about 20 mg/day.

In some embodiments, the statin is rosuvastatin or a pharmaceutically acceptable salt thereof. In some embodiments, the statin is rosuvastatin calcium. In some embodiments, a method of the disclosure comprises administering rosuvastatin or a pharmaceutically acceptable salt thereof in an amount of from about 5 mg/day to about 45 mg/day. In some embodiments, a method of the disclosure comprises administering rosuvastatin or a pharmaceutically acceptable salt thereof in an amount of from about 5 mg/day to about 10 mg/day, from about 10 mg/day to about 15 mg/day, from about 15 mg/day to about 20 mg/day, from about 20 mg/day to about 25 mg/day, from about 25 mg/day to about 30 mg/day, from about 30 mg/day to about 35 mg/day, from about 35 mg/day to about 40 mg/day, or from about 40 mg/day to about 45 mg/day. In some embodiments, a method of the disclosure comprises administering rosuvastatin or a pharmaceutically acceptable salt thereof in an amount of from about 10 mg/day to about 20 mg/day. In some embodiments, a method of the disclosure comprises administering rosuvastatin or a pharmaceutically acceptable salt thereof in an amount of about 5 mg/day, about 10 mg/day, about 15 mg/day, about 20 mg/day, about 25 mg/day, about 30 mg/day, about 35 mg/day, about 40 mg/day, or about 45 mg/day. In some embodiments, a method of the disclosure comprises administering rosuvastatin or a pharmaceutically acceptable salt thereof in an amount of about 10 mg/day. In some embodiments, a method of the disclosure comprises administering rosuvastatin or a pharmaceutically acceptable salt thereof in an amount of about 15 mg/day. In some embodiments, a method of the disclosure comprises administering rosuvastatin or a pharmaceutically acceptable salt thereof in an amount of about 20 mg/day.

In some embodiments, the statin is lovastatin or a pharmaceutically acceptable salt thereof. In some embodiments, the statin is lovastatin sodium. In some embodiments, a method of the disclosure comprises administering lovastatin or a pharmaceutically acceptable salt thereof in an amount of from about 15 mg/day to about 20 mg/day, from about 20 mg/day to about 25 mg/day, from about 25 mg/day to about 30 mg/day, from about 30 mg/day to about 35 mg/day, from about 35 mg/day to about 40 mg/day, from about 40 mg/day to about 45 mg/day, from about 45 mg/day to about 50 mg/day, from about 50 mg/day to about 55 mg/day, from about 55 mg/day to about 60 mg/day, or from about 60 mg/day to about 65 mg/day. In some embodiments, a method of the disclosure comprises administering lovastatin or a pharmaceutically acceptable salt thereof in an amount of from about 20 mg/day to about 30 mg/day. In some embodiments, a method of the disclosure comprises administering lovastatin or a pharmaceutically acceptable salt thereof in an amount of from about 30 mg/day to about 40 mg/day. In some embodiments, a method of the disclosure comprises administering lovastatin or a pharmaceutically acceptable salt thereof in an amount of from about 40 mg/day to about 50 mg/day. In some embodiments, a method of the disclosure comprises administering lovastatin or a pharmaceutically acceptable salt thereof in an amount of from about 50 to about 60 mg/day.

In some embodiments, the statin is pravastatin or a pharmaceutically acceptable salt thereof. In some embodiments, the statin is pravastatin sodium. In some embodiments, a method of the disclosure comprises administering pravastatin or a pharmaceutically acceptable salt thereof in an amount of from about 5 mg/day to about 80 mg/day. In some embodiments, a method of the disclosure comprises administering pravastatin or a pharmaceutically acceptable salt thereof in an amount of from about 5 mg/day to about 10 mg/day, from about 10 mg/day to about 15 mg/day, from about 15 mg/day to about 20 mg/day, from about 20 mg/day to about 25 mg/day, from about 25 mg/day to about 30 mg/day, from about 30 mg/day to about 35 mg/day, from about 35 mg/day to about 40 mg/day, from about 40 mg/day to about 45 mg/day, from about 45 mg/day to about 50 mg/day, from about 50 mg/day to about 55 mg/day, from about 55 mg/day to about 60 mg/day, from about 60 mg/day to about 65 mg/day, from about 65 mg/day to about 70 mg/day, from about 70 mg/day to about 75 mg/day, or from about 75 mg/day to about 80 mg/day. In some embodiments, a method of the disclosure comprises administering pravastatin or a pharmaceutically acceptable salt thereof in an amount of from about 10 mg/day to about 20 mg/day. In some embodiments, a method of the disclosure comprises administering pravastatin or a pharmaceutically acceptable salt thereof in an amount of from about 20 mg/day to about 30 mg/day. In some embodiments, a method of the disclosure comprises administering pravastatin or a pharmaceutically acceptable salt thereof in an amount of from about 30 mg/day to about 40 mg/day. In some embodiments, a method of the disclosure comprises administering pravastatin or a pharmaceutically acceptable salt thereof in an amount of about 5 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, or about 80 mg/day. In some embodiments, a method of the disclosure comprises administering pravastatin or a pharmaceutically acceptable salt thereof in an amount of about 10 mg/day. In some embodiments, a method of the disclosure comprises administering pravastatin or a pharmaceutically acceptable salt thereof in an amount of about 15 mg/day. In some embodiments, a method of the disclosure comprises administering pravastatin or a pharmaceutically acceptable salt thereof in an amount of about 20 mg/day.

In some embodiments, the statin is pitavastatin or a pharmaceutically acceptable salt thereof. In some embodiments, the statin is pitavastatin calcium. In some embodiments, the statin is pitavastatin magnesium. In some embodiments, pitavastatin or a pharmaceutically acceptable salt thereof can be administered in an amount of about 0.5 mg/day, about 1 mg/day, about 1.5 mg/day, about 2 mg/day, about 2.5 mg/day, about 3 mg/day, about 3.5 mg/day, about 4 mg/day, or about 4.5 mg/day. In some embodiments, pitavastatin or a pharmaceutically acceptable salt thereof can be administered in an amount of about 1 mg/day. In some embodiments, pitavastatin or a pharmaceutically acceptable salt thereof can be administered in an amount of about 2 mg/day. In some embodiments, pitavastatin or a pharmaceutically acceptable salt thereof can be administered in an amount of about 4 mg/day.

A method of the disclosure can comprise administering a diet. In some embodiments, a method of the disclosure can comprise administering a vegetarian diet. In some embodiments, a method of the disclosure can comprise administering a vegan diet. In some embodiments, a method of the disclosure can comprise administering a diet substantially devoid of animal-based fats, e.g., no more than 1%, no more than 0.5%, or no more than 0.1% of total fat in the diet. In some embodiments, a method of the disclosure can comprise administering a diet that does not comprise animal-based fats. In some embodiments, a method of the disclosure can comprise administering a diet devoid of animal-based fats. In some embodiments, a method of the disclosure can comprise replacing animal-based fats in a diet with plant-based fats. In some embodiments, a method of the disclosure can comprise administering a diet that does not comprise animal-based fat. In some embodiments, a method of the disclosure can comprise administering a diet where greater than 10%, 20%, 30%, 40%, 50%, 60%, 70%, 80%, 90%, 95%, or 99% of the animal-based fat is replaced with plant-based fats. In some embodiments, a method of the disclosure can comprise administering a diet devoid of or restricted in lipids. In some embodiments, a method of the disclosure can comprise administering a diet devoid of or restricted in cholesterol. In some embodiments, a method of the disclosure can comprise administering a diet devoid of or restricted in fat.

In some embodiments, a method of the disclosure can comprise administering a diet that is restricted in total lipid intake, e.g., a daily total lipid intake. In some embodiments, a method of the disclosure can comprise administering a diet that is restricted in a daily recommended dietary lipid intake. In some embodiments, a diet comprises no more than 80%, no more than 70%, no more than 75%, no more than 60%, no more than 50%, no more than 40%, no more than 30%, no more than 25%, no more than 20%, no more than 10%, no more than 5%, no more than 4%, no more than 3%, no more than 2%, no more than 1%, or no more than 0.5% of a subject's average daily lipid intake prior to start of the diet. In some embodiments, a diet comprises less than 90%, less than 80%, less than 70%, less than 75%, less than 60%, less than 50%, less than 40%, less than 30%, less than 25%, less than 20%, less than 10%, less than 5%, less than 4%, less than 3%, less than 2%, less than 1%, or less than 0.5% of a subject's average daily lipid intake prior to start of the diet. In some embodiments, a method of the disclosure can comprise administering a diet that does not comprise lipids. A subject's lipid intake can be according to a dietary guideline published by a federal government agency, e.g., the United States Departments of Agriculture and Health and Human Services or the National Health and Nutrition Examination Survey (NHANES). A subject's average daily lipid intake can be according to the Dietary Guidelines for Americans published by the United States Departments of Agriculture and Health and Human Services. A subject's average daily recommended lipid intake can be according to the Dietary Guidelines for Americans published by the United States Departments of Agriculture and Health and Human Services.

In some embodiments, a method of the disclosure can comprise administering a diet comprising no more than 100 g/day, no more than 90 g/day, no more than 80 g/day, no more than 70 g/day, no more than 60 g/day, no more than 50 g/day, no more than 40 g/day, no more than 30 g/day, no more than 20 g/day, no more than 10 g/day, no more than 5 g/day, or no more than 1 g/day of total lipids, e.g., based on a 2000 kcal/day diet. In some embodiments, a method of the disclosure can comprise administering a diet comprising a restricted amount of lipids, e.g., less than about 40% of total calories from lipids, e.g., based on a 2000 kcal/day diet. In some embodiments, a method of the disclosure can comprise administering a diet comprising less than about 40%, less than about 35%, less than about 30%, less than about 25%, less than about 20%, less than about 15%, less than about 10%, or less than about 5% of total daily calories from lipids, e.g., based on a 2000 kcal/day diet. In some embodiments, a method of the disclosure can comprise administering a diet comprising less than about 30% of total daily calories from lipids, e.g., based on a 2000 kcal/day diet. In some embodiments, a method of the disclosure can comprise administering a diet comprising less than about 25% of total daily calories from lipids, e.g., based on a 2000 kcal/day diet. In some embodiments, a method of the disclosure can comprise administering a diet comprising less than about 20% of total daily calories from lipids, e.g., based on a 2000 kcal/day diet. In some embodiments, a method of the disclosure can comprise administering a diet comprising less than about 15% of total daily calories from lipids, e.g., based on a 2000 kcal/day diet. In some embodiments, a method of the disclosure can comprise administering a diet comprising less than about 10% of total daily calories from lipids, e.g., based on a 2000 kcal/day diet. In some embodiments, a method of the disclosure can comprise administering a diet comprising less than about 5% of total daily calories from lipids, e.g., based on a 2000 kcal/day diet. The amount of lipids in the diet can vary in a proportionate amount for subjects who consume less than 2000 kcal/day diet or more than 2000 kcal/day diet.

In some embodiments, a method of the disclosure can comprise administering a diet comprising less than about 80 g/day, less than about 75 g/day, less than about 70 g/day, less than about 65 g/day, less than about 60 g/day, less than about 55 g/day, less than about 50 g/day, less than about 45 g/day, less than about 40 g/day, less than about 35 g/day, less than about 30 g/day, less than about 25 g/day, less than about 20 g/day, less than about 15 g/day, less than about 10 g/day, or less than about 5 g/day of lipids, e.g., based on a 2000 kcal/day diet. In some embodiments, a method of the disclosure can comprise administering diet comprising less than about 70 g/day of lipids, e.g., based on a 2000 kcal/day diet. In some embodiments, a method of the disclosure can comprise administering a diet comprising less than about 60 g/day of lipids, e.g., based on a 2000 kcal/day diet. In some embodiments, a method of the disclosure can comprise administering a diet comprising less than about 50 g/day of lipids, e.g., based on a 2000 kcal/day diet. In some embodiments, a method of the disclosure can comprise administering a diet comprising less than about 40 g/day of lipids, e.g., based on a 2000 kcal/day diet. In some embodiments, a method of the disclosure can comprise administering a diet comprising less than about 30 g/day of lipids, e.g., based on a 2000 kcal/day diet. In some embodiments, a method of the disclosure can comprise administering a diet comprising less than about 20 g/day of lipids, e.g., based on a 2000 kcal/day diet. The amount of lipids in the diet can vary in a proportionate amount for subjects who consume less than 2000 kcal/day diet or more than 2000 kcal/day diet.

In some embodiments, a method of the disclosure can comprise administering a diet that is restricted in total dietary cholesterol intake, e.g., a daily total cholesterol intake. In some embodiments, a method of the disclosure can comprise administering a diet comprising no more than 500 mg/day, no more than 450 mg/day, no more than 400 mg/day, no more than 350 mg/day, no more than 300 mg/day, no more than 250 mg/day, no more than 200 mg/day, no more than 150 mg/day, no more than 100 mg/day, no more than 75 mg/day, or no more than 50 mg/day of cholesterol, e.g., based on a 2000 kcal/day diet. In some embodiments, a method of the disclosure can comprise administering a diet devoid of cholesterol. In some embodiments, a method of the disclosure can comprise administering a diet comprising no more than 250 mg/day of cholesterol, e.g., based on a 2000 kcal/day diet. In some embodiments, a method of the disclosure can comprise administering a diet comprising no more than 200 mg/day of cholesterol, e.g., based on a 2000 kcal/day diet. In some embodiments, a method of the disclosure can comprise administering a diet comprising no more than 150 mg/day of cholesterol, e.g., based on a 2000 kcal/day diet. In some embodiments, a method of the disclosure can comprise administering a diet comprising no more than 100 mg/day of cholesterol, e.g., based on a 2000 kcal/day diet. Diets low in or devoid of cholesterol can exclude food products containing high levels of cholesterol, such as animal fat, egg yolks, shrimp, whole milk dairy, butter, cream, and cheese.

In some embodiments, a method of the disclosure can comprise administering a diet that is restricted in cholesterol intake, e.g., a daily cholesterol intake. Cholesterol dietary intake by men can average by about 350 mg/day; cholesterol dietary intake by women can average by about 240 mg/day. In some embodiments, a method of the disclosure can comprise administering a diet that is restricted in a daily recommended dietary cholesterol intake. In some embodiments, a diet comprises no more than 80%, no more than 70%, no more than 75%, no more than 60%, no more than 50%, no more than 40%, no more than 30%, no more than 25%, no more than 20%, no more than 10%, no more than 5%, no more than 4%, no more than 3%, no more than 2%, no more than 1%, or no more than 0.5% of a subject's average daily cholesterol intake prior to start of the diet. In some embodiments, a diet comprises less than 90%, less than 80%, less than 70%, less than 75%, less than 60%, less than 50%, less than 40%, less than 30%, less than 25%, less than 20%, less than 10%, less than 5%, less than 4%, less than 3%, less than 2%, less than 1%, or less than 0.5% of a subject's average daily cholesterol intake prior to start of the diet. A subject's cholesterol intake can be according to a dietary guideline published by a federal government agency, e.g., the United States Departments of Agriculture and Health and Human Services or the National Health and Nutrition Examination Survey (NHANES). A subject's average daily cholesterol intake can be according to the Dietary Guidelines for Americans published by the United States Departments of Agriculture and Health and Human Services. A subject's average daily recommended cholesterol intake can be according to the Dietary Guidelines for Americans published by the United States Departments of Agriculture and Health and Human Services. For example, a daily recommended cholesterol intake is about 300 mg/day according to the 2010 Dietary Guidelines for Americans published by the United States Departments of Agriculture and Health and Human Services.

In some cases, a subject's typical cholesterol intake exceeds a recommended cholesterol intake amount. In some embodiments, a method of the disclosure can comprise administering a diet comprising no more than 170%, no more than 160%, no more than 150%, no more than 140%, no more than 130%, no more than 120%, no more than 110%, no more than 100%, no more than 90%, no more than 80%, no more than 70%, no more than 60%, no more than 50%, no more than 40%, no more than 30%, no more than 20%, no more than 10% of a daily recommended cholesterol intake amount. In some embodiments, a method of the disclosure can comprise administering a diet that does not comprise cholesterol. In some embodiments, a method of the disclosure can comprise administering a diet comprising no more than 70% of a daily recommend daily cholesterol intake value. In some embodiments, a method of the disclosure comprise administering a diet comprising no more than 50% of a daily recommended cholesterol intake value. In some embodiments, a method of the disclosure can comprise administering a diet comprising no more than 30% of a daily recommended cholesterol intake value. In some embodiments, a method of the disclosure can comprise administering a diet comprising no more than 25% of a daily recommended cholesterol intake value.

In some embodiments, a method of the disclosure can comprise administering a diet that is restricted in total fat intake, e.g., a daily total fat intake. In some embodiments, a method of the disclosure can comprise administering a diet that is restricted in total fat intake, e.g., a daily total fat intake. In some embodiments, a method of the disclosure can comprise administering a diet that is restricted in a daily recommended dietary fat intake. In some embodiments, a diet comprises no more than 80%, no more than 70%, no more than 75%, no more than 60%, no more than 50%, no more than 40%, no more than 30%, no more than 25%, no more than 20%, no more than 10%, no more than 5%, no more than 4%, no more than 3%, no more than 2%, no more than 1%, or no more than 0.5% of a subject's average daily fat intake prior to start of the diet. In some embodiments, a diet comprises less than 90%, less than 80%, less than 70%, less than 75%, less than 60%, less than 50%, less than 40%, less than 30%, less than 25%, less than 20%, less than 10%, less than 5%, less than 4%, less than 3%, less than 2%, less than 1%, or less than 0.5% of a subject's average daily fat intake prior to start of the diet. In some embodiments, a method of the disclosure can comprise administering a diet that does not comprise fat. A subject's fat intake can be according to a dietary guideline published by a federal government agency, e.g., the United States Department of Agriculture and Health and Human Services or the National Health and Nutrition Examination Survey (NHANES). A subject's average daily fat intake can be according to the Dietary Guidelines for Americans published by the United States Departments of Agriculture and Health and Human Services. A subject's average daily recommended fat intake can be according to the Dietary Guidelines for Americans published by the United States Departments of Agriculture and Health and Human Services.

In some embodiments, a method of the disclosure can comprise administering a diet comprising no more than 100 g/day, no more than 90 g/day, no more than 80 g/day, no more than 70 g/day, no more than 60 g/day, no more than 50 g/day, no more than 40 g/day, no more than 30 g/day, no more than 20 g/day, no more than 10 g/day, no more than 5 g/day, or no more than 1 g/day of total fat, e.g., based on a 2000 kcal/day diet. In some embodiments, a method of the disclosure can comprise administering a diet comprising a restricted amount of fat, e.g., less than about 40% of total calories from fat, e.g., based on a 2000 kcal/day diet. In some embodiments, a method of the disclosure can comprise administering a diet comprising less than about 40%, less than about 35%, less than about 30%, less than about 25%, less than about 20%, less than about 15%, less than about 10%, or less than about 5% of total daily calories from fat, e.g., based on a 2000 kcal/day diet. In some embodiments, a method of the disclosure can comprise administering a diet comprising less than about 30% of total daily calories from fat, e.g., based on a 2000 kcal/day diet. In some embodiments, a method of the disclosure can comprise administering a diet comprising less than about 25% of total daily calories from fat, e.g., based on a 2000 kcal/day diet. In some embodiments, a method of the disclosure can comprise administering a diet comprising less than about 20% of total daily calories from fat, e.g., based on a 2000 kcal/day diet. In some embodiments, a method of the disclosure can comprise administering a diet comprising less than about 15% of total daily calories from fat, e.g., based on a 2000 kcal/day diet. In some embodiments, a method of the disclosure can comprise administering a diet comprising less than about 10% of total daily calories from fat, e.g., based on a 2000 kcal/day diet. In some embodiments, a method of the disclosure can comprise administering a diet comprising less than about 5% of total daily calories from fat, e.g., based on a 2000 kcal/day diet. The amount of fat in the diet can vary in a proportionate amount for subjects who consume less than 2000 kcal/day diet or more than 2000 kcal/day diet.

In some embodiments, a method of the disclosure can comprise administering a diet comprising less than about 80 g/day, less than about 75 g/day, less than about 70 g/day, less than about 65 g/day, less than about 60 g/day, less than about 55 g/day, less than about 50 g/day, less than about 45 g/day, less than about 40 g/day, less than about 35 g/day, less than about 30 g/day, less than about 25 g/day, less than about 20 g/day, less than about 15 g/day, less than about 10 g/day, or less than about 5 g/day of fat, e.g., based on a 2000 kcal/day diet. In some embodiments, a method of the disclosure can comprise administering diet comprising less than about 70 g/day of fat, e.g., based on a 2000 kcal/day diet. In some embodiments, a method of the disclosure can comprise administering a diet comprising less than about 60 g/day of fat, e.g., based on a 2000 kcal/day diet. In some embodiments, a method of the disclosure can comprise administering a diet comprising less than about 50 g/day of fat, e.g., based on a 2000 kcal/day diet. In some embodiments, a method of the disclosure can comprise administering a diet comprising less than about 40 g/day of fat, e.g., based on a 2000 kcal/day diet. In some embodiments, a method of the disclosure can comprise administering a diet comprising less than about 30 g/day of fat, e.g., based on a 2000 kcal/day diet. In some embodiments, a method of the disclosure can comprise administering a diet comprising less than about 20 g/day of fat, e.g., based on a 2000 kcal/day diet. The amount of fat in the diet can vary in a proportionate amount for subjects who consume less than 2000 kcal/day diet or more than 2000 kcal/day diet.

The amount of fat in the diet can be based on the weight of a subject, e.g., a weight in kilograms (kg). In some embodiments, a method of the disclosure can comprise administering a diet comprising less than about 1500 mg/kg/day, less than about 1400 mg/kg/day, less than about 1300 mg/kg/day, less than about 1200 mg/kg/day, less than about 1100 mg/kg/day, less than about 1000 mg/kg/day, less than about 900 mg/kg/day, less than about 800 mg/kg/day, less than about 700 mg/kg/day, less than about 600 mg/kg/day, less than about 500 mg/kg/day, less than about 400 mg/kg/day, less than about 300 mg/kg/day, less than about 200 mg/kg/day, or less than about 100 mg/kg/day of fat based on a subject's weight.

In some embodiments, a method of the disclosure can comprise administering a diet comprising less than 110%, less than 100%, less than 90%, less than 80%, less than 70%, less than 60%, less than 50%, less than 40%, less than 30%, less than 20%, less than 10%, less than 5%, less than 4%, less than 3%, less than 2%, or less than 1% of a recommended daily fat intake, e.g., based on a 2000 kcal/day diet. A daily recommended intake for fat can be based on the United States Food and Drug Administration Rules and Regulations-Revision of the Nutrition and Supplemental Facts Label. For example, a daily recommended intake is about 78 g/day of fat, based on the United States Food and Drug Administration Rules and Regulations-Revision of the Nutrition and Supplemental Facts Label dated May 27, 2016. In some embodiments, a method of the disclosure can comprise administering diet comprising less than 90% of a daily recommended fat intake, e.g., based on a 2000 kcal/day diet. In some embodiments, a method of the disclosure can comprise administering a diet comprising less than 80% of a daily recommended fat intake, e.g., based on a 2000 kcal/day diet. In some embodiments, a method of the disclosure can comprise administering a diet comprising less than 70% of a daily recommended fat intake, e.g., based on a 2000 kcal/day diet. In some embodiments, a method of the disclosure can comprise administering a diet comprising less than 60% of a daily recommended fat intake, e.g., based on a 2000 kcal/day diet. In some embodiments, a method of the disclosure can comprise administering a diet comprising less than 50% of a daily recommended fat intake, e.g., based on a 2000 kcal/day diet. In some embodiments, a method of the disclosure can comprise administering a diet comprising less than 40% of a daily recommended fat intake, e.g., based on a 2000 kcal/day diet. In some embodiments, a method of the disclosure can comprise administering a diet comprising less than 30% of a daily recommended fat intake, e.g., based on a 2000 kcal/day diet. In some embodiments, a method of the disclosure can comprise administering a diet comprising less than 20% of a daily recommended fat intake, e.g., based on a 2000 kcal/day diet. The amount of fat in the diet can vary in a proportionate amount for subjects who consume less than 2000 kcal/day diet or more than 2000 kcal/day diet.

In some embodiments, a method of the disclosure can comprise administering a diet that is restricted in saturated fat intake, e.g., a daily saturated fat intake. In some embodiments, a method of the disclosure can comprise administering a diet comprising no more than 100 g/day, no more than 90 g/day, no more than 80 g/day, no more than 70 g/day, no more than 60 g/day, no more than 50 g/day, no more than 40 g/day, no more than 30 g/day, no more than 20 g/day, no more than 10 g/day, no more than 5 g/day, or no more than 1 g/day of saturated fat, e.g., based on a 2000 kcal/day diet. In some embodiments, a method of the disclosure can comprise administering a diet comprising less than about 15% of total kcals/day in saturated fat. In some embodiments, a method of the disclosure can comprise administering a diet comprising less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, less than about 1% of total kcals/day in saturated fat, e.g. based on a 2000 kcal/day diet. In some embodiments, a method of the disclosure can comprise administering a diet comprising less than about 15% of total kcals/day in saturated fat, e.g. based on a 2000 kcal/day diet. In some embodiments, a method of the disclosure can comprise administering a diet comprising less than about 10% of total kcals/day in saturated fat, e.g. based on a 2000 kcal/day diet. In some embodiments, a method of the disclosure can comprise administering a diet comprising less than about 7% of total kcals/day in saturated fat, e.g., based on a 2000 kcal/day diet. In some embodiments, a method of the disclosure can comprise administering a diet comprising less than about 5% of total kcals/day in saturated fat, e.g. based on a 2000 kcal/day diet. In some embodiments, a method of the disclosure can comprise administering a diet comprising less than a daily recommend intake value of saturated fat. For example, a daily recommended saturated fat intake value is 20 g/day based on the United States Food and Drug Administration Rules and Regulations-Revision of the Nutrition and Supplemental Facts Label dated May 27, 2016. In some embodiments, a method of the disclosure can comprise administering a diet comprising less than 20 g/day, less than 15 g/day, less than 10 g/day, less than 5 g/day, less than 4 g/day, less than 3 g/day, less than 2 g/day, less than 1 g/day, or less than 500 mg/day in saturated fat, e.g., based on a 2000 kcal/day diet. The amount of fat in the diet can vary in a proportionate amount for subjects who consume less than 2000 kcal/day diet or more than 2000 kcal/day diet. A diet low in saturated fats can exclude foods of animal origin (e.g., liver and other organ meats) and oils (e.g., coconut oil, palm oil, palm kernel oil).

In some embodiments, a method of the disclosure can comprise administering a diet that is restricted in trans fat intake, e.g., a daily trans fat intake. In some embodiments, a method of the disclosure can comprise administering a diet comprising no more than 100 g/day, no more than 90 g/day, no more than 80 g/day, no more than 70 g/day, no more than 60 g/day, no more than 50 g/day, no more than 40 g/day, no more than 30 g/day, no more than 20 g/day, no more than 10 g/day, no more than 5 g/day, or no more than 1 g/day of trans fat, e.g., based on a 2000 kcal/day diet. In some embodiments, a method of the disclosure can comprise administering a diet comprising less than about 5% of total kcals/day in trans fat, e.g., based on a 2000 kcal/day diet. In some embodiments, a method of the disclosure can comprise administering a diet comprising less than about 5%, less than about 4%, less than about 3%, less than about 2%, less than about 1%, or less than about 0.5% of total kcals/day in trans fat, e.g., based on a 2000 kcal/day diet. In some embodiments, a method of the disclosure can comprise administering a diet comprising less than 20 g/day, less than 15 g/day, less than 10 g/day, less than 5 g/day, less than 4 g/day, less than 3 g/day, less than 2 g/day, less than 1 g/day, or less than 500 mg/day in trans fat, e.g., based on 2000 kcal/day diet. The amount of fat in the diet can vary in a proportionate amount for subjects who consume less than 2000 kcal/day diet or more than 2000 kcal/day diet. Diets low in trans fat can exclude foods such as margarine, baked products (e.g., crackers, cookies, doughnuts, breads), and fried foods.

In some embodiments, a method of the disclosure can comprise administering a diet high in stanols or sterols. A diet high in stanols and sterols can include fruits, vegetables, and supplements, such as benecol (3-4 grams/day).

In some embodiments, a method of the disclosure can comprise administering a diet comprising from about 10 g to about 40 g of fiber/day. In some embodiments, a method of the disclosure can comprise administering a diet comprising from about 10 g to about 20 g, from about 20 g to about 30 g, or from about 30 g to about 40 g of fiber/day. In some embodiments, a method of the disclosure can comprise administering a diet comprising from about 10 g to about 20 g of fiber/day. In some embodiments, a method of the disclosure can comprise administering a diet comprising from about 20 g to about 30 g of fiber/day. In some embodiments, a method of the disclosure can comprise administering a diet comprising from about 30 g to about 40 g of fiber/day. In some embodiments, a method of the disclosure can comprise administering a diet high in high-fiber carbohydrates comprising, for example, oats, bran, barley, nuts and seeds, beans, lentils, peas, and vegetables.

In some embodiments, a method of the disclosure can comprise administering a diet that is restricted in polyunsaturated fat intake. In some embodiments, a method of the disclosure can comprise administering a diet comprising less than about 15% in total kcals/day in polyunsaturated fats, e.g., based on a 2000 kcal/day diet. In some embodiments, a method of the disclosure can comprise administering a diet comprising less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, less than about 1% in total kcals/day in polyunsaturated fats, e.g., based on a 2000 kcal/day diet. In some embodiments, a method of the disclosure can comprise administering a diet comprising less than about 15% in total kcals/day in polyunsaturated fats, e.g., based on a 2000 kcal/day diet. In some embodiments, a method of the disclosure can comprise administering a diet comprising less than about 10% in total kcals/day in polyunsaturated fats, e.g., based on a 2000 kcal/day diet. In some embodiments, a method of the disclosure can comprise administering a diet comprising less than about 8% in total kcals/day in polyunsaturated fats, e.g., based on a 2000 kcal/day diet. The amount of fat in the diet can vary in a proportionate amount for subjects who consume less than 2000 kcal/day diet or more than 2000 kcal/day diet.

In some embodiments, a method of the disclosure can comprise administering a diet that is restricted in monounsaturated fat intake. In some embodiments, a method of the disclosure can comprise administering a diet comprising less than about 25% total kcals/day in monounsaturated fats, e.g., based on a 2000 kcal/day diet. In some embodiments, a method of the disclosure can comprise administering a diet comprising less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, less than about 1% in total kcals/day in monounsaturated fats, e.g., based on a 2000 kcal/day diet. In some embodiments, a method of the disclosure can comprise administering a diet comprising less than about 25% in total kcals/day in monounsaturated fats, e.g., based on a 2000 kcal/day diet. In some embodiments, a method of the disclosure can comprise administering a diet comprising less than about 20% in total kcals/day in monounsaturated fats, e.g., based on a 2000 kcal/day diet. In some embodiments, a method of the disclosure can comprise administering a diet comprising less than about 15% in total kcals/day in monounsaturated fats, e.g., based on a 2000 kcal/day diet. The amount of fat in the diet can vary in a proportionate amount for subjects who consume less than 2000 kcal/day diet or more than 2000 kcal/day diet.

In some embodiments, a method of the disclosure can comprise administering a diet formulated to reduce a level of fat in a subject. In some embodiments, a method of the disclosure can comprise administering a diet formulated to reduce a level of fat in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can comprise administering a diet formulated to reduce a level of fat in a subject by about 10%. In some embodiments, a method of the disclosure can comprise administering a diet formulated to reduce a level of fat in a subject by about 20%. In some embodiments, a method of the disclosure can comprise administering a diet formulated to reduce a level of fat in a subject by about 30%. In some embodiments, a method of the disclosure can comprise administering a diet formulated to reduce a level of fat in a subject by about 50%. In some embodiments a method of the disclosure can comprise administering a diet formulated to reduce a level of fat in a subject by about 75%.

In some embodiments, a method of the disclosure can comprise administering a diet formulated to reduce a blood level of fat in a subject. In some embodiments, a method of the disclosure can comprise administering a diet formulated to reduce a blood level of fat in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can comprise administering a diet formulated to reduce a blood level of fat in a subject by about 10%. In some embodiments, a method of the disclosure can comprise administering a diet formulated to reduce a blood level of fat in a subject by about 20%. In some embodiments, a method of the disclosure can comprise administering a diet formulated to reduce a blood level of fat in a subject by about 30%. In some embodiments, a method of the disclosure can comprise administering a diet formulated to reduce a blood level of fat in a subject by about 50%. In some embodiments a method of the disclosure can comprise administering a diet formulated to reduce a blood level of fat in a subject by about 75%.

In some embodiments, a method of the disclosure can comprise administering a diet that reduces a level of fat in a subject. In some embodiments, a method of the disclosure can comprise administering a diet that reduces a level of fat in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can comprise administering a diet that reduces a level of fat in a subject by about 10%. In some embodiments, a method of the disclosure can comprise administering a diet that reduces a level of fat in a subject by about 20%. In some embodiments, a method of the disclosure can comprise administering a diet that reduces a level of fat in a subject by about 30%. In some embodiments, a method of the disclosure can comprise administering a diet that reduces a level of fat in a subject by about 50%. In some embodiments a method of the disclosure can comprise administering a diet that reduces a level of fat in a subject by about 75%.

In some embodiments, a method of the disclosure can comprise administering a diet that reduces a blood level of fat in a subject. In some embodiments, a method of the disclosure can comprise administering a diet that reduces a blood level of fat in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can comprise administering a diet that reduces a blood level of fat in a subject by about 10%. In some embodiments, a method of the disclosure can comprise administering a diet that reduces a blood level of fat in a subject by about 20%. In some embodiments, a method of the disclosure can comprise administering a diet that reduces a blood level of fat in a subject by about 30%. In some embodiments, a method of the disclosure can comprise administering a diet that reduces a blood level of fat in a subject by about 50%. In some embodiments a method of the disclosure can comprise administering a diet formulated to reduce a blood level of fat in a subject by about 75%.

In some embodiments, a method of the disclosure can comprise administering a diet formulated to reduce a level of cholesterol in a subject. In some embodiments, a method of the disclosure can comprise administering a diet formulated to reduce a level of cholesterol in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can comprise administering a diet formulated to reduce a level of cholesterol in a subject by about 10%. In some embodiments, a method of the disclosure can comprise administering a diet formulated to reduce a level of cholesterol in a subject by about 20%. In some embodiments, a method of the disclosure can comprise administering a diet formulated to reduce a level of cholesterol in a subject by about 30%. In some embodiments, a method of the disclosure can comprise administering a diet formulated to reduce a level of cholesterol in a subject by about 50%. In some embodiments a method of the disclosure can comprise administering a diet formulated to reduce a level of cholesterol in a subject by about 75%.

In some embodiments, a method of the disclosure can comprise administering a diet formulated to reduce a blood level of cholesterol in a subject. In some embodiments, a method of the disclosure can comprise administering a diet formulated to reduce a blood level of cholesterol in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can comprise administering a diet formulated to reduce a blood level of cholesterol in a subject by about 10%. In some embodiments, a method of the disclosure can comprise administering a diet formulated to reduce a blood level of cholesterol in a subject by about 20%. In some embodiments, a method of the disclosure can comprise administering a diet formulated to reduce a blood level of cholesterol in a subject by about 30%. In some embodiments, a method of the disclosure can comprise administering a diet formulated to reduce a blood level of cholesterol in a subject by about 50%. In some embodiments a method of the disclosure can comprise administering a diet formulated to reduce a blood level of cholesterol in a subject by about 75%.

In some embodiments, a method of the disclosure can comprise administering a diet that reduces a level of cholesterol in a subject. In some embodiments, a method of the disclosure can comprise administering a diet that reduces a level of cholesterol in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can comprise administering a diet that reduces a level of cholesterol in a subject by about 10%. In some embodiments, a method of the disclosure can comprise administering a diet that reduces a level of cholesterol in a subject by about 20%. In some embodiments, a method of the disclosure can comprise administering a diet that reduces a level of cholesterol in a subject by about 30%. In some embodiments, a method of the disclosure can comprise administering a diet that reduces a level of cholesterol in a subject by about 50%. In some embodiments a method of the disclosure can comprise administering a diet that reduces a level of cholesterol in a subject by about 75%.

In some embodiments, a method of the disclosure can comprise administering a diet that reduces a blood level of cholesterol in a subject. In some embodiments, a method of the disclosure can comprise administering a diet that reduces a blood level of cholesterol in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can comprise administering a diet that reduces a blood level of cholesterol in a subject by about 10%. In some embodiments, a method of the disclosure can comprise administering a diet that reduces a blood level of cholesterol in a subject by about 20%. In some embodiments, a method of the disclosure c can comprise administering a diet that reduces a blood level of cholesterol in a subject by about 30%. In some embodiments, a method of the disclosure can comprise administering a diet that reduces a blood level of cholesterol in a subject by about 50%. In some embodiments a method of the disclosure can comprise administering a diet that reduces a blood level of cholesterol in a subject by about 75%.

A nutrient amount disclosed herein can be based on a recommended amount of the nutrient for an adult subject consuming on average a 2000 kcal/day diet. Any nutrient amount disclosed herein can be scaled appropriately based on the dietary intake of the subject. For example, a subject consuming on average a 2500 kcal/day diet can have a 25% increase in the amount of a nutrient amount or percentage disclosed herein. In another example, a subject consuming on average a 1500 kcal/day diet can have a 25% decrease in the amount of a nutrient amount or percentage disclosed herein.

In some embodiments, a method of the disclosure comprises a subject participating in physical activity. In some embodiments, a method of the disclosure comprises a subject including from about 30 min to about 200 min per week of moderate exercise. In some embodiments, a method of the disclosure comprises a subject including from about 30 min to about 60 min, from about 60 min to about 90 min, from about 90 min to about 120 min, from about 120 min to about 150 min, or from about 150 min to about 200 min per week of moderate exercise. In some embodiments, a method of the disclosure comprises a subject including from about 60 min to about 90 min per week of moderate exercise. In some embodiments, a method of the disclosure comprises a subject including from about 90 min to about 120 min per week of moderate exercise. In some embodiments, a method of the disclosure comprises a subject including from about 120 min to about 150 min per week of moderate exercise.

### Methods of Use

A method of the disclosure can be used to treat a condition. In some embodiments, the condition is a cholesterol-dependent condition. In some embodiments, the condition is a cancer.

In some embodiments, the condition is a cholesterol-dependent cancer. In some embodiments, the condition is a cancer that consumes exogenous cholesterol.

A method of the disclosure can slow the proliferation of cancer cell lines, or kill cancer cells. Non-limiting examples of cancer that can be treated by methods disclosed herein include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, blood cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, clear renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

A method of the disclosure can decrease cancer cell proliferation in a subject. In some embodiments, a therapeutic agent or composition of the disclosure can decrease cancer cell proliferation in a subject by from about 5% to about 10%, from about 10% to about 15%, from about 15% to about 20%, from about 20% to about 25%, from about 25% to about 30%, from about 35% to about 40%, from about 40% to about 45%, from about 45% to about 50%, from about 50% to about 75%, from about 75% to about 100%, from about 100% to about 125%, from about 125% to about 150%, from about 150% to about 175%, or from about 175% to about 200% compared to a subject that is not administered the therapeutic agent or composition of the disclosure. In some embodiments, a therapeutic agent or composition of the disclosure can decrease cancer cell proliferation in a subject by from about 20% to about 25% compared to a subject that is not administered the therapeutic agent or composition of the disclosure. In some embodiments, a therapeutic agent or composition of the disclosure can decrease cancer cell proliferation in a subject by from about 50% to about 75% compared to a subject that is not administered the therapeutic agent or composition of the disclosure.

In some embodiments, a method of the disclosure can decrease cancer cell proliferation in a subject by about 5%, about 10%, about 15%, about 20%, about 25%, about 30%, about 35%, about 40%, about 45%, about 50%, about 75%, about 100%, about 125%, about 150%, about 175%, or about 200% compared to a subject that is not administered the method. In some embodiments, a method of the disclosure can decrease cancer cell proliferation in a subject by about 20% compared to a subject that is not administered the method. In some embodiments, a method of the disclosure can decrease cancer cell proliferation in a subject by about 30% compared to a subject that is not administered the method. In some embodiments, a method of the disclosure can decrease cancer cell proliferation in a subject by about 50% compared to a subject that is not administered the method. In some embodiments, a method of the disclosure can decrease cancer cell proliferation in a subject by about 70% compared to a subject that is not administered the method.

In some embodiments, a method of the disclosure can decrease a tumor size in a subject by from about 5% to about 10%, from about 10% to about 15%, from about 15% to about 20%, from about 20% to about 25%, from about 25% to about 30%, from about 35% to about 40%, from about 40% to about 45%, from about 45% to about 50%, from about 50% to about 75%, from about 75% to about 100%, from about 100% to about 125%, from about 125% to about 150%, from about 150% to about 175%, or from about 175% to about 200%. In some embodiments, a method of the disclosure can decrease a tumor size in a subject by from about 20% to about 25%. In some embodiments, a method of the disclosure can decrease a tumor size in a subject by from about 45% to about 50%.

In some embodiments, a method of the disclosure can decrease a tumor size in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can decrease a tumor size in a subject by about 20%. In some embodiments, a method of the disclosure can decrease a tumor size in a subject by about 30%. In some embodiments, a method of the disclosure can decrease a tumor size in a subject by about 50%.

A method of the disclosure can reduce low-density lipoprotein-cholesterol (LDL-C) levels in a subject's blood. In some embodiments, a method of the disclosure can reduce LDL-C levels in a subject's blood by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can reduce LDL-C levels in a subject's blood by about 10%. In some embodiments, a method of the disclosure can reduce LDL-C levels in a subject's blood by about 20%. In some embodiments, a method of the disclosure can reduce LDL-C levels in a subject's blood by about 30%. In some embodiments, a method of the disclosure can reduce LDL-C levels in a subject's blood by about 50%. In some embodiments, a method of the disclosure can reduce LDL-C levels in a subject's blood by about 75%.

A method of the disclosure can reduce triglyceride levels in a subject's blood. In some embodiments, a method of the disclosure can reduce triglyceride levels in a subject's blood by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can reduce triglyceride levels in a subject's blood by about 10%. In some embodiments, a method of the disclosure can reduce triglyceride levels in a subject's blood by about 20%. In some embodiments, a method of the disclosure can reduce triglyceride levels in a subject's blood by about 30%. In some embodiments, a method of the disclosure can reduce triglyceride levels in a subject's blood by about 50%. In some embodiments, a method of the disclosure can reduce triglyceride levels in a subject's blood by about 75%.

A method of the disclosure can reduce a level of fat in a subject. In some embodiments, a method of the disclosure can reduce a level of fat in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can reduce a level of fat in a subject by about 10%. In some embodiments, a method of the disclosure can reduce a level of fat in a subject by about 20%. In some embodiments, a method of the disclosure can reduce a level of fat in a subject by about 30%. In some embodiments, a method of the disclosure can reduce a level of fat in a subject by about 50%. In some embodiments, a method of the disclosure can reduce a level of fat in a subject by about 75%.

A method of the disclosure can reduce a level of cholesterol in a subject. In some embodiments, a method of the disclosure can reduce a level of cholesterol in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can reduce a level of cholesterol in a subject by about 10%. In some embodiments, a method of the disclosure can reduce a level of cholesterol in a subject by about 20%. In some embodiments, a method of the disclosure can reduce a level of cholesterol in a subject by about 30%. In some embodiments, a method of the disclosure can reduce a level of cholesterol in a subject by about 50%. In some embodiments, a method of the disclosure can reduce a level of cholesterol in a subject by about 75%.

A method of the disclosure can reduce a blood level of fat in a subject. In some embodiments, a method of the disclosure can reduce a blood level of fat in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can reduce a blood level of fat in a subject by about 10%. In some embodiments, a method of the disclosure can reduce a blood level of fat in a subject by about 20%. In some embodiments, a method of the disclosure can reduce a blood level of fat in a subject by about 30%. In some embodiments, a method of the disclosure can reduce a blood level of fat in a subject by about 50%. In some embodiments, a method of the disclosure can reduce a blood level of fat in a subject by about 75%.

A method of the disclosure can reduce a blood level of cholesterol in a subject. In some embodiments, a method of the disclosure can reduce a blood level of cholesterol in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can reduce a blood level of cholesterol in a subject by about 10%. In some embodiments, a method of the disclosure can reduce a blood level of cholesterol in a subject by about 20%. In some embodiments, a method of the disclosure can reduce a blood level of cholesterol in a subject by about 30%. In some embodiments, a method of the disclosure can reduce a blood level of cholesterol in a subject by about 50%. In some embodiments, a method of the disclosure can reduce a blood level of cholesterol in a subject by about 75%.

A method of the disclosure can increase high-density lipoprotein-cholesterol (HDL-C) levels in a subject's blood. In some embodiments, a method of the disclosure can increase HDL levels in a subject's blood by about 5%, about 10%, about 15%, about 20%, about 25%, about 30%, about 35%, about 40%, about 45%, about 50%, about 75%, about 100%, about 125%, about 150%, about 175%, or about 200%. In some embodiments, a method of the disclosure can increase HDL levels in a subject's blood by about 5%. In some embodiments, a method of the disclosure can increase HDL levels in a subject's blood by about 10%. In some embodiments, a method of the disclosure can increase HDL levels in a subject's blood by about 20%. In some embodiments, a method of the disclosure can increase HDL levels in a subject's blood by about 30%. In some embodiments, a method of the disclosure can increase HDL levels in a subject's blood by about 50%.

A method of the disclosure can increase overall survival of a subject. In some embodiments, a method of the disclosure can increase overall survival of a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, a method of the disclosure can increase overall survival of a subject by at least about 10%. In some embodiments, a method of the disclosure can increase overall survival of a subject by at least about 20%. In some embodiments, a method of the disclosure can increase overall survival of a subject by at least about 30%.

A method of the disclosure can increase progression free survival of a subject. In some embodiments, a method of the disclosure can increase progression free survival of a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, a method of the disclosure can increase progression free survival of a subject by at least about 10%. In some embodiments, a method of the disclosure can increase progression free survival of a subject by at least about 20%. In some embodiments, a method of the disclosure can increase progression free survival of a subject by at least about 30%.

A method of the disclosure can increase percentage of cancer cell death. In some embodiments, a method of the disclosure can increase percentage of cancer cell death by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, a method of the disclosure can increase percentage of cancer cell death by at least about 10%. In some embodiments, a method of the disclosure can increase percentage of cancer cell death by at least about 20%. In some embodiments, a method of the disclosure can increase percentage of cancer cell death by at least about 30%.

A method of the disclosure can increase sensitivity to a cancer therapy in a subject. In some embodiments, a method of the disclosure can increase sensitivity to a cancer therapy in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, a method of the disclosure can increase sensitivity to a cancer therapy in a subject by at least about 10%. In some embodiments, a method of the disclosure can increase sensitivity to a cancer therapy in a subject by at least about 20%. In some embodiments, a method of the disclosure can increase sensitivity to a cancer therapy in a subject by at least about 30%.

A method of the disclosure can increase a treatment response rate of a therapeutic agent. In some embodiments, a method of the disclosure can increase a treatment response rate of a therapeutic agent by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, a method of the disclosure can increase a treatment response rate of a therapeutic agent by at least about 10%. In some embodiments, a method of the disclosure can increase a treatment response rate of a therapeutic agent by at least about 20%. In some embodiments, a method of the disclosure can increase a treatment response rate of a therapeutic agent by at least about 30%.

A method of the disclosure can increase the efficacy of an additional therapeutic agent in a subject compared to a subject treated only with the method of the disclosure. In some embodiments, administering a method of the disclosure with a therapeutic agent can increase the efficacy of the therapeutic agent in a subject by at least about at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% compared to a subject treated only with the method of the disclosure. In some embodiments, administering a method of the disclosure with a therapeutic agent can increase the efficacy of the therapeutic agent in a subject by at least about at least about 10% compared to a subject treated only with the method of the disclosure. In some embodiments, administering a method of the disclosure with a therapeutic agent can increase the efficacy of the therapeutic agent in a subject by at least about at least about 20% compared to a subject treated only with the method of the disclosure. In some embodiments, administering a method of the disclosure can increase the efficacy of the therapeutic agent in a subject by at least about at least about 30% compared to a subject treated only with the method of the disclosure.

Administering a method with a therapeutic agent to a subject can have a decreased dose of the therapeutic agent compared to a subject treated with the method alone to achieve the same outcome. In some embodiments, administering a method of the disclosure with a therapeutic agent to a subject can decrease the required dose of the therapeutic agent by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% compared to a subject treated with the therapeutic agent alone to achieve the same outcome. In some embodiments, administering a method of the disclosure with a therapeutic agent to a subject can decrease the required dose of the therapeutic agent by at least about 10% compared to a subject treated with the therapeutic agent alone to achieve the same outcome. In some embodiments, administering a method of the disclosure with a therapeutic agent to a subject can decrease the required dose of the therapeutic agent by at least about 20% compared to a subject treated with the therapeutic agent alone to achieve the same outcome. In some embodiments, administering a method of the disclosure with a therapeutic agent to a subject can decrease the required dose of the therapeutic agent by at least about 30% compared to a subject treated with the therapeutic agent alone to achieve the same outcome.

Administering a method of the disclosure with a therapeutic agent to a subject can decrease adverse events associated with an additional therapy compared to a subject treated with the additional therapy alone. In some embodiments, administering a method of the disclosure with a therapeutic agent to a subject can decrease adverse events associated with an additional therapy by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% compared to a subject treated with the additional therapy alone. In some embodiments, administering a method of the disclosure with a therapeutic agent to a subject can decrease adverse events associated with an additional therapy by at least about 10% compared to a subject treated with the additional therapy alone. In some embodiments, administering a method of the disclosure with a therapeutic agent to a subject can decrease adverse events associated with an additional therapy by at least about 20% compared to a subject treated with the additional therapy alone. In some embodiments, administering a method of the disclosure with a therapeutic agent to a subject can decrease adverse events associated with an additional therapy by at least about 30% compared to a subject treated with the additional therapy alone.

In some embodiments, disclosed herein is a method of identifying gene-expression signature to predict cells, tumors, or subjects that will be most responsive to a treatment method disclosed herein. In some embodiments, disclosed herein is a method of identifying gene-mutation signature to predict cells, tumors, or subjects that will be most responsive to a treatment method disclosed herein. In some embodiments, a method disclosed herein can comprise assessing the expression of genes in the mevalonate pathway. In some embodiments, a method disclosed herein can comprise assessing genes that regulate the mevalonate pathway. In some embodiments, a method disclosed herein can comprise assessing genes that modulate cholesterol uptake or cholesterol transport. In some embodiments, a method disclosed herein can comprise assessing the assessing genes related to Apolipoproteins.

### EXAMPLES

### Example 1: Effect of Statins and Cholesterol-Restricted Diet on Cancer Cell Growth

An experiment was designed to assess the growth of cancer cells in the presence of varying doses of statins. The added role of diet was studied by growing cells with statins in the presence or absence of cholesterol.

In human cell culture, cholesterol is supplied by the supplementation of fetal bovine serum (FBS). The amount of cholesterol supplied by the serum is undefined and subject to batch variation. FBS was replaced by supplementing the growth media. RPMI or DMEM with a defined amount of cholesterol, fatty acids, vitamins, minerals and growth factors were used for cell culture, which allowed for completed defined media. Cancer cell growth was measured in complete media containing 56 μM of cholesterol or cholesterol-free media subjected to varying doses of statins.

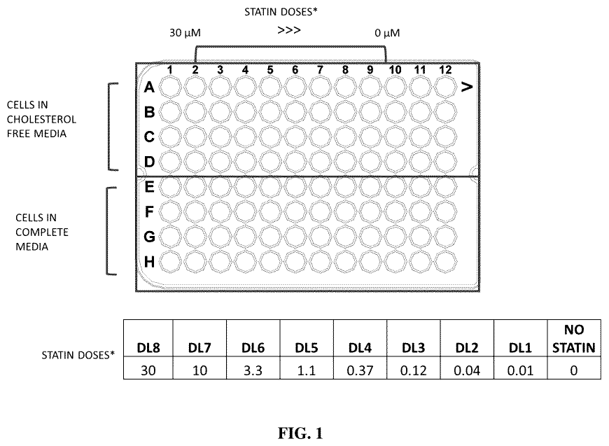

FIG. 1 shows the experimental layout of the statin and complete/cholesterol-free media study. The following cell lines were used for the study: 1) Lung Cancer: H292, A549, PC9, H358, HC827, H1963; 2) Pancreatic Cancer: PSN1, PANC1, BXPC3, CFPAC1, PATU8902, MIAPACA2; 3) Head and Neck Cancer: SCC9, CAL33; 4) Colorectal Cancer: DLD-1, LS180, SW480, HCT116; 5) Breast Cancer: T47D, MCF7; 6) Blood Cancer: Jurkat; 7) Kidney Cancer: CAKI2. Simvastatin and fluvastatin sodium were used as statins.

On Day 1, cells were seeded at a density of around 7500 cells/well in a Corning 96-well flat bottom culture plate in complete media containing 10% FBS. On Day 2, the plates were washed with PBS and test media, and varying doses of statins were added to the wells (FIG. 1). Cell growth was measured on Day 5. For absorbance growth measurements, spent media was discarded, and wells were stained with a crystal violet staining solution, washed and dried. 100 μL of 10% acetic acid was added to each well to release the crystal violet taken up by the cells. Plates were read using a Biotek Powerwave plate reader to determine the absorbance of the crystal violet released as an indicator of cell growth at a wavelength of 595 nm. For fluorescence growth measurements, 100 μL of a resazurin solution was added to each well and the plate incubated at 37° C. for four hours. Plates were read using a Biotek Powerwave plate reader to determine the fluorescence of reosurfin as an indicator of cell growth at a wavelength of 590 nm.

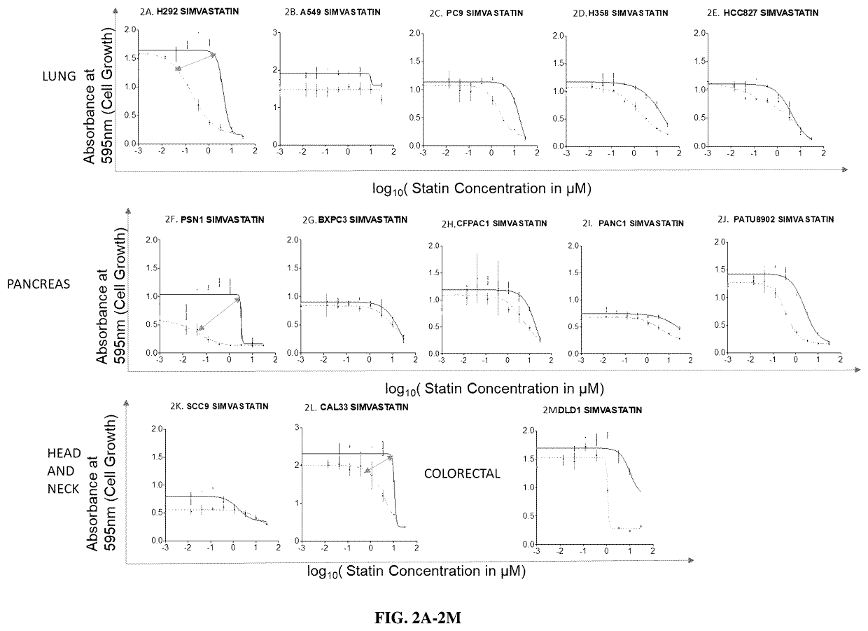

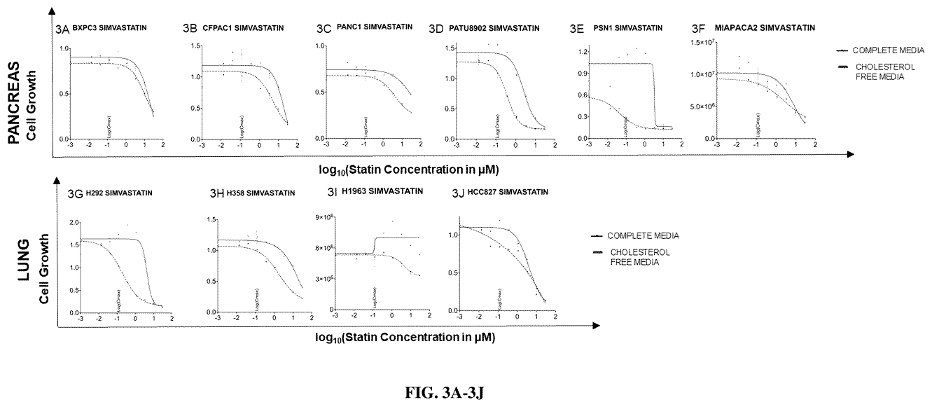

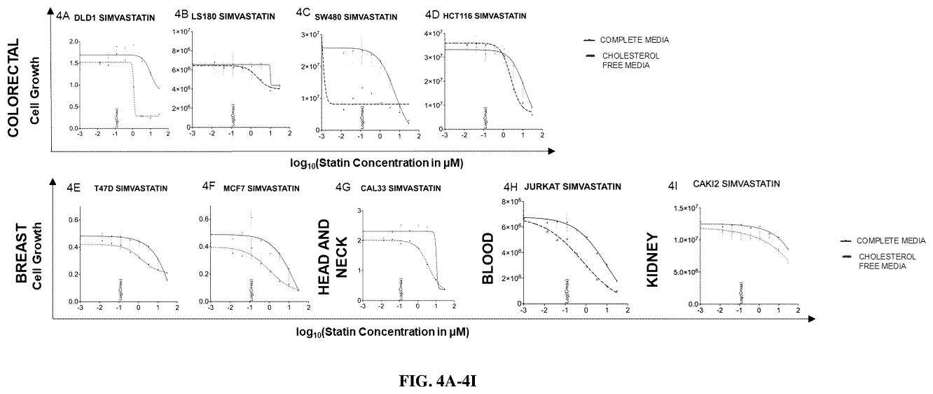

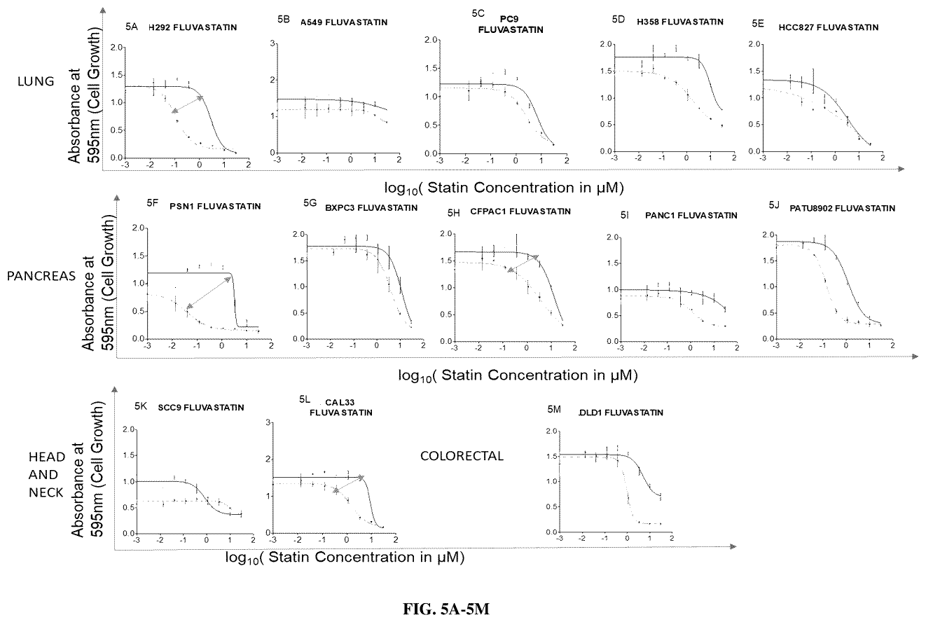

Growth curves showing absorbance of crystal violet at 595 nm or fluorescence of reosurfin at 590 nM were plotted from test wells against the logarithmic statin concentration. FIG. 2A-2M, FIG. 3A-3J, and FIG. 4A-AI show growth of cancer cell lines in complete and cholesterol-free media subjected to increasing doses of simvastatin. FIG. 5A-5M show growth of cancer cell lines in complete and cholesterol-free media subjected to increasing doses of fluvastatin sodium. Increasing statin concentration led to decreased cell growth. The bold lines show cell growth in complete media, and the dashed lines show cell growth in cholesterol-free media.

Cells grown in cholesterol-free media exhibited less cell growth than cells grown in complete media. The difference in cell growth was more pronounced for cell lines such as pancreatic PSN1 and PATU8902; lung H358, HCC287, and H292; colorectal DLD1 and SW480; breast T47D and MCF7; head and neck cancers SCC9 and CAL33, indicating an increased dependence on dietary cholesterol for cell proliferation in the cell lines. A significant left-shift was observed when growth curves of cells grown with statins were compared (shown as arrows). The data demonstrate that cells grown in cholesterol free media required a lower dose of statin to kill cells compared to cells grown in complete media. The data indicate that a lack of dietary cholesterol may require reduced statin doses to induce cancer cell death. The combination effect was highly pronounced for the lung cancer cell line H292, and pancreatic cell lines PSN1 and PATU8902. Responses of the cell lines to simvastatin and fluvastatin were similar. The maximum achievable serum concentration of minimum dose of either statin (20 mg) was 0.39 μM. Cell death in all the cell lines were achieved at concentrations lower than the Cₘₐₓ value. Cell death was observed at the highest concentration of statins (30 μM) for lung cell line A549 and pancreatic cell line PANC1. Overall, the data demonstrate that modulating dietary cholesterol can alter cholesterol biosynthesis, and cholesterol modulation can be used with administration of statins to control cancer cell growth.

### Example 2: FDA Drug Screening Method

NCI-H292 lung cancer cells derived from a cervical node metastasis of a pulmonary mucoepidermoid carcinoma of a 32 year old African-American female were used for the study. The FDA drug library was purchased from Medchem Express. The cells were cultured in RPMI with 10% FBS to 90-100% confluency. One Day 0, spent media was removed from culture, and cells were dissociated using TrypLE. Viable cells were counted with a haemocytometer using Trypan blue and resuspended in fresh media at a concentration of 75000 cells/mL. Using an Integra Mini 96 channel electronic pipette, 100 μL of the cell suspension was plated into each well of a 96-well plate. The plates were incubated overnight at 37° C. at 5% CO₂ in a humidified cell culture incubator. On Day 1, spent media was removed and each plate was washed with PBS. Half the plate (48 wells) had 115 μL of Complete media added to each of the wells, while the wells in the other half had 115 μL of cholesterol-free Media without statin (in screening for drug activity modulated by dietary cholesterol only) or with 0.04 μL of simvastatin (in screening for drug activity modulated by decreasing both cholesterol biosynthesis and dietary cholesterol). The concentration of simvastatin used was the minimum dose at which killing was observed in cholesterol free media. The FDA library was so formatted to include each drug twice so each drug could be tested in both media conditions on the same plate, along with negative controls (vehicle) and positive controls (disulfiram+pemetrexed+paclitaxel). 10 μL of each drug solution was added to both complete and experimental media accounting for a final 2 μM of drug concentration in an experimental volume of 125 μL per well. The plates were placed back at 37° C. at 5% CO₂ in a humidified cell culture incubator for 4 additional days. On Day 5, spent media was removed and 100 μL of staining solution containing 0.015% crystal violet, 1% formaldehyde and 1% methanol in PBS. 15 minutes later, the plates were washed with tap water and dried to remove any liquid residue. The wells were then treated with 100 μL of 10% acetic acid, and absorbance was read at 595 nm using a Biotek Powerwave Plate Reader. Absorbance at 595 nm was used as a proxy for cell number.

TABLE 1 shows the list of drugs that were tested for anti-cancer activity when paired with cholesterol-free media. TABLE 2 shows the list of drugs tested for anti-cancer activity when paired with cholesterol-free media and a statin (simvastatin). The drugs are listed from strongest to lowest anti-cancer activity. Bolded drugs indicate drugs that worked better in cholesterol-free or cholesterol-free+statin conditions. Normal text indicates drug that showed no difference between cholesterol-free or cholesterol-free+statin conditions. Italicized drugs indicate drugs that worked better in complete media. Drugs with “difference” values greater than 0 exhibited enhanced anti-cancer activity; drugs with “difference” values of about 0 were considered neutral; and drugs with “difference” values below 0 were considered antagonists.

TABLE 1

| Drug Name | Complete | Cholesterol-free | Difference |
|---|---|---|---|
| Baclofen | 1.886253843 | 0.554138535 | 1.33 |
| Gestrinone | 2.026236763 | 0.889803162 | 1.14 |
| Sumatriptan (succinate) | 2.035132819 | 0.955756748 | 1.08 |
| Quinidine | 1.951156812 | 1.01725316 | 0.93 |
| Bedaquiline | 1.472422062 | 0.60867679 | 0.86 |
| Topiroxostat | 1.602877698 | 0.763991323 | 0.84 |
| Nefazodone (hydrochloride) | 1.655309033 | 0.825627026 | 0.83 |
| Cephradine | 1.755784062 | 0.929791595 | 0.83 |
| Clindamycin palmitate (hydrochloride) | 1.4781491 | 0.677656303 | 0.80 |
| D-Mannitol | 1.662083136 | 0.862052275 | 0.80 |
| Rimantadine (hydrochloride) | 1.648969889 | 0.85087869 | 0.80 |
| Abiraterone acetate | 1.306769494 | 0.523231978 | 0.78 |
| Meprednisone | 1.522187005 | 0.764886538 | 0.76 |
| Diosmin | 1.465134707 | 0.717795598 | 0.75 |
| Isocarboxazid | 1.428297362 | 0.685032538 | 0.74 |
| Coumarin | 1.716366752 | 0.974205671 | 0.74 |
| Nateglinide | 1.887150308 | 1.149241691 | 0.74 |
| Fluralaner | 1.374580336 | 0.643383948 | 0.73 |
| Ticarcillin (disodium) | 1.430269414 | 0.700051186 | 0.73 |
| Apremilast | 1.856171961 | 1.126653759 | 0.73 |
| Ethambutol (dihydrochloride) | 1.205755396 | 0.482863341 | 0.72 |
| Buclizine (dihydrochloride) | 1.362758015 | 0.656164041 | 0.71 |
| Oxcarbazepine | 1.538131962 | 0.834130509 | 0.70 |
| Indacaterol | 1.37587092 | 0.677560708 | 0.70 |
| Triamterene | 1.303597122 | 0.60867679 | 0.69 |
| Isoniazid | 1.357314149 | 0.663340564 | 0.69 |
| Proglumide | 1.407194245 | 0.715401302 | 0.69 |
| Amezinium (methylsulfate) | 1.932669512 | 1.241529526 | 0.69 |
| Thiabendazole | 1.483799589 | 0.802032914 | 0.68 |
| Megestrol | 1.622107969 | 0.940724291 | 0.68 |
| Cephalothin (sodium) | 1.227722772 | 0.560708417 | 0.67 |
| Levamisole (hydrochloride) | 1.664952871 | 1.000854117 | 0.66 |
| DL-alpha-Tocopherol | 1.458795563 | 0.794915543 | 0.66 |
| Butylphthalide | 1.343106181 | 0.696638799 | 0.65 |
| Amfenac (Sodium Hydrate) | 1.79896259 | 1.154600571 | 0.64 |
| Levobunolol (hydrochloride) | 1.133676093 | 0.490433891 | 0.64 |
| Latanoprostene bunod | 1.463549921 | 0.822897117 | 0.64 |
| Fesoterodine | 1.534865293 | 0.897287152 | 0.64 |
| Amlodipine | 1.434052758 | 0.796963124 | 0.64 |
| Diflunisal | 1.674473436 | 1.041191155 | 0.63 |
| Iopromide | 1.57793765 | 0.946203905 | 0.63 |
| Imiquimod | 1.325867369 | 0.696174044 | 0.63 |
| Guanfacine (hydrochloride) | 1.600959233 | 0.980911063 | 0.62 |
| Niflumic acid | 1.485736926 | 0.872035489 | 0.61 |
| Vemurafenib | 1.257069409 | 0.644174923 | 0.61 |
| Toceranib | 1.361001318 | 0.749187297 | 0.61 |
| Paromomycin (sulfate) | 1.149981665 | 0.540989593 | 0.61 |
| Nebivolol (hydrochloride) | 1.173534073 | 0.566968094 | 0.61 |
| Lecithin | 1.410188845 | 0.8052013 | 0.60 |
| Fluphenazine decanoate | 1.068551842 | 0.463785446 | 0.60 |
| 6-Aminocaproic acid | 1.714556662 | 1.109874153 | 0.60 |
| Felbinac | 1.33518225 | 0.733492578 | 0.60 |
| Vinburnine | 1.423930269 | 0.822897117 | 0.60 |
| Alectinib (Hydrochloride) | 1.394173093 | 0.794499488 | 0.60 |
| Ceftibuten (dihydrate) | 1.241679873 | 0.646135472 | 0.60 |
| Mephenesin | 1.458992806 | 0.863774403 | 0.60 |
| Atropine methyl bromide | 1.708477509 | 1.11423658 | 0.59 |
| Acyclovir | 1.434052758 | 0.842950108 | 0.59 |
| Docosahexaenoic Acid | 1.76438227 | 1.17457204 | 0.59 |
| Talniflumate | 1.267146283 | 0.678958785 | 0.59 |
| Cyproheptadine (hydrochloride) | 1.570263789 | 0.982646421 | 0.59 |
| Trospium (chloride) | 1.858700806 | 1.277670216 | 0.58 |
| Sulfamethizole | 1.186212361 | 0.605869306 | 0.58 |
| Acetophenazine (dimaleate) | 1.148177496 | 0.572427913 | 0.58 |
| Menadione | 1.341521395 | 0.768298925 | 0.57 |
| Bekanamycin | 1.44586692 | 0.873023556 | 0.57 |
| Clioquinol | 1.174917492 | 0.608909987 | 0.57 |
| Troglitazone | 1.358954041 | 0.795598021 | 0.56 |
| Tolmetin (sodium dihydrate) | 1.262210797 | 0.702254868 | 0.56 |
| Cinacalcet | 1.377971474 | 0.818119775 | 0.56 |
| Flubendazole | 1.107444078 | 0.549023188 | 0.56 |
| Abacavir | 1.2 | 0.642516269 | 0.56 |
| Cyproheptadine (hydrochloride sesquihydrate) | 1.398573693 | 0.841324006 | 0.56 |
| Proxyphylline | 1.327335047 | 0.771267509 | 0.56 |
| Moguisteine | 1.262210797 | 0.70840451 | 0.55 |
| Desfesoterodine | 1.29991431 | 0.750085412 | 0.55 |
| Lobeline (hydrochloride) | 1.201918465 | 0.653796095 | 0.55 |
| DL-Norepinephrine (hydrochloride) | 1.652139461 | 1.10476028 | 0.55 |
| Cinepazide (Maleate) | 1.265451664 | 0.719160553 | 0.55 |
| Piperidolate (hydrochloride) | 1.097686375 | 0.553980184 | 0.54 |
| Josamycin | 1.215938303 | 0.679706184 | 0.54 |
| Trimethoprim | 1.095879556 | 0.566968094 | 0.53 |
| Nicorandil | 1.354199683 | 0.828356936 | 0.53 |
| Pranoprofen | 1.137083994 | 0.613376557 | 0.52 |
| Fesoterodine (L-mandelate) | 1.263924593 | 0.74188589 | 0.52 |
| Ethynyl Estradiol | 1.392906574 | 0.873479916 | 0.52 |
| Midecamycin | 1.536418166 | 1.020669628 | 0.52 |
| Piribedil | 1.36707004 | 0.852108819 | 0.51 |
| Idoxuridine | 1.387317909 | 0.875128118 | 0.51 |
| Amprenavir | 1.403357314 | 0.89154013 | 0.51 |
| Nicergoline | 1.444532488 | 0.935505886 | 0.51 |
| Imidapril (hydrochloride) | 1.457583548 | 0.948923813 | 0.51 |
| Mirogabalin | 1.015034837 | 0.507394559 | 0.51 |
| Itraconazole | 1.25735617 | 0.750187547 | 0.51 |
| Bendazac L-Lysine | 1.114824336 | 0.610693543 | 0.50 |
| Erythromycin estolate | 1.129446278 | 0.627898485 | 0.50 |
| Benzyl benzoate | 1.131962296 | 0.631875641 | 0.50 |
| Ethylparaben | 1.563396894 | 1.064960047 | 0.50 |
| Vigabatrin | 1.221079692 | 0.725486847 | 0.50 |
| L-Glutamine | 1.521691292 | 1.026212553 | 0.50 |
| Esomeprazole magnesium | 1.174807198 | 0.679706184 | 0.50 |
| Choline (chloride) | 1.217266187 | 0.722342733 | 0.49 |
| Orlistat | 1.512881302 | 1.018231688 | 0.49 |
| Amitriptyline (hydrochloride) | 1.344473008 | 0.856679194 | 0.49 |
| Vincamine | 1.177851118 | 0.692167245 | 0.49 |
| Vitamin K1 | 1.41154815 | 0.927282104 | 0.48 |
| L-Leucine | 1.836686577 | 1.354315264 | 0.48 |
| Fadrozole | 1.015827338 | 0.534924078 | 0.48 |
| Cevimeline (hydrochloride hemihydrate) | 1.046272494 | 0.565596174 | 0.48 |
| Sacubitril hemicalcium salt | 1.13406723 | 0.653595738 | 0.48 |
| Deoxycorticosterone acetate | 1.214224507 | 0.741202597 | 0.47 |
| Pyridoxal phosphate | 1.512425021 | 1.045268193 | 0.47 |
| Epristeride | 1.029134533 | 0.566279467 | 0.46 |
| Amantadine (hydrochloride) | 1.418512111 | 0.956516398 | 0.46 |
| D-α-Tocopherol acetate | 1.233187135 | 0.772930649 | 0.46 |
| Febuxostat | 1.415051903 | 0.955042378 | 0.46 |
| γ-Oryzanol | 1.531944171 | 1.073838414 | 0.46 |
| Anisindione | 1.349640288 | 0.89154013 | 0.46 |
| Tafamidis | 0.85645933 | 0.398955273 | 0.46 |
| Chlorothiazide | 1.320934256 | 0.866109815 | 0.45 |
| Danoprevir | 1.256578947 | 0.802013423 | 0.45 |
| Gilteritinib | 0.749670619 | 0.296074019 | 0.45 |
| Saquinavir (Mesylate) | 1.205755396 | 0.753579176 | 0.45 |
| Betrixaban | 1.061972864 | 0.610370641 | 0.45 |
| Imatinib (Mesylate) | 1.016640254 | 0.567650572 | 0.45 |
| Ipriflavone | 1.135313531 | 0.687054957 | 0.45 |
| 4-Methylumbelliferone | 1.119178585 | 0.673909074 | 0.45 |
| Clorprenaline hydrochloride | 1.061870504 | 0.618221258 | 0.44 |
| Mezlocillin (sodium) | 1.263924593 | 0.82251452 | 0.44 |
| Enzalutamide | 1.325673534 | 0.885002559 | 0.44 |
| Clonixin | 1.3479006 | 0.907926204 | 0.44 |
| Linaclotide | 1.765011003 | 1.32649786 | 0.44 |
| Amodiaquine (dihydrochloride dihydrate) | 1.353477218 | 0.915835141 | 0.44 |
| Clidinium (bromide) | 1.604285433 | 1.169724771 | 0.43 |
| Ampicillin | 1.363324764 | 0.931841476 | 0.43 |
| Dienestrol | 1.078107811 | 0.650538616 | 0.43 |
| Ornidazole | 1.081616482 | 0.6543252 | 0.43 |
| Flecainide (acetate) | 1.125179856 | 0.701518438 | 0.42 |
| Secnidazole | 1.528012581 | 1.104616751 | 0.42 |
| Linoleic acid | 1.557136083 | 1.134398193 | 0.42 |
| Davercin | 0.962229556 | 0.540259266 | 0.42 |
| Meisoindigo | 1.238369305 | 0.81691974 | 0.42 |
| Sulbenicillin (disodium) | 1.226255959 | 0.806098229 | 0.42 |
| Olmesartan medoxomil | 1.033093525 | 0.613015184 | 0.42 |
| Diethylstilbestrol | 0.979831316 | 0.564360051 | 0.42 |
| Chenodeoxycholic Acid | 1.210796915 | 0.797232661 | 0.41 |
| Nimorazole | 0.988632197 | 0.57604528 | 0.41 |
| 4-Deoxypyridoxine 5′-phosphate | 1.149981665 | 0.738908161 | 0.41 |
| Vortioxetine | 0.993032637 | 0.585539529 | 0.41 |
| Acetylcholine (chloride) | 1.724143351 | 1.320078459 | 0.40 |
| Phenindione | 1.152038369 | 0.748373102 | 0.40 |
| Magnolol | 1.053090333 | 0.649547859 | 0.40 |
| Rifampicin | 1.142647598 | 0.739638488 | 0.40 |
| Tenofovir (hydrate) | 1.28057554 | 0.877657267 | 0.40 |
| Brigatinib | 0.948177426 | 0.553138285 | 0.40 |
| Diclazuril | 1.130744849 | 0.736222488 | 0.39 |
| Adefovir | 1.199385156 | 0.810202551 | 0.39 |
| Medrysone | 1.282756441 | 0.894966118 | 0.39 |
| Tobramycin | 1.801515698 | 1.416403018 | 0.39 |
| Acemetacin | 1.479260861 | 1.09869784 | 0.38 |
| Lesinurad (sodium) | 1.262653254 | 0.885699001 | 0.38 |
| N-Acetylprocainamide | 1.655611443 | 1.278708987 | 0.38 |
| Fusidic acid (sodium salt) | 1.106973059 | 0.730080191 | 0.38 |
| Quinapril (hydrochloride) | 0.99449945 | 0.619134563 | 0.38 |
| Camostat (mesylate) | 0.982764943 | 0.610370641 | 0.37 |
| Aniracetam | 1.061972864 | 0.689976264 | 0.37 |
| Nevirapine | 1.136957182 | 0.765847348 | 0.37 |
| Diclofenac (diethylamine) | 1.145075683 | 0.783811779 | 0.36 |
| Loteprednol Etabonate | 0.896882494 | 0.538394794 | 0.36 |
| Mupirocin | 1.062599049 | 0.708923392 | 0.35 |
| Furazolidone | 1.149762282 | 0.796962976 | 0.35 |
| Meropenem (trihydrate) | 1.521626298 | 1.169266675 | 0.35 |
| Erdafitinib | 1.176703645 | 0.827674458 | 0.35 |
| Gilteritinib hemifumarate | 0.719806763 | 0.372093023 | 0.35 |
| Rifaximin | 1.163268532 | 0.815585673 | 0.35 |
| Roxithromycin | 1.33615917 | 0.990418867 | 0.35 |
| Budesonide | 1.034356725 | 0.692393736 | 0.34 |
| Cefmetazole (sodium) | 0.989698891 | 0.650230336 | 0.34 |
| (S)-Glutamic acid | 1.634863251 | 1.298680456 | 0.34 |
| Sodium 4-phenylbutyrate | 1.098788927 | 0.762928387 | 0.34 |
| Adapalene | 1.021972318 | 0.69758015 | 0.32 |
| Axitinib | 1.03410341 | 0.710425415 | 0.32 |
| Ribociclib hydrochloride | 1.499685633 | 1.177425107 | 0.32 |
| Hydroxyzine (pamoate) | 0.962229556 | 0.640314041 | 0.32 |
| Obeticholic acid | 0.912357902 | 0.590651817 | 0.32 |
| Empagliflozin | 1.111844518 | 0.793682673 | 0.32 |
| Rosiglitazone (maleate) | 1.422485727 | 1.107276819 | 0.32 |
| Uridine triacetate | 1.370379319 | 1.055240793 | 0.32 |
| Flurbiprofen | 1.387370242 | 1.072472669 | 0.31 |
| D-Sorbitol | 1.319550173 | 1.006141752 | 0.31 |
| Cefoperazone | 1.132290185 | 0.823330106 | 0.31 |
| Selexipag | 1.028236157 | 0.720649991 | 0.31 |
| Voriconazole | 0.989717224 | 0.682439358 | 0.31 |
| Sinomenine hydrochloride | 1.523577491 | 1.216654779 | 0.31 |
| Chlormezanone | 0.988114105 | 0.687084115 | 0.30 |
| Citalopram (hydrobromide) | 1.105995204 | 0.809110629 | 0.30 |
| Squalene | 1.566331342 | 1.273002853 | 0.29 |
| Rucaparib (phosphate) | 0.944844125 | 0.653796095 | 0.29 |
| Emtricitabine | 1.235347707 | 0.945666235 | 0.29 |
| Citicoline | 1.062003488 | 0.772563177 | 0.29 |
| Carbimazole | 1.469038726 | 1.182746375 | 0.29 |
| Prasugrel | 1.768783401 | 1.482703281 | 0.29 |
| Saralasin (TFA) | 1.396472578 | 1.110481586 | 0.29 |
| Erlotinib (Hydrochloride) | 0.492086331 | 0.207809111 | 0.28 |
| Sodium citrate (dihydrate) | 0.996669257 | 0.714432566 | 0.28 |
| Haloperidol | 0.759809314 | 0.478181486 | 0.28 |
| Raloxifene (hydrochloride) | 1.184391586 | 0.902781888 | 0.28 |
| Minocycline (hydrochloride) | 1.176978417 | 0.895878525 | 0.28 |
| Fructose | 1.328879441 | 1.047865459 | 0.28 |
| Dronedarone (Hydrochloride) | 0.741216795 | 0.460368978 | 0.28 |
| Silibinin | 1.219337338 | 0.938628159 | 0.28 |
| Imiquimod (hydrochloride) | 1.160015726 | 0.881473809 | 0.28 |
| Etoricoxib | 1.320906433 | 1.044742729 | 0.28 |
| Bepotastine | 1.25594653 | 0.980911512 | 0.28 |
| Pioglitazone | 1.168539326 | 0.893919793 | 0.27 |
| Eplerenone | 1.291133124 | 1.018413598 | 0.27 |
| Ziprasidone (hydrochloride monohydrate) | 0.855152182 | 0.584809202 | 0.27 |
| Cefadroxil | 1.685474949 | 1.423523717 | 0.26 |
| Maraviroc | 0.893289329 | 0.633741099 | 0.26 |
| Ertugliflozin L-pyroglutamic acid | 1.575762339 | 1.318651926 | 0.26 |
| Pitolisant (hydrochloride) | 1.232352941 | 0.979118044 | 0.25 |
| Darunavir (Ethanolate) | 1.146674765 | 0.895213454 | 0.25 |
| Abiraterone | 1.370748968 | 1.119414028 | 0.25 |
| Merbromin | 1.543846987 | 1.293745437 | 0.25 |
| Ripasudil | 0.906490649 | 0.657111557 | 0.25 |
| Mosapride (citrate) | 1.240657439 | 0.995332269 | 0.25 |
| Guanidine (hydrochloride) | 1.23650519 | 0.991892888 | 0.24 |
| (+)-Camphor | 1.190360715 | 0.945761916 | 0.24 |
| Acalabrutinib | 0.988968825 | 0.744902386 | 0.24 |
| Droxidopa | 1.239789196 | 0.997249312 | 0.24 |
| Dichlorisone acetate | 1.095740009 | 0.853213303 | 0.24 |
| Vonoprazan (Fumarate) | 1.405170331 | 1.164305949 | 0.24 |
| Olmutinib | 0.79795715 | 0.557112069 | 0.24 |
| Articaine (hydrochloride) | 1.285334622 | 1.045325779 | 0.24 |
| Mebendazole | 0.384822521 | 0.145842217 | 0.24 |
| Avibactam (sodium hydrate) | 0.883021635 | 0.644696001 | 0.24 |
| Bretylium (tosylate) | 0.751008434 | 0.515428154 | 0.24 |
| Crizotinib | 0.373453608 | 0.14107717 | 0.23 |
| Mebhydrolin (napadisylate) | 1.238499184 | 1.007654321 | 0.23 |
| Pyrimethamine | 1.426124567 | 1.196781722 | 0.23 |
| Dabrafenib (Mesylate) | 0.633661153 | 0.404396876 | 0.23 |
| Tyloxapol | 1.116746699 | 0.887636364 | 0.23 |
| Simeprevir | 0.795012835 | 0.566551032 | 0.23 |
| Pyrithioxin (dihydrochloride) | 1.062443165 | 0.834937779 | 0.23 |
| Cefpodoxime Proxetil | 1.310895111 | 1.084605321 | 0.23 |
| Norethindrone | 1.412423825 | 1.186297721 | 0.23 |
| Ospemifene | 1.254628422 | 1.02863589 | 0.23 |
| Angiotensin II 5-valine | 1.089583966 | 0.86545925 | 0.22 |
| Osalmid | 1.432300163 | 1.209135802 | 0.22 |
| Tacrine hydrochloride hydrate | 1.476901907 | 1.253773306 | 0.22 |
| Gefitinib (hydrochloride) | 0.871819188 | 0.649725718 | 0.22 |
| Vesnarinone | 1.196775723 | 0.975637302 | 0.22 |
| Lumefantrine | 1.173034695 | 0.95523881 | 0.22 |
| (−)-Limonene | 1.515431492 | 1.298757029 | 0.22 |
| Reboxetine (mesylate) | 1.321125432 | 1.104671897 | 0.22 |
| Nedocromil | 1.892840209 | 1.67779929 | 0.22 |
| Larotrectinib sulfate | 1.739212897 | 1.525492094 | 0.21 |
| Polyvinylpyrrolidone | 1.230192077 | 1.016606061 | 0.21 |
| Sorafenib | 1.014546884 | 0.804527967 | 0.21 |
| Esmolol (hydrochloride) | 0.932893289 | 0.724301625 | 0.21 |
| Amoxicillin | 1.15943711 | 0.95551679 | 0.20 |
| Midodrine (hydrochloride) | 0.864268585 | 0.660737527 | 0.20 |
| Chloroxine | 1.288109075 | 1.085912641 | 0.20 |
| Lysipressin | 1.080964798 | 0.884935786 | 0.20 |
| Broxyquinoline | 1.23983013 | 1.044258027 | 0.20 |
| Bicyclol | 0.745641838 | 0.551953592 | 0.19 |
| Terbinafine | 1.421318846 | 1.228627259 | 0.19 |
| Biperiden (Hydrochloride) | 1.311205943 | 1.119554695 | 0.19 |
| Tetrandrine | 1.143170839 | 0.95223806 | 0.19 |
| Aceclofenac | 1.139571457 | 0.951316958 | 0.19 |
| Eravacycline (dihydrochloride) | 0.401918465 | 0.213882863 | 0.19 |
| Istradefylline | 1.167301038 | 0.982066085 | 0.19 |
| Menthol | 1.129401089 | 0.944311218 | 0.19 |
| DHEA | 1.634234517 | 1.449179743 | 0.19 |
| S-Adenosyl-L-methionine (tosylate) | 1.043320141 | 0.858315489 | 0.19 |
| Afoxolaner | 1.190403887 | 1.007761966 | 0.18 |
| Nitroxoline | 1.027126526 | 0.844765343 | 0.18 |
| Sulbutiamine | 1.437520392 | 1.25654321 | 0.18 |
| Cyclobenzaprine (hydrochloride) | 1.260019984 | 1.079564145 | 0.18 |
| Cyclosporin A | 0.799040767 | 0.619088937 | 0.18 |
| Cysteamine hydrochloride | 1.430524992 | 1.255171184 | 0.18 |
| Amisulpride | 1.349418422 | 1.17457204 | 0.17 |
| Tenapanor | 0.862774223 | 0.68835968 | 0.17 |
| Casanthranol | 1.108343337 | 0.934666667 | 0.17 |
| Edoxaban (tosylate monohydrate) | 0.938544474 | 0.764965986 | 0.17 |
| Vandetanib | 0.363280294 | 0.190191898 | 0.17 |
| (±)-Darifenacin | 1.180449827 | 1.008598452 | 0.17 |
| Ferulic acid (sodium) | 1.309179503 | 1.137482168 | 0.17 |
| Cefoperazone (sodium salt) | 1.173529412 | 1.002211031 | 0.17 |
| Apronal | 1.458030273 | 1.286919207 | 0.17 |
| Umeclidinium (bromide) | 0.755408874 | 0.584809202 | 0.17 |
| Sertaconazole (nitrate) | 0.859468723 | 0.689272292 | 0.17 |
| (−)-Securinine | 0.927097058 | 0.757189297 | 0.17 |
| Vilazodone | 1.053806228 | 0.884289399 | 0.17 |
| Suplatast (Tosilate) | 1.127239599 | 0.958602846 | 0.17 |
| Benznidazol | 1.21546212 | 1.046931408 | 0.17 |
| Bethanechol (chloride) | 1.407302039 | 1.240238787 | 0.17 |
| Sonidegib | 0.818814433 | 0.65233119 | 0.17 |
| Imidafenacin | 1.001166181 | 0.836828575 | 0.16 |
| Polidocanol | 0.923469388 | 0.760606061 | 0.16 |
| Succinic acid | 1.143024772 | 0.980735856 | 0.16 |
| Cefoselis (sulfate) | 1.116484906 | 0.954985479 | 0.16 |
| Entacapone | 0.919346979 | 0.762863535 | 0.16 |
| Amiloride hydrochloride dihydrate | 1.060125168 | 0.904252242 | 0.16 |
| Oxytetracycline | 1.085939872 | 0.930142303 | 0.16 |
| Edoxaban | 1.758878956 | 1.603399433 | 0.16 |
| Fenspiride (Hydrochloride) | 0.751980983 | 0.596997099 | 0.15 |
| Agomelatine | 0.813847555 | 0.661060802 | 0.15 |
| Alendronate (sodium hydrate) | 0.27764761 | 0.124872406 | 0.15 |
| Micafungin (sodium) | 1.188335594 | 1.035585353 | 0.15 |
| Revaprazan (hydrochloride) | 1.344463668 | 1.192359661 | 0.15 |
| Deoxycholic acid sodium salt | 0.954272428 | 0.804538422 | 0.15 |
| Cilazapril (monohydrate) | 0.989563673 | 0.841077303 | 0.15 |
| Atovaquone | 1.101879539 | 0.954319762 | 0.15 |
| Benzethonium chloride | 0.219664269 | 0.07505423 | 0.14 |
| Ethacrynic acid | 0.956038079 | 0.812633833 | 0.14 |
| Felbamate | 1.102941176 | 0.96093846 | 0.14 |
| Pirenzepine (dihydrochloride) | 1.606406894 | 1.465070835 | 0.14 |
| Cephapirin (sodium) | 1.130883693 | 0.989650712 | 0.14 |
| Pimavanserin | 1.126070991 | 0.986780384 | 0.14 |
| Vanillin | 0.862983425 | 0.724110981 | 0.14 |
| Hydrocortisone | 1.030022006 | 0.891405136 | 0.14 |
| Deflazacort | 0.765072247 | 0.628951149 | 0.14 |
| Nitrofurazone | 1.415520784 | 1.280897064 | 0.13 |
| Cilnidipine | 1.574293308 | 1.440509915 | 0.13 |
| 5-Acetylsalicylic acid | 1.044640146 | 0.912031048 | 0.13 |
| Trioxsalen | 1.239151713 | 1.107407407 | 0.13 |
| L-Arginine | 1.114706452 | 0.984012378 | 0.13 |
| Resveratrol | 1.165828356 | 1.036911555 | 0.13 |
| Gatifloxacin | 1.266139657 | 1.137284321 | 0.13 |
| Abemaciclib | 0.849802372 | 0.721180295 | 0.13 |
| Mianserin (hydrochloride) | 1.174913495 | 1.048888343 | 0.13 |
| Dutasteride | 0.844298246 | 0.720357942 | 0.12 |
| Pyrantel (pamoate) | 1.202550865 | 1.078913325 | 0.12 |
| Tenofovir alafenamide hemifumarate | 0.549171271 | 0.425556858 | 0.12 |
| Bendazol | 1.354236289 | 1.233649009 | 0.12 |
| Acetazolamide | 0.941436464 | 0.821023837 | 0.12 |
| Chlorhexidine (digluconate) | 0.943004023 | 0.824414232 | 0.12 |
| Taurine | 1.309416159 | 1.192216632 | 0.12 |
| Pemirolast (potassium) | 1.01141925 | 0.895061728 | 0.12 |
| Nitazoxanide | 1.148761408 | 1.033495314 | 0.12 |
| Amoxicillin (trihydrate) | 0.977126703 | 0.862253289 | 0.11 |
| Bufexamac | 1.458816591 | 1.346108316 | 0.11 |
| Glycerol | 1.096099339 | 0.984024412 | 0.11 |
| Thymopentin (acetate) | 1.094608605 | 0.98381715 | 0.11 |
| Bambuterol hydrochloride | 1.47227426 | 1.362481681 | 0.11 |
| Pasiniazid | 1.274031846 | 1.164397751 | 0.11 |
| Rofecoxib | 1.148868005 | 1.040714995 | 0.11 |
| Ixazomib citrate | 0.101835985 | −0.005970149 | 0.11 |
| Diphenmanil (methylsulfate) | 0.924385059 | 0.81759379 | 0.11 |
| Terconazole | 1.158265698 | 1.05163853 | 0.11 |
| Prasugrel (hydrochloride) | 1.044117647 | 0.939319494 | 0.10 |
| Duloxetine (hydrochloride) | 1.4340577 | 1.331216414 | 0.10 |
| Quizartinib | 0.765676568 | 0.662954172 | 0.10 |
| Prulifloxacin | 1.213322965 | 1.110610611 | 0.10 |
| Oxybuprocaine hydrochloride | 0.902103401 | 0.799792838 | 0.10 |
| Thiamphenicol | 0.910512129 | 0.808503401 | 0.10 |
| Asenapine (hydrochloride) | 1.388347696 | 1.288226673 | 0.10 |
| Crizotinib (hydrochloride) | 0.279639175 | 0.181270096 | 0.10 |
| Triflusal | 1.116768233 | 1.018792542 | 0.10 |
| Bivalirudin (TFA) | 1.117686467 | 1.019811321 | 0.10 |
| Andrographolide | 0.986824259 | 0.891181021 | 0.10 |
| L-Tryptophan | 1.102733874 | 1.008937438 | 0.09 |
| Etofylline | 1.354649266 | 1.261481481 | 0.09 |
| Spectinomycin (dihydrochloride) | 1.077112204 | 0.984090252 | 0.09 |
| Metformin (hydrochloride) | 1.069277846 | 0.976545842 | 0.09 |
| Calcium dobesilate | 1.417809324 | 1.325144908 | 0.09 |
| Fudosteine | 1.176528406 | 1.083900562 | 0.09 |
| Phenytoin | 0.896495957 | 0.804421769 | 0.09 |
| Almitrine mesylate | 0.817679558 | 0.725674091 | 0.09 |
| Metaproterenol (hemisulfate) | 0.933675748 | 0.84245283 | 0.09 |
| Ozagrel (hydrochloride) | 1.021499626 | 0.930406852 | 0.09 |
| Amoxapine | 1.390232338 | 1.299612778 | 0.09 |
| Deprodone propionate | 0.785999003 | 0.696479885 | 0.09 |
| Rifabutin | 0.821135742 | 0.73221216 | 0.09 |
| Belinostat | 0.16049672 | 0.071793127 | 0.09 |
| Pindolol | 0.972979008 | 0.885220126 | 0.09 |
| Bleomycin (hydrochloride) | 0.519490726 | 0.432774608 | 0.09 |
| Albendazole | 0.986249407 | 0.900129074 | 0.09 |
| Oxprenolol (hydrochloride) | 1.26217656 | 1.176519741 | 0.09 |
| Frovatriptan (succinate hydrate) | 1.110526316 | 1.025313083 | 0.09 |
| Atropine (sulfate monohydrate) | 1.139399142 | 1.05629478 | 0.08 |
| Sorafenib (Tosylate) | 0.961897356 | 0.879713046 | 0.08 |
| Fenticonazole (Nitrate) | 1.071277895 | 0.989925987 | 0.08 |
| Norfloxacin | 1.123207301 | 1.042519958 | 0.08 |
| Tamoxifen (Citrate) | 0.575984165 | 0.496243169 | 0.08 |
| Azelastine (hydrochloride) | 1.039113104 | 0.95941092 | 0.08 |
| Rutin | 1.308759477 | 1.229091913 | 0.08 |
| Thioridazine (hydrochloride) | 1.12365416 | 1.045185185 | 0.08 |
| Colistin (sulfate) | 1.114013841 | 1.0361135 | 0.08 |
| Doxorubicin (hydrochloride) | −0.006006588 | −0.082516761 | 0.08 |
| Trimethobenzamide hydrochloride | 0.9013058 | 0.825355757 | 0.08 |
| Iodixanol | 1.054036736 | 0.978152929 | 0.08 |
| Dirithromycin | 0.993799651 | 0.917998969 | 0.08 |
| Tolterodine | 1.051022652 | 0.975751366 | 0.08 |
| Pemetrexed (disodium hemipenta hydrate) | 0.169329897 | 0.094453376 | 0.07 |
| (R)-(+)-Atenolol | 0.92137031 | 0.846666667 | 0.07 |
| Cladribine | 0.064901593 | −0.008506295 | 0.07 |
| Clavulanate (lithium) | 1.617196144 | 1.544853178 | 0.07 |
| Homatropine (Bromide) | 0.903650587 | 0.831482124 | 0.07 |
| Busulfan | 1.71898214 | 1.648112294 | 0.07 |
| Ganciclovir | 0.96384083 | 0.893133522 | 0.07 |
| Gimeracil | 0.923170361 | 0.852522639 | 0.07 |
| Ensulizole | 0.931381314 | 0.861440555 | 0.07 |
| Dexamethasone | 0.810683952 | 0.741105004 | 0.07 |
| Gentamicin (sulfate) | 0.979891957 | 0.910424242 | 0.07 |
| Rotigotine | 0.968548276 | 0.900430707 | 0.07 |
| Moxisylyte (hydrochloride) | 1.095899188 | 1.027805362 | 0.07 |
| Hydroxyfasudil | 1.010962099 | 0.943985929 | 0.07 |
| Riboflavin Tetrabutyrate | 0.84315515 | 0.776640056 | 0.07 |
| Tolperisone (hydrochloride) | 1.436215334 | 1.370123457 | 0.07 |
| Ropivacaine (hydrochloride) | 1.265054528 | 1.19958051 | 0.07 |
| Flunarizine (dihydrochloride) | 1.036153653 | 0.970960536 | 0.07 |
| Abacavir (sulfate) | 0.487225459 | 0.423272727 | 0.06 |
| Sulconazole (mononitrate) | 0.920326679 | 0.856914468 | 0.06 |
| Vaborbactam | 0.964790542 | 0.901726974 | 0.06 |
| Rilpivirine | 0.8865177 | 0.823529412 | 0.06 |
| Desogestrel | 0.964470088 | 0.901681759 | 0.06 |
| Hydroquinidine | 0.88149636 | 0.819061802 | 0.06 |
| Midostaurin | 0.395768025 | 0.333551913 | 0.06 |
| Estradiol | 0.767782651 | 0.706995004 | 0.06 |
| Terpin (hydrate) | 0.92884097 | 0.868367347 | 0.06 |
| Cefodizime (sodium) | 1.06922675 | 1.009398907 | 0.06 |
| Neratinib | 0.126810787 | 0.067071116 | 0.06 |
| L-Isoleucine | 1.082295779 | 1.023285899 | 0.06 |
| Doravirine | 0.87336775 | 0.816300129 | 0.06 |
| Erdosteine | 1.515736231 | 1.459208598 | 0.06 |
| Flumazenil | 1.127786418 | 1.073239907 | 0.05 |
| Icotinib (Hydrochloride) | 0.323969072 | 0.269694534 | 0.05 |
| Azithromycin | 1.302686895 | 1.248715869 | 0.05 |
| Pyridostigmine (bromide) | 0.935309973 | 0.881972789 | 0.05 |
| Alimemazine hemitartrate | 1.239358146 | 1.186046512 | 0.05 |
| Zafirlukast | 0.900091102 | 0.847347995 | 0.05 |
| Talazoparib tosylate | 0.192361762 | 0.139843484 | 0.05 |
| (+)-Catechin hydrate | 1.110144058 | 1.057818182 | 0.05 |
| Alosetron (Hydrochloride) | 1.450015718 | 1.397824536 | 0.05 |
| Moxonidine | 0.965714286 | 0.913678799 | 0.05 |
| D-Fructose | 1.202595156 | 1.15059575 | 0.05 |
| Domiphen (bromide) | 0.016281063 | −0.035702084 | 0.05 |
| Dihydroartemisinin | 0.648905251 | 0.597215059 | 0.05 |
| Cefixime | 1.188335594 | 1.136668386 | 0.05 |
| Histamine | 0.98128142 | 0.931834404 | 0.05 |
| Metipranolol hydrochloride | 0.893022561 | 0.844121212 | 0.05 |
| Melatonin | 0.996899826 | 0.948942754 | 0.05 |
| Methenamine (hippurate) | 0.143144261 | 0.095283019 | 0.05 |
| Pramipexole (dihydrochloride hydrate) | 0.873034549 | 0.825481799 | 0.05 |
| (S)-Rasagiline | 1.048081714 | 1.001077586 | 0.05 |
| Oxymatrine | 0.995838433 | 0.949253731 | 0.05 |
| Tamoxifen | 0.706888472 | 0.661109221 | 0.05 |
| Amphotericin B | 1.127854671 | 1.082299472 | 0.05 |
| Bosutinib | 0.454160438 | 0.409123563 | 0.05 |
| Sulconazole (nitrate) | 0.966686675 | 0.922060606 | 0.04 |
| Chlorhexidine | 0.125919322 | 0.08190091 | 0.04 |
| Regorafenib (Hydrochloride) | 0.873597165 | 0.829667429 | 0.04 |
| Gliclazide | 1.121541081 | 1.079068342 | 0.04 |
| Bumetanide | 0.740624533 | 0.698666667 | 0.04 |
| Afloqualone | 1.10358821 | 1.062562066 | 0.04 |
| Daclatasvir | 0.743650325 | 0.702973781 | 0.04 |
| Linezolid | 0.988050609 | 0.948281728 | 0.04 |
| Vinblastine (sulfate) | 0.091143393 | 0.05137802 | 0.04 |
| Docetaxel | 0.205524862 | 0.1660805 | 0.04 |
| Ibudilast | 1.164982791 | 1.125616776 | 0.04 |
| Digitoxin | 0.019795658 | −0.019307386 | 0.04 |
| Cepharanthine | 1.159120034 | 1.120158888 | 0.04 |
| Sulfacarbamide | 0.946486066 | 0.908511513 | 0.04 |
| Sulfacetamide (Sodium) | 0.904693611 | 0.866886498 | 0.04 |
| Chlormethine (hydrochloride) | 0.758752998 | 0.721475054 | 0.04 |
| Pyrazinamide | 1.011389013 | 0.974528302 | 0.04 |
| Alpelisib | 0.533527045 | 0.496962684 | 0.04 |
| Rapacuronium bromide | 0.91581063 | 0.880188679 | 0.04 |
| Ioxilan | 1.050447816 | 1.015139227 | 0.04 |
| Quinine (hydrochloride dihydrate) | 1.120128789 | 1.085320724 | 0.03 |
| Donepezil | 0.988135593 | 0.953661923 | 0.03 |
| Ketanserin (tartrate) | 1.01935342 | 0.985314208 | 0.03 |
| Ramosetron (Hydrochloride) | 0.95451895 | 0.922337979 | 0.03 |
| Spironolactone | 1.134159061 | 1.102221451 | 0.03 |
| Pinaverium bromide | 0.426306387 | 0.394654088 | 0.03 |
| Succinylsulfathiazole | 0.883557951 | 0.852040816 | 0.03 |
| Cetylpyridinium (chloride monohydrate) | 0.014486294 | −0.016514998 | 0.03 |
| Tipiracil (hydrochloride) | 0.905165767 | 0.875411184 | 0.03 |
| Pralatrexate | 0.077936579 | 0.048742138 | 0.03 |
| Dexrazoxane (hydrochloride) | 0.979891957 | 0.951636364 | 0.03 |
| Acamprosate (calcium) | 1.778756088 | 1.751343429 | 0.03 |
| Ornipressin | 1.050612245 | 1.023542146 | 0.03 |
| Tricaprilin | 0.974585635 | 0.947635795 | 0.03 |
| Afatinib (dimaleate) | 0.105610561 | 0.078692715 | 0.03 |
| Atorvastatin | 0.862250454 | 0.835598188 | 0.03 |
| Gemcitabine (elaidate) | 0.01387818 | −0.012746711 | 0.03 |
| Menadione bisulfite (sodium) | 1.491512103 | 1.465584879 | 0.03 |
| Ceftizoxime | 1.057105985 | 1.031465636 | 0.03 |
| Methylbenactyzium Bromide | 0.945013477 | 0.920068027 | 0.02 |
| Candesartan | 0.974110787 | 0.949397916 | 0.02 |
| Metoprolol (Succinate) | 0.983697578 | 0.959176491 | 0.02 |
| 6α-Methylprednisolone 21-hemisuccinate (sodium salt) | 0.631266846 | 0.607142857 | 0.02 |
| Fruquintinib | 0.993965132 | 0.97020538 | 0.02 |
| Tigecycline (tetramesylate) | 0.142355009 | 0.119136262 | 0.02 |
| Setiptiline (maleate) | 0.983225044 | 0.960584198 | 0.02 |
| Copanlisib (dihydrochloride) | 0.095625201 | 0.073162027 | 0.02 |
| Propafenone (hydrochloride) | 1.086377262 | 1.063939145 | 0.02 |
| Vincristine (sulfate) | 0.067217508 | 0.044968553 | 0.02 |
| (S)-Crizotinib | 1.27549386 | 1.253485425 | 0.02 |
| Etofenamate | 1.036137261 | 1.014230272 | 0.02 |
| L-Eflornithine (monohydrochloride) | 0.846935501 | 0.825481799 | 0.02 |
| Daclatasvir (dihydrochloride) | 1.045854844 | 1.02457956 | 0.02 |
| Levocetirizine (dihydrochloride) | 0.890135854 | 0.869083231 | 0.02 |
| Inosine | 0.941003672 | 0.920255864 | 0.02 |
| Pazopanib | 1.104532164 | 1.083892617 | 0.02 |
| Rizatriptan (benzoate) | 0.446997085 | 0.426599919 | 0.02 |
| Valdecoxib | 0.92239608 | 0.902065182 | 0.02 |
| Valrubicin | 0.066237113 | 0.046221865 | 0.02 |
| Tizanidine (hydrochloride) | 1.089602926 | 1.069726776 | 0.02 |
| Emetine (dihydrochloride hydrate) | 0.042882457 | 0.023051205 | 0.02 |
| Ifenprodil (tartrate) | 0.911459036 | 0.892119907 | 0.02 |
| Moexipril (hydrochloride) | 1.023719677 | 1.004421769 | 0.02 |
| Stiripentol | 0.941491069 | 0.922555318 | 0.02 |
| Glycopyrrolate | 0.829638627 | 0.811125485 | 0.02 |
| Irinotecan (hydrochloride) | 1.369614088 | 1.351734245 | 0.02 |
| Riluzole | 1.032538444 | 1.014829794 | 0.02 |
| (R)-Verapamil (hydrochloride) | 0.886725853 | 0.869735903 | 0.02 |
| Nintedanib esylate | 0.527881912 | 0.511398435 | 0.02 |
| Chlorobutanol | 0.579941691 | 0.564064403 | 0.02 |
| Emamectin (Benzoate) | 0.016728387 | 0.001259129 | 0.02 |
| Epinastine | 0.979162571 | 0.964338562 | 0.01 |
| Chlorquinaldol | 0.018491024 | 0.003949022 | 0.01 |
| Auranofin | 0.004900181 | −0.008526512 | 0.01 |
| Saccharin | 0.941454908 | 0.929238506 | 0.01 |
| Gadobutrol | 0.951050514 | 0.93896442 | 0.01 |
| Flavoxate (hydrochloride) | 0.995687332 | 0.984013605 | 0.01 |
| Quinidine hydrochloride monohydrate | 1.222134854 | 1.210565256 | 0.01 |
| 10-Undecenoic acid | 0.989950871 | 0.979559748 | 0.01 |
| Octenidine (dihydrochloride) | 0.014833127 | 0.004560426 | 0.01 |
| Fluocinolone (Acetonide) | 0.561724749 | 0.552349111 | 0.01 |
| Binimetinib | 0.182472372 | 0.173108553 | 0.01 |
| Ouabain (Octahydrate) | −0.000668598 | −0.009774183 | 0.01 |
| Dicloxacillin (Sodium hydrate) | 0.854967198 | 0.846206193 | 0.01 |
| Benserazide (hydrochloride) | 1.0469155 | 1.038538539 | 0.01 |
| Levomepromazine | 0.904105614 | 0.896205844 | 0.01 |
| (S)-10-Hydroxycamptothecin | 0.027417381 | 0.019616205 | 0.01 |
| Lafutidine | 1.371112776 | 1.36443576 | 0.01 |
| Caffeic acid | 0.891493897 | 0.884992264 | 0.01 |
| (S)-Terazosin | 0.916347382 | 0.909898866 | 0.01 |
| Cobimetinib (hemifumarate) | 0.291731669 | 0.285473542 | 0.01 |
| Dobutamine (hydrochloride) | 1.131490974 | 1.125379171 | 0.01 |
| Rilmenidine (phosphate) | 0.995036573 | 0.989289617 | 0.01 |
| Carcainium (chloride) | 1.004086324 | 0.998489046 | 0.01 |
| Ivermectin | 0.150341131 | 0.14541387 | 0.00 |
| Belotecan (hydrochloride) | 0.010267693 | 0.005660033 | 0.00 |
| Clodronic acid (disodium salt) | 0.954985423 | 0.950480314 | 0.00 |
| Amsacrine (hydrochloride) | 0.241640543 | 0.237377049 | 0.00 |
| Afatinib | 0.03090379 | 0.026654039 | 0.00 |
| Cyclic somatostatin | 1.022623907 | 1.019753755 | 0.00 |
| Seratrodast | 0.990388914 | 0.987561469 | 0.00 |
| Brinzolamide | 0.876010782 | 0.873809524 | 0.00 |
| Hexylresorcinol | 1.036151603 | 1.034366121 | 0.00 |
| Fostamatinib (disodium hexahydrate) | 0.546626054 | 0.545423613 | 0.00 |
| Temocapril (hydrochloride) | 0.607930029 | 0.606819104 | 0.00 |
| Mitoxantrone (dihydrochloride) | −0.002011623 | −0.002892682 | 0.00 |
| Agomelatine (hydrochloride) | 0.748253872 | 0.747736093 | 0.00 |
| Perphenazine | 0.910279477 | 0.909818875 | 0.00 |
| Bortezomib | 0.006969876 | 0.006862573 | 0.00 |
| Hexaminolevulinate (hydrochloride) | 0.737636336 | 0.738424242 | 0.00 |
| Trametinib (DMSO solvate) | 0.282434597 | 0.2834812 | 0.00 |
| Bicalutamide | 1.01645768 | 1.018142077 | 0.00 |
| Oxiconazole nitrate | 0.740138177 | 0.742500843 | 0.00 |
| Tebipenem pivoxil | 0.998626609 | 1.001023541 | 0.00 |
| Dipotassium glycyrrhizinate | 1.007439446 | 1.010072473 | 0.00 |
| Rufinamide | 0.97850149 | 0.982224946 | 0.00 |
| Cefonicid (sodium) | 0.828571429 | 0.832993197 | 0.00 |
| (−)-Huperzine A | 1.616897714 | 1.621397167 | 0.00 |
| Nimustine (hydrochloride) | 1.015829888 | 1.020411754 | 0.00 |
| Podofilox | 0.04847071 | 0.053553554 | −0.01 |
| Zidovudine | 1.205084746 | 1.210517182 | −0.01 |
| Aclacinomycin A hydrochloride | 0.007534159 | 0.013346764 | −0.01 |
| Daunorubicin (Hydrochloride) | 0.008412017 | 0.01432958 | −0.01 |
| Tedizolid (phosphate) | 0.834474886 | 0.840609985 | −0.01 |
| Plicamycin | −0.001958384 | 0.004264392 | −0.01 |
| Pemetrexed | 0.159598346 | 0.165933486 | −0.01 |
| Cabazitaxel | 0.197995749 | 0.204398448 | −0.01 |
| Zinc Pyrithione | 0.007475438 | 0.013902681 | −0.01 |
| Idarubicin (hydrochloride) | −0.029411765 | −0.02296043 | −0.01 |
| Brimonidine | 0.965854883 | 0.972449739 | −0.01 |
| Selpercatinib | 0.915790676 | 0.922397865 | −0.01 |
| Siponimod | 0.925103367 | 0.932430055 | −0.01 |
| AZD7545 | 0.712535485 | 0.720969697 | −0.01 |
| Sulfogaiacol | 0.881138976 | 0.889617486 | −0.01 |
| Exemestane | 1.047589712 | 1.05615662 | −0.01 |
| Camptothecin | 0.005875153 | 0.014498934 | −0.01 |
| Ruxolitinib | 0.610125932 | 0.618832237 | −0.01 |
| Pantoprazole (sodium) | 0.810671846 | 0.819749538 | −0.01 |
| Paclitaxel | 0.057054742 | 0.066200658 | −0.01 |
| Salmeterol | 0.854827634 | 0.86496073 | −0.01 |
| Sunitinib | 0.522972634 | 0.533134773 | −0.01 |
| Ixabepilone | 0.091553455 | 0.101882808 | −0.01 |
| Triptolide | 0.030488789 | 0.041126139 | −0.01 |
| Floxuridine | 0.080468361 | 0.091594828 | −0.01 |
| Selamectin | −0.012098726 | −0.000828043 | −0.01 |
| Harringtonine | 0.006817682 | 0.018101093 | −0.01 |
| Triptonide | 0.005807419 | 0.018075232 | −0.01 |
| Epirubicin (hydrochloride) | −0.018867925 | −0.006462585 | −0.01 |
| i-Inositol | 1.539924552 | 1.552603424 | −0.01 |
| (S)-(+)-Ibuprofen | 0.998214531 | 1.01089435 | −0.01 |
| D-Tyrosine | 1.068135629 | 1.081013562 | −0.01 |
| Nicardipine (hydrochloride) | 0.906164592 | 0.919793014 | −0.01 |
| Mycophenolate Mofetil | 0.323224469 | 0.337170504 | −0.01 |
| Azaperone | 0.916347382 | 0.930738584 | −0.01 |
| Phenazine (methylsulfate) | 0.021606589 | 0.036045682 | −0.01 |
| Escitalopram (oxalate) | 1.269307424 | 1.284123563 | −0.01 |
| Ceritinib dihydrochloride | 0.348899987 | 0.364387004 | −0.02 |
| Probenecid | 0.908865711 | 0.92450538 | −0.02 |
| Disulfiram | −0.001922255 | 0.013902681 | −0.02 |
| Guaiacol | 1.26506491 | 1.281072718 | −0.02 |
| Propantheline (bromide) | 1.294476116 | 1.310594851 | −0.02 |
| Inulin | 0.905462185 | 0.922060606 | −0.02 |
| Solriamfetol (hydrochloride) | 1.000213584 | 1.016881827 | −0.02 |
| Faropenem sodium | 0.852210249 | 0.869193989 | −0.02 |
| Prednisolone | 0.476845573 | 0.494069108 | −0.02 |
| Daidzein | 1.082154621 | 1.099535843 | −0.02 |
| Fexofenadine (hydrochloride) | 1.034028683 | 1.051544603 | −0.02 |
| Treprostinil (sodium) | 0.999776686 | 1.017295597 | −0.02 |
| Norepinephrine | 0.963378176 | 0.980962644 | −0.02 |
| Buflomedil (hydrochloride) | 0.583673469 | 0.601407117 | −0.02 |
| 17-Hydroxyprogesterone | 1.184530387 | 1.202422821 | −0.02 |
| Betaxolol (hydrochloride) | 0.873854447 | 0.892857143 | −0.02 |
| Oxacillin (sodium monohydrate) | 0.906199461 | 0.925510204 | −0.02 |
| Dexamethasone phosphate disodium | 0.722685789 | 0.742624089 | −0.02 |
| Sulfapyridine | 0.790886726 | 0.811206281 | −0.02 |
| Lanatoside C | −0.02111588 | 7.10225E−18 | −0.02 |
| Puromycin (dihydrochloride) | −0.016445963 | 0.005958292 | −0.02 |
| Clopidogrel | 0.95801105 | 0.980461118 | −0.02 |
| Homoharringtonine | −0.042641326 | −0.020134228 | −0.02 |
| Rucaparib | 0.880973649 | 0.90408805 | −0.02 |
| Cobimetinib (racemate) | 0.275063499 | 0.298181818 | −0.02 |
| Nelfinavir (Mesylate) | 1.169372063 | 1.19265144 | −0.02 |
| Fipexide | 1.176528406 | 1.200503107 | −0.02 |
| Eslicarbazepine | 0.858744395 | 0.883261494 | −0.02 |
| Panobinostat | −0.02997076 | −0.004474273 | −0.03 |
| Diclofenac (potassium) | 1.107436901 | 1.133369234 | −0.03 |
| Ibandronate (Sodium Monohydrate) | 1.073843904 | 1.100567721 | −0.03 |
| Paritaprevir | 0.919758605 | 0.947063928 | −0.03 |
| Hyperoside | 0.890632785 | 0.919181034 | −0.03 |
| Fursultiamine | 1.162374681 | 1.19103285 | −0.03 |
| Topotecan (Hydrochloride) | 0.026093259 | 0.055240793 | −0.03 |
| Tetrahydrozoline (hydrochloride) | 0.954109866 | 0.983306408 | −0.03 |
| Hydrocortisone 17-butyrate | 1.132505501 | 1.162446505 | −0.03 |
| Medroxyprogesterone | 0.978309038 | 1.008388581 | −0.03 |
| Tafamidis meglumine | 0.808839779 | 0.839781165 | −0.03 |
| Zofenopril (calcium) | 1.250329381 | 1.28132033 | −0.03 |
| Metronidazole | 0.975402369 | 1.006468305 | −0.03 |
| Ozanimod | 1.004235177 | 1.035560345 | −0.03 |
| Ampiroxicam | 0.959611297 | 0.990944373 | −0.03 |
| Abemaciclib (methanesulfonate) | 0.65344544 | 0.685320357 | −0.03 |
| Ledipasvir (D-tartrate) | 0.572800357 | 0.604716981 | −0.03 |
| 9-Aminoacridine | 0.564502349 | 0.596822244 | −0.03 |
| D-Pantothenic acid (sodium) | 0.987867178 | 1.020226785 | −0.03 |
| Peficitinib | 1.303351226 | 1.335804623 | −0.03 |
| Gemcitabine | 0.013259669 | 0.045720985 | −0.03 |
| Polymyxin B (Sulfate) | 0.946278511 | 0.979272727 | −0.03 |
| Sulfalene | 1.066883963 | 1.100138841 | −0.03 |
| Nadifloxacin | 0.955801825 | 0.989066296 | −0.03 |
| Telbivudine | 0.827506166 | 0.861317052 | −0.03 |
| Imipenem monohydrate | 1.065517241 | 1.099920064 | −0.03 |
| Cetylpyridinium (chloride) | 0.057974115 | 0.092433976 | −0.03 |
| L-Glutamic acid monosodium salt | 1.058678577 | 1.093250902 | −0.03 |
| Valganciclovir (hydrochloride) | 1.006668687 | 1.041559051 | −0.03 |
| Betamethasone | 0.61134823 | 0.646314222 | −0.03 |
| D-Pantothenic acid (hemicalcium salt) | 0.843441194 | 0.878803507 | −0.04 |
| Trimetrexate | 0.095829428 | 0.131677441 | −0.04 |
| Itopride (hydrochloride) | 1.104432855 | 1.141096841 | −0.04 |
| Hemin | 0.959991059 | 0.99681805 | −0.04 |
| Rolapitant | 0.952306664 | 0.989214695 | −0.04 |
| Cinobufotalin | −0.0340729 | 0.003241768 | −0.04 |
| Triamcinolone (acetonide) | 0.52884097 | 0.566326531 | −0.04 |
| Sisomicin (sulfate) | 0.891796789 | 0.929303279 | −0.04 |
| Octisalate | 2.150277845 | 2.188385269 | −0.04 |
| Cefozopran (hydrochloride) | 0.975897565 | 1.014147431 | −0.04 |
| Carbocisteine | 1.447131 | 1.485519591 | −0.04 |
| Gemcitabine (hydrochloride) | 0.014033825 | 0.052621544 | −0.04 |
| Sulbactam | 1.055716514 | 1.094371419 | −0.04 |
| Milrinone | 0.940677966 | 0.979347449 | −0.04 |
| Baicalin | 1.025142561 | 1.063897231 | −0.04 |
| Ponatinib | 0.075292398 | 0.11409396 | −0.04 |
| Mitoxantrone | −0.031589067 | 0.007289294 | −0.04 |
| Glecaprevir | 0.847784446 | 0.886678419 | −0.04 |
| Indirubin | 0.733727112 | 0.772655288 | −0.04 |
| Beclometasone | 1.20528072 | 1.244221544 | −0.04 |
| Tavaborole | 0.97885519 | 1.017874876 | −0.04 |
| Morinidazole (R enantiomer) | 1.064139541 | 1.104129719 | −0.04 |
| Solriamfetol | 0.965355387 | 1.006074631 | −0.04 |
| Fluocinonide | 0.544016046 | 0.584765757 | −0.04 |
| Dexamethasone acetate | 0.640346767 | 0.681524328 | −0.04 |
| Verteporfin | 0.218814433 | 0.260048232 | −0.04 |
| Nifuroxazide | 0.913925673 | 0.95531281 | −0.04 |
| L-Cysteine (hydrochloride) | 1.124092268 | 1.165839126 | −0.04 |
| Altretamine | 0.943447932 | 0.985272989 | −0.04 |
| Olanzapine | 0.921667085 | 0.96351452 | −0.04 |
| Saxagliptin | 0.923849933 | 0.96572327 | −0.04 |
| Naftidrofuryl (oxalate) | 0.973302229 | 1.015181849 | −0.04 |
| Colchicine | 0.070726691 | 0.113529049 | −0.04 |
| Sertindole | 0.503514527 | 0.546784621 | −0.04 |
| Adefovir dipivoxil | 0.137459807 | 0.181147346 | −0.04 |
| Desipramine hydrochloride | 1.005830904 | 1.049519686 | −0.04 |
| Bisacodyl | 0.755256065 | 0.798979592 | −0.04 |
| Ozagrel | 1.034028683 | 1.078618535 | −0.04 |
| Ruxolitinib (phosphate) | 0.631539611 | 0.676364943 | −0.04 |
| (S)-Timolol (Maleate) | 1.190520794 | 1.235466536 | −0.04 |
| Ondansetron | 1.123305745 | 1.16835081 | −0.05 |
| Vismodegib | 1.173896499 | 1.219135158 | −0.05 |
| Cefditoren (Pivoxil) | 0.966532198 | 1.011878788 | −0.05 |
| Cinoxacin | 0.950964117 | 0.99647444 | −0.05 |
| Clodronate (disodium tetrahydrate) | 1.034065442 | 1.079757576 | −0.05 |
| Ceritinib | 0.057593479 | 0.103819785 | −0.05 |
| Dexmedetomidine (hydrochloride) | 0.908388004 | 0.955086764 | −0.05 |
| Dexchlorpheniramine (maleate) | 1.02903752 | 1.075802469 | −0.05 |
| Triclocarban | 0.760195759 | 0.807160494 | −0.05 |
| Lappaconitine (hydrobromide) | 0.970379437 | 1.017484009 | −0.05 |
| Hydroxocobalamin (monohydrochloride) | 1.151366228 | 1.198727434 | −0.05 |
| Piroctone olamine | 0.000261233 | 0.047650273 | −0.05 |
| Cytarabine | 0.04198895 | 0.089488081 | −0.05 |
| Docetaxel (Trihydrate) | 0.202947572 | 0.250708215 | −0.05 |
| Bosentan | 0.990388914 | 1.038472664 | −0.05 |
| Levoleucovorin (Calcium) | 1.486835569 | 1.53536085 | −0.05 |
| Ethacridine (lactate monohydrate) | 0.15193133 | 0.200614125 | −0.05 |
| DL-Arginine | 1.476001444 | 1.525431979 | −0.05 |
| Methotrexate (disodium) | 0.016821491 | 0.067014147 | −0.05 |
| Isosorbide | 1.123439229 | 1.173765731 | −0.05 |
| Icotinib | 0.377436647 | 0.429530201 | −0.05 |
| Pramiracetam | 1.007171672 | 1.059393939 | −0.05 |
| Hyaluronidase | 0.850840336 | 0.903636364 | −0.05 |
| Rocuronium (Bromide) | 0.909158823 | 0.962424242 | −0.05 |
| Ceftazidime | 0.894344023 | 0.94777432 | −0.05 |
| Carbenoxolone (disodium) | 1.095045632 | 1.148733079 | −0.05 |
| Entrectinib | 0.382660944 | 0.437052201 | −0.05 |
| Osimertinib (dimesylate) | 0.137126965 | 0.191648822 | −0.05 |
| Bleomycin (sulfate) | −0.00441989 | 0.050410317 | −0.05 |
| Evans Blue | 1.110426613 | 1.165378671 | −0.05 |
| Aspartame | 0.988662438 | 1.044336744 | −0.06 |
| Talazoparib | 0.178961959 | 0.234930448 | −0.06 |
| Blonanserin | 1.087560064 | 1.143641741 | −0.06 |
| DL-Methionine | 1.325153374 | 1.381357995 | −0.06 |
| Benzoic acid | 1.062986003 | 1.119285953 | −0.06 |
| Tafenoquine (Succinate) | 1.19567474 | 1.252303157 | −0.06 |
| Roflumilast | 0.892802915 | 0.949547219 | −0.06 |
| Trametinib | 0.359481828 | 0.416396568 | −0.06 |
| Protirelin (Acetate) | 1.075293776 | 1.13257741 | −0.06 |
| Visomitin | 0.011939571 | 0.06935123 | −0.06 |
| Laurocapram | 0.887024221 | 0.944724235 | −0.06 |
| Grazoprevir potassium salt | 1.0302267 | 1.089221248 | −0.06 |
| Cefodizime | 0.92970123 | 0.988830975 | −0.06 |
| Iloperidone | 0.886277187 | 0.945472062 | −0.06 |
| Aripiprazole Lauroxil | 0.87046743 | 0.929693076 | −0.06 |
| Terazosin (hydrochloride dihydrate) | 0.820994475 | 0.88042204 | −0.06 |
| Rifapentine | 0.996686472 | 1.056251283 | −0.06 |
| Cobimetinib | 0.236132711 | 0.295795796 | −0.06 |
| Artemisinin | 0.789188142 | 0.848891181 | −0.06 |
| D-Glucose | 0.301657459 | 0.361469324 | −0.06 |
| Cimetropium (Bromide) | 0.818227467 | 0.878629933 | −0.06 |
| Tolvaptan | 0.812632858 | 0.873221216 | −0.06 |
| Flunisolide | 0.793474715 | 0.854567901 | −0.06 |
| Gadopentetate (dimeglumine) | 1.070598911 | 1.131894484 | −0.06 |
| Fosamprenavir | 0.724141619 | 0.78551035 | −0.06 |
| Fenofibrate | 0.793197692 | 0.855109961 | −0.06 |
| Sonidegib (diphosphate) | 0.877399452 | 0.939642784 | −0.06 |
| Mycophenolic acid | 0.283737761 | 0.346000613 | −0.06 |
| Clevudine | 0.912744659 | 0.975030303 | −0.06 |
| Kasugamycin (hydrochloride hydrate) | 1.193827403 | 1.25614087 | −0.06 |
| Favipiravir | 0.996647295 | 1.059299971 | −0.06 |
| Methicillin (sodium salt) | 1.051238592 | 1.114022909 | −0.06 |
| Temsirolimus | 0.354412002 | 0.417609716 | −0.06 |
| Phenazopyridine (hydrochloride) | 1.130508475 | 1.1938563 | −0.06 |
| Mestranol | 0.927655554 | 0.991204452 | −0.06 |
| Ixazomib | −0.013361676 | 0.050452781 | −0.06 |
| Levosimendan | 1.131314879 | 1.195307702 | −0.06 |
| Telmisartan | 1.255324357 | 1.319402985 | −0.06 |
| Desoximetasone | 0.771637317 | 0.835857352 | −0.06 |
| Ligustrazine (hydrochloride) | 0.982893157 | 1.047151515 | −0.06 |
| Danazol | 0.760449321 | 0.824918033 | −0.06 |
| Selumetinib (sulfate) | 0.328705681 | 0.393247269 | −0.06 |
| Apixaban | 0.934939492 | 0.999794408 | −0.06 |
| Tofacitinib | 1.237729487 | 1.302882267 | −0.07 |
| Omeprazole (sodium) | 0.698898278 | 0.764097074 | −0.07 |
| Ozagrel (sodium) | 1.068366312 | 1.133904324 | −0.07 |
| L-Thyroxine (sodium) | 1.070889657 | 1.136768902 | −0.07 |
| Azasetron (hydrochloride) | 0.761243896 | 0.827713816 | −0.07 |
| Almonertinib (hydrochloride) | 0.361842105 | 0.428411633 | −0.07 |
| Chloramphenicol succinate (sodium) | 0.844743935 | 0.911904762 | −0.07 |
| Flopropione | 0.951499531 | 1.0190541 | −0.07 |
| Lacidipine | 1.012646675 | 1.080701145 | −0.07 |
| 2-Ethoxybenzamide | 0.955912649 | 1.024068913 | −0.07 |
| Lapatinib | 0.275334049 | 0.343545279 | −0.07 |
| Miltefosine | 0.934487021 | 1.002786927 | −0.07 |
| Isosulfan blue | 1.087355831 | 1.15590864 | −0.07 |
| Ibrutinib | 0.376548484 | 0.445878848 | −0.07 |
| Toltrazuril | 0.901244168 | 0.971805138 | −0.07 |
| Benztropine (mesylate) | 0.788338317 | 0.85898709 | −0.07 |
| Carboxin | 1.32593801 | 1.396790123 | −0.07 |
| Desonide | 0.581141869 | 0.652376858 | −0.07 |
| Pitavastatin (Calcium) | 0.369319608 | 0.44080417 | −0.07 |
| Natamycin | 0.989321943 | 1.060836502 | −0.07 |
| Estradiol dipropionate | 0.92467989 | 0.996276992 | −0.07 |
| Norvancomycin (hydrochloride) | 1.172546152 | 1.244390376 | −0.07 |
| Diatrizoic acid | 1.003965015 | 1.076579624 | −0.07 |
| Penicillin G Procaine | 1.02266436 | 1.095565655 | −0.07 |
| DL-Glutamine | 1.6975821 | 1.77074714 | −0.07 |
| Vonoprazan | 1.031278469 | 1.104786546 | −0.07 |
| Famotidine | 0.942390671 | 1.015965363 | −0.07 |
| Pralidoxime (iodide) | 0.975690152 | 1.049404611 | −0.07 |
| Tanshinone I | 0.78304787 | 0.857214304 | −0.07 |
| Sofalcone | 0.845342707 | 0.920327625 | −0.07 |
| Dichlorphenamide | 0.971386152 | 1.046490756 | −0.08 |
| Lanolin | 1.01974045 | 1.095160854 | −0.08 |
| Hesperidin | 1.0302267 | 1.1057246 | −0.08 |
| Lisinopril (dihydrate) | 1.657163479 | 1.733755172 | −0.08 |
| Iodipamide | 0.876010782 | 0.952721088 | −0.08 |
| Vancomycin (hydrochloride) | 0.753734791 | 0.830499742 | −0.08 |
| Argatroban (monohydrate) | 1.154782073 | 1.232354456 | −0.08 |
| Parecoxib (Sodium) | 0.855147282 | 0.932729625 | −0.08 |
| Acitretin | 0.728534814 | 0.806355511 | −0.08 |
| Alogliptin (Benzoate) | 0.807364836 | 0.885381551 | −0.08 |
| Lamotrigine | 0.964420485 | 1.042517007 | −0.08 |
| Fimasartan | 1.248917509 | 1.327097039 | −0.08 |
| Manidipine (dihydrochloride) | 0.73571755 | 0.814001232 | −0.08 |
| Nitisinone | 0.796300423 | 0.874620829 | −0.08 |
| Setiptiline | 1.023862965 | 1.102762625 | −0.08 |
| Amenamevir | 0.871823204 | 0.950762016 | −0.08 |
| Gallic acid | 1.221662469 | 1.300670449 | −0.08 |
| Cabozantinib | 0.942962056 | 1.022601279 | −0.08 |
| Dimemorfan (phosphate) | 0.855248619 | 0.935130911 | −0.08 |
| Sitagliptin | 0.914473684 | 0.994407159 | −0.08 |
| Ertugliflozin | 0.990699477 | 1.070654977 | −0.08 |
| Mildronate | 0.914575547 | 0.994930292 | −0.08 |
| Repaglinide | 1.007734807 | 1.088315748 | −0.08 |
| Clindamycin (hydrochloride) | 0.927640156 | 1.008503992 | −0.08 |
| Benzthiazide | 0.875066738 | 0.956062526 | −0.08 |
| Lenampicillin (hydrochloride) | 1.020151134 | 1.101239276 | −0.08 |
| Larotrectinib | 0.93048726 | 1.011859994 | −0.08 |
| Almotriptan (malate) | 0.760466472 | 0.842240563 | −0.08 |
| Diethyltoluamide | 1.014546884 | 1.096330275 | −0.08 |
| Cytarabine (hydrochloride) | 0.106375686 | 0.188257031 | −0.08 |
| Irbesartan | 0.788679245 | 0.871088435 | −0.08 |
| Sodium diatrizoate | 1.044862079 | 1.127964311 | −0.08 |
| Irinotecan | 0.184234033 | 0.267610063 | −0.08 |
| Plerixafor (octahydrochloride) | 1.13747282 | 1.220963173 | −0.08 |
| Fluorometholone | 1.165498913 | 1.249291785 | −0.08 |
| Fumagillin | 0.518955561 | 0.603127327 | −0.08 |
| Glycyrrhizic acid | 1.061263318 | 1.145602674 | −0.08 |
| (+)-Kavain | 1.053862787 | 1.138354543 | −0.08 |
| Rotigotine (Hydrochloride) | 0.794318667 | 0.878848064 | −0.08 |
| Teniposide | 0.097392438 | 0.183096147 | −0.09 |
| Phenacetin | 0.868797497 | 0.955163437 | −0.09 |
| Gatifloxacin (hydrochloride) | 0.951242961 | 1.037813212 | −0.09 |
| Bithionol | 0.844215002 | 0.931435964 | −0.09 |
| Talipexole dihydrochloride | 0.92816092 | 1.015519126 | −0.09 |
| Berberine (chloride) | 0.187288272 | 0.27518631 | −0.09 |
| Pirarubicin (Hydrochloride) | 0.560502283 | 0.648422812 | −0.09 |
| Allantoin | 0.482270878 | 0.570397112 | −0.09 |
| Amikacin (disulfate) | 0.868509026 | 0.95685878 | −0.09 |
| Eperisone (Hydrochloride) | 0.888948211 | 0.978031263 | −0.09 |
| Proguanil | 1.058838777 | 1.147981315 | −0.09 |
| 18β-Glycyrrhetinic acid | 1.102326312 | 1.191690442 | −0.09 |
| Bromhexine (hydrochloride) | 1.254409278 | 1.344192635 | −0.09 |
| Acetylleucine | 0.820608649 | 0.91043515 | −0.09 |
| Quetiapine | 0.758132002 | 0.848321666 | −0.09 |
| Mepivacaine | 1.156273398 | 1.246609565 | −0.09 |
| Asunaprevir | 0.990233384 | 1.081261751 | −0.09 |
| Vinpocetine | 0.820876931 | 0.911997126 | −0.09 |
| Pericyazine | 0.835285848 | 0.926505614 | −0.09 |
| Regorafenib | 0.600984067 | 0.692412385 | −0.09 |
| Panaxatriol | 1.044032186 | 1.135666763 | −0.09 |
| Valsartan | 0.806476484 | 0.8984375 | −0.09 |
| Stavudine | 0.899401965 | 0.992055611 | −0.09 |
| Fenoterol (hydrobromide) | 1.310929853 | 1.403703704 | −0.09 |
| D-Panthenol | 0.908684547 | 1.001838799 | −0.09 |
| Eprazinone (dihydrochloride) | 1.171941272 | 1.265432099 | −0.09 |
| Guacetisal | 0.74339508 | 0.836998706 | −0.09 |
| Dabigatran etexilate | 0.873827602 | 0.968238994 | −0.09 |
| Salicylic acid | 0.882872928 | 0.977334896 | −0.09 |
| Grazoprevir | 0.962643678 | 1.057486339 | −0.09 |
| Tetrahydrobiopterin | 0.85449266 | 0.949765388 | −0.10 |
| Etoposide phosphate | 0.691651543 | 0.787636557 | −0.10 |
| LCZ696 | 1.053160766 | 1.149536046 | −0.10 |
| Melitracen (hydrochloride) | 1.186750541 | 1.28336786 | −0.10 |
| Temoporfin | 0.751382041 | 0.848 | −0.10 |
| Carglumic Acid | 0.9389347 | 1.036400404 | −0.10 |
| Ioversol | 1.064288765 | 1.161866931 | −0.10 |
| Fondaparinux (sodium) | 1.000181488 | 1.097788436 | −0.10 |
| sn-Glycero-3-phosphocholine | 0.81243945 | 0.910778752 | −0.10 |
| Ciclopirox | 0.174117121 | 0.272490599 | −0.10 |
| Delavirdine (mesylate) | 0.97287484 | 1.071499503 | −0.10 |
| Dasatinib (hydrochloride) | 0.284113916 | 0.383055107 | −0.10 |
| Fludarabine (phosphate) | 0.976931052 | 1.075909243 | −0.10 |
| Oxybutynin | 0.976084041 | 1.075498988 | −0.10 |
| Mizoribine | 0.779471089 | 0.879030303 | −0.10 |
| Pranlukast (hemihydrate) | 0.975484734 | 1.075497135 | −0.10 |
| Medetomidine | 0.934244373 | 1.034277553 | −0.10 |
| Vecuronium (bromide) | 0.94380704 | 1.044602569 | −0.10 |
| Iopanoic acid | 0.919067055 | 1.020294953 | −0.10 |
| Trandolapril | 0.967113703 | 1.068461643 | −0.10 |
| Orphenadrine (citrate) | 0.76801406 | 0.869694713 | −0.10 |
| Diflorasone | 0.651804124 | 0.753617363 | −0.10 |
| Pranlukast | 0.895252953 | 0.997303674 | −0.10 |
| Metyrosine | 1.504150126 | 1.606230226 | −0.10 |
| Eicosapentaenoic Acid | 0.968979592 | 1.071708835 | −0.10 |
| L-5-Hydroxytryptophan | 0.924528302 | 1.02755102 | −0.10 |
| Tazobactam | 0.900577454 | 1.003723008 | −0.10 |
| Ramelteon | 0.840896957 | 0.944233207 | −0.10 |
| Irinotecan (hydrochloride trihydrate) | 0.280816785 | 0.384465071 | −0.10 |
| Betaxolol | 1.078706199 | 1.182653061 | −0.10 |
| ATP | 1.465734046 | 1.569721826 | −0.10 |
| Clopidogrel (hydrogen sulfate) | 1.052641199 | 1.156799135 | −0.10 |
| Betahistine | 1.087295826 | 1.191580069 | −0.10 |
| Tedizolid | 0.807261209 | 0.91163311 | −0.10 |
| Ivacaftor | 0.877986233 | 0.982588404 | −0.10 |
| trans-Tranilast | 0.840570456 | 0.945448973 | −0.10 |
| Lenvatinib (mesylate) | 0.740073145 | 0.845027322 | −0.10 |
| Lenvatinib | 1.076980568 | 1.182112069 | −0.11 |
| Lovastatin | 0.603022563 | 0.70885688 | −0.11 |
| Phenothiazine | 1.002351097 | 1.108196721 | −0.11 |
| L-Ornithine (hydrochloride) | 1.327868852 | 1.433985035 | −0.11 |
| Biapenem | 0.873267769 | 0.979461961 | −0.11 |
| Dorzolamide (hydrochloride) | 0.937847074 | 1.04468719 | −0.11 |
| S-Adenosyl-L-methionine (disulfate tosylate) | 1.004497316 | 1.111644134 | −0.11 |
| Crocin | 0.991361857 | 1.098905044 | −0.11 |
| Roxadustat | 0.966213822 | 1.073904628 | −0.11 |
| Fluvoxamine (maleate) | 1.033532678 | 1.142005958 | −0.11 |
| Lipoic acid | 0.752761093 | 0.861268695 | −0.11 |
| Ceftezole (sodium) | 0.980589963 | 1.089146311 | −0.11 |
| Sugammadex (sodium) | 1.48682786 | 1.595522025 | −0.11 |
| Cefaclor | 0.911055837 | 1.019782777 | −0.11 |
| Doxepin (Hydrochloride) | 1.121545064 | 1.230296827 | −0.11 |
| Enfuvirtide (acetate) | 0.876457375 | 0.985367595 | −0.11 |
| 6-Mercaptopurine hydrate | 0.279631919 | 0.388649607 | −0.11 |
| Sertraline (hydrochloride) | 0.998380348 | 1.108239095 | −0.11 |
| Miconazole (nitrate) | 0.813561486 | 0.923948628 | −0.11 |
| Oxyphenbutazone | 1.170820409 | 1.281584582 | −0.11 |
| Darifenacin (hydrobromide) | 1.074830687 | 1.185649671 | −0.11 |
| Pimobendan | 0.993236152 | 1.10418076 | −0.11 |
| Gestodene | 1.023194698 | 1.134264011 | −0.11 |
| Miglitol | 0.804851752 | 0.915986395 | −0.11 |
| Sotagliflozin | 0.989549839 | 1.10092835 | −0.11 |
| Glafenine (hydrochloride) | 1.255464927 | 1.367160494 | −0.11 |
| Fosamprenavir (Calcium Salt) | 0.949544765 | 1.061427591 | −0.11 |
| Clemizole (hydrochloride) | 1.012656822 | 1.124794408 | −0.11 |
| Ribavirin | 0.728056704 | 0.840225233 | −0.11 |
| Pregnenolone monosulfate | 0.92017051 | 1.032490975 | −0.11 |
| L-Carnitine | 0.940177638 | 1.053114754 | −0.11 |
| Tazarotene | 0.617725574 | 0.73130545 | −0.11 |
| Tinoridine hydrochloride | 1.039113104 | 1.153376437 | −0.11 |
| Oxaprozin | 1.010797678 | 1.12547119 | −0.11 |
| Dacarbazine | 1.076643599 | 1.191376981 | −0.11 |
| Ciprofibrate | 0.974577259 | 1.089568394 | −0.11 |
| Omeprazole | 0.879752243 | 0.995034757 | −0.12 |
| Sodium gualenate | 0.854223272 | 0.970438149 | −0.12 |
| Pazopanib (Hydrochloride) | 0.972337821 | 1.0891258 | −0.12 |
| Tasimelteon | 0.822832618 | 0.940634596 | −0.12 |
| Povidone iodine | 0.828631453 | 0.946787879 | −0.12 |
| Glafenine | 1.169983687 | 1.288148148 | −0.12 |
| Tenofovir alafenamide fumarate | 0.707787201 | 0.826069079 | −0.12 |
| Phenformin (hydrochloride) | 0.847178683 | 0.96568306 | −0.12 |
| Clonidine (hydrochloride) | 0.816226024 | 0.935101768 | −0.12 |
| L-Methionine | 0.782212749 | 0.901495616 | −0.12 |
| Metadoxine | 1.009751408 | 1.129465417 | −0.12 |
| Clofibrate | 1.017783505 | 1.137861736 | −0.12 |
| Lesinurad | 0.971613768 | 1.091698004 | −0.12 |
| Voglibose | 1.026868178 | 1.14699714 | −0.12 |
| Taurochenodeoxycholic acid | 0.846932043 | 0.967554645 | −0.12 |
| Flavin adenine dinucleotide (disodium salt) | 0.803314917 | 0.924189136 | −0.12 |
| Fasudil (Hydrochloride) | 1.000213584 | 1.121151936 | −0.12 |
| Novobiocin (Sodium) | 1.071721994 | 1.193050987 | −0.12 |
| Norethindrone acetate | 0.911603499 | 1.033283723 | −0.12 |
| Sulfadiazine (sodium) | 1.095281307 | 1.217159606 | −0.12 |
| Pamidronate (disodium pentahydrate) | 1.183217993 | 1.305367891 | −0.12 |
| Fertirelin | 0.864150943 | 0.986734694 | −0.12 |
| Cefamandole (sodium) | 1.119913505 | 1.242527375 | −0.12 |
| Dacomitinib | 0.237101336 | 0.35973025 | −0.12 |
| Lomerizine dihydrochloride | 0.899876391 | 1.023055485 | −0.12 |
| Bromocriptine (mesylate) | 0.812658549 | 0.935867976 | −0.12 |
| Propyphenazone | 1.004555117 | 1.128072445 | −0.12 |
| Taltirelin (acetate) | 0.967938488 | 1.091583079 | −0.12 |
| Deferoxamine (mesylate) | 0.873854447 | 0.997619048 | −0.12 |
| Folinic acid | 0.981306715 | 1.105249134 | −0.12 |
| Fenoldopam (mesylate) | 0.688165177 | 0.81305547 | −0.12 |
| Tigecycline | 1.106013526 | 1.231413896 | −0.13 |
| Methotrexate | 0.099436248 | 0.224976168 | −0.13 |
| Olopatadine (hydrochloride) | 0.891656024 | 1.017775054 | −0.13 |
| Lodoxamide (tromethamine) | 0.758553713 | 0.884848485 | −0.13 |
| Zaltoprofen | 1.024956268 | 1.151265052 | −0.13 |
| Mebrofenin | 0.961366682 | 1.087735849 | −0.13 |
| Bromperidol | 1.050417973 | 1.17726776 | −0.13 |
| Opicapone | 0.759093174 | 0.886135057 | −0.13 |
| Thonzonium (bromide) | 0.051549755 | 0.179012346 | −0.13 |
| Fedratinib (hydrochloride hydrate) | 0.341016334 | 0.468958167 | −0.13 |
| Florfenicol | 0.726212945 | 0.854235197 | −0.13 |
| Ciclopirox (olamine) | 0.269058869 | 0.397129187 | −0.13 |
| Bosentan (hydrate) | 1.181414455 | 1.309827303 | −0.13 |
| 4-Phenylbutyric acid | 1.079165167 | 1.208007626 | −0.13 |
| Dimethyl fumarate | 0.732596685 | 0.861664713 | −0.13 |
| Econazole (nitrate) | 0.837505588 | 0.966734163 | −0.13 |
| Idelalisib | 0.96715889 | 1.096427943 | −0.13 |
| Troxerutin | 1.020600858 | 1.150460594 | −0.13 |
| Trimebutine (maleate) | 0.814159292 | 0.944177933 | −0.13 |
| Salbutamol (hemisulfate) | 1.464201043 | 1.594546628 | −0.13 |
| Cefoxitin | 0.754958574 | 0.886075949 | −0.13 |
| Verapamil (hydrochloride) | 0.855821126 | 0.987253289 | −0.13 |
| Bucladesine (calcium) | 0.762937595 | 0.894923752 | −0.13 |
| Safinamide | 0.953715149 | 1.085888016 | −0.13 |
| Thio-TEPA | 0.794117647 | 0.926722032 | −0.13 |
| 5-Fluorouracil | 0.340096103 | 0.472750317 | −0.13 |
| Rapamycin | 0.218679387 | 0.351451973 | −0.13 |
| Acetohexamide | 1.030326594 | 1.163329997 | −0.13 |
| Tinidazole | 0.987639485 | 1.120777892 | −0.13 |
| Triprolidine (hydrochloride monohydrate) | 1.107040417 | 1.240367928 | −0.13 |
| Diacerein | 0.944444444 | 1.077842476 | −0.13 |
| Sulfameter | 0.782403269 | 0.915890204 | −0.13 |
| Sodium nitroprusside | 0.970135948 | 1.103808561 | −0.13 |
| Tripelennamine (hydrochloride) | 1.050692273 | 1.184591195 | −0.13 |
| Ledipasvir (acetone) | 0.433223993 | 0.567199728 | −0.13 |
| Acarbose | 0.921613981 | 1.056332237 | −0.13 |
| Silver sulfadiazine | 0.870895283 | 1.006067095 | −0.14 |
| Ilaprazole | 1.190302144 | 1.325503356 | −0.14 |
| L-Epinephrine (Bitartrate) | 1.111715915 | 1.247414081 | −0.14 |
| Perhexiline maleate | 1.003031222 | 1.139234562 | −0.14 |
| Tamsulosin | 0.869541779 | 1.005782313 | −0.14 |
| Stearic acid | 1.022476264 | 1.159360495 | −0.14 |
| Cariprazine (hydrochloride) | 1.020711501 | 1.157718121 | −0.14 |
| Masitinib (mesylate) | 0.889904168 | 1.026963263 | −0.14 |
| Penfluridol | 0.604651163 | 0.741885626 | −0.14 |
| Alfuzosin | 0.837196765 | 0.974489796 | −0.14 |
| Risperidone | 0.961566707 | 1.099360341 | −0.14 |
| Sulfacetamide | 1.008498224 | 1.14643827 | −0.14 |
| Benzyl alcohol | 0.894504925 | 1.032532533 | −0.14 |
| Esaxerenone | 1.152093079 | 1.290371783 | −0.14 |
| Brimonidine (tartrate) | 0.95451895 | 1.092815587 | −0.14 |
| Palmatine (chloride) | 0.625516834 | 0.764209045 | −0.14 |
| L-Ascorbic acid | 0.861856109 | 1.000612933 | −0.14 |
| Dronedarone | 0.867436438 | 1.006373355 | −0.14 |
| Iohexol | 0.922798834 | 1.061967257 | −0.14 |
| Cangrelor (tetrasodium) | 1.050272232 | 1.189448441 | −0.14 |
| Adenosine | 0.805464057 | 0.944816916 | −0.14 |
| Crisaborole | 1.059221349 | 1.198972695 | −0.14 |
| Dihydroergotoxine (mesylate) | 0.812644564 | 0.952713816 | −0.14 |
| Gramicidin | 0.04897582 | 0.19062339 | −0.14 |
| Loperamide (hydrochloride) | 0.963148789 | 1.104901118 | −0.14 |
| Carboprost (tromethamine) | 1.079687667 | 1.221627409 | −0.14 |
| Alpha-Estradiol | 0.856060606 | 0.998032787 | −0.14 |
| Lomustine | 0.964978541 | 1.107471853 | −0.14 |
| Trifluridine | 0.145016193 | 0.287511916 | −0.14 |
| Aminophylline | 0.758082252 | 0.900614754 | −0.14 |
| Nomegestrol acetate | 0.773390558 | 0.916069601 | −0.14 |
| L-Ascorbic acid (sodium salt) | 0.82320442 | 0.966393122 | −0.14 |
| Osimertinib | 0.333218884 | 0.476970317 | −0.14 |
| Lofexidine | 0.736696467 | 0.880478088 | −0.14 |
| Domperidone | 0.941161223 | 1.085251919 | −0.14 |
| Piperacillin (sodium) | 1.13795776 | 1.282104177 | −0.14 |
| Gabapentin enacarbil | 0.872695566 | 1.016882184 | −0.14 |
| Osimertinib mesylate | 0.199226804 | 0.343649518 | −0.14 |
| Memantine (hydrochloride) | 0.656045298 | 0.80078125 | −0.14 |
| Carbazochrome | 0.966823406 | 1.111693989 | −0.14 |
| Sodium 4-aminosalicylate (dihydrate) | 0.947677523 | 1.092944656 | −0.15 |
| Avatrombopag (hydrochloride) | 0.767268041 | 0.91278135 | −0.15 |
| Levodropropizine | 0.799890125 | 0.946034963 | −0.15 |
| Isoconazole (nitrate) | 1.383370009 | 1.530492154 | −0.15 |
| Dasatinib | 0.333304743 | 0.48060512 | −0.15 |
| Laquinimod | 0.979709526 | 1.127110228 | −0.15 |
| α-Lipoic Acid | 1.04414838 | 1.192131148 | −0.15 |
| Disopyramide | 0.95747471 | 1.105520274 | −0.15 |
| Cisatracurium (besylate) | 1.173285875 | 1.321446019 | −0.15 |
| Bupivacaine (hydrochloride) | 1.010257795 | 1.158499372 | −0.15 |
| Mecillinam | 0.752807787 | 0.901142041 | −0.15 |
| Pirmenol (hydrochloride) | 0.723826262 | 0.87267312 | −0.15 |
| Iopamidol | 0.987061995 | 1.136394558 | −0.15 |
| Omadacycline (hydrochloride) | 0.9530845 | 1.102602603 | −0.15 |
| Racanisodamine | 1.034258712 | 1.184409643 | −0.15 |
| Danthron | 0.792116495 | 0.942665674 | −0.15 |
| Lapatinib (ditosylate) | 0.284726688 | 0.43537253 | −0.15 |
| Azelnidipine | 0.556669237 | 0.707648026 | −0.15 |
| Efonidipine (hydrochloride monoethanolate) | 0.832474227 | 0.983667914 | −0.15 |
| Edrophonium (chloride) | 0.982115086 | 1.133967301 | −0.15 |
| Fludarabine | 1.022032141 | 1.174007341 | −0.15 |
| Gabexate (mesylate) | 0.973045822 | 1.125510204 | −0.15 |
| Methacholine (chloride) | 0.897096941 | 1.049883217 | −0.15 |
| L-Thyroxine (sodium salt pentahydrate) | 1.084922634 | 1.237750238 | −0.15 |
| Trifluridine/tipiracil hydrochloride mixture | 0.162540193 | 0.315401095 | −0.15 |
| Quinagolide (hydrochloride) | 0.975341426 | 1.128689087 | −0.15 |
| Alprenolol (hydrochloride) | 1.044323484 | 1.198031365 | −0.15 |
| Naloxegol (oxalate) | 0.871241326 | 1.025082237 | −0.15 |
| Lifitegrast | 0.955152162 | 1.109843684 | −0.15 |
| Doxycycline (hyclate) | 0.935462842 | 1.090419993 | −0.15 |
| Carbidopa | 0.994574695 | 1.150077359 | −0.16 |
| Torsemide | 0.914013841 | 1.069524628 | −0.16 |
| Rasagiline (mesylate) | 0.725632615 | 0.881592378 | −0.16 |
| Diquafosol (tetrasodium) | 0.753580925 | 0.90961986 | −0.16 |
| Azatadine (dimaleate) | 1.130112026 | 1.286304075 | −0.16 |
| DL-Methionine methylsulfonium (chloride) | 0.945440957 | 1.101652299 | −0.16 |
| Cefetamet pivoxil (hydrochloride) | 0.919557295 | 1.075928918 | −0.16 |
| Pamabrom | 0.771701778 | 0.928484848 | −0.16 |
| 4-Aminosalicylic acid | 0.937459807 | 1.094263271 | −0.16 |
| Azlocillin (sodium salt) | 1.060173836 | 1.217054264 | −0.16 |
| Sivelestat | 1.016733901 | 1.174787879 | −0.16 |
| Nitrofurantoin | 0.95734917 | 1.115419135 | −0.16 |
| L-Arginine (hydrochloride) | 1.25573719 | 1.414229672 | −0.16 |
| Lenalidomide | 0.873857811 | 1.032355915 | −0.16 |
| Neticonazole | 0.790431187 | 0.949322541 | −0.16 |
| Paliperidone palmitate | 1.074954628 | 1.23421263 | −0.16 |
| Ambrisentan | 0.970311833 | 1.130089374 | −0.16 |
| Leflunomide | 0.713955281 | 0.873766447 | −0.16 |
| Teriflunomide | 0.931812256 | 1.0918084 | −0.16 |
| Hexythiazox | 1.395758564 | 1.556790123 | −0.16 |
| N-Acetyl-L-tyrosine | 1.058838777 | 1.220720721 | −0.16 |
| Mafenide (Acetate) | 1.016733901 | 1.178666667 | −0.16 |
| Amcinonide | 0.918760196 | 1.080740741 | −0.16 |
| Oclacitinib (maleate) | 0.547583365 | 0.709819248 | −0.16 |
| Candesartan Cilexetil | 0.930385852 | 1.093311116 | −0.16 |
| Oseltamivir (phosphate) | 1.116539668 | 1.279515152 | −0.16 |
| Lactulose | 0.945614821 | 1.108848485 | −0.16 |
| Oxytocin (acetate) | 1.570438366 | 1.733756717 | −0.16 |
| Amoxicillin (sodium) | 0.732864675 | 0.896500372 | −0.16 |
| Aripiprazole | 0.833035315 | 0.99681805 | −0.16 |
| Valbenazine | 1.134527509 | 1.298459043 | −0.16 |
| Ajmaline | 0.94299194 | 1.106984315 | −0.16 |
| Dihydroergocristine (mesylate) | 1.058722858 | 1.224482512 | −0.17 |
| Isocorydine | 0.996111975 | 1.161995329 | −0.17 |
| Erlotinib | 0.442880643 | 0.609624223 | −0.17 |
| Uracil | 0.865261814 | 1.032485443 | −0.17 |
| Ombitasvir | 0.833133047 | 1.001023541 | −0.17 |
| Ledipasvir | 0.414211965 | 0.582147477 | −0.17 |
| Lactitol (monohydrate) | 0.890795632 | 1.059319178 | −0.17 |
| Crotamiton | 0.91554559 | 1.08441092 | −0.17 |
| Glucosamine (hydrochloride) | 0.946447231 | 1.116065574 | −0.17 |
| Naratriptan (hydrochloride) | 0.907679849 | 1.077364926 | −0.17 |
| Bulleyaconitine A | 1.09143149 | 1.261395115 | −0.17 |
| Mirtazapine | 1.005345502 | 1.175807011 | −0.17 |
| Neomycin (sulfate) | 1.4211476 | 1.591628133 | −0.17 |
| Nintedanib | 0.536173633 | 0.706736491 | −0.17 |
| Vorinostat | 0.315363881 | 0.486054422 | −0.17 |
| Mepyramine maleate | 1.356003986 | 1.526939655 | −0.17 |
| Amiloride (hydrochloride) | 1.469920271 | 1.641643059 | −0.17 |
| Nicotinamide | 0.716022099 | 0.888237593 | −0.17 |
| Pyrantel (tartrate) | 0.981490159 | 1.153793807 | −0.17 |
| Glycerol phenylbutyrate | 0.881749271 | 1.054390475 | −0.17 |
| Sulfachloropyridazine | 0.884676989 | 1.057456696 | −0.17 |
| Chloropyramine hydrochloride | 1.384665579 | 1.557777778 | −0.17 |
| Mizolastine | 1.272771201 | 1.446175637 | −0.17 |
| Citric acid | 0.746588694 | 0.920013059 | −0.17 |
| Ivosidenib | 0.732570926 | 0.906079235 | −0.17 |
| Arterolane | 0.993443237 | 1.167073766 | −0.17 |
| Atrasentan (hydrochloride) | 1.024787852 | 1.198427673 | −0.17 |
| Hydrocortisone buteprate | 0.638239175 | 0.812202097 | −0.17 |
| Pentamidine (isethionate) | 0.468825353 | 0.643220956 | −0.17 |
| Amifostine | 1.212558643 | 1.387198832 | −0.17 |
| Nikethamide | 0.895276968 | 1.070626438 | −0.18 |
| Sulfaphenazole | 0.998920232 | 1.174295459 | −0.18 |
| Carvedilol | 0.957887875 | 1.133460604 | −0.18 |
| Dimesna | 0.896995708 | 1.072671443 | −0.18 |
| Milnacipran ((1S-cis)hydrochloride) | 0.893986522 | 1.069903237 | −0.18 |
| Methylene blue (trihydrate) | 0.257350712 | 0.433904672 | −0.18 |
| Arotinolol | 0.988754325 | 1.165335954 | −0.18 |
| Dapagliflozin | 0.874977152 | 1.051905921 | −0.18 |
| Ibuprofen piconol | 1.081963129 | 1.259698276 | −0.18 |
| Methylcobalamin | 0.843498542 | 1.021377351 | −0.18 |
| (R)-Lansoprazole | 0.805412371 | 0.9835209 | −0.18 |
| Promazine (hydrochloride) | 1.064261898 | 1.243484386 | −0.18 |
| Retapamulin | 0.822097806 | 1.002052967 | −0.18 |
| Dimercaprol | 0.811455315 | 0.991911021 | −0.18 |
| Tafluprost | 0.773584906 | 0.954081633 | −0.18 |
| Anethole (trithione) | 1.382013593 | 1.562923524 | −0.18 |
| Nabumetone | 0.853294461 | 1.034366121 | −0.18 |
| Clofibric acid | 0.812066204 | 0.993240389 | −0.18 |
| Monocrotaline | 0.828856624 | 1.010391687 | −0.18 |
| Halobetasol (propionate) | 0.56405378 | 0.746187591 | −0.18 |
| Dabrafenib | 0.543295924 | 0.725497097 | −0.18 |
| Paliperidone | 0.656916648 | 0.839139344 | −0.18 |
| Pamidronic acid | 0.630939227 | 0.813208284 | −0.18 |
| Safinamide (mesylate) | 1.040834846 | 1.22355449 | −0.18 |
| Neostigmine (Bromide) | 0.825244785 | 1.007968127 | −0.18 |
| Clobetasone butyrate | 0.500446942 | 0.683203223 | −0.18 |
| Halcinonide | 0.711701984 | 0.894991922 | −0.18 |
| Lercanidipine (hydrochloride) | 0.706094421 | 0.889457523 | −0.18 |
| Berberine (chloride hydrate) | 0.079558011 | 0.262993357 | −0.18 |
| Cefuroxime axetil | 0.676672104 | 0.860493827 | −0.18 |
| Edaravone | 1.052328065 | 1.236345581 | −0.18 |
| Cabozantinib (S-malate) | 0.826850984 | 1.010888057 | −0.18 |
| Atazanavir (sulfate) | 1.022426314 | 1.206554121 | −0.18 |
| Bacampicillin (hydrochloride) | 1.027770535 | 1.211905588 | −0.18 |
| Selumetinib | 0.289909237 | 0.47444022 | −0.18 |
| Carprofen | 0.886008584 | 1.07062436 | −0.18 |
| Nitroprusside (disodium dihydrate) | 0.816646065 | 1.001366743 | −0.18 |
| Propofol | 0.789757412 | 0.974489796 | −0.18 |
| Raltegravir | 0.950492483 | 1.135301969 | −0.18 |
| Salicylanilide | 1.046347715 | 1.231380338 | −0.19 |
| Beclometasone dipropionate | 0.820290534 | 1.005444029 | −0.19 |
| Carteolol hydrochloride | 0.928411105 | 1.114024283 | −0.19 |
| Teriparatide | 0.875708155 | 1.061412487 | −0.19 |
| Tetrahydropalmatine | 0.898373241 | 1.084347121 | −0.19 |
| Tolcapone | 0.962712484 | 1.149229521 | −0.19 |
| Butoconazole (nitrate) | 0.797692625 | 0.984545224 | −0.19 |
| Triflupromazine (hydrochloride) | 0.80664723 | 0.993776214 | −0.19 |
| 10-Undecenoic acid (zinc salt) | 1.075836988 | 1.26302641 | −0.19 |
| Cloxacillin (sodium monohydrate) | 0.926645931 | 1.113947281 | −0.19 |
| Peretinoin | 1.084079693 | 1.271424709 | −0.19 |
| Cytisinicline | 0.967606965 | 1.155627356 | −0.19 |
| Ganciclovir (sodium) | 0.721817725 | 0.909903202 | −0.19 |
| Vilazodone carboxylic acid | 0.769556497 | 0.958206943 | −0.19 |
| Diphenhydramine (hydrochloride) | 0.893562232 | 1.082906858 | −0.19 |
| Masitinib | 0.68814433 | 0.877509357 | −0.19 |
| Uridine | 1.214951563 | 1.404433631 | −0.19 |
| Lumacaftor | 0.854044834 | 1.043624161 | −0.19 |
| Oleanolic Acid | 0.81109339 | 1.000979432 | −0.19 |
| Felypressin | 0.800946687 | 0.991020115 | −0.19 |
| Chloroxylenol | 1.028430161 | 1.218647074 | −0.19 |
| Encorafenib | 0.31160221 | 0.502149277 | −0.19 |
| Almonertinib | 0.273496014 | 0.464589235 | −0.19 |
| Xanthinol Nicotinate | 0.66835276 | 0.859631148 | −0.19 |
| Vitamin B12 | 0.919905493 | 1.111208869 | −0.19 |
| Pemetrexed (disodium) | 0.125053607 | 0.316912335 | −0.19 |
| Felodipine | 1.010300783 | 1.202432227 | −0.19 |
| Cefuroxime (sodium) | 0.953431017 | 1.145660706 | −0.19 |
| Bismuth subgallate | 0.933607194 | 1.126439145 | −0.19 |
| Captopril | 0.704007994 | 0.896998355 | −0.19 |
| Protriptyline (hydrochloride) | 1.153416704 | 1.346855346 | −0.19 |
| Bepridil hydrochloride | 0.815721649 | 1.009244373 | −0.19 |
| Lenalidomide (hemihydrate) | 0.986118526 | 1.180819603 | −0.19 |
| Mozavaptan | 0.633164724 | 0.828210383 | −0.20 |
| Acetylspiramycin | 1.224456224 | 1.419572858 | −0.20 |
| Baricitinib (phosphate) | 0.581054833 | 0.776242424 | −0.20 |
| (±)-Bisoprolol (hemifumarate) | 0.88131426 | 1.07665606 | −0.20 |
| Urea | 1.13795776 | 1.333677153 | −0.20 |
| Sulfasalazine | 0.908135896 | 1.104425803 | −0.20 |
| Amsacrine | 0.151662928 | 0.348169557 | −0.20 |
| Dolutegravir | 0.706179899 | 0.903346995 | −0.20 |
| Azathioprine | 0.38928245 | 0.58653254 | −0.20 |
| Mitotane | 0.910927348 | 1.108393454 | −0.20 |
| Prednisone acetate | 0.780649295 | 0.978152929 | −0.20 |
| Indacaterol (maleate) | 1.115550482 | 1.313557552 | −0.20 |
| (S)-Verapamil (hydrochloride) | 0.971143376 | 1.169197975 | −0.20 |
| Aprepitant | 0.799226804 | 0.997990354 | −0.20 |
| Methimazole | 0.824537724 | 1.023514629 | −0.20 |
| Thiamine (hydrochloride) | 1.124631413 | 1.323616455 | −0.20 |
| Helicid | 1.003424658 | 1.20242323 | −0.20 |
| Dihydroergotamine (mesylate) | 0.821298421 | 1.020642614 | −0.20 |
| Butamben | 0.971169247 | 1.170680186 | −0.20 |
| Hydrocortisone acetate | 0.626395712 | 0.826100629 | −0.20 |
| Noscapine | 1.088417618 | 1.288148148 | −0.20 |
| Tromantadine (hydrochloride) | 0.741436464 | 0.941383353 | −0.20 |
| Minoxidil | 1.001694915 | 1.202186741 | −0.20 |
| Carbazochrome (sodium sulfonate) | 0.961982507 | 1.162630226 | −0.20 |
| Aztreonam | 0.892010309 | 1.092845659 | −0.20 |
| Canrenone | 0.845826688 | 1.046686255 | −0.20 |
| Mexiletine (hydrochloride) | 0.884813043 | 1.085863532 | −0.20 |
| Chlorphenesin | 1.023411978 | 1.224620304 | −0.20 |
| Clobetasol propionate | 1.280139272 | 1.481465329 | −0.20 |
| Citric acid (trilithium salt tetrahydrate) | 1.574160953 | 1.775614505 | −0.20 |
| Adiphenine (hydrochloride) | 0.886413994 | 1.087944798 | −0.20 |
| Vorapaxar | 0.983963494 | 1.185525859 | −0.20 |
| Fostamatinib Disodium | 0.900408163 | 1.102015965 | −0.20 |
| Capecitabine | 0.936409834 | 1.138041002 | −0.20 |
| Darolutamide | 1.061444308 | 1.263113006 | −0.20 |
| Dolutegravir (sodium) | 0.635364807 | 0.837256909 | −0.20 |
| Ropivacaine | 0.89491833 | 1.097788436 | −0.20 |
| Tiopronin | 0.720754717 | 0.92414966 | −0.20 |
| Sapropterin (dihydrochloride) | 1.038383111 | 1.242326025 | −0.20 |
| Parecoxib | 0.71307562 | 0.917053065 | −0.20 |
| Varenicline (Hydrochloride) | 0.89114886 | 1.095169222 | −0.20 |
| Choline theophyllinate | 0.903839983 | 1.108137045 | −0.20 |
| Triamcinolone hexacetonide | 0.528512182 | 0.732899566 | −0.20 |
| Phenoxybenzamine (hydrochloride) | 0.737460815 | 0.942076503 | −0.20 |
| Carbinoxamine maleate salt | 0.924671273 | 1.12942366 | −0.20 |
| Rosiglitazone | 1.016796161 | 1.222130979 | −0.21 |
| 20(S)-Ginsenoside Rg3 | 0.856389656 | 1.061801936 | −0.21 |
| Pergolide (mesylate) | 0.821412606 | 1.026901938 | −0.21 |
| Guaiazulene | 1.024801395 | 1.230531202 | −0.21 |
| Artemotil | 0.55135254 | 0.757172131 | −0.21 |
| Mivacurium (dichloride) | 0.717800653 | 0.924795235 | −0.21 |
| Carbetapentane (citrate) | 0.973590539 | 1.18069367 | −0.21 |
| Moxidectin | 0.281600485 | 0.489316741 | −0.21 |
| Levonorgestrel | 0.899226804 | 1.107315113 | −0.21 |
| Sulfisomidin | 0.847136382 | 1.055485179 | −0.21 |
| Bendamustine (hydrochloride) | 0.986565911 | 1.195042898 | −0.21 |
| Vitamin K4 | 0.772582619 | 0.981663113 | −0.21 |
| Regadenoson | 1.216413818 | 1.426061098 | −0.21 |
| Sodium Salicylate | 0.942985638 | 1.152638465 | −0.21 |
| Alectinib | 0.914575547 | 1.125052809 | −0.21 |
| Difluprednate | 0.716752577 | 0.927250804 | −0.21 |
| Idebenone | 1.028604265 | 1.23923445 | −0.21 |
| Eslicarbazepine acetate | 0.787385781 | 0.998651837 | −0.21 |
| Rotundine | 0.85875153 | 1.070362473 | −0.21 |
| Ribociclib succinate hydrate | 0.722575107 | 0.934493347 | −0.21 |
| Carbasalate calcium | 0.875397797 | 1.087420043 | −0.21 |
| Bergenin | 0.782713877 | 0.994877049 | −0.21 |
| Zileuton | 1.238415546 | 1.45079023 | −0.21 |
| Imipramine (hydrochloride) | 0.839548453 | 1.052015876 | −0.21 |
| L-Cysteine | 0.732309804 | 0.945306439 | −0.21 |
| Atorvastatin (hemicalcium salt) | 0.581652473 | 0.794666667 | −0.21 |
| Glutathione oxidized | 1.010075567 | 1.223310985 | −0.21 |
| Pretomanid | 1.260338814 | 1.473778736 | −0.21 |
| Aprotinin | 0.486254673 | 0.701161202 | −0.21 |
| Decamethonium (Bromide) | 0.906236651 | 1.121151936 | −0.21 |
| Levetiracetam | 0.882541377 | 1.098014364 | −0.22 |
| Chlorthalidone | 0.99272078 | 1.208512795 | −0.22 |
| Paroxetine (hydrochloride) | 0.873930074 | 1.089838639 | −0.22 |
| Droperidol | 1.002002156 | 1.217928903 | −0.22 |
| Zalcitabine | 0.94047075 | 1.15643605 | −0.22 |
| Baricitinib | 0.570056232 | 0.786321878 | −0.22 |
| Fipronil | 1.000261233 | 1.216612022 | −0.22 |
| Finafloxacin | 0.789915147 | 1.006358382 | −0.22 |
| Decloxizine (dihydrochloride) | 0.968686732 | 1.185783522 | −0.22 |
| Levamlodipine besylate | 1.043425606 | 1.260655939 | −0.22 |
| Trelagliptin | 0.705014058 | 0.922422593 | −0.22 |
| Sulfathiazole (sodium) | 1.04654498 | 1.263970843 | −0.22 |
| Fosfomycin (sodium) | 0.878221416 | 1.095656808 | −0.22 |
| Gemifloxacin (mesylate) | 0.900906653 | 1.118358096 | −0.22 |
| Bexarotene | 1.123702422 | 1.34123572 | −0.22 |
| Diphenylpyraline (hydrochloride) | 0.744595498 | 0.962251432 | −0.22 |
| Heparin (Lithium salt) | 0.956637843 | 1.174323216 | −0.22 |
| Heparin (Lithium salt) | 0.956637843 | 1.174323216 | −0.22 |
| Fenitrothion | 0.644489079 | 0.862248697 | −0.22 |
| Methylprednisolone | 0.617783505 | 0.835610932 | −0.22 |
| Ribostamycin (sulfate) | 1.494783905 | 1.71397538 | −0.22 |
| Escin | 1.057096248 | 1.276296296 | −0.22 |
| Homatropine (methylbromide) | 1.05002592 | 1.269436103 | −0.22 |
| Amlodipine (maleate) | 0.906878071 | 1.12672956 | −0.22 |
| Estradiol (cypionate) | 0.906590801 | 1.126653759 | −0.22 |
| Alfuzosin (hydrochloride) | 0.98266846 | 1.202909091 | −0.22 |
| Taurochenodeoxycholic acid (sodium salt) | 0.72985187 | 0.95011169 | −0.22 |
| Lidocaine (hydrochloride) | 0.98832618 | 1.209825998 | −0.22 |
| Lamivudine | 0.994660972 | 1.216307562 | −0.22 |
| Fluvastatin (sodium) | 0.167955801 | 0.389605315 | −0.22 |
| Oxiracetam | 0.882571075 | 1.104328018 | −0.22 |
| Loxoprofen (sodium) | 0.755374906 | 0.97751606 | −0.22 |
| Ambroxol | 0.912090251 | 1.134232302 | −0.22 |
| Quetiapine (hemifumarate) | 0.711166049 | 0.933940774 | −0.22 |
| Delamanid | 0.882731959 | 1.105707395 | −0.22 |
| Eflornithine (hydrochloride hydrate) | 0.741903088 | 0.965003723 | −0.22 |
| Riociguat | 0.700328022 | 0.9237836 | −0.22 |
| Sulpiride | 1.030438484 | 1.254367147 | −0.22 |
| Meclocycline (Sulfosalicylate Salt) | 0.988829247 | 1.213693851 | −0.22 |
| Angiotensin II human (acetate) | 0.715792116 | 0.941176471 | −0.23 |
| Zoledronic acid (monohydrate) | 0.806928907 | 1.032854709 | −0.23 |
| Cyclandelate | 0.934952978 | 1.161530055 | −0.23 |
| Everolimus | 0.596824898 | 0.823644793 | −0.23 |
| Nizatidine | 1.02623266 | 1.253103623 | −0.23 |
| Fenoprofen (Calcium hydrate) | 0.853858521 | 1.080933111 | −0.23 |
| Trometamol (hydrochloride) | 0.694702486 | 0.921816828 | −0.23 |
| Diclofenac (Sodium) | 0.859020619 | 1.086414791 | −0.23 |
| Cytidine | 1.090381546 | 1.318360227 | −0.23 |
| Trimethadione | 0.856978674 | 1.085117099 | −0.23 |
| Histamine (phosphate) | 0.803392444 | 1.031661184 | −0.23 |
| Donepezil (Hydrochloride) | 0.703474797 | 0.932118451 | −0.23 |
| Ranolazine (dihydrochloride) | 0.919383653 | 1.148113208 | −0.23 |
| Levofloxacin | 0.932958199 | 1.161866222 | −0.23 |
| Naproxen | 0.925726034 | 1.155016447 | −0.23 |
| Didanosine | 0.767504346 | 0.996934495 | −0.23 |
| Furagin | 0.986128279 | 1.21560679 | −0.23 |
| Ribociclib succinate | 0.755536481 | 0.98567042 | −0.23 |
| Anagliptin | 1.003259452 | 1.234120097 | −0.23 |
| Dabigatran etexilate (mesylate) | 0.983130391 | 1.214957176 | −0.23 |
| Bentiromide | 0.872924649 | 1.104811523 | −0.23 |
| Linagliptin | 0.840431267 | 1.07244898 | −0.23 |
| Tiotropium (Bromide) | 0.868508958 | 1.100905563 | −0.23 |
| Clozapine | 1.113661689 | 1.346477924 | −0.23 |
| Taurodeoxycholic acid (sodium hydrate) | 0.982915109 | 1.216307562 | −0.23 |
| Bucladesine (sodium) | 0.876802637 | 1.110210286 | −0.23 |
| Nifuratel | 1.019426336 | 1.252863589 | −0.23 |
| Anidulafungin | 1.247384155 | 1.480962644 | −0.23 |
| Lefamulin (acetate) | 0.861860146 | 1.095668241 | −0.23 |
| Rupatadine (Fumarate) | 1.073535986 | 1.308070581 | −0.23 |
| Propylthiouracil | 0.847327249 | 1.082089552 | −0.23 |
| Thiamine monochloride | 0.71378358 | 0.948622487 | −0.23 |
| Trilostane | 1.00273794 | 1.237591114 | −0.23 |
| Ferulic acid | 0.851581064 | 1.086509868 | −0.23 |
| Orotic acid | 0.765673981 | 1.000655738 | −0.23 |
| Antazoline (hydrochloride) | 0.770350404 | 1.005782313 | −0.24 |
| Dehydroandrographolide | 0.733868943 | 0.969471333 | −0.24 |
| Rosuvastatin (Calcium) | 0.920096463 | 1.156153297 | −0.24 |
| Flufenamic acid | 1.02123412 | 1.257660538 | −0.24 |
| Inosine pranobex | 0.859856951 | 1.096326294 | −0.24 |
| L-Lysine | 0.859247856 | 1.095969945 | −0.24 |
| Tamibarotene | 0.53919301 | 0.776726974 | −0.24 |
| Ramipril | 0.893622322 | 1.131824713 | −0.24 |
| Raltitrexed | 0.111673385 | 0.350271528 | −0.24 |
| Eliglustat (hemitartrate) | 0.595350052 | 0.834535519 | −0.24 |
| Losartan (potassium) | 0.810201892 | 1.049518304 | −0.24 |
| Ethosuximide | 0.883996424 | 1.124096037 | −0.24 |
| Antipyrine | 0.825557284 | 1.065899233 | −0.24 |
| Methyclothiazide | 0.855998768 | 1.096342092 | −0.24 |
| Pentagastrin | 0.866585067 | 1.107889126 | −0.24 |
| Lanreotide (acetate) | 1.04536029 | 1.287454121 | −0.24 |
| L-Phenylalanine | 0.860712387 | 1.102840353 | −0.24 |
| Methyl Salicylate | 0.722014515 | 0.964822404 | −0.24 |
| Mometasone furoate | 0.569889841 | 0.812793177 | −0.24 |
| (+)-Ketoconazole | 0.99341985 | 1.236820762 | −0.24 |
| Cefdinir | 0.999839228 | 1.243751488 | −0.24 |
| Fesoterodine (fumarate) | 1.003031222 | 1.247241136 | −0.24 |
| Sulfaguanidine | 0.939035284 | 1.183333333 | −0.24 |
| Ritonavir | 1.287267456 | 1.532577904 | −0.25 |
| Lomitapide | 0.709700094 | 0.955086764 | −0.25 |
| Daidzin | 0.838133374 | 1.083977331 | −0.25 |
| Laropiprant | 1.091542892 | 1.338342334 | −0.25 |
| Tiagabine (hydrochloride) | 1.158836011 | 1.405963841 | −0.25 |
| Nedaplatin | 1.005231689 | 1.252514368 | −0.25 |
| Estramustine (phosphate sodium) | 0.948745328 | 1.196028728 | −0.25 |
| Decitabine | 0.558996263 | 0.807351077 | −0.25 |
| Gastrodenol | 1.380526577 | 1.629012793 | −0.25 |
| Metixene hydrochloride hydrate | 1.044181926 | 1.292803097 | −0.25 |
| Doripenem (monohydrate) | 0.761793675 | 1.010510511 | −0.25 |
| Nelfinavir | 0.84631232 | 1.095216401 | −0.25 |
| Oxolamine (citrate) | 0.9127897 | 1.16171955 | −0.25 |
| Bempedoic acid | 1.093855377 | 1.342940889 | −0.25 |
| Oleic acid | 0.964291845 | 1.213920164 | −0.25 |
| Diclofenac | 0.917043444 | 1.166765315 | −0.25 |
| Alibendol | 0.797046663 | 1.047158191 | −0.25 |
| Dehydrocholic acid | 0.901182176 | 1.151444001 | −0.25 |
| Olsalazine (Disodium) | 0.96038864 | 1.210847701 | −0.25 |
| Tolazoline (hydrochloride) | 0.998185588 | 1.248748749 | −0.25 |
| Carmustine | 0.725927314 | 0.97655105 | −0.25 |
| Reldesemtiv | 0.844101633 | 1.095656808 | −0.25 |
| Galanthamine (hydrobromide) | 1.032585901 | 1.284294783 | −0.25 |
| Tolbutamide | 0.847374095 | 1.099433282 | −0.25 |
| Clomiphene (citrate) | 0.748414091 | 1.000538503 | −0.25 |
| Gefitinib | 0.447183625 | 0.699636364 | −0.25 |
| Perindopril (erbumine) | 0.898874598 | 1.151392526 | −0.25 |
| Ramatroban | 0.652405279 | 0.904995403 | −0.25 |
| Aminohippurate (sodium) | 0.996111975 | 1.248748749 | −0.25 |
| L-Lysine hydrochloride | 0.751924346 | 1.005806011 | −0.25 |
| Meticrane | 1.108753908 | 1.363207547 | −0.25 |
| Tegafur | 0.713730763 | 0.968242424 | −0.25 |
| Bepridil (hydrochloride hydrate) | 0.896138026 | 1.150687744 | −0.25 |
| Palbociclib (isethionate) | 0.650218453 | 0.905016447 | −0.25 |
| Valproic acid (sodium salt) | 0.726730118 | 0.981783053 | −0.26 |
| Tegaserod (maleate) | 0.727190058 | 0.982390232 | −0.26 |
| Mequinol | 0.627713921 | 0.882929819 | −0.26 |
| Anagrelide (hydrochloride) | 0.732570926 | 0.988046448 | −0.26 |
| L-Valine | 1.126669073 | 1.382331468 | −0.26 |
| Hydroxyfasudil (hydrochloride) | 0.788435679 | 1.044848485 | −0.26 |
| Loxapine | 1.056051809 | 1.312893082 | −0.26 |
| Thiamine nitrate | 0.717526139 | 0.974861247 | −0.26 |
| Lodoxamide | 0.885784533 | 1.143766271 | −0.26 |
| Pentoxifylline | 0.884487312 | 1.142857143 | −0.26 |
| Tacrolimus (monohydrate) | 0.753336893 | 1.01182932 | −0.26 |
| Meglumine | 0.969349895 | 1.227987855 | −0.26 |
| Nimesulide | 0.898302626 | 1.156999564 | −0.26 |
| L-Hyoscyamine | 0.971386152 | 1.230299767 | −0.26 |
| Tandospirone | 1.191606413 | 1.450606277 | −0.26 |
| Iproniazid (phosphate) | 0.84681182 | 1.105939273 | −0.26 |
| Granisetron (Hydrochloride) | 0.863897597 | 1.123060109 | −0.26 |
| Pinacidil monohydrate | 0.833741921 | 1.093023256 | −0.26 |
| Atenolol | 1.168176255 | 1.428571429 | −0.26 |
| Megestrol acetate | 0.928883639 | 1.189336618 | −0.26 |
| Palbociclib (hydrochloride) | 1.027782855 | 1.288726683 | −0.26 |
| Xylitol | 1.122793383 | 1.383840461 | −0.26 |
| Gadoxetate (Disodium) | 0.902319588 | 1.163585209 | −0.26 |
| Naphazoline (hydrochloride) | 0.927896996 | 1.189355169 | −0.26 |
| Fomepizole | 0.977126703 | 1.238898026 | −0.26 |
| Solifenacin | 0.879736058 | 1.141587496 | −0.26 |
| Carbetocin | 1.020909506 | 1.282898789 | −0.26 |
| Phenylbutazone | 1.113857987 | 1.375854214 | −0.26 |
| Artemether | 0.796051502 | 1.059365404 | −0.26 |
| Tofacitinib (citrate) | 0.547254462 | 0.810597303 | −0.26 |
| Rucaparib (Camsylate) | 0.799637118 | 1.063229897 | −0.26 |
| Vernakalant (Hydrochloride) | 0.987573099 | 1.251677852 | −0.26 |
| Goserelin (acetate) | 0.908388004 | 1.172847907 | −0.26 |
| Pimecrolimus | 0.757553606 | 1.022371365 | −0.26 |
| Liothyronine | 0.907842329 | 1.172665148 | −0.26 |
| Lusutrombopag | 0.882872928 | 1.147713951 | −0.26 |
| Cefotetan | 0.930894858 | 1.195835577 | −0.26 |
| Aclidinium (Bromide) | 0.987271103 | 1.252515723 | −0.27 |
| Tioconazole | 0.941318328 | 1.206617472 | −0.27 |
| Mirodenafil (dihydrochloride) | 1.169613734 | 1.435005118 | −0.27 |
| Cholesterol | 1.074125575 | 1.340114204 | −0.27 |
| Flutamide | 0.78258481 | 1.048747153 | −0.27 |
| Niacin | 0.999437992 | 1.265754763 | −0.27 |
| Ethylenediaminetetraacetic acid (trisodium salt) | 1.397329484 | 1.664638598 | −0.27 |
| Olprinone (Hydrochloride) | 0.874512671 | 1.142058166 | −0.27 |
| Guanabenz (Acetate) | 0.607334439 | 0.875337187 | −0.27 |
| Phenytoin (sodium) | 0.92833587 | 1.196574055 | −0.27 |
| Moclobemide | 0.821315753 | 1.089941728 | −0.27 |
| Bilastine | 1.165778792 | 1.434719031 | −0.27 |
| Heptaminol (hydrochloride) | 0.589399745 | 0.858412504 | −0.27 |
| Fosfomycin (calcium) | 1.000731128 | 1.270343336 | −0.27 |
| Tipranavir | 0.670337395 | 0.940115686 | −0.27 |
| Fedratinib | 0.292641326 | 0.562639821 | −0.27 |
| Diiodohydroxyquinoline | 0.828393136 | 1.098415908 | −0.27 |
| Valpromide | 0.804357149 | 1.074864236 | −0.27 |
| Famciclovir | 0.750937207 | 1.021776114 | −0.27 |
| Tranilast | 0.554285714 | 0.825463401 | −0.27 |
| Daphnetin | 0.935911602 | 1.207112153 | −0.27 |
| Emedastine | 0.718222528 | 0.989570701 | −0.27 |
| Nilvadipine | 0.787546102 | 1.059348332 | −0.27 |
| Puerarin | 0.930633973 | 1.202594535 | −0.27 |
| Desloratadine | 0.841891106 | 1.113976276 | −0.27 |
| Lodenafil | 0.569853475 | 0.842128216 | −0.27 |
| Lurasidone (Hydrochloride) | 0.842629243 | 1.114966635 | −0.27 |
| L-Asparagine | 1.437062092 | 1.709631728 | −0.27 |
| Oxaceprol | 0.683848797 | 0.956606061 | −0.27 |
| Avanafil | 0.684759472 | 0.957707631 | −0.27 |
| Minoxidil sulfate | 1.056079855 | 1.329070077 | −0.27 |
| Dalbavancin (hydrochloride) | 1.365765483 | 1.638908702 | −0.27 |
| Triamcinolone | 0.615516453 | 0.8895504 | −0.27 |
| Sulfadoxine | 0.985963018 | 1.260455933 | −0.27 |
| Riluzole hydrochloride | 0.876899502 | 1.151599093 | −0.27 |
| Pomalidomide | 1.095023937 | 1.369821195 | −0.27 |
| Ethacridine (lactate) | 0.044834308 | 0.320600718 | −0.28 |
| Rufloxacin hydrochloride | 1.015553869 | 1.291352093 | −0.28 |
| Acetohydroxamic acid | 0.89784191 | 1.173759758 | −0.28 |
| Molindone (hydrochloride) | 0.861856109 | 1.137909899 | −0.28 |
| Irsogladine | 0.994093325 | 1.270279782 | −0.28 |
| Cetirizine (dihydrochloride) | 0.909158823 | 1.185454545 | −0.28 |
| Urapidil (hydrochloride) | 0.733450627 | 1.009904372 | −0.28 |
| Meclofenoxate (hydrochloride) | 1.221052632 | 1.498507463 | −0.28 |
| Udenafil | 1.194584095 | 1.472592897 | −0.28 |
| Chloroprocaine (hydrochloride) | 0.883141762 | 1.161201348 | −0.28 |
| Ciclesonide | 0.42335515 | 0.701430997 | −0.28 |
| Losartan | 1.271248401 | 1.549337659 | −0.28 |
| Toremifene (citrate) | 0.765554182 | 1.044191344 | −0.28 |
| Anastrozole | 0.984652461 | 1.263421767 | −0.28 |
| L-Thyroxine | 0.8302189 | 1.109843684 | −0.28 |
| Suprofen | 0.82832618 | 1.108495394 | −0.28 |
| 6-Mercaptopurine | 0.428537957 | 0.708744471 | −0.28 |
| Proglumide (sodium) | 0.978066167 | 1.258448229 | −0.28 |
| Pazufloxacin (mesylate) | 0.705067921 | 0.98579235 | −0.28 |
| Lubiprostone | 0.78328841 | 1.064285714 | −0.28 |
| Gallamine Triethiodide | 1.029450167 | 1.310510249 | −0.28 |
| Reserpine | 0.759076274 | 1.041735197 | −0.28 |
| Capsaicin | 0.914235086 | 1.197079008 | −0.28 |
| Guanethidine (sulfate) | 1.336313959 | 1.619357953 | −0.28 |
| Betamethasone dipropionate | 0.631714212 | 0.914884868 | −0.28 |
| Acenocoumarol | 1.60455192 | 1.889480478 | −0.28 |
| Cloperastine (hydrochloride) | 0.802159177 | 1.087118392 | −0.28 |
| Talaporfin (sodium) | 0.931889236 | 1.219811321 | −0.29 |
| Piperonyl butoxide | 1.202379225 | 1.491059747 | −0.29 |
| Allopurinol | 0.909490455 | 1.198177677 | −0.29 |
| Fostamatinib | 0.412524366 | 0.701342282 | −0.29 |
| Oxybenzone | 1.093558834 | 1.382650143 | −0.29 |
| Terbutaline (sulfate) | 0.97376734 | 1.262870159 | −0.29 |
| Bazedoxifene (acetate) | 0.863621778 | 1.153104925 | −0.29 |
| Clebopride (malate) | 0.680668573 | 0.970286885 | −0.29 |
| Fulvestrant | 1.074367278 | 1.364346997 | −0.29 |
| Mesna | 0.85359857 | 1.144923344 | −0.29 |
| Nalfurafine (hydrochloride) | 0.93099598 | 1.222327044 | −0.29 |
| Etoposide | 0.415448343 | 0.706935123 | −0.29 |
| Epalrestat | 0.942500557 | 1.234580384 | −0.29 |
| Valproic acid | 0.664088398 | 0.957014459 | −0.29 |
| Paeonol | 0.780416157 | 1.073773987 | −0.29 |
| Demecarium Bromide | 0.818568878 | 1.112237142 | −0.29 |
| Iotalamic acid | 1.090961845 | 1.384648932 | −0.29 |
| Methocarbamol | 1.062876988 | 1.356853204 | −0.29 |
| (Z)-Thiothixene | 1.050101471 | 1.344474619 | −0.29 |
| Ingenol | 0.637569061 | 0.932004689 | −0.29 |
| Flucloxacillin sodium | 0.969292634 | 1.263855096 | −0.29 |
| Phthalylsulfacetamide | 0.9792318 | 1.273899371 | −0.29 |
| Heparin (sodium salt) (MW 15 kDa) | 1.156027467 | 1.450779327 | −0.29 |
| Heparin (sodium salt) (MW 15 kDa) | 1.156027467 | 1.450779327 | −0.29 |
| Phensuximide | 0.833422317 | 1.128432615 | −0.30 |
| Cefmenoxime (hydrochloride) | 0.929957082 | 1.22517912 | −0.30 |
| β-Pinene | 0.672608586 | 0.96798213 | −0.30 |
| Desvenlafaxine (succinate hydrate) | 0.923265306 | 1.218914896 | −0.30 |
| Loratadine | 0.826428461 | 1.122102009 | −0.30 |
| Benzamil (hydrochloride) | 0.668569254 | 0.964278254 | −0.30 |
| Risedronate (sodium) | 0.72641302 | 1.022199454 | −0.30 |
| Darunavir | 0.806714924 | 1.102524472 | −0.30 |
| Benzocaine | 0.647907929 | 0.943930636 | −0.30 |
| Velpatasvir | 0.717316599 | 1.013818182 | −0.30 |
| Ribociclib | 0.853733906 | 1.150460594 | −0.30 |
| Metronidazole Benzoate | 1.000959578 | 1.297998093 | −0.30 |
| Cefathiamidine | 0.978944527 | 1.276252019 | −0.30 |
| Doxylamine (succinate) | 0.803392444 | 1.100740132 | −0.30 |
| Trimetazidine (dihydrochloride) | 0.835950454 | 1.133526012 | −0.30 |
| Sivelestat (sodium tetrahydrate) | 0.992765273 | 1.290407046 | −0.30 |
| Phenolphthalein | 0.872073223 | 1.169782409 | −0.30 |
| Fludrocortisone acetate | 0.437027881 | 0.735307517 | −0.30 |
| Niraparib | 0.991670226 | 1.289970209 | −0.30 |
| Pioglitazone (hydrochloride) | 0.966894566 | 1.265204957 | −0.30 |
| Boceprevir | 1.001218324 | 1.299776286 | −0.30 |
| Rivaroxaban | 0.777308044 | 1.075959762 | −0.30 |
| Furosemide | 0.887942122 | 1.186622233 | −0.30 |
| Enasidenib | 1.013148065 | 1.312484848 | −0.30 |
| Eliglustat | 0.855236703 | 1.154636388 | −0.30 |
| Eltrombopag | 1.170352769 | 1.470397405 | −0.30 |
| Nadolol | 1.069147005 | 1.36957101 | −0.30 |
| Diroximel fumarate | 0.745823696 | 1.046463816 | −0.30 |
| Tilorone (dihydrochloride) | 1.103650085 | 1.404430706 | −0.30 |
| Gabapentin | 0.935708288 | 1.236987607 | −0.30 |
| Sulfadimethoxine (sodium) | 0.728847602 | 1.030528667 | −0.30 |
| Pipobroman | 1.077652733 | 1.379909545 | −0.30 |
| Deserpidine | 0.661878453 | 0.964830012 | −0.30 |
| Vortioxetine (hydrobromide) | 0.41837232 | 0.72147651 | −0.30 |
| Tropisetron (Hydrochloride) | 0.862780377 | 1.166062917 | −0.30 |
| Delapril (hydrochloride) | 0.918188737 | 1.221641947 | −0.30 |
| Trapidil | 0.776713495 | 1.080294208 | −0.30 |
| Gastrodin | 1.162374681 | 1.466262208 | −0.30 |
| Sacubitril | 0.704922027 | 1.008948546 | −0.30 |
| Cortisone | 0.91174907 | 1.216454456 | −0.30 |
| Levocarnitine propionate (hydrochloride) | 0.951415199 | 1.256150994 | −0.30 |
| Mebhydrolin | 1.507610534 | 1.813031161 | −0.31 |
| Acefylline | 1.148995226 | 1.454564145 | −0.31 |
| Pralidoxime (chloride) | 0.897572213 | 1.203395364 | −0.31 |
| Malotilate | 0.856750446 | 1.162642369 | −0.31 |
| Solifenacin (hydrochloride) | 0.822185971 | 1.128148148 | −0.31 |
| Cefotaxime (sodium salt) | 0.962230463 | 1.26833713 | −0.31 |
| Kalii Dehydrographolidi Succinas | 1.081183612 | 1.388126287 | −0.31 |
| Ingenol Mebutate | 0.391837393 | 0.699144356 | −0.31 |
| Methylergometrine (maleate) | 0.715156701 | 1.02248394 | −0.31 |
| Chloroquine | 0.918128655 | 1.225595821 | −0.31 |
| Berbamine | 0.967414099 | 1.275462324 | −0.31 |
| Bestatin (hydrochloride) | 0.90830721 | 1.216612022 | −0.31 |
| Tandospirone (citrate) | 0.856543121 | 1.166825548 | −0.31 |
| L-Carnitine (hydrochloride) | 1.166721044 | 1.477777778 | −0.31 |
| Sulfanilamide | 1.005339598 | 1.316782522 | −0.31 |
| Ulipristal acetate | 0.818555207 | 1.130311615 | −0.31 |
| Dapoxetine (hydrochloride) | 0.779362603 | 1.091675093 | −0.31 |
| Cinacalcet (hydrochloride) | 0.786232681 | 1.098702186 | −0.31 |
| Trifarotene | 0.786663445 | 1.099150142 | −0.31 |
| Leuprolide (Acetate) | 0.68770618 | 1.00034153 | −0.31 |
| Chlorphenoxamine | 0.990334378 | 1.303169399 | −0.31 |
| Thymol | 1.040808344 | 1.354217286 | −0.31 |
| Abarelix (Acetate) | 0.926454201 | 1.239973037 | −0.31 |
| Medetomidine (hydrochloride) | 1.138816036 | 1.452369321 | −0.31 |
| Ropinirole (hydrochloride) | 0.787385781 | 1.101112235 | −0.31 |
| Temozolomide | 0.950321543 | 1.264698881 | −0.31 |
| Teprenone | 1.037931034 | 1.352517986 | −0.31 |
| Naproxen (sodium) | 0.846874086 | 1.162042389 | −0.32 |
| Moroxydine (hydrochloride) | 0.857222161 | 1.172491776 | −0.32 |
| Sultamicillin (tosylate) | 1.021453199 | 1.336942836 | −0.32 |
| Liothyronine (sodium) | 0.907842329 | 1.223690205 | −0.32 |
| Alizapride hydrochloride | 0.906193781 | 1.222450658 | −0.32 |
| Venlafaxine (hydrochloride) | 0.732614555 | 1.049319728 | −0.32 |
| Talc | 0.949611517 | 1.266475056 | −0.32 |
| Diltiazem (hydrochloride) | 0.684395501 | 1.001361007 | −0.32 |
| Estrone | 1.108868959 | 1.426364943 | −0.32 |
| Dienogest | 1.069089601 | 1.387225929 | −0.32 |
| Besifovir | 0.912103004 | 1.230296827 | −0.32 |
| Trovafloxacin | 0.867800815 | 1.186418544 | −0.32 |
| (−)-Menthol | 1.114882033 | 1.433519851 | −0.32 |
| Dapagliflozin ((2S)-1,2-propanediol, hydrate) | 0.625876011 | 0.944557823 | −0.32 |
| Ibrutinib (Racemate) | 0.923255477 | 1.242477104 | −0.32 |
| Drospirenone | 1.061932728 | 1.38191804 | −0.32 |
| Furosemide (sodium) | 0.872928177 | 1.193044158 | −0.32 |
| Tocofersolan | 0.460929329 | 0.781090909 | −0.32 |
| Pefloxacin (mesylate) | 0.862668299 | 1.182942431 | −0.32 |
| Flupentixol dihydrochloride | 0.780393028 | 1.100945724 | −0.32 |
| D-(+)-Glucono-1,5-lactone | 0.905979249 | 1.22662384 | −0.32 |
| Vilazodone (Hydrochloride) | 0.698387383 | 1.019262161 | −0.32 |
| Efavirenz | 0.786551078 | 1.107519564 | −0.32 |
| Penciclovir | 0.63940956 | 0.960530793 | −0.32 |
| Reserpine (hydrochloride) | 0.856778062 | 1.178248355 | −0.32 |
| Ozenoxacin | 0.758878742 | 1.080426154 | −0.32 |
| Letrozole | 1.011939571 | 1.334451902 | −0.32 |
| Etomidate | 0.799280391 | 1.122121711 | −0.32 |
| Terfenadine | 0.821896685 | 1.145148026 | −0.32 |
| Dithranol | 0.989693847 | 1.313923456 | −0.32 |
| Telotristat ethyl | 0.928169208 | 1.25284738 | −0.32 |
| Otilonium (bromide) | 0.807080854 | 1.131984586 | −0.32 |
| Riboflavin (phosphate sodium) | 0.982758621 | 1.307753797 | −0.32 |
| Eletriptan (hydrobromide) | 1.080424054 | 1.405515004 | −0.33 |
| Pancuronium (dibromide) | 0.867816092 | 1.193073858 | −0.33 |
| Balsalazide | 0.919773683 | 1.245093764 | −0.33 |
| Umifenovir (hydrochloride) | 0.762501072 | 1.088052754 | −0.33 |
| Amlodipine (besylate) | 1.033279743 | 1.358962152 | −0.33 |
| Nortriptyline (hydrochloride) | 1.009905774 | 1.335694051 | −0.33 |
| Prednisolone acetate | 0.600338826 | 0.926326636 | −0.33 |
| Bacitracin | 1.094081942 | 1.420384351 | −0.33 |
| Homosalate | 0.583068874 | 0.90961986 | −0.33 |
| Cilostazol | 1.173913043 | 1.500606061 | −0.33 |
| Dantrolene (sodium hemiheptahydrate) | 0.946813496 | 1.273562436 | −0.33 |
| Tadalafil | 0.970958622 | 1.298108553 | −0.33 |
| Desvenlafaxine | 0.830929817 | 1.15808656 | −0.33 |
| Norgestrel | 1.022261655 | 1.349434171 | −0.33 |
| Salmeterol (xinafoate) | 1.368115399 | 1.695652174 | −0.33 |
| S-(+)-Ketoprofen | 0.984013051 | 1.311851852 | −0.33 |
| Rivastigmine (tartrate) | 0.913659794 | 1.242363344 | −0.33 |
| Benorilate | 0.883231334 | 1.211940299 | −0.33 |
| Butenafine (Hydrochloride) | 1.364368677 | 1.693698095 | −0.33 |
| Levobupivacaine (hydrochloride) | 0.968325116 | 1.297939394 | −0.33 |
| Baicalein | 0.93131156 | 1.261428095 | −0.33 |
| Dinoprost (tromethamine salt) | 0.960491191 | 1.290663287 | −0.33 |
| Perospirone | 0.715410813 | 1.045969966 | −0.33 |
| Nafamostat (mesylate) | 0.883924956 | 1.21450368 | −0.33 |
| (Z)-Capsaicin | 0.895144378 | 1.225779741 | −0.33 |
| Toceranib (phosphate) | 0.946889226 | 1.278311599 | −0.33 |
| Cephalexin | 0.840818569 | 1.172665148 | −0.33 |
| Etodolac | 0.919295109 | 1.251322085 | −0.33 |
| Niclosamide | 0.627079935 | 0.959259259 | −0.33 |
| 5-Aminosalicylic Acid | 0.877495463 | 1.209698908 | −0.33 |
| Isosorbide mononitrate | 1.126705062 | 1.459497535 | −0.33 |
| 5-Hydroxytryptophan | 0.855998768 | 1.189077795 | −0.33 |
| Cilastatin | 0.851496204 | 1.184591195 | −0.33 |
| Asenapine | 1.302730128 | 1.635977337 | −0.33 |
| Cefprozil (monohydrate) | 1.108877086 | 1.442347289 | −0.33 |
| Avapritinib | 0.600181488 | 0.933653078 | −0.33 |
| Trelagliptin (succinate) | 1.135309278 | 1.469051447 | −0.33 |
| Morinidazole | 0.81636504 | 1.150116369 | −0.33 |
| Xylose | 0.904440545 | 1.23819781 | −0.33 |
| (+)-Penbutolol | 1.014200299 | 1.348778736 | −0.33 |
| L-Serine | 0.964905867 | 1.299540416 | −0.33 |
| Amifampridine | 1.141447846 | 1.47623201 | −0.33 |
| Galanthamine | 0.829007005 | 1.163921966 | −0.33 |
| Netupitant | 0.915022762 | 1.250171585 | −0.34 |
| Lincomycin (hydrochloride monohydrate) | 0.77811458 | 1.113876497 | −0.34 |
| Ipratropium (bromide) | 0.896854828 | 1.232801822 | −0.34 |
| Dyclonine (hydrochloride) | 0.782392894 | 1.118688525 | −0.34 |
| 6-Acetamidohexanoic acid | 1.010495757 | 1.348113208 | −0.34 |
| Lorlatinib | 0.805215066 | 1.142905292 | −0.34 |
| Bifendate | 1.15610081 | 1.494037829 | −0.34 |
| Trometamol | 1.132563562 | 1.471011513 | −0.34 |
| Canagliflozin | 0.820563035 | 1.159061834 | −0.34 |
| Citicoline (sodium) | 0.812511076 | 1.151158994 | −0.34 |
| Bepotastine (besilate) | 0.898467433 | 1.237205026 | −0.34 |
| Bromfenac (sodium) | 0.993469175 | 1.332896175 | −0.34 |
| Ethoxzolamide | 0.99089597 | 1.330386513 | −0.34 |
| Oxybutynin (chloride) | 0.831695612 | 1.171217409 | −0.34 |
| Enalapril (maleate) | 1.056380721 | 1.396571965 | −0.34 |
| Clinofibrate | 1.006530825 | 1.346885246 | −0.34 |
| Primaquine (Diphosphate) | 0.851359465 | 1.19200931 | −0.34 |
| Nifurtimox | 1.101920728 | 1.443050987 | −0.34 |
| Foscarnet (sodium) | 0.919600726 | 1.26085798 | −0.34 |
| Tosufloxacin (tosylate hydrate) | 0.867862969 | 1.210123457 | −0.34 |
| Teneligliptin | 0.985159817 | 1.327762691 | −0.34 |
| Etidronic acid | 0.852704515 | 1.195834539 | −0.34 |
| Artesunate | 0.629961962 | 0.973217726 | −0.34 |
| Lemborexant | 1.206909157 | 1.550419032 | −0.34 |
| Bifonazole | 1.228072933 | 1.571861626 | −0.34 |
| [Sar1, Ile8]-Angiotensin II (TFA) | 0.947858017 | 1.292110874 | −0.34 |
| Valacyclovir (hydrochloride) | 0.798083352 | 1.142905292 | −0.34 |
| Bictegravir | 0.7122571 | 1.057112069 | −0.34 |
| Dimetridazole | 0.873819242 | 1.218914896 | −0.35 |
| Hydroxyzine (dihydrochloride) | 1.12822186 | 1.47382716 | −0.35 |
| Pizotifen | 0.78109984 | 1.126742712 | −0.35 |
| Acipimox | 1.007680779 | 1.353688324 | −0.35 |
| Prednisolone Tebutate | 0.663389095 | 1.009569378 | −0.35 |
| Methylthiouracil | 0.708294022 | 1.055469356 | −0.35 |
| Maprotiline (hydrochloride) | 0.999537964 | 1.34672849 | −0.35 |
| Cloperastine fendizoate | 0.844566353 | 1.192131148 | −0.35 |
| Iguratimod | 0.904381443 | 1.252009646 | −0.35 |
| Carmofur | 0.200881286 | 0.548715382 | −0.35 |
| Doxofylline | 0.989699217 | 1.337585421 | −0.35 |
| Vidarabine | 1.193525006 | 1.542492918 | −0.35 |
| Pimethixene maleate | 1.142461112 | 1.492014426 | −0.35 |
| Netilmicin (sulfate) | 1.427719821 | 1.777697321 | −0.35 |
| Tranylcypromine (hemisulfate) | 0.824939521 | 1.175204918 | −0.35 |
| Angiotensin III (TFA) | 0.987845304 | 1.338413443 | −0.35 |
| 5-Azacytidine | 0.806426332 | 1.15715847 | −0.35 |
| Acetylcholine (iodide) | 1.000243665 | 1.351230425 | −0.35 |
| Brexpiprazole | 0.640495465 | 0.991714836 | −0.35 |
| Pirfenidone | 0.97706359 | 1.328473804 | −0.35 |
| Guaifenesin | 0.85226234 | 1.204064874 | −0.35 |
| Menaquinone-4 | 0.776999275 | 1.128895184 | −0.35 |
| Icatibant (acetate) | 0.769328494 | 1.121236344 | −0.35 |
| Clofarabine | 0.447410416 | 0.802006106 | −0.35 |
| Pipemidic acid | 0.82669726 | 1.181475498 | −0.35 |
| Tropisetron | 0.886661527 | 1.2421875 | −0.36 |
| Neticonazole (hydrochloride) | 0.83585209 | 1.192335158 | −0.36 |
| Isradipine | 0.803392444 | 1.159950658 | −0.36 |
| Erythromycin Ethylsuccinate | 0.890701468 | 1.247654321 | −0.36 |
| Metyrapone | 0.738627602 | 1.095805921 | −0.36 |
| Capreomycin (sulfate) | 1.370624323 | 1.727914334 | −0.36 |
| Diazoxide | 0.903777701 | 1.26121756 | −0.36 |
| Mirabegron | 0.773148148 | 1.130872483 | −0.36 |
| Sulfadimethoxine | 0.876326159 | 1.23409251 | −0.36 |
| Methylprednisolone succinate | 0.46609717 | 0.824250106 | −0.36 |
| Bromfenac (sodium hydrate) | 0.715963695 | 1.074355725 | −0.36 |
| Sulfinpyrazone | 0.941715051 | 1.300314465 | −0.36 |
| Benidipine (hydrochloride) | 0.588894821 | 0.947613012 | −0.36 |
| Fumaric acid | 0.669147005 | 1.028510525 | −0.36 |
| Ropivacaine (mesylate) | 0.795408959 | 1.15503876 | −0.36 |
| Plerixafor | 0.93502658 | 1.295618511 | −0.36 |
| Pimavanserin tartrate | 0.900531601 | 1.261129685 | −0.36 |
| Ibuprofen | 1.057661506 | 1.419095902 | −0.36 |
| Dicyclomine (hydrochloride) | 0.849084717 | 1.211461845 | −0.36 |
| Estradiol benzoate | 0.922126082 | 1.284738041 | −0.36 |
| Imatinib | 0.91750306 | 1.280170576 | −0.36 |
| Nalidixic acid | 0.969179706 | 1.331881252 | −0.36 |
| Lornoxicam | 0.811049724 | 1.174286831 | −0.36 |
| Clevidipine | 0.92888092 | 1.292121212 | −0.36 |
| Betahistine (mesylate) | 0.871345029 | 1.234737186 | −0.36 |
| Indomethacin (sodium hydrate) | 0.800460748 | 1.164218087 | −0.36 |
| Ofloxacin | 0.856237199 | 1.22061657 | −0.36 |
| Kanamycin (sulfate) | 1.486338798 | 1.851074101 | −0.36 |
| Tolmetin | 0.614361034 | 0.979895756 | −0.37 |
| Mequitazine | 1.338336052 | 1.703950617 | −0.37 |
| Ropivacaine (hydrochloride monohydrate) | 0.606055408 | 0.971683476 | −0.37 |
| Lomefloxacin (hydrochloride) | 1.165165165 | 1.531300419 | −0.37 |
| Vardenafil (hydrochloride) | 0.985024586 | 1.35203934 | −0.37 |
| Apalutamide | 0.8273676 | 1.195183044 | −0.37 |
| Trazodone (hydrochloride) | 1.032351661 | 1.400348888 | −0.37 |
| Solifenacin (Succinate) | 1.243778691 | 1.611898017 | −0.37 |
| Sarpogrelate (hydrochloride) | 0.647239938 | 1.015368852 | −0.37 |
| Ecabet (sodium) | 0.801814215 | 1.170021829 | −0.37 |
| D-Cysteine | 0.731578947 | 1.099920064 | −0.37 |
| Carbamazepine | 0.933662958 | 1.302050114 | −0.37 |
| D-Cycloserine | 1.146335612 | 1.516110582 | −0.37 |
| Roxatidine (Acetate Hydrochloride) | 1.140867547 | 1.511120802 | −0.37 |
| Pantoprazole (sodium hydrate) | 0.943670018 | 1.31410388 | −0.37 |
| Sodium Picosulfate | 1.042944081 | 1.413533835 | −0.37 |
| Eugenol | 1.047537031 | 1.418384671 | −0.37 |
| Etripamil | 0.972112761 | 1.343038272 | −0.37 |
| Benfotiamine | 0.791475666 | 1.162813102 | −0.37 |
| Pramocaine (hydrochloride) | 0.92981509 | 1.301382158 | −0.37 |
| Tenoxicam | 0.982145923 | 1.354145343 | −0.37 |
| Tropicamide | 0.804134672 | 1.176667253 | −0.37 |
| (R)-Serine | 1.344640924 | 1.718179606 | −0.37 |
| Oxytocin | 1.211097139 | 1.584700428 | −0.37 |
| Quinine | 1.124173303 | 1.49811524 | −0.37 |
| Palonosetron (Hydrochloride) | 0.700936569 | 1.075390745 | −0.37 |
| Isoxsuprine hydrochloride | 0.939035284 | 1.314150943 | −0.38 |
| Quetiapine sulfoxide (dihydrochloride) | 0.775862069 | 1.151079137 | −0.38 |
| Chlortetracycline (hydrochloride) | 1.205446902 | 1.582860232 | −0.38 |
| Fenofibric acid | 1.05759321 | 1.435078657 | −0.38 |
| Daptomycin | 0.647624132 | 1.025348542 | −0.38 |
| Proparacaine (Hydrochloride) | 0.876932404 | 1.254754637 | −0.38 |
| Nimodipine | 0.890653565 | 1.268938616 | −0.38 |
| Nitrendipine | 1.208060397 | 1.586965461 | −0.38 |
| Diethylcarbamazine (citrate) | 0.945064378 | 1.324462641 | −0.38 |
| L-Proline | 0.990856844 | 1.370491803 | −0.38 |
| Phenylephrine | 0.816985774 | 1.197359029 | −0.38 |
| Berbamine (dihydrochloride) | 1.400974536 | 1.781562054 | −0.38 |
| Tofogliflozin (hydrate) | 1.029807204 | 1.411322331 | −0.38 |
| Brivudine | 0.726676658 | 1.108451392 | −0.38 |
| Atosiban (acetate) | 0.81621893 | 1.198808637 | −0.38 |
| Catechin | 0.726364522 | 1.109619687 | −0.38 |
| Lansoprazole | 0.956402591 | 1.340158046 | −0.38 |
| Ilaprazole (sodium) | 0.778337531 | 1.162454874 | −0.38 |
| Maltitol | 0.708819002 | 1.093237705 | −0.38 |
| Glipizide | 1.459535407 | 1.844162189 | −0.38 |
| Dimenhydrinate | 0.852337071 | 1.237458882 | −0.39 |
| Telaprevir | 1.16456759 | 1.55042898 | −0.39 |
| L-Ornithine | 1.042437102 | 1.428504344 | −0.39 |
| Nestoron | 1.095046609 | 1.481211138 | −0.39 |
| Testosterone propionate | 0.921170165 | 1.307861635 | −0.39 |
| Ketoconazole | 0.614789447 | 1.002715688 | −0.39 |
| Amorolfine (hydrochloride) | 0.844909903 | 1.233384853 | −0.39 |
| Cefsulodin (sodium) | 0.656916648 | 1.045423497 | −0.39 |
| Sulfabenzamide | 1.023485785 | 1.412211806 | −0.39 |
| Betahistine (dihydrochloride) | 1.139405654 | 1.528328612 | −0.39 |
| Saquinavir | 0.925241158 | 1.314210902 | −0.39 |
| Meloxicam | 0.786178382 | 1.175259546 | −0.39 |
| Ketoprofen | 0.893755587 | 1.283555779 | −0.39 |
| 6-Thioguanine | 0.315299878 | 0.70533049 | −0.39 |
| Progesterone | 0.664088398 | 1.057053537 | −0.39 |
| Hydroxyprogesterone caproate | 0.739250745 | 1.133006436 | −0.39 |
| Fabomotizole (hydrochloride) | 1.067985167 | 1.462883202 | −0.39 |
| Estriol | 0.744751381 | 1.139898398 | −0.40 |
| Estradiol valerianate | 0.825309667 | 1.221102997 | −0.40 |
| Dasabuvir | 0.828725398 | 1.224566179 | −0.40 |
| Fluconazole | 0.833676693 | 1.230068337 | −0.40 |
| Azilsartan | 1.020515589 | 1.416980793 | −0.40 |
| Fluoxetine (hydrochloride) | 0.986402967 | 1.383143508 | −0.40 |
| Glucosamine | 0.772489036 | 1.16952177 | −0.40 |
| Nilotinib | 1.113230179 | 1.510772164 | −0.40 |
| Regorafenib (monohydrate) | 0.628163074 | 1.025859136 | −0.40 |
| Deferiprone | 1.044013191 | 1.443074995 | −0.40 |
| Flibanserin | 0.964755459 | 1.364140015 | −0.40 |
| Aliskiren (hemifumarate) | 0.954678112 | 1.354145343 | −0.40 |
| Piperaquine (tetraphosphate tetrahydrate) | 0.825493171 | 1.226149623 | −0.40 |
| Tolfenamic Acid | 0.930888148 | 1.331768021 | −0.40 |
| Isopropamide (iodide) | 0.918529941 | 1.319490132 | −0.40 |
| Sitagliptin (phosphate monohydrate) | 0.645480537 | 1.046789617 | −0.40 |
| Flucytosine | 0.845618557 | 1.247186495 | −0.40 |
| Bimatoprost | 1.142461112 | 1.544564657 | −0.40 |
| Permethrin | 0.949638072 | 1.351880474 | −0.40 |
| VAL-083 | 0.63392283 | 1.036181861 | −0.40 |
| Racecadotril | 0.891080921 | 1.293506951 | −0.40 |
| Sulfisoxazole | 0.870281869 | 1.273025048 | −0.40 |
| Trichlormethiazide | 0.9481828 | 1.351382269 | −0.40 |
| Prednisolone (hemisuccinate) | 0.475080464 | 0.878420039 | −0.40 |
| EC5026 | 0.742502343 | 1.148349779 | −0.41 |
| Apraclonidine (hydrochloride) | 1.213404904 | 1.619276058 | −0.41 |
| Vilanterol (trifenatate) | 1.047057331 | 1.453102152 | −0.41 |
| Hydrocortisone cypionate | 0.691388546 | 1.098861048 | −0.41 |
| DL-Menthol | 0.890718853 | 1.29860753 | −0.41 |
| Rimonabant (Hydrochloride) | 0.882007406 | 1.2906069 | −0.41 |
| Ursodiol | 0.983225044 | 1.392046454 | −0.41 |
| Lopinavir | 0.872287503 | 1.281225756 | −0.41 |
| Amlexanox | 0.855137063 | 1.264239973 | −0.41 |
| Castor oil | 0.833258628 | 1.242666667 | −0.41 |
| (R)-Terazosin | 0.755079383 | 1.165090461 | −0.41 |
| Chlorzoxazone | 1.000731128 | 1.410921871 | −0.41 |
| Aceglutamide | 0.767411976 | 1.178042763 | −0.41 |
| Fluorometholone acetate | 0.575757576 | 0.98661443 | −0.41 |
| Dexpramipexole (dihydrochloride) | 0.579859555 | 0.991515152 | −0.41 |
| Nefopam (hydrochloride) | 0.967309411 | 1.379249559 | −0.41 |
| Fluspirilene | 0.906739812 | 1.318907104 | −0.41 |
| Valemetostat (tosylate) | 1.051902048 | 1.464193715 | −0.41 |
| Pidotimod | 0.970834769 | 1.383458647 | −0.41 |
| Raltegravir (potassium salt) | 0.918784319 | 1.331906321 | −0.41 |
| Silodosin | 0.881595631 | 1.295375723 | −0.41 |
| Rauwolscine (hydrochloride) | 0.962946362 | 1.377454545 | −0.41 |
| Glimepiride | 0.831594099 | 1.24613918 | −0.41 |
| Ranolazine | 1.359123267 | 1.77381534 | −0.41 |
| Celecoxib | 0.947244767 | 1.362462761 | −0.42 |
| Glasdegib | 0.982129743 | 1.397867804 | −0.42 |
| Propyl gallate | 1.125013878 | 1.540912829 | −0.42 |
| Pargyline (hydrochloride) | 0.787529186 | 1.203644647 | −0.42 |
| Theophylline | 1.176082524 | 1.592373689 | −0.42 |
| α-Vitamin E | 1.081626069 | 1.499585978 | −0.42 |
| Folic acid | 1.312323169 | 1.730920114 | −0.42 |
| Nonivamide | 1.201011615 | 1.620420127 | −0.42 |
| Atropine | 0.89772544 | 1.317416506 | −0.42 |
| Gadodiamide | 1.243550835 | 1.663349348 | −0.42 |
| Letermovir | 1.079690273 | 1.499585978 | −0.42 |
| Chromocarb | 0.801154908 | 1.221146687 | −0.42 |
| Efloxate | 0.92899327 | 1.349379275 | −0.42 |
| Tenofovir (Disoproxil Fumarate) | 0.742502343 | 1.163320857 | −0.42 |
| Finasteride | 0.787768031 | 1.20917226 | −0.42 |
| Tocainide (hydrochloride) | 0.954446461 | 1.375965894 | −0.42 |
| (−)-Sparteine (sulfate pentahydrate) | 0.85794347 | 1.279642058 | −0.42 |
| Dolasetron | 0.978175205 | 1.400328716 | −0.42 |
| Diphenidol (hydrochloride) | 0.8776267 | 1.30022779 | −0.42 |
| Cinnarizine | 0.921338271 | 1.343993956 | −0.42 |
| Delafloxacin (meglumine) | 1.0086362 | 1.432221163 | −0.42 |
| Ketorolac (tromethamine salt) | 0.687132942 | 1.111533587 | −0.42 |
| Prucalopride (succinate) | 0.904980429 | 1.329384312 | −0.42 |
| Azaphen (dihydrochloride monohydrate) | 0.778315373 | 1.203893443 | −0.43 |
| Alendronic acid | 0.780223746 | 1.207106352 | −0.43 |
| Sotalol (hydrochloride) | 0.962918455 | 1.389969294 | −0.43 |
| Bephenium (hydroxynaphthoate) | 0.723440546 | 1.151006711 | −0.43 |
| Chlorprothixene | 1.175912755 | 1.605517909 | −0.43 |
| Tivozanib | 0.868044865 | 1.298155738 | −0.43 |
| Halofantrine hydrochloride | 0.744646098 | 1.175592859 | −0.43 |
| Octreotide (acetate) | 0.725791234 | 1.157098526 | −0.43 |
| Metergoline | 0.732646256 | 1.164592627 | −0.43 |
| Fluticasone furoate | 0.614353212 | 1.046839587 | −0.43 |
| Buspirone (hydrochloride) | 0.852892351 | 1.285570385 | −0.43 |
| Lasofoxifene (Tartrate) | 0.756896029 | 1.189950693 | −0.43 |
| Clofazimine | 0.406629834 | 0.839781165 | −0.43 |
| Taltirelin | 0.871034199 | 1.304783599 | −0.43 |
| Topiramate | 0.97242268 | 1.406350482 | −0.43 |
| Bestatin | 0.819131833 | 1.255177339 | −0.44 |
| Benazepril (hydrochloride) | 0.877411867 | 1.313999128 | −0.44 |
| Benfluorex (hydrochloride) | 0.669433802 | 1.106037389 | −0.44 |
| Sulfamethoxazole | 0.864723468 | 1.301364943 | −0.44 |
| Rifamycin (sodium) | 1.877748248 | 2.314447592 | −0.44 |
| Ciprofloxacin | 1.066009105 | 1.502745367 | −0.44 |
| Neostigmine (methyl sulfate) | 1.28091354 | 1.717777778 | −0.44 |
| Oxaliplatin | 0.921092564 | 1.35861359 | −0.44 |
| Amodiaquine (dihydrochloride) | 1.036902342 | 1.475017844 | −0.44 |
| Sitafloxacin (hydrate) | 0.707939301 | 1.146516393 | −0.44 |
| Phenprocoumon | 0.872934346 | 1.31163522 | −0.44 |
| Minaprine (dihydrochloride) | 0.902081727 | 1.340871711 | −0.44 |
| Pentostatin | 0.802455953 | 1.241656105 | −0.44 |
| Levomefolic acid | 1.368107303 | 1.807627323 | −0.44 |
| Trihexyphenidyl (hydrochloride) | 0.967619473 | 1.407232704 | −0.44 |
| Rilmenidine (hemifumarate) | 0.771026864 | 1.210722264 | −0.44 |
| Zanubrutinib | 0.349636718 | 0.789422135 | −0.44 |
| Liranaftate | 0.838806289 | 1.279443255 | −0.44 |
| Dolasetron (Mesylate hydrate) | 0.844368382 | 1.286581014 | −0.44 |
| Praziquantel | 1.042712226 | 1.485002112 | −0.44 |
| Benzydamine (hydrochloride) | 1.001554347 | 1.443873355 | −0.44 |
| Oseltamivir acid | 0.975415465 | 1.418678815 | −0.44 |
| Tetramisole (hydrochloride) | 0.918529941 | 1.362253289 | −0.44 |
| Cephalexin (monohydrate) | 1.062268108 | 1.506210323 | −0.44 |
| Voxelotor | 0.70128866 | 1.145900322 | −0.44 |
| Prednisolone (disodium phosphate) | 0.64223176 | 1.087001024 | −0.44 |
| Clindamycin (phosphate) | 0.896888276 | 1.34174145 | −0.44 |
| Pixantrone (dimaleate) | 0.884813043 | 1.33006856 | −0.45 |
| Hyodeoxycholic acid | 1.015845677 | 1.462042202 | −0.45 |
| Docusate (Sodium) | 0.838990621 | 1.285220126 | −0.45 |
| Sulfadiazine | 0.763139854 | 1.20981318 | −0.45 |
| Alcaftadine | 1.002627971 | 1.449599672 | −0.45 |
| Propranolol (hydrochloride) | 0.568316322 | 1.015903801 | −0.45 |
| Hydralazine (hydrochloride) | 1.081096765 | 1.529437418 | −0.45 |
| Salsalate | 1.132137031 | 1.580493827 | −0.45 |
| Succimer | 0.957560775 | 1.406131239 | −0.45 |
| Gemfibrozil | 0.919845361 | 1.36937299 | −0.45 |
| Sofosbuvir | 0.687541787 | 1.137512639 | −0.45 |
| Ambroxol (hydrochloride) | 0.834664247 | 1.285371703 | −0.45 |
| Deferasirox | 0.989367986 | 1.440612353 | −0.45 |
| Limaprost | 0.949022024 | 1.400309119 | −0.45 |
| L-Lactic acid | 0.814486207 | 1.266629865 | −0.45 |
| Medroxyprogesterone acetate | 0.60577392 | 1.057996146 | −0.45 |
| Pexidartinib (hydrochloride) | 0.751381215 | 1.203985932 | −0.45 |
| Tetrabenazine | 1.161558602 | 1.614631633 | −0.45 |
| Ampicillin (sodium) | 0.903172641 | 1.356473271 | −0.45 |
| Efinaconazole | 0.853403141 | 1.306723747 | −0.45 |
| Aspirin | 1.020082693 | 1.473693472 | −0.45 |
| Milnacipran (hydrochloride) | 1.089948454 | 1.544614148 | −0.45 |
| Peramivir (trihydrate) | 1.605563835 | 2.061549602 | −0.46 |
| Fluorescein | 0.695828012 | 1.152620288 | −0.46 |
| Tetracycline (hydrochloride) | 0.929957082 | 1.386898669 | −0.46 |
| Tacrine (hydrochloride) | 0.760056707 | 1.217760366 | −0.46 |
| Fingolimod (hydrochloride) | 0.874418605 | 1.334754797 | −0.46 |
| Biotin | 0.606741573 | 1.067106284 | −0.46 |
| Procyclidine (hydrochloride) | 0.996953431 | 1.458787614 | −0.46 |
| Albendazole sulfoxide | 1.004607478 | 1.46718903 | −0.46 |
| Ritodrine (hydrochloride) | 0.971986636 | 1.434621711 | −0.46 |
| Pimozide | 0.636098427 | 1.099644872 | −0.46 |
| Gamithromycin | 0.886010807 | 1.350271528 | −0.46 |
| Olmesartan | 1.465105313 | 1.929921541 | −0.46 |
| Chlorprothixene (hydrochloride) | 1.02693475 | 1.491763898 | −0.46 |
| Sophoridine | 1.042329074 | 1.507321773 | −0.46 |
| Quinestrol | 1.012049616 | 1.477916593 | −0.47 |
| Ivabradine (hydrochloride) | 1.148557512 | 1.615534929 | −0.47 |
| Landiolol (hydrochloride) | 0.884210526 | 1.351812367 | −0.47 |
| Bedaquiline (fumarate) | 1.11943013 | 1.587227049 | −0.47 |
| Garenoxacin (Mesylate hydrate) | 1.299925066 | 1.768930142 | −0.47 |
| Cidofovir | 1.556116925 | 2.026770504 | −0.47 |
| Ronidazole | 1.069111852 | 1.540267668 | −0.47 |
| Rimegepant | 0.984405145 | 1.45703404 | −0.47 |
| Acotiamide (monohydrochloride trihydrate) | 0.945295871 | 1.41863316 | −0.47 |
| Ketanserin | 0.70932327 | 1.182699767 | −0.47 |
| Bromisoval | 0.860528626 | 1.333911368 | −0.47 |
| Detomidine (hydrochloride) | 0.632216495 | 1.105707395 | −0.47 |
| Tazemetostat (hydrobromide) | 1.209154289 | 1.683024184 | −0.47 |
| Chondroitin (sulfate) | 0.771490336 | 1.246513536 | −0.48 |
| Chondroitin (sulfate) | 0.771490336 | 1.246513536 | −0.48 |
| Toloxatone | 1.098807712 | 1.574263631 | −0.48 |
| Pravastatin (sodium) | 1.237729487 | 1.713238886 | −0.48 |
| Ruxolitinib (S enantiomer) | 0.554079939 | 1.029715437 | −0.48 |
| Dapsone | 0.998392468 | 1.474654378 | −0.48 |
| Lactose | 0.655157247 | 1.132855191 | −0.48 |
| Latanoprost | 1.005356407 | 1.483151761 | −0.48 |
| Dofetilide | 0.797224364 | 1.275082237 | −0.48 |
| Amrinone | 0.861007258 | 1.339139344 | −0.48 |
| Macitentan | 1.113304094 | 1.591722595 | −0.48 |
| Avobenzone | 0.976688103 | 1.45703404 | −0.48 |
| Camphor | 0.890943447 | 1.371746545 | −0.48 |
| Sulfamonomethoxine | 0.897052255 | 1.378301887 | −0.48 |
| Niraparib (hydrochloride) | 0.953384133 | 1.434666667 | −0.48 |
| Cefoxitin (sodium) | 0.81605529 | 1.298726738 | −0.48 |
| Estropipate | 0.992725068 | 1.475463724 | −0.48 |
| Fingolimod | 0.998214531 | 1.481124905 | −0.48 |
| Sodium stibogluconate | 1.025417299 | 1.508922443 | −0.48 |
| Primidone | 0.978781449 | 1.462315097 | −0.48 |
| Embelin | 1.08238705 | 1.566572238 | −0.48 |
| Levofloxacin (hydrate) | 0.824276527 | 1.308497977 | −0.48 |
| Sulindac | 0.851945772 | 1.336223507 | −0.48 |
| Pivmecillinam (hydrochloride) | 1.161178007 | 1.646314871 | −0.49 |
| Azilsartan medoxomil | 0.632797271 | 1.118568233 | −0.49 |
| Prilocaine | 0.879581994 | 1.365627232 | −0.49 |
| Mefloquine (hydrochloride) | 0.952250804 | 1.438943109 | −0.49 |
| Tenofovir alafenamide | 0.72268202 | 1.209532888 | −0.49 |
| Enoxacin (hydrate) | 1.045144158 | 1.532258065 | −0.49 |
| Etravirine | 0.936188077 | 1.423832221 | −0.49 |
| Dapiprazole (hydrochloride) | 1.043136538 | 1.532035685 | −0.49 |
| Prucalopride | 1.033358701 | 1.522456653 | −0.49 |
| Sulfamerazine | 1.00852548 | 1.497679216 | −0.49 |
| Pramipexole (dihydrochloride) | 1.014343344 | 1.503515152 | −0.49 |
| Metaxalone | 1.077776067 | 1.567233385 | −0.49 |
| Doxifluridine | 0.839170444 | 1.329384966 | −0.49 |
| Atracurium (besylate) | 0.927586207 | 1.418598455 | −0.49 |
| Tazemetostat | 1.227947863 | 1.721530876 | −0.49 |
| (R)-Elagolix | 0.899601703 | 1.394077449 | −0.49 |
| Piracetam | 0.906173803 | 1.402504817 | −0.50 |
| Treosulfan | 0.821061093 | 1.318971673 | −0.50 |
| Perflubron | 1.407504078 | 1.905432099 | −0.50 |
| Etamivan | 0.984752811 | 1.482740855 | −0.50 |
| Ethynodiol diacetate | 1.155398121 | 1.653786708 | −0.50 |
| Treprostinil | 0.951242961 | 1.449658314 | −0.50 |
| Procarbazine (Hydrochloride) | 1.11908646 | 1.618024691 | −0.50 |
| Vildagliptin | 0.713783343 | 1.21295578 | −0.50 |
| Gefarnate | 0.913457415 | 1.413110939 | −0.50 |
| Deracoxib | 1.180872943 | 1.681995483 | −0.50 |
| Tolazamide | 0.910630747 | 1.412365443 | −0.50 |
| Lofexidine (hydrochloride) | 0.950026582 | 1.452824029 | −0.50 |
| Isoprenaline (hydrochloride) | 1.172608485 | 1.676235164 | −0.50 |
| Phentolamine (mesylate) | 0.898150955 | 1.404085466 | −0.51 |
| Deferasirox (Fe3+ chelate) | 1.082916599 | 1.589014629 | −0.51 |
| Dipyridamole | 0.972928106 | 1.479979854 | −0.51 |
| Oxantel (pamoate) | 1.018601447 | 1.526073248 | −0.51 |
| Chlorpheniramine (maleate) | 0.942755941 | 1.451242045 | −0.51 |
| Zonisamide | 1.108938037 | 1.61746418 | −0.51 |
| Piperidolate | 0.681876662 | 1.190457719 | −0.51 |
| Telotristat etiprate | 0.814493319 | 1.323121387 | −0.51 |
| Montelukast (sodium) | 1.090182648 | 1.599331523 | −0.51 |
| Flumethasone | 0.714525609 | 1.224022879 | −0.51 |
| Duvelisib | 0.934929629 | 1.444444444 | −0.51 |
| Chlorpropamide | 0.980124764 | 1.490187527 | −0.51 |
| 2-(Phosphonooxy)benzoic acid | 1.004858955 | 1.515347334 | −0.51 |
| Canagliflozin (hemihydrate) | 0.835902889 | 1.34704538 | −0.51 |
| Upadacitinib | 0.700310024 | 1.212709192 | −0.51 |
| Benactyzine hydrochloride | 0.878033206 | 1.390438247 | −0.51 |
| Miglustat (hydrochloride) | 1.323024885 | 1.835694051 | −0.51 |
| Procainamide (hydrochloride) | 0.88312045 | 1.396810934 | −0.51 |
| Tucidinostat | 0.730838431 | 1.244613918 | −0.51 |
| Telithromycin | 0.801878433 | 1.315703559 | −0.51 |
| Trimipramine (maleate) | 1.022172397 | 1.536997126 | −0.51 |
| Gabapentin (hydrochloride) | 0.996935257 | 1.513220851 | −0.52 |
| Fosinopril (sodium) | 1.313462494 | 1.830798938 | −0.52 |
| Varenicline (Tartrate) | 0.815699269 | 1.333812359 | −0.52 |
| Ziprasidone | 0.889987639 | 1.408158095 | −0.52 |
| Entecavir (monohydrate) | 1 | 1.519161106 | −0.52 |
| Bevantolol (hydrochloride) | 0.707939301 | 1.227117486 | −0.52 |
| Prednisone | 1.001532371 | 1.522286578 | −0.52 |
| Prazosin (hydrochloride) | 1.105498229 | 1.626996394 | −0.52 |
| Bronopol | 0.93888412 | 1.460593654 | −0.52 |
| Cefepime (Dihydrochloride Monohydrate) | 0.687365983 | 1.209076804 | −0.52 |
| Sparfloxacin | 0.92754173 | 1.449897049 | −0.52 |
| Nifedipine | 0.922938144 | 1.449758842 | −0.53 |
| Acrivastine | 0.776555971 | 1.303620219 | −0.53 |
| Calcifediol | 0.897635173 | 1.424958882 | −0.53 |
| Nylidrin (hydrochloride) | 0.927345145 | 1.454775779 | −0.53 |
| Santonin | 0.857168173 | 1.388834476 | −0.53 |
| Tolnaftate | 1.191184634 | 1.723478618 | −0.53 |
| Ethamsylate | 1.117837569 | 1.651939655 | −0.53 |
| Migalastat (hydrochloride) | 0.916223176 | 1.45035824 | −0.53 |
| Desoxycorticosterone pivalate | 1.040105119 | 1.574420037 | −0.53 |
| Epinastine (hydrochloride) | 0.880559986 | 1.414952661 | −0.53 |
| Brompheniramine (maleate) | 0.906533471 | 1.442638855 | −0.54 |
| Penicillin G benzathine (tetrahydrate) | 1.063728211 | 1.600123355 | −0.54 |
| Levosulpiride | 0.749755302 | 1.287160494 | −0.54 |
| Anamorelin | 0.482699805 | 1.020134228 | −0.54 |
| Cefazolin | 1.065047022 | 1.603060109 | −0.54 |
| Varenicline | 0.814630225 | 1.353249226 | −0.54 |
| Levobetaxolol (hydrochloride) | 0.97701443 | 1.517250063 | −0.54 |
| Imrecoxib | 1.221474838 | 1.762571839 | −0.54 |
| Fadrozole hydrochloride | 1.100837139 | 1.642782536 | −0.54 |
| Clomipramine (hydrochloride) | 0.92807639 | 1.470376095 | −0.54 |
| Nilotinib (monohydrochloride monohydrate) | 0.68565057 | 1.230023274 | −0.54 |
| Faropenem daloxate | 0.904198303 | 1.448742138 | −0.54 |
| Eprosartan (mesylate) | 0.869714627 | 1.415272727 | −0.55 |
| Drofenine (hydrochloride) | 0.890758741 | 1.436982119 | −0.55 |
| Ranitidine (hydrochloride) | 0.638424437 | 1.185670079 | −0.55 |
| Batilol | 0.770376176 | 1.320655738 | −0.55 |
| Tiratricol | 1.318107667 | 1.868888889 | −0.55 |
| Penicillamine | 1.004807032 | 1.556118571 | −0.55 |
| Oxeladin (citrate) | 0.920954993 | 1.475296715 | −0.55 |
| Asenapine (maleate) | 0.932190942 | 1.489978678 | −0.56 |
| Allylestrenol | 1.180779318 | 1.738641915 | −0.56 |
| Iproniazid | 0.781321992 | 1.339209925 | −0.56 |
| Aristolochic acid A | 0.724149056 | 1.282086669 | −0.56 |
| Glycine | 1.010646879 | 1.569141595 | −0.56 |
| Dropropizine | 1.000154012 | 1.558990211 | −0.56 |
| (S)-Flurbiprofen | 0.945402299 | 1.504262295 | −0.56 |
| Cyclopentolate (hydrochloride) | 0.852704515 | 1.412207116 | −0.56 |
| Mozavaptan (hydrochloride) | 0.687710753 | 1.248168051 | −0.56 |
| Flupirtine (Maleate) | 1.02629613 | 1.588848485 | −0.56 |
| Argipressin | 0.795148248 | 1.358163265 | −0.56 |
| Urapidil | 1.094574113 | 1.657666119 | −0.56 |
| Pregnenolone | 0.965478303 | 1.529395529 | −0.56 |
| Teneligliptin (hydrobromide) | 0.88584117 | 1.450054645 | −0.56 |
| Acebutolol (hydrochloride) | 0.97625459 | 1.541151386 | −0.56 |
| Hydrocortisone hemisuccinate | 0.510214996 | 1.075303355 | −0.57 |
| Cefotaxime | 0.659963632 | 1.225196288 | −0.57 |
| Sodium copper chlorophyllin A | 1.34674615 | 1.912865074 | −0.57 |
| Terbinafine hydrochloride | 1.221531713 | 1.792646661 | −0.57 |
| Methazolamide | 0.906938846 | 1.478277735 | −0.57 |
| Methacycline (hydrochloride) | 0.904666564 | 1.478619268 | −0.57 |
| Mifepristone | 0.999587969 | 1.57357631 | −0.57 |
| Luliconazole | 0.956240487 | 1.531648214 | −0.58 |
| Ornidazole (Levo-) | 1.058199454 | 1.634186429 | −0.58 |
| Enalaprilat (dihydrate) | 0.949045461 | 1.525284738 | −0.58 |
| Latrepirdine (dihydrochloride) | 1.082572298 | 1.660330061 | −0.58 |
| Octocrylene | 0.962096677 | 1.540514776 | −0.58 |
| Salicylamide | 0.906954062 | 1.485741811 | −0.58 |
| Iloprost | 0.89437299 | 1.473220662 | −0.58 |
| Venetoclax | 0.933247423 | 1.517282958 | −0.58 |
| Mefenamic acid | 1.551099299 | 2.135977337 | −0.58 |
| Cabergoline | 1.183860836 | 1.769121813 | −0.59 |
| Nelarabine | 0.704922027 | 1.29082774 | −0.59 |
| Methyl aminolevulinate (hydrochloride) | 0.886010807 | 1.472847168 | −0.59 |
| Demeclocycline (hydrochloride) | 0.939497717 | 1.527470232 | −0.59 |
| Xylometazoline (hydrochloride) | 0.815339596 | 1.406491499 | −0.59 |
| Relebactam | 0.844987944 | 1.437788018 | −0.59 |
| Alverine (citrate) | 1.10995106 | 1.702962963 | −0.59 |
| Meclofenamate (sodium) | 0.857878034 | 1.452676493 | −0.59 |
| (−)-Sparteine | 0.836611731 | 1.431929481 | −0.60 |
| Posaconazole | 0.885169104 | 1.480804763 | −0.60 |
| Etomidate (hydrochloride) | 0.873297353 | 1.469161184 | −0.60 |
| Tannic acid | 0.794064671 | 1.391388673 | −0.60 |
| Azelaic acid | 1.10372842 | 1.702136939 | −0.60 |
| Bezafibrate | 1.052743256 | 1.652030993 | −0.60 |
| Ebastine | 0.638154686 | 1.237572254 | −0.60 |
| Molsidomine | 0.741916116 | 1.342513576 | −0.60 |
| Rebamipide | 1.131675364 | 1.734991776 | −0.60 |
| Fluticasone (propionate) | 0.861077555 | 1.466005666 | −0.60 |
| Clemastine (fumarate) | 1.110836424 | 1.716240182 | −0.61 |
| Doxazosin (mesylate) | 1.083292695 | 1.689980732 | −0.61 |
| Mepivacaine (hydrochloride) | 0.805043314 | 1.413110939 | −0.61 |
| Rivastigmine | 1.132821281 | 1.741573034 | −0.61 |
| Pyridoxine (hydrochloride) | 0.886755928 | 1.496273806 | −0.61 |
| Enasidenib (mesylate) | 0.928092784 | 1.53818328 | −0.61 |
| (S)-(−)-Propranolol (hydrochloride) | 0.887911335 | 1.499692055 | −0.61 |
| Prostaglandin E2 | 0.934550465 | 1.546446762 | −0.61 |
| Phenylephrine (hydrochloride) | 0.755803651 | 1.370551747 | −0.61 |
| Prothionamide | 0.775762993 | 1.391337195 | −0.62 |
| Mephenytoin | 0.98784029 | 1.604050093 | −0.62 |
| Vitamin D2 | 0.792748249 | 1.409171523 | −0.62 |
| Tulobuterol (hydrochloride) | 0.998787511 | 1.616341864 | −0.62 |
| Tenofovir | 1.013277693 | 1.63726836 | −0.62 |
| Gluconate (sodium) | 0.949991934 | 1.574661882 | −0.62 |
| Metolazone | 0.84406861 | 1.468827455 | −0.62 |
| Niraparib (tosylate) | 1.044845943 | 1.670714877 | −0.63 |
| Sildenafil | 1.034092558 | 1.660270388 | −0.63 |
| Histamine (dihydrochloride) | 1.081236738 | 1.708382249 | −0.63 |
| Cromolyn (sodium) | 1.032343988 | 1.660330061 | −0.63 |
| Indapamide | 0.759324759 | 1.387526779 | −0.63 |
| Tezacaftor | 0.859730722 | 1.488272921 | −0.63 |
| Griseofulvin | 0.696286131 | 1.326221877 | −0.63 |
| Omarigliptin | 0.844587629 | 1.475482315 | −0.63 |
| Nilutamide | 0.934212333 | 1.565375854 | −0.63 |
| Selenomethionine | 1.265275708 | 1.898382814 | −0.63 |
| Dibucaine | 0.748995292 | 1.382488479 | −0.63 |
| Sildenafil (citrate) | 0.86961326 | 1.504103165 | −0.63 |
| Clonidine | 0.824919614 | 1.459890502 | −0.63 |
| Labetalol (hydrochloride) | 0.981611544 | 1.616973055 | −0.64 |
| Serotonin hydrochloride | 1.002903694 | 1.638697212 | −0.64 |
| Acetylcysteine | 0.732407238 | 1.36874831 | −0.64 |
| Metformin | 0.965621124 | 1.603003591 | −0.64 |
| Oritavancin (diphosphate) | 0.987684187 | 1.62602459 | −0.64 |
| Chloroquine (phosphate) | 0.917070289 | 1.556748196 | −0.64 |
| Relugolix | 0.977974277 | 1.617948108 | −0.64 |
| Glibenclamide | 0.979848866 | 1.619828408 | −0.64 |
| Triclabendazole | 1.027887239 | 1.67269312 | −0.64 |
| Choline Fenofibrate | 0.604532164 | 1.251677852 | −0.65 |
| Pheniramine (Maleate) | 0.879149155 | 1.526377036 | −0.65 |
| Tenofovir (Disoproxil) | 0.700919503 | 1.348330113 | −0.65 |
| Conivaptan (hydrochloride) | 0.989033084 | 1.641254393 | −0.65 |
| Gadodiamide (hydrate) | 0.734244373 | 1.386574625 | −0.65 |
| Revefenacin | 0.964153276 | 1.616924246 | −0.65 |
| Indinavir (sulfate) | 0.94603284 | 1.598835799 | −0.65 |
| Deoxycholic acid | 1.028069043 | 1.682859509 | −0.65 |
| Nifenazone | 0.808812729 | 1.466098081 | −0.66 |
| Ethionamide | 0.856958763 | 1.515675241 | −0.66 |
| Ezetimibe | 1.208505058 | 1.870542257 | −0.66 |
| Retinoic acid | 0.584126472 | 1.247860889 | −0.66 |
| Amiodarone (hydrochloride) | 0.885784533 | 1.554527047 | −0.67 |
| Cefazedone | 0.940904023 | 1.610162917 | −0.67 |
| Aliskiren | 1.083956153 | 1.753951149 | −0.67 |
| β-Carotene | 0.650588805 | 1.321832735 | −0.67 |
| Atomoxetine (hydrochloride) | 0.988002742 | 1.663108191 | −0.68 |
| 4-(Aminomethyl)benzoic acid | 1.285643318 | 1.961139271 | −0.68 |
| Dydrogesterone | 1.064203904 | 1.744686724 | −0.68 |
| Pramipexole | 0.835250918 | 1.518976546 | −0.68 |
| Gliquidone | 1.038965039 | 1.723853684 | −0.68 |
| Diphylline | 0.873151125 | 1.558914544 | −0.69 |
| L-(−)-α-Methyldopa (hydrate) | 0.972972973 | 1.664891901 | −0.69 |
| Cimetidine | 0.808258019 | 1.503754489 | −0.70 |
| Elbasvir | 0.634565916 | 1.334206141 | −0.70 |
| Suramin (sodium salt) | 0.940397351 | 1.640391551 | −0.70 |
| Troxipide | 1.111308276 | 1.812034226 | −0.70 |
| DL-Xylose | 0.799280391 | 1.500411184 | −0.70 |
| Rabeprazole (sodium) | 0.932569769 | 1.634280983 | −0.70 |
| Isoxicam | 1.015245159 | 1.720293894 | −0.71 |
| Ketotifen (fumarate) | 0.83943299 | 1.544614148 | −0.71 |
| Ceftaroline fosamil | 0.824353709 | 1.529745042 | −0.71 |
| Alarelin (Acetate) | 1.426431505 | 2.133144476 | −0.71 |
| Piroxicam | 0.842664534 | 1.552247998 | −0.71 |
| Fidaxomicin | 0.695103093 | 1.404742765 | −0.71 |
| Osilodrostat | 0.935043402 | 1.646820809 | −0.71 |
| Etretinate | 0.746851709 | 1.459292763 | −0.71 |
| Dibucaine (hydrochloride) | 0.719615747 | 1.433281614 | −0.71 |
| Nepafenac | 0.898423218 | 1.612194621 | −0.71 |
| Idramantone | 0.92372964 | 1.637572254 | −0.71 |
| Penbutolol (sulfate) | 1.218651848 | 1.933427762 | −0.71 |
| Ticagrelor | 0.870636186 | 1.58863859 | −0.72 |
| Olaparib | 0.716871087 | 1.435608999 | −0.72 |
| Oxytetracycline (hydrochloride) | 1.00744285 | 1.728370878 | −0.72 |
| Hydroxychloroquine sulfate | 0.89498376 | 1.617911901 | −0.72 |
| Methoxsalen | 0.790208078 | 1.513859275 | −0.72 |
| Loxapine (succinate) | 0.87757732 | 1.602491961 | −0.72 |
| D-Gluconic acid | 1.115323011 | 1.844951415 | −0.73 |
| Sucrose | 0.842991317 | 1.582109043 | −0.74 |
| Adenine (hemisulfate) | 0.917161792 | 1.656538698 | −0.74 |
| Avibactam (sodium) | 0.774309958 | 1.514258189 | −0.74 |
| Yohimbine (Hydrochloride) | 0.818004486 | 1.558188825 | −0.74 |
| Gluconate (Calcium) | 0.878601276 | 1.620560109 | −0.74 |
| Chlormadinone acetate | 0.950453327 | 1.694535382 | −0.74 |
| Vigabatrin (hydrochloride) | 1.024469234 | 1.769304099 | −0.74 |
| Clarithromycin | 0.861575563 | 1.606522257 | −0.74 |
| Omidenepag isopropyl | 0.815346447 | 1.562520405 | −0.75 |
| 5-Aminolevulinic acid (hydrochloride) | 0.855847069 | 1.604431599 | −0.75 |
| Mangafodipir (trisodium) | 1.338797814 | 2.087617668 | −0.75 |
| Ceforanide | 0.979387693 | 1.73561869 | −0.76 |
| Isosorbide dinitrate | 1.305629379 | 2.067988669 | −0.76 |
| Bemegride | 0.893964111 | 1.657530864 | −0.76 |
| Betaine (hydrochloride) | 0.829523303 | 1.593862227 | −0.76 |
| Clotrimazole | 0.718843709 | 1.484842707 | −0.77 |
| Phillyrin | 0.818221384 | 1.584387266 | −0.77 |
| Sulfamethazine | 0.894469911 | 1.660693642 | −0.77 |
| Tranexamic acid | 1.522106309 | 2.28843833 | −0.77 |
| Promestriene | 0.969456884 | 1.7414504 | −0.77 |
| Danofloxacin (mesylate) | 1.012557078 | 1.785669522 | −0.77 |
| Pexidartinib | 0.835309278 | 1.60892283 | −0.77 |
| Lidocaine | 0.993471638 | 1.774531182 | −0.78 |
| L-Cycloserine | 0.674589505 | 1.458161533 | −0.78 |
| Choline (bitartrate) | 0.860321384 | 1.646313656 | −0.79 |
| Hydroxyurea | 0.932718996 | 1.720189145 | −0.79 |
| Cefazolin (sodium) | 0.986600955 | 1.776403916 | −0.79 |
| Moxalactam (sodium salt) | 1.057689268 | 1.84946757 | −0.79 |
| Potassium guaiacolsulfonate (hemihydrate) | 0.947783364 | 1.740299733 | −0.79 |
| Fidarestat | 0.685993653 | 1.490690458 | −0.80 |
| Mitiglinide (Calcium) | 0.855918647 | 1.668035311 | −0.81 |
| Degarelix | 0.859020619 | 1.673231511 | −0.81 |
| DL-Panthenol | 0.999537964 | 1.81555899 | −0.82 |
| Disodium succinate | 0.817051205 | 1.635764158 | −0.82 |
| Indomethacin | 0.754610172 | 1.573700543 | −0.82 |
| Piperacetazine | 0.945475077 | 1.768975987 | −0.82 |
| Elvitegravir | 0.944796645 | 1.777071291 | −0.83 |
| Isavuconazole | 0.912607813 | 1.74639649 | −0.83 |
| Lumateperone (tosylate) | 0.776828617 | 1.61512463 | −0.84 |
| Clofoctol | 0.967160409 | 1.815183119 | −0.85 |
| Zanamivir | 1.096873556 | 1.947449768 | −0.85 |
| Probucol | 0.882488834 | 1.749613601 | −0.87 |
| Ipragliflozin | 0.899923896 | 1.768957593 | −0.87 |
| Monobenzone | 1.076607083 | 1.950452303 | −0.87 |
| Loxoprofen | 1.038810426 | 1.914140189 | −0.88 |
| L-Histidine | 1.253849975 | 2.131064446 | −0.88 |
| Ticlopidine (hydrochloride) | 0.7613117 | 1.640114966 | −0.88 |
| Naftifine (hydrochloride) | 1.037432541 | 1.920931361 | −0.88 |
| Bremelanotide (Acetate) | 0.739500266 | 1.638263141 | −0.90 |
| Triclosan | 1.080606269 | 1.985932573 | −0.91 |
| Cisapride | 0.686530771 | 1.595183044 | −0.91 |
| (R)-Baclofen | 1.140089419 | 2.051894762 | −0.91 |
| Thalidomide | 0.94964485 | 1.86505118 | −0.92 |
| Benzbromarone | 0.983695028 | 1.902498181 | −0.92 |
| Tolterodine (tartrate) | 0.952893891 | 1.873125446 | −0.92 |
| L-SelenoMethionine | 1.32687531 | 2.25174994 | −0.92 |
| Spectinomycin (dihydrochloride pentahydrate) | 0.959352394 | 1.888915838 | −0.93 |
| Elagolix sodium | 0.79783858 | 1.728083786 | −0.93 |
| Anamorelin (hydrochloride) | 0.987741029 | 1.922185847 | −0.93 |
| Dicoumarol | 1.114134803 | 2.048993451 | −0.93 |
| Sodium tauroglycocholate | 0.763360457 | 1.712090164 | −0.95 |
| Creatine | 1.347739692 | 2.302920589 | −0.96 |
| Eltrombopag (Olamine) | 0.78562372 | 1.745472062 | −0.96 |
| Fenbufen | 0.811000115 | 1.771525588 | −0.96 |
| Tirofiban (hydrochloride monohydrate) | 0.878770567 | 1.842126873 | −0.96 |
| (R)-(—)-Ibuprofen | 0.777430996 | 1.741618497 | −0.96 |
| Adenine | 0.891376737 | 1.857870483 | −0.97 |
| Cholic acid | 0.934108032 | 1.903047091 | −0.97 |
| Naftopidil | 0.959234006 | 1.940252944 | −0.98 |
| Erythromycin | 0.926850678 | 1.912716763 | −0.99 |
| Betamipron | 0.956137329 | 1.951976716 | −1.00 |
| Nafcillin (sodium monohydrate) | 0.953840854 | 1.956827553 | −1.00 |
| Doxapram (hydrochloride hydrate) | 0.992421633 | 2.003395586 | −1.01 |
| Travoprost | 0.907452061 | 1.919961193 | −1.01 |
| Eptifibatide | 0.960730279 | 1.974290565 | −1.01 |
| Alvimopan (dihydrate) | 0.938332647 | 1.958449455 | −1.02 |
| Streptomycin (sulfate) | 1.265275708 | 2.317402848 | −1.05 |
| Ibutilide (fumarate) | 0.839101009 | 1.968521783 | −1.13 |
| Cetrorelix (Acetate) | 0.745879851 | 1.888997715 | −1.14 |
| Spiramycin | 0.842105263 | 2.037526652 | −1.20 |
| Dehydroandrographolide succinate | 0.985532208 | 2.195488722 | −1.21 |
| Fosphenytoin (disodium) | 1.267759563 | 2.489258991 | −1.22 |
| Cholic acid (sodium) | 1.026328862 | 2.27395607 | −1.25 |
| Cortisone acetate | 0.983695028 | 2.332282319 | −1.35 |
| Oxethazaine | 0.996555288 | 2.44191123 | −1.45 |
| Cyproterone acetate | 1.015386382 | 2.754305118 | −1.74 |

TABLE 2

| | | Cholesterol-free media + | |
|---|---|---|---|
| Drug Name | Complete Media | simvastatin (0.04 μM) | Difference |
| Triflupromazine | 1.475008501 | 0.025417299 | 1.45 |
| (hydrochloride) | | | |
| Nikethamide | 1.430125808 | −0.004931715 | 1.44 |
| Cepharanthine | 1.399537661 | 0.124898785 | 1.27 |
| Berbamine | 1.667020712 | 0.492417484 | 1.17 |
| Biperiden (Hydrochloride) | 1.741783029 | 0.82573659 | 0.92 |
| Neticonazole | 1.261942206 | 0.362845063 | 0.90 |
| Citicoline | 1.556571112 | 0.667683961 | 0.89 |
| Econazole (nitrate) | 1.072727273 | 0.197077059 | 0.88 |
| Clomiphene (citrate) | 1.000548847 | 0.146211786 | 0.85 |
| Bromperidol | 1.317713215 | 0.475790405 | 0.84 |
| Ebastine | 1.066027214 | 0.263787161 | 0.80 |
| Sulfapyridine | 1.844453799 | 1.044645097 | 0.80 |
| Buflomedil (hydrochloride) | 1.555253315 | 0.762898331 | 0.79 |
| Alendronate (sodium hydrate) | 1.169073028 | 0.387978142 | 0.78 |
| Nateglinide | 1.822556391 | 1.052379753 | 0.77 |
| Oxeladin (citrate) | 1.466277217 | 0.7029438 | 0.76 |
| Oxiconazole nitrate | 1.066752412 | 0.30714772 | 0.76 |
| Berbamine (dihydrochloride) | 1.039532535 | 0.285714286 | 0.75 |
| Butoconazole (nitrate) | 0.923167501 | 0.183826028 | 0.74 |
| Diphenylpyraline | 1.366688103 | 0.640980259 | 0.73 |
| (hydrochloride) | | | |
| Flecainide (acetate) | 2.118569636 | 1.398919077 | 0.72 |
| D-Mannitol | 1.747798067 | 1.033240997 | 0.71 |
| Meclofenamate (sodium) | 1.718041407 | 1.009126467 | 0.71 |
| Mecillinam | 1.740054725 | 1.032605969 | 0.71 |
| Cefoperazone | 1.747798067 | 1.06144548 | 0.69 |
| Gestrinone | 1.692803437 | 1.007051121 | 0.69 |
| Pazopanib (Hydrochloride) | 1.20583442 | 0.532499066 | 0.67 |
| Sulconazole (nitrate) | 0.975260975 | 0.302151284 | 0.67 |
| Niclosamide | 1.031396126 | 0.365045942 | 0.67 |
| Milrinone | 1.82480315 | 1.159956866 | 0.66 |
| Oleanolic Acid | 1.721284343 | 1.063229206 | 0.66 |
| Tazemetostat | 1.590452261 | 0.933543397 | 0.66 |
| Alverine (citrate) | 1.12758851 | 0.476071975 | 0.65 |
| Amorolfine (hydrochloride) | 1.0208354 | 0.407816818 | 0.61 |
| Toremifene (citrate) | 1.06190901 | 0.449078341 | 0.61 |
| Donepezil | 1.62007874 | 1.008986341 | 0.61 |
| S-Adenosyl-L-methionine | 1.841928015 | 1.239277652 | 0.60 |
| (tosylate) | | | |
| Benzydamine (hydrochloride) | 1.10751274 | 0.507392076 | 0.60 |
| Ketoconazole | 0.800974026 | 0.201165574 | 0.60 |
| Silver sulfadiazine | 1.641549148 | 1.044645097 | 0.60 |
| Dibucaine (hydrochloride) | 1.390732829 | 0.796610169 | 0.59 |
| Isosorbide | 1.514930183 | 0.92143037 | 0.59 |
| Piperidolate | 1.486690766 | 0.895697523 | 0.59 |
| Tazemetostat (hydrobromide) | 1.468732552 | 0.87854483 | 0.59 |
| Pitolisant (hydrochloride) | 1.031273338 | 0.442008639 | 0.59 |
| Riboflavin Tetrabutyrate | 1.747375328 | 1.162832495 | 0.58 |
| Norethindrone | 1.458308218 | 0.874616172 | 0.58 |
| Ifenprodil (tartrate) | 1.064946945 | 0.491803279 | 0.57 |
| Dexamethasone phosphate | 1.229002625 | 0.662473041 | 0.57 |
| disodium | | | |
| Bufexamac | 1.594133012 | 1.029170931 | 0.56 |
| Guanfacine (hydrochloride) | 1.965495609 | 1.401256208 | 0.56 |
| Tulobuterol (hydrochloride) | 1.604969291 | 1.041248926 | 0.56 |
| Laropiprant | 1.455332217 | 0.895731882 | 0.56 |
| Flupentixol dihydrochloride | 1.08384021 | 0.525527301 | 0.56 |
| LCZ696 | 1.743422437 | 1.186104841 | 0.56 |
| Rupatadine (Fumarate) | 1.535885167 | 0.97918512 | 0.56 |
| Oxybuprocaine hydrochloride | 1.021900743 | 0.466223132 | 0.56 |
| Cefadroxil | 1.587969925 | 1.041299421 | 0.55 |
| Meisoindigo | 1.461104141 | 0.914548642 | 0.55 |
| Iproniazid | 1.75041377 | 1.21076402 | 0.54 |
| Loxoprofen (sodium) | 1.619659019 | 1.086782042 | 0.53 |
| Hydroxyzine (dihydrochloride) | 1.173012692 | 0.640696784 | 0.53 |
| L-Ornithine | 1.469849246 | 0.948438843 | 0.52 |
| 6-Aminocaproic acid | 1.745220193 | 1.22563586 | 0.52 |
| Phenazopyridine | 1.801181102 | 1.283608914 | 0.52 |
| (hydrochloride) | | | |
| Fenticonazole (Nitrate) | 1.166694065 | 0.656416322 | 0.51 |
| Dolasetron (Mesylate hydrate) | 1.816670175 | 1.309505894 | 0.51 |
| Luliconazole | 0.846428571 | 0.353106463 | 0.49 |
| Abarelix (Acetate) | 1.048745981 | 0.559292035 | 0.49 |
| Neostigmine (methyl sulfate) | 1.307503897 | 0.820635528 | 0.49 |
| Efinaconazole | 1.081604679 | 0.595261868 | 0.49 |
| Proparacaine (Hydrochloride) | 1.020938023 | 0.543970209 | 0.48 |
| (+)-Ketoconazole | 0.951111513 | 0.474340699 | 0.48 |
| Sertaconazole (nitrate) | 0.843569688 | 0.370216408 | 0.47 |
| Ciclopirox | 0.680382775 | 0.221877768 | 0.46 |
| Noscapine | 1.210420842 | 0.758614089 | 0.45 |
| Molsidomine | 1.599737102 | 1.149934811 | 0.45 |
| Neticonazole (hydrochloride) | 0.983914729 | 0.535411081 | 0.45 |
| D-(+)-Glucono-1,5-lactone | 1.644074932 | 1.196137447 | 0.45 |
| Linoleic acid | 1.846616541 | 1.398891967 | 0.45 |
| Clotrimazole | 0.858211281 | 0.416578108 | 0.44 |
| Ozagrel | 1.94160105 | 1.503594536 | 0.44 |
| Cefazedone | 1.590535656 | 1.152542373 | 0.44 |
| Procarbazine (Hydrochloride) | 1.193498107 | 0.755551302 | 0.44 |
| Minaprine (dihydrochloride) | 1.277884492 | 0.843891734 | 0.43 |
| Isoconazole (nitrate) | 0.733660366 | 0.300147614 | 0.43 |
| Orlistat | 1.622341568 | 1.190380257 | 0.43 |
| Pranlukast | 1.229839228 | 0.799455412 | 0.43 |
| Hydroxocobalamin | 1.455897127 | 1.027123849 | 0.43 |
| (monohydrochloride) | | | |
| Tiratricol | 1.145401915 | 0.717266462 | 0.43 |
| Sulconazole (mononitrate) | 0.836329627 | 0.409249564 | 0.43 |
| Histamine | 1.573352978 | 1.149987459 | 0.42 |
| Amezinium (methylsulfate) | 1.743501611 | 1.322336943 | 0.42 |
| Ticagrelor | 1.275074479 | 0.853970644 | 0.42 |
| Apremilast | 1.812244898 | 1.395870058 | 0.42 |
| Emamectin (Benzoate) | 0.418897926 | 0.006353817 | 0.41 |
| Diiodohydroxyquinoline | 1.35022508 | 0.939959156 | 0.41 |
| Amikacin (disulfate) | 1.245273312 | 0.839210347 | 0.41 |
| Seratrodast | 1.33492823 | 0.930469442 | 0.40 |
| Posaconazole | 1.904209202 | 1.5 | 0.40 |
| Elvitegravir | 1.434976578 | 1.03242137 | 0.40 |
| Dobutamine (hydrochloride) | 1.236527331 | 0.834309054 | 0.40 |
| Isavuconazole | 0.925649351 | 0.524612343 | 0.40 |
| Bazedoxifene (acetate) | 0.848452957 | 0.447705041 | 0.40 |
| Pramocaine (hydrochloride) | 1.046652031 | 0.646876385 | 0.40 |
| Amodiaquine (dihydrochloride | 1.606022585 | 1.209027169 | 0.40 |
| dihydrate) | | | |
| Amoxicillin | 1.385890767 | 0.994845361 | 0.39 |
| Oxybutynin | 1.133014354 | 0.743578388 | 0.39 |
| Miconazole (nitrate) | 0.800877273 | 0.413724244 | 0.39 |
| Cefepime (Dihydrochloride | 1.536641472 | 1.149934811 | 0.39 |
| Monohydrate) | | | |
| Carbetapentane (citrate) | 1.194903581 | 0.809220004 | 0.39 |
| Amlexanox | 1.160900322 | 0.776582709 | 0.38 |
| Dronedarone | 0.874075292 | 0.490833826 | 0.38 |
| (−)-Limonene | 1.422141853 | 1.043500512 | 0.38 |
| Orphenadrine (citrate) | 1.524859881 | 1.14690027 | 0.38 |
| Terconazole | 1.002696976 | 0.629352227 | 0.37 |
| DL-Xylose | 1.202586759 | 0.832187271 | 0.37 |
| Minocycline (hydrochloride) | 1.957967378 | 1.589395267 | 0.37 |
| Pizotifen | 1.215502555 | 0.848985891 | 0.37 |
| Grazoprevir potassium salt | 1.826893782 | 1.460495083 | 0.37 |
| Carbinoxamine maleate salt | 1.119742765 | 0.756977536 | 0.36 |
| Gamithromycin | 1.765363128 | 1.402868318 | 0.36 |
| Raloxifene (hydrochloride) | 0.748643761 | 0.388433982 | 0.36 |
| Ronidazole | 1.334729202 | 0.974792323 | 0.36 |
| Rifapentine | 1.938330303 | 1.581884464 | 0.36 |
| Methyl aminolevulinate | 1.627341439 | 1.272490222 | 0.35 |
| (hydrochloride) | | | |
| Canrenone | 1.452499172 | 1.097854723 | 0.35 |
| Gadodiamide | 1.18407386 | 0.83597519 | 0.35 |
| Netilmicin (sulfate) | 1.537824286 | 1.190047793 | 0.35 |
| Perflubron | 1.247828991 | 0.901033691 | 0.35 |
| Bifonazole | 0.784446689 | 0.437920289 | 0.35 |
| Eicosapentaenoic Acid | 1.470928256 | 1.130121396 | 0.34 |
| Dimesna | 1.057233704 | 0.716547066 | 0.34 |
| Alimemazine hemitartrate | 1.335305466 | 0.997140912 | 0.34 |
| Retinoic acid | 1.252788104 | 0.919417187 | 0.33 |
| Tamoxifen | 1.083699519 | 0.752608048 | 0.33 |
| Indacaterol | 1.100243871 | 0.769593297 | 0.33 |
| Chlormethine (hydrochloride) | 0.977415307 | 0.646947122 | 0.33 |
| Sertindole | 1.128060264 | 0.798310979 | 0.33 |
| Amprenavir | 1.995608532 | 1.668857727 | 0.33 |
| Fluorometholone acetate | 0.944058259 | 0.61761385 | 0.33 |
| Piperidolate (hydrochloride) | 0.90101295 | 0.575131434 | 0.33 |
| Teneligliptin | 1.011796537 | 0.686127589 | 0.33 |
| Cariprazine (hydrochloride) | 1.452159076 | 1.127294613 | 0.32 |
| Umifenovir (hydrochloride) | 1.225106802 | 0.900912647 | 0.32 |
| Vandetanib | 0.739080834 | 0.417444901 | 0.32 |
| Anisindione | 1.616687578 | 1.296085305 | 0.32 |
| Valacyclovir (hydrochloride) | 1.259678457 | 0.943226685 | 0.32 |
| Acalabrutinib | 1.617942284 | 1.301928133 | 0.32 |
| Thonzonium (bromide) | 0.563794255 | 0.249425727 | 0.31 |
| Verteporfin | 0.929764113 | 0.616015846 | 0.31 |
| Valpromide | 1.716726914 | 1.404172099 | 0.31 |
| Isradipine | 1.060699193 | 0.748500366 | 0.31 |
| Acarbose | 1.152900499 | 0.840965618 | 0.31 |
| Mephenesin | 1.534504391 | 1.224802805 | 0.31 |
| Daidzein | 1.614180861 | 1.305866395 | 0.31 |
| Ropinirole (hydrochloride) | 1.184565916 | 0.878965283 | 0.31 |
| Avapritinib | 0.749038761 | 0.443571844 | 0.31 |
| Nafcillin (sodium monohydrate) | 1.173783287 | 0.869314674 | 0.30 |
| Estradiol | 1.409177015 | 1.105843993 | 0.30 |
| Pazopanib | 1.457409109 | 1.155582305 | 0.30 |
| Fesoterodine (fumarate) | 1.277777778 | 0.97708393 | 0.30 |
| Prednisolone (disodium | 0.737451737 | 0.437948501 | 0.30 |
| phosphate) | | | |
| Lodoxamide (tromethamine) | 1.28176202 | 0.983500135 | 0.30 |
| Tioconazole | 0.967248062 | 0.669012511 | 0.30 |
| Alpha-Estradiol | 1.407685098 | 1.109882821 | 0.30 |
| Ceforanide | 1.331379118 | 1.034374105 | 0.30 |
| Doxepin (Hydrochloride) | 1.15807404 | 0.861756015 | 0.30 |
| Brompheniramine (maleate) | 1.189393939 | 0.893187899 | 0.30 |
| Diflorasone | 1.173601908 | 0.877475948 | 0.30 |
| L-Arginine | 1.484004985 | 1.189216684 | 0.29 |
| Tropisetron | 1.138558074 | 0.844476957 | 0.29 |
| Lasofoxifene (Tartrate) | 0.942769403 | 0.649384131 | 0.29 |
| Kalii Dehydrographolidi | 1.183304482 | 0.8904204 | 0.29 |
| Succinas | | | |
| Temoporfin | 0.920762826 | 0.628617798 | 0.29 |
| Sertraline (hydrochloride) | 1.199597512 | 0.908285335 | 0.29 |
| Fosinopril (sodium) | 1.198604753 | 0.910036548 | 0.29 |
| Medroxyprogesterone | 1.173070384 | 0.885811836 | 0.29 |
| Tocofersolan | 0.628525383 | 0.34622667 | 0.28 |
| Amlodipine | 1.705144291 | 1.424043237 | 0.28 |
| Lovastatin | 0.799207725 | 0.518407576 | 0.28 |
| Pasiniazid | 1.324894515 | 1.046571136 | 0.28 |
| Trimebutine (maleate) | 0.928153446 | 0.651607035 | 0.28 |
| Tenofovir (hydrate) | 2.099121706 | 1.82427695 | 0.27 |
| AZD7545 | 1.088369594 | 0.813632675 | 0.27 |
| Atorvastatin (hemicalcium salt) | 0.729519205 | 0.455504463 | 0.27 |
| Bepridil hydrochloride | 1.583885502 | 1.312110922 | 0.27 |
| Loteprednol Etabonate | 1.184441656 | 0.912795793 | 0.27 |
| Carglumic Acid | 1.206688103 | 0.938325391 | 0.27 |
| Lipoic acid | 1.012048193 | 0.744320109 | 0.27 |
| Diphenidol (hydrochloride) | 1.206066012 | 0.94124424 | 0.26 |
| Fomepizole | 1.120630042 | 0.856181419 | 0.26 |
| Aceglutamide | 1.025867589 | 0.761960497 | 0.26 |
| Cyclobenzaprine | 1.299523262 | 1.035679085 | 0.26 |
| (hydrochloride) | | | |
| Bendazol | 1.471971067 | 1.208290686 | 0.26 |
| Amcinonide | 0.765976397 | 0.502871363 | 0.26 |
| Ramosetron (Hydrochloride) | 1.513090785 | 1.25 | 0.26 |
| Bacitracin | 1.203308329 | 0.940730531 | 0.26 |
| Riluzole | 1.258649518 | 0.996596324 | 0.26 |
| Tegafur | 1.26349718 | 1.001893427 | 0.26 |
| Phenylephrine | 1.517785729 | 1.256333083 | 0.26 |
| Bepridil (hydrochloride | 1.761571808 | 1.500560852 | 0.26 |
| hydrate) | | | |
| Cisatracurium (besylate) | 1.348875181 | 1.090005733 | 0.26 |
| Melitracen (hydrochloride) | 1.49527828 | 1.240020471 | 0.26 |
| Pimavanserin | 1.132822686 | 0.878408666 | 0.25 |
| Amoxicillin (trihydrate) | 1.020233064 | 0.766642282 | 0.25 |
| Salmeterol (xinafoate) | 1.186320755 | 0.934072234 | 0.25 |
| Melatonin | 1.753219776 | 1.501186843 | 0.25 |
| Sulfacetamide (Sodium) | 1.383858268 | 1.134076204 | 0.25 |
| Atorvastatin | 0.865842253 | 0.616928447 | 0.25 |
| Perphenazine | 0.955437831 | 0.707049238 | 0.25 |
| Clozapine | 1.093504823 | 0.845200817 | 0.25 |
| Rosiglitazone | 1.955111173 | 1.706954571 | 0.25 |
| Guaifenesin | 1.325828555 | 1.077678071 | 0.25 |
| Triclabendazole | 1.192908989 | 0.945001432 | 0.25 |
| Rocuronium (Bromide) | 1.096964813 | 0.849337301 | 0.25 |
| Dapagliflozin ((2S)-1,2- | 1.177443609 | 0.931220616 | 0.25 |
| propanediol, hydrate) | | | |
| Nitisinone | 1.188167203 | 0.942682097 | 0.25 |
| Trazodone (hydrochloride) | 1.052990897 | 0.810491207 | 0.24 |
| Hyodeoxycholic acid | 0.996556474 | 0.754887837 | 0.24 |
| Tannic acid | 2.065067368 | 1.82398957 | 0.24 |
| Fasudil (Hydrochloride) | 1.233095743 | 0.992105263 | 0.24 |
| Cefprozil (monohydrate) | 1.216387767 | 0.977257064 | 0.24 |
| Iopanoic acid | 1.352601156 | 1.114946889 | 0.24 |
| γ-Oryzanol | 1.389993972 | 1.154042989 | 0.24 |
| Etomidate | 1.133948009 | 0.900073153 | 0.23 |
| Doxapram (hydrochloride | 1.127869605 | 0.894011113 | 0.23 |
| hydrate) | | | |
| Thiabendazole | 1.186680988 | 0.953664064 | 0.23 |
| Fluphenazine decanoate | 0.975894345 | 0.74336716 | 0.23 |
| Primaquine (Diphosphate) | 1.652316793 | 1.421121252 | 0.23 |
| Etretinate | 1.055064669 | 0.823994148 | 0.23 |
| Alfuzosin (hydrochloride) | 1.006715015 | 0.775764133 | 0.23 |
| Naftifine (hydrochloride) | 1.136134068 | 0.905536119 | 0.23 |
| Levomepromazine | 1.08420026 | 0.855366889 | 0.23 |
| Quetiapine (hemifumarate) | 0.842818912 | 0.6140553 | 0.23 |
| Heparin (sodium salt) (MW | 1.338764426 | 1.110107049 | 0.23 |
| 15 kDa) | | | |
| Gastrodenol | 1.775016351 | 1.546527973 | 0.23 |
| Tenofovir | 1.065589536 | 0.837353549 | 0.23 |
| Etomidate (hydrochloride) | 1.097067486 | 0.87139722 | 0.23 |
| L-Thyroxine | 1.284327087 | 1.058862434 | 0.23 |
| Vilazodone (Hydrochloride) | 0.803707382 | 0.578471961 | 0.23 |
| Glucosamine (hydrochloride) | 1.177507029 | 0.952465178 | 0.23 |
| Gadoxetate (Disodium) | 1.777895574 | 1.55319751 | 0.22 |
| Levoleucovorin (Calcium) | 1.758447787 | 1.534157998 | 0.22 |
| Gastrodin | 1.354631304 | 1.130501535 | 0.22 |
| Pantoprazole (sodium hydrate) | 1.717941547 | 1.493830623 | 0.22 |
| Acetophenazine (dimaleate) | 1.167614282 | 0.945287958 | 0.22 |
| Ropivacaine (mesylate) | 1.150610932 | 0.930156569 | 0.22 |
| Valganciclovir (hydrochloride) | 1.17839196 | 0.959896878 | 0.22 |
| Chlorprothixene | 1.055587613 | 0.837353549 | 0.22 |
| (hydrochloride) | | | |
| Isoniazid | 1.671894605 | 1.455010225 | 0.22 |
| Azelnidipine | 1.012549622 | 0.798244331 | 0.21 |
| Dabrafenib (Mesylate) | 0.64784689 | 0.433569531 | 0.21 |
| Elagolix sodium | 1.627341439 | 1.413298566 | 0.21 |
| Dimercaprol | 1.093504823 | 0.880054459 | 0.21 |
| (±)-Bisoprolol (hemifumarate) | 1.154066986 | 0.942869796 | 0.21 |
| Plicamycin | 0.243644068 | 0.033432947 | 0.21 |
| Angiotensin II human (acetate) | 1.354908696 | 1.146001797 | 0.21 |
| Prulifloxacin | 1.174772783 | 0.966000596 | 0.21 |
| Naloxegol (oxalate) | 1.061723652 | 0.85442575 | 0.21 |
| Sulfacarbamide | 1.143679106 | 0.937906564 | 0.21 |
| Lubiprostone | 1.26657553 | 1.061466133 | 0.21 |
| Indomethacin | 1.473545843 | 1.268578879 | 0.20 |
| Lorlatinib | 1.150096463 | 0.94758339 | 0.20 |
| Phenformin (hydrochloride) | 1.373195876 | 1.170904267 | 0.20 |
| Sulfacetamide | 0.879329004 | 0.677802061 | 0.20 |
| Ambrisentan | 1.496628781 | 1.295748988 | 0.20 |
| 9-Aminoacridine | 0.525717588 | 0.325708502 | 0.20 |
| Lappaconitine (hydrobromide) | 1.321870926 | 1.122711991 | 0.20 |
| Tenoxicam | 0.881898705 | 0.683621781 | 0.20 |
| Fluocinonide | 0.898006431 | 0.699795779 | 0.20 |
| Methylprednisolone | 1.211767824 | 1.015563101 | 0.20 |
| Tafamidis meglumine | 1.150888797 | 0.955060219 | 0.20 |
| Hydrocortisone 17-butyrate | 0.669600346 | 0.474659669 | 0.19 |
| Epristeride | 0.896396974 | 0.701797286 | 0.19 |
| Choline Fenofibrate | 1.294658092 | 1.101414385 | 0.19 |
| Dicoumarol | 1.089302112 | 0.897303972 | 0.19 |
| Salmeterol | 0.83446178 | 0.64276815 | 0.19 |
| Gefarnate | 1.906013802 | 1.714471969 | 0.19 |
| Tivozanib | 1.059566578 | 0.868035191 | 0.19 |
| Ethylparaben | 1.516978099 | 1.325997953 | 0.19 |
| Daclatasvir | 1.136131315 | 0.946282019 | 0.19 |
| Artemether | 0.841017488 | 0.652384973 | 0.19 |
| Isosorbide mononitrate | 1.020938023 | 0.833858493 | 0.19 |
| Dexamethasone | 0.797129187 | 0.610717449 | 0.19 |
| Vinburnine | 1.234932909 | 1.04895288 | 0.19 |
| Sodium stibogluconate | 1.148682439 | 0.962784287 | 0.19 |
| Benzyl alcohol | 1.062269713 | 0.876528482 | 0.19 |
| Dasatinib | 0.51396648 | 0.328552803 | 0.19 |
| Enalapril (maleate) | 1.316862088 | 1.131767402 | 0.19 |
| Arterolane | 1.31404209 | 1.128989108 | 0.19 |
| Camptothecin | 0.074152542 | −0.110011207 | 0.18 |
| Daptomycin | 1.329982964 | 1.147045855 | 0.18 |
| Glasdegib | 0.980932203 | 0.79921554 | 0.18 |
| Iloperidone | 1.133838947 | 0.952283498 | 0.18 |
| Mirodenafil (dihydrochloride) | 0.92732228 | 0.746095399 | 0.18 |
| Sulbactam | 1.241157556 | 1.060313138 | 0.18 |
| Flumethasone | 0.777189156 | 0.596528516 | 0.18 |
| Tamibarotene | 0.736457933 | 0.55596196 | 0.18 |
| Sorafenib | 0.915009042 | 0.736438076 | 0.18 |
| Pramipexole (dihydrochloride | 1.350242054 | 1.173062453 | 0.18 |
| hydrate) | | | |
| Sodium Picosulfate | 1.163682277 | 0.987034369 | 0.18 |
| Levosulpiride | 1.091961701 | 0.91558193 | 0.18 |
| Abacavir | 2.026348808 | 1.850569676 | 0.18 |
| Carmustine | 1.246552975 | 1.070896235 | 0.18 |
| Eugenol | 1.009412305 | 0.834739658 | 0.17 |
| Clofoctol | 1.231634527 | 1.057007615 | 0.17 |
| Urapidil | 1.27107761 | 1.097393297 | 0.17 |
| Afloqualone | 1.332498555 | 1.159716599 | 0.17 |
| Cyproheptadine (hydrochloride) | 0.781681305 | 0.609553024 | 0.17 |
| Diroximel fumarate | 1.101165322 | 0.929919532 | 0.17 |
| Nimesulide | 1.153120936 | 0.982716798 | 0.17 |
| Cloperastine fendizoate | 1.079287723 | 0.909131108 | 0.17 |
| Deracoxib | 1.820864215 | 1.650869321 | 0.17 |
| Octreotide (acetate) | 1.580019717 | 1.410691004 | 0.17 |
| Oxaliplatin | 1.041738796 | 0.872501723 | 0.17 |
| Sparfloxacin | 1.11790729 | 0.949000689 | 0.17 |
| Esomeprazole magnesium | 1.003077318 | 0.834331825 | 0.17 |
| Zoledronic acid (monohydrate) | 0.894452626 | 0.726246472 | 0.17 |
| Neomycin (sulfate) | 1.021436753 | 0.854938852 | 0.17 |
| Clevudine | 0.881009938 | 0.715174466 | 0.17 |
| Atovaquone | 1.492775958 | 1.327327935 | 0.17 |
| Nylidrin (hydrochloride) | 0.857619468 | 0.692603919 | 0.17 |
| Pimozide | 0.968073593 | 0.803101259 | 0.16 |
| Sodium diatrizoate | 1.036571748 | 0.871670009 | 0.16 |
| Diphenhydramine | 0.742902566 | 0.57893626 | 0.16 |
| (hydrochloride) | | | |
| Oxolamine (citrate) | 1.009993186 | 0.84655973 | 0.16 |
| Diquafosol (tetrasodium) | 1.601051594 | 1.438070404 | 0.16 |
| Dapsone | 1.1315427 | 0.968923647 | 0.16 |
| Carbetocin | 1.760453083 | 1.599831744 | 0.16 |
| Inosine | 1.08458279 | 0.923982069 | 0.16 |
| Bromfenac (sodium hydrate) | 1.292504259 | 1.132054674 | 0.16 |
| Trimipramine (maleate) | 1.193066998 | 1.032721468 | 0.16 |
| Tazobactam | 1.299222564 | 1.140610961 | 0.16 |
| Dofetilide | 0.839416058 | 0.681199707 | 0.16 |
| DL-Menthol | 1.050270046 | 0.892166836 | 0.16 |
| Cilostazol | 0.980929358 | 0.8233703 | 0.16 |
| Abacavir (sulfate) | 0.402900886 | 0.245604544 | 0.16 |
| Etofylline | 1.042084168 | 0.884954058 | 0.16 |
| Carbazochrome | 1.238238051 | 1.08158302 | 0.16 |
| Amenamevir | 1.060493317 | 0.904727665 | 0.16 |
| Risperidone | 1.151075619 | 0.995704146 | 0.16 |
| Vemurafenib | 0.955891781 | 0.800586869 | 0.16 |
| Fluoxetine (hydrochloride) | 1.043354148 | 0.888709677 | 0.15 |
| Ramelteon | 1.317717206 | 1.166446208 | 0.15 |
| Etodolac | 1.144841451 | 0.99391849 | 0.15 |
| Adenine | 1.256427916 | 1.106400494 | 0.15 |
| Bemegride | 1.012692051 | 0.862748851 | 0.15 |
| Sulfadimethoxine | 0.968453378 | 0.818963048 | 0.15 |
| Spectinomycin (dihydrochloride | 0.922176309 | 0.772998559 | 0.15 |
| pentahydrate) | | | |
| Eptifibatide | 0.992883379 | 0.84379502 | 0.15 |
| Rufloxacin hydrochloride | 1.097903443 | 0.949000689 | 0.15 |
| Gemifloxacin (mesylate) | 1.098419081 | 0.949623309 | 0.15 |
| Pyrithioxin (dihydrochloride) | 1.11027359 | 0.962188485 | 0.15 |
| Donepezil (Hydrochloride) | 0.816413916 | 0.669354839 | 0.15 |
| Broxyquinoline | 1.219138756 | 1.072187777 | 0.15 |
| Neostigmine (Bromide) | 1.0656103 | 0.918832737 | 0.15 |
| Lefamulin (acetate) | 1.575304153 | 1.428771733 | 0.15 |
| Piperaquine (tetraphosphate | 1.047124447 | 0.900758098 | 0.15 |
| tetrahydrate) | | | |
| Tipiracil (hydrochloride) | 1.030477654 | 0.884272129 | 0.15 |
| Estropipate | 1.076772753 | 0.93125179 | 0.15 |
| Oxantel (pamoate) | 1.050734619 | 0.907182548 | 0.14 |
| Mepivacaine (hydrochloride) | 1.395990799 | 1.252933507 | 0.14 |
| Toceranib (phosphate) | 1.042508175 | 0.900068918 | 0.14 |
| Morinidazole | 1.675977654 | 1.534550196 | 0.14 |
| Selpercatinib | 1.626765487 | 1.485417835 | 0.14 |
| Solriamfetol (hydrochloride) | 1.137545752 | 0.996963563 | 0.14 |
| Cobimetinib (racemate) | 0.405049691 | 0.266161753 | 0.14 |
| Bempedoic acid | 1.280660377 | 1.142747946 | 0.14 |
| Promazine (hydrochloride) | 0.978503629 | 0.840733314 | 0.14 |
| Protirelin (Acetate) | 1.222041612 | 1.084596725 | 0.14 |
| Apixaban | 1.135130692 | 0.997831658 | 0.14 |
| Doravirine | 1.562262787 | 1.425160163 | 0.14 |
| Clorprenaline hydrochloride | 0.819949812 | 0.683172656 | 0.14 |
| Ribociclib succinate hydrate | 0.632069044 | 0.495356691 | 0.14 |
| Carcainium (chloride) | 1.155076304 | 1.018970682 | 0.14 |
| Potassium guaiacolsulfonate | 1.837645085 | 1.701906898 | 0.14 |
| (hemihydrate) | | | |
| Desonide | 0.765776699 | 0.630345572 | 0.14 |
| Puerarin | 1.080722252 | 0.9455843 | 0.14 |
| Irinotecan (hydrochloride) | 1.062953556 | 0.927957195 | 0.13 |
| Cloxacillin (sodium | 1.179685581 | 1.044736057 | 0.13 |
| monohydrate) | | | |
| Hydroxyfasudil (hydrochloride) | 0.978780553 | 0.846091425 | 0.13 |
| Thio-TEPA | 0.817670537 | 0.686413147 | 0.13 |
| Homatropine (Bromide) | 1.530839895 | 1.400071891 | 0.13 |
| Tolazamide | 1.177376527 | 1.046684508 | 0.13 |
| (+)-Camphor | 1.049972083 | 0.919793755 | 0.13 |
| Sacubitril | 1.621735136 | 1.492025278 | 0.13 |
| Dipyridamole | 1.094763776 | 0.965235545 | 0.13 |
| Dienestrol | 0.990886921 | 0.861755978 | 0.13 |
| Brexpiprazole | 1.023644881 | 0.894549763 | 0.13 |
| Tiagabine (hydrochloride) | 0.975153546 | 0.846462332 | 0.13 |
| Mequitazine | 0.984190603 | 0.85585758 | 0.13 |
| Montelukast (sodium) | 0.9745671 | 0.846394006 | 0.13 |
| Hexythiazox | 0.991315965 | 0.864280245 | 0.13 |
| Mometasone furoate | 0.887711864 | 0.761113186 | 0.13 |
| Pentamidine (isethionate) | 0.454775023 | 0.328462647 | 0.13 |
| Roxadustat | 1.327206605 | 1.200945039 | 0.13 |
| Pantoprazole (sodium) | 1.780590127 | 1.654795289 | 0.13 |
| Tiopronin | 1.150649351 | 1.025944628 | 0.12 |
| Quetiapine | 0.928100988 | 0.803721755 | 0.12 |
| Chloroxine | 1.011483254 | 0.888839681 | 0.12 |
| Meloxicam | 1.234027914 | 1.111415071 | 0.12 |
| Crizotinib | 0.52690167 | 0.404357668 | 0.12 |
| Vitamin K4 | 0.877933507 | 0.755883452 | 0.12 |
| Pergolide (mesylate) | 1.079425837 | 0.957927369 | 0.12 |
| Loxoprofen | 1.115013774 | 0.993620086 | 0.12 |
| Trapidil | 1.067921103 | 0.947048024 | 0.12 |
| Ingenol Mebutate | 0.416887945 | 0.296164139 | 0.12 |
| Glafenine | 1.067913605 | 0.947741194 | 0.12 |
| Cetylpyridinium (chloride | 0.271382637 | 0.151940095 | 0.12 |
| monohydrate) | | | |
| Sultamicillin (tosylate) | 1.190898291 | 1.071979668 | 0.12 |
| Promestriene | 0.92768595 | 0.809220004 | 0.12 |
| Niacin | 1.361937591 | 1.243646092 | 0.12 |
| Ciclopirox (olamine) | 0.714977611 | 0.597077244 | 0.12 |
| Clomipramine (hydrochloride) | 1.027978966 | 0.910514541 | 0.12 |
| Mebendazole | 0.58458279 | 0.467500934 | 0.12 |
| Carprofen | 0.980013627 | 0.863444491 | 0.12 |
| Clemastine (fumarate) | 0.998920616 | 0.882885944 | 0.12 |
| Rivastigmine | 1.344521045 | 1.229122874 | 0.12 |
| (−)-Securinine | 0.986513029 | 0.871169481 | 0.12 |
| Clemizole (hydrochloride) | 1.070031235 | 0.955253302 | 0.11 |
| Ribociclib | 0.733817851 | 0.619459688 | 0.11 |
| Betamipron | 0.90748393 | 0.793578926 | 0.11 |
| Fenofibric acid | 1.061139028 | 0.947293039 | 0.11 |
| Solifenacin (hydrochloride) | 0.977955912 | 0.865045942 | 0.11 |
| Ethacrynic acid | 1.267733109 | 1.155003762 | 0.11 |
| Fludrocortisone acetate | 0.56235504 | 0.45 | 0.11 |
| Clindamycin (phosphate) | 1.128787879 | 1.016670097 | 0.11 |
| Dydrogesterone | 1.2952735 | 1.183467142 | 0.11 |
| Benidipine (hydrochloride) | 1.165076661 | 1.053571429 | 0.11 |
| Risedronate (sodium) | 0.996059798 | 0.88470443 | 0.11 |
| Pramiracetam | 0.937953263 | 0.826616175 | 0.11 |
| Nitroprusside (disodium | 0.809991079 | 0.698847926 | 0.11 |
| dihydrate) | | | |
| Buspirone (hydrochloride) | 0.993511834 | 0.882817464 | 0.11 |
| Prucalopride | 0.98538961 | 0.875117078 | 0.11 |
| Temsirolimus | 0.55708975 | 0.447219744 | 0.11 |
| Metyrapone | 1.065821488 | 0.956254572 | 0.11 |
| Valrubicin | 0.184468593 | 0.074985852 | 0.11 |
| (Z)-Capsaicin | 1.242166755 | 1.133511745 | 0.11 |
| Peficitinib | 0.776007802 | 0.667374166 | 0.11 |
| Oseltamivir acid | 1.052631579 | 0.944009217 | 0.11 |
| Aprepitant | 1.143917307 | 1.035936616 | 0.11 |
| Cinnarizine | 1.167869871 | 1.060724335 | 0.11 |
| Halcinonide | 0.682217344 | 0.57509969 | 0.11 |
| Dacomitinib | 0.436984162 | 0.330137872 | 0.11 |
| Fidarestat | 1.736444298 | 1.629726206 | 0.11 |
| Aliskiren (hemifumarate) | 0.833749716 | 0.727522161 | 0.11 |
| D-α-Tocopherol acetate | 1.003281271 | 0.897381884 | 0.11 |
| Vilazodone | 0.910660941 | 0.804859611 | 0.11 |
| Cyproheptadine (hydrochloride | 1.192176484 | 1.086649215 | 0.11 |
| sesquihydrate) | | | |
| Glecaprevir | 1.125833866 | 1.020786058 | 0.11 |
| Lofexidine (hydrochloride) | 1.375703409 | 1.270982311 | 0.10 |
| (−)-Sparteine (sulfate | 0.934505841 | 0.829972916 | 0.10 |
| pentahydrate) | | | |
| Ertugliflozin | 1.807505886 | 1.703289251 | 0.10 |
| Dehydroandrographolide | 1.003902663 | 0.899773616 | 0.10 |
| succinate | | | |
| Dyclonine (hydrochloride) | 0.966073102 | 0.862259562 | 0.10 |
| Oleic acid | 0.83011583 | 0.726677923 | 0.10 |
| Nifuroxazide | 1.264688885 | 1.161336032 | 0.10 |
| Brivudine | 1.259615385 | 1.156506784 | 0.10 |
| Cefaclor | 1.695695038 | 1.593220339 | 0.10 |
| Danofloxacin (mesylate) | 1.081060606 | 0.978769903 | 0.10 |
| Diethylstilbestrol | 1.094596329 | 0.993017979 | 0.10 |
| Mangafodipir (trisodium) | 1.892740353 | 1.791678381 | 0.10 |
| Adenine (hemisulfate) | 1.604390994 | 1.503365115 | 0.10 |
| Avibactam (sodium) | 1.132054428 | 1.031128824 | 0.10 |
| Ornidazole (Levo-) | 1.072305974 | 0.972500716 | 0.10 |
| Isosulfan blue | 1.246965903 | 1.14757085 | 0.10 |
| Tebipenem pivoxil | 0.835566659 | 0.73680878 | 0.10 |
| Bedaquiline | 1.59033877 | 1.492404324 | 0.10 |
| Cortisone acetate | 1.101239669 | 1.003498662 | 0.10 |
| Clevidipine | 0.903572388 | 0.806058967 | 0.10 |
| Troxerutin | 0.94549171 | 0.848248206 | 0.10 |
| Baclofen | 1.019510279 | 0.922451532 | 0.10 |
| Pranlukast (hemihydrate) | 1.28488746 | 1.188835943 | 0.10 |
| Niraparib (hydrochloride) | 0.838033844 | 0.742223424 | 0.10 |
| Allylestrenol | 1.383708273 | 1.287980126 | 0.10 |
| Delapril (hydrochloride) | 0.877164502 | 0.781454886 | 0.10 |
| Anamorelin | 1.358183489 | 1.262714415 | 0.10 |
| Gefitinib | 0.602739726 | 0.507438464 | 0.10 |
| Ilaprazole (sodium) | 1.428057056 | 1.332994235 | 0.10 |
| Loratadine | 0.909713265 | 0.814712462 | 0.10 |
| Emtricitabine | 1.441532622 | 1.347117065 | 0.09 |
| Betamethasone dipropionate | 0.618132924 | 0.523774689 | 0.09 |
| Diazoxide | 1.066345271 | 0.972216505 | 0.09 |
| Tacrolimus (monohydrate) | 1.225042589 | 1.13117284 | 0.09 |
| Loxapine | 0.912521903 | 0.819434629 | 0.09 |
| Cromolyn (sodium) | 0.913095238 | 0.821001145 | 0.09 |
| Brinzolamide | 1.197129187 | 1.105345638 | 0.09 |
| L-Cycloserine | 0.906565657 | 0.814982507 | 0.09 |
| Bumetanide | 1.137792103 | 1.046253719 | 0.09 |
| Rauwolscine (hydrochloride) | 0.836959441 | 0.745469299 | 0.09 |
| Trelagliptin | 1.233521657 | 1.142076503 | 0.09 |
| Vitamin B12 | 1.289070179 | 1.197960706 | 0.09 |
| Quinagolide (hydrochloride) | 1.019426813 | 0.928325293 | 0.09 |
| Moxonidine | 0.928255695 | 0.837253414 | 0.09 |
| Gentamicin (sulfate) | 0.957528958 | 0.867869535 | 0.09 |
| Enasidenib (mesylate) | 1.615690432 | 1.526032824 | 0.09 |
| Sodium copper chlorophyllin A | 1.794201003 | 1.705088558 | 0.09 |
| Darolutamide | 0.996577575 | 0.90754576 | 0.09 |
| Clodronate (disodium | 0.932581252 | 0.843927509 | 0.09 |
| tetrahydrate) | | | |
| Sugammadex (sodium) | 0.994599902 | 0.90611477 | 0.09 |
| Dibucaine | 0.932277319 | 0.84379502 | 0.09 |
| (R)-Serine | 1.051546392 | 0.963311383 | 0.09 |
| Loxapine (succinate) | 1.361250994 | 1.273627617 | 0.09 |
| Efavirenz | 1.301318267 | 1.214605067 | 0.09 |
| Decloxizine (dihydrochloride) | 1.041529455 | 0.955250332 | 0.09 |
| Bronopol | 0.908244379 | 0.822076826 | 0.09 |
| Ioversol | 1.097476401 | 1.011538462 | 0.09 |
| (+)-Kavain | 1.093429777 | 1.008700102 | 0.08 |
| Toceranib | 1.022129108 | 0.937460913 | 0.08 |
| Allopurinol | 0.973416592 | 0.889631336 | 0.08 |
| Riociguat | 1.184975936 | 1.101341282 | 0.08 |
| Selexipag | 1.012963676 | 0.929481585 | 0.08 |
| Naproxen | 1.007427327 | 0.924067301 | 0.08 |
| Rosuvastatin (Calcium) | 1.001356589 | 0.918789097 | 0.08 |
| Sulbutiamine | 0.918281006 | 0.835949464 | 0.08 |
| Delavirdine (mesylate) | 1.034290118 | 0.953238866 | 0.08 |
| Phenothiazine | 1.260731022 | 1.179747955 | 0.08 |
| Udenafil | 2.002097609 | 1.921200224 | 0.08 |
| Dimenhydrinate | 0.975341115 | 0.89453972 | 0.08 |
| Heparin (Lithium salt) | 1.136184657 | 1.05560962 | 0.08 |
| Tofogliflozin (hydrate) | 1.040367965 | 0.960453741 | 0.08 |
| Nonivamide | 1.139150943 | 1.059430537 | 0.08 |
| Flibanserin | 1.088915288 | 1.009530725 | 0.08 |
| Copanlisib (dihydrochloride) | 0.369395917 | 0.290193128 | 0.08 |
| Ciprofloxacin | 1.043277553 | 0.964162646 | 0.08 |
| Lactulose | 0.801504163 | 0.722748174 | 0.08 |
| Gatifloxacin | 1.020034045 | 0.941838649 | 0.08 |
| Domperidone | 1.061287153 | 0.983298539 | 0.08 |
| Triclosan | 1.092975207 | 1.015023667 | 0.08 |
| Metergoline | 0.866834171 | 0.789172157 | 0.08 |
| Dithranol | 0.924902289 | 0.847608135 | 0.08 |
| Hydrocortisone hemisuccinate | 1.019364344 | 0.942312516 | 0.08 |
| Cetrorelix (Acetate) | 1.106918239 | 1.030109146 | 0.08 |
| Idebenone | 1.125468336 | 1.049105927 | 0.08 |
| Ethylenediaminetetraacetic acid | 0.931107838 | 0.855691439 | 0.08 |
| (trisodium salt) | | | |
| Capreomycin (sulfate) | 0.975617739 | 0.90084666 | 0.07 |
| Bergenin | 0.749449531 | 0.675412872 | 0.07 |
| Nicorandil | 1.054810098 | 0.980890052 | 0.07 |
| Naftopidil | 0.763003901 | 0.689205579 | 0.07 |
| (Z)-Thiothixene | 1.061580087 | 0.988344261 | 0.07 |
| Phenylbutazone | 1.14897413 | 1.075806452 | 0.07 |
| Mafenide (Acetate) | 0.898200376 | 0.825534217 | 0.07 |
| Methyl Salicylate | 0.895932321 | 0.823583886 | 0.07 |
| Eslicarbazepine acetate | 1.104308682 | 1.031994554 | 0.07 |
| Besifovir | 0.984555985 | 0.9124103 | 0.07 |
| Benztropine (mesylate) | 0.858601426 | 0.787246964 | 0.07 |
| Sivelestat | 0.865968305 | 0.795239383 | 0.07 |
| Ampiroxicam | 1.381529008 | 1.311007571 | 0.07 |
| Erdafitinib | 0.960200136 | 0.889790576 | 0.07 |
| Orotic acid | 1.260731022 | 1.191244749 | 0.07 |
| Medetomidine (hydrochloride) | 0.944666183 | 0.875214982 | 0.07 |
| Bedaquiline (fumarate) | 0.978503629 | 0.909481524 | 0.07 |
| Doxylamine (succinate) | 1.092457421 | 1.02355523 | 0.07 |
| Sonidegib | 1.188444209 | 1.119694397 | 0.07 |
| Prednisone | 1.027871699 | 0.959789416 | 0.07 |
| Talc | 1.797930359 | 1.729949523 | 0.07 |
| Ziprasidone | 0.983220489 | 0.91550572 | 0.07 |
| Flunisolide | 0.604765086 | 0.537327718 | 0.07 |
| Hydrocortisone | 0.495888039 | 0.428735444 | 0.07 |
| Sodium citrate (dihydrate) | 1.10751274 | 1.040410014 | 0.07 |
| Difluprednate | 1.263715876 | 1.196661007 | 0.07 |
| Prostaglandin E2 | 0.956152433 | 0.88989504 | 0.07 |
| Ipragliflozin | 0.95465368 | 0.888854199 | 0.07 |
| Cyproterone acetate | 1.068181818 | 1.002675448 | 0.07 |
| Bosutinib | 0.352401679 | 0.287310455 | 0.07 |
| Tedizolid | 1.183882399 | 1.118868492 | 0.07 |
| Finasteride | 1.636435228 | 1.571471562 | 0.06 |
| Zalcitabine | 1.889665781 | 1.824733595 | 0.06 |
| Lumefantrine | 0.918423465 | 0.853658537 | 0.06 |
| Atenolol | 1.005704042 | 0.941165484 | 0.06 |
| Cinacalcet | 0.924721401 | 0.860471204 | 0.06 |
| Hydralazine (hydrochloride) | 1.112808843 | 1.049423893 | 0.06 |
| Trimethadione | 1.080142557 | 1.01679223 | 0.06 |
| Amisulpride | 0.933342952 | 0.870264064 | 0.06 |
| Alendronic acid | 0.954671903 | 0.892568203 | 0.06 |
| Ticlopidine (hydrochloride) | 1.477975108 | 1.416432978 | 0.06 |
| Etripamil | 0.872417644 | 0.810942423 | 0.06 |
| Dronedarone (Hydrochloride) | 0.823054238 | 0.761951339 | 0.06 |
| Netupitant | 0.915560685 | 0.854583046 | 0.06 |
| Chondroitin (sulfate) | 1.157909029 | 1.097316836 | 0.06 |
| Relebactam | 0.94513315 | 0.884955752 | 0.06 |
| Irbesartan | 0.969651401 | 0.909629114 | 0.06 |
| Prothionamide | 1.098784611 | 1.03893982 | 0.06 |
| Guacetisal | 1.399602386 | 1.34012813 | 0.06 |
| Asenapine (hydrochloride) | 1.30243106 | 1.243646092 | 0.06 |
| Vernakalant (Hydrochloride) | 1.613860087 | 1.555221186 | 0.06 |
| Metformin (hydrochloride) | 0.894230769 | 0.835823683 | 0.06 |
| Metipranolol hydrochloride | 0.861670696 | 0.80389505 | 0.06 |
| Cinacalcet (hydrochloride) | 0.987715842 | 0.930390492 | 0.06 |
| Isopropamide (iodide) | 1.047381227 | 0.990782736 | 0.06 |
| Choline (chloride) | 0.849435383 | 0.793017821 | 0.06 |
| Chloropyramine hydrochloride | 1.055444222 | 0.999042879 | 0.06 |
| Clofibric acid | 1.071039182 | 1.014770723 | 0.06 |
| Castor oil | 0.802578566 | 0.746551258 | 0.06 |
| Salsalate | 0.936094411 | 0.880359877 | 0.06 |
| Sulfadiazine | 1.555167109 | 1.499439148 | 0.06 |
| Prednisolone (hemisuccinate) | 0.777381218 | 0.721779405 | 0.06 |
| Clindamycin (hydrochloride) | 1.29855643 | 1.243350108 | 0.06 |
| Leuprolide (Acetate) | 1.087379766 | 1.032258065 | 0.06 |
| Ropivacaine (hydrochloride | 1.432796582 | 1.37809648 | 0.05 |
| monohydrate) | | | |
| Desvenlafaxine | 1.101873327 | 1.047235023 | 0.05 |
| Xanthinol Nicotinate | 0.883416387 | 0.829757679 | 0.05 |
| Duloxetine (hydrochloride) | 1.14277939 | 1.089241353 | 0.05 |
| Methylene blue (trihydrate) | 0.390005583 | 0.336579777 | 0.05 |
| Beclometasone | 0.765604681 | 0.712249848 | 0.05 |
| Cefditoren (Pivoxil) | 0.826215418 | 0.773600216 | 0.05 |
| Dolutegravir (sodium) | 0.501249148 | 0.448923596 | 0.05 |
| Memantine (hydrochloride) | 0.853033043 | 0.800709639 | 0.05 |
| Ivosidenib | 0.926526828 | 0.874208983 | 0.05 |
| (−)-Sparteine | 1.105594174 | 1.054196462 | 0.05 |
| Larotrectinib | 1.142583732 | 1.091674048 | 0.05 |
| Tolperisone (hydrochloride) | 0.966377199 | 0.91558193 | 0.05 |
| Carbimazole | 1.518585493 | 1.468270215 | 0.05 |
| Cefpodoxime Proxetil | 1.162223667 | 1.11249242 | 0.05 |
| Ziprasidone (hydrochloride | 0.622769863 | 0.573398499 | 0.05 |
| monohydrate) | | | |
| Omidenepag isopropyl | 1.285666998 | 1.236356793 | 0.05 |
| Deprodone propionate | 0.690657547 | 0.641660016 | 0.05 |
| Dolasetron | 1.019821329 | 0.971354913 | 0.05 |
| (S)-Timolol (Maleate) | 1.285014514 | 1.236766673 | 0.05 |
| Lercanidipine (hydrochloride) | 0.856461504 | 0.808569016 | 0.05 |
| Ceftezole (sodium) | 1.099515672 | 1.052010529 | 0.05 |
| Atropine (sulfate monohydrate) | 0.693845106 | 0.646475306 | 0.05 |
| Otilonium (bromide) | 1.023644881 | 0.97641103 | 0.05 |
| Vardenafil (hydrochloride) | 1.24784689 | 1.200620018 | 0.05 |
| Hydroxychloroquine sulfate | 1.024054983 | 0.976857949 | 0.05 |
| Dehydrocholic acid | 0.900335008 | 0.853337153 | 0.05 |
| Buclizine (dihydrochloride) | 1.018462747 | 0.971857411 | 0.05 |
| Rosiglitazone (maleate) | 0.894330234 | 0.848030019 | 0.05 |
| L-Methionine | 0.886857776 | 0.84062394 | 0.05 |
| Lapatinib | 0.453490966 | 0.407259802 | 0.05 |
| Osimertinib | 0.312287077 | 0.266568172 | 0.05 |
| L-Asparagine | 0.703969 | 0.658823529 | 0.05 |
| Citric acid (trilithium salt | 0.908852888 | 0.863969897 | 0.04 |
| tetrahydrate) | | | |
| Icotinib | 0.692479328 | 0.647607584 | 0.04 |
| Talazoparib | 0.348943348 | 0.304118963 | 0.04 |
| Flunarizine (dihydrochloride) | 0.861688664 | 0.817160827 | 0.04 |
| Tobramycin | 0.906889216 | 0.862464722 | 0.04 |
| Levobupivacaine | 0.915390814 | 0.870976467 | 0.04 |
| (hydrochloride) | | | |
| Edrophonium (chloride) | 1.199336772 | 1.155084999 | 0.04 |
| Benzthiazide | 1.151448041 | 1.107363316 | 0.04 |
| Afatinib | 0.219653179 | 0.175644917 | 0.04 |
| Escin | 0.823869962 | 0.780053599 | 0.04 |
| Tucidinostat | 0.617637893 | 0.573857598 | 0.04 |
| Cholic acid | 1.10536416 | 1.062176636 | 0.04 |
| Mitoxantrone (dihydrochloride) | 0.049760766 | 0.006643047 | 0.04 |
| Hydrocortisone buteprate | 0.653786226 | 0.610697839 | 0.04 |
| Bleomycin (sulfate) | 0.220476781 | 0.177781772 | 0.04 |
| Fludarabine (phosphate) | 1.137926799 | 1.095436922 | 0.04 |
| Bambuterol hydrochloride | 1.226233672 | 1.18402446 | 0.04 |
| Faropenem sodium | 0.984007417 | 0.942120698 | 0.04 |
| Pravastatin (sodium) | 1.234941945 | 1.193197019 | 0.04 |
| Oxprenolol (hydrochloride) | 0.913528139 | 0.871786866 | 0.04 |
| Erdosteine | 1.237844702 | 1.196254539 | 0.04 |
| Vinpocetine | 0.895227732 | 0.853950519 | 0.04 |
| Oxytocin (acetate) | 1.031748911 | 0.990636346 | 0.04 |
| Telmisartan | 0.905964798 | 0.865707882 | 0.04 |
| Berberine (chloride) | 0.394583112 | 0.354445436 | 0.04 |
| L-Epinephrine (Bitartrate) | 1.13596168 | 1.096033403 | 0.04 |
| Decitabine | 0.773253833 | 0.733465608 | 0.04 |
| Disodium succinate | 1.675977654 | 1.636245111 | 0.04 |
| Enasidenib | 0.855224281 | 0.815796592 | 0.04 |
| Topiramate | 1.7058044 | 1.666383701 | 0.04 |
| Flupirtine (Maleate) | 0.941176471 | 0.902353259 | 0.04 |
| Salicylanilide | 1.359468311 | 1.320850202 | 0.04 |
| Fluticasone furoate | 0.748606415 | 0.710356721 | 0.04 |
| Alizapride hydrochloride | 1.057113587 | 1.019458669 | 0.04 |
| Sisomicin (sulfate) | 0.73461583 | 0.697021145 | 0.04 |
| Enfuvirtide (acetate) | 0.963797095 | 0.926260346 | 0.04 |
| Escitalopram (oxalate) | 1.035131354 | 0.997605746 | 0.04 |
| Cefoselis (sulfate) | 1.106766917 | 1.069503903 | 0.04 |
| Terfenadine | 1.013061852 | 0.976152158 | 0.04 |
| Propantheline (bromide) | 1.449467551 | 1.412998976 | 0.04 |
| Raltegravir (potassium salt) | 0.905053459 | 0.868657529 | 0.04 |
| Tofacitinib | 1.224056604 | 1.18784636 | 0.04 |
| Tedizolid (phosphate) | 0.809632035 | 0.773545634 | 0.04 |
| Balsalazide | 1.263654096 | 1.227713766 | 0.04 |
| Fingolimod (hydrochloride) | 1.022653194 | 0.986738887 | 0.04 |
| Acitretin | 0.759969177 | 0.724089069 | 0.04 |
| Nilotinib | 0.979121222 | 0.943676939 | 0.04 |
| Nifenazone | 1.031779661 | 0.996451251 | 0.04 |
| Oseltamivir (phosphate) | 0.862745098 | 0.827698134 | 0.04 |
| Desoxycorticosterone pivalate | 1.76213117 | 1.727706113 | 0.03 |
| Almonertinib (hydrochloride) | 0.719779499 | 0.686126994 | 0.03 |
| Latrepirdine (dihydrochloride) | 1.069805195 | 1.036216047 | 0.03 |
| Saralasin (TFA) | 0.819516205 | 0.786196078 | 0.03 |
| Drofenine (hydrochloride) | 1.144018205 | 1.111279563 | 0.03 |
| Bulleyaconitine A | 1.108653934 | 1.076336571 | 0.03 |
| Lactitol (monohydrate) | 1.214919614 | 1.182845473 | 0.03 |
| Haloperidol | 0.843024002 | 0.811485425 | 0.03 |
| Velpatasvir | 0.776792909 | 0.745469299 | 0.03 |
| Homosalate | 1.441998028 | 1.410691004 | 0.03 |
| Erlotinib (Hydrochloride) | 0.670012547 | 0.638767163 | 0.03 |
| Celecoxib | 1.302446542 | 1.27145749 | 0.03 |
| Betaxolol (hydrochloride) | 1.110184552 | 1.079575135 | 0.03 |
| Crizotinib (hydrochloride) | 0.447389345 | 0.416808149 | 0.03 |
| Lesinurad | 0.938755981 | 0.908325952 | 0.03 |
| Valdecoxib | 0.953813104 | 0.923444976 | 0.03 |
| Pemetrexed (disodium) | 0.14985212 | 0.119947849 | 0.03 |
| Lodenafil | 0.848829649 | 0.818981201 | 0.03 |
| Glafenine (hydrochloride) | 0.924515698 | 0.895673813 | 0.03 |
| Azasetron (hydrochloride) | 0.955179921 | 0.926408193 | 0.03 |
| Manidipine (dihydrochloride) | 1.050622291 | 1.022714526 | 0.03 |
| Ibudilast | 1.27322045 | 1.245416913 | 0.03 |
| L-Valine | 0.867615775 | 0.839887112 | 0.03 |
| Maraviroc | 1.069439096 | 1.041892128 | 0.03 |
| Piroctone olamine | 0.027366448 | 0 | 0.03 |
| Atomoxetine (hydrochloride) | 1.80296462 | 1.775939428 | 0.03 |
| Cetirizine (dihydrochloride) | 0.931506849 | 0.904517176 | 0.03 |
| Mozavaptan (hydrochloride) | 1.196480406 | 1.169501242 | 0.03 |
| Fosamprenavir (Calcium Salt) | 0.877861127 | 0.851137147 | 0.03 |
| Dabigatran etexilate | 0.881789999 | 0.855477032 | 0.03 |
| Clobetasone butyrate | 0.569862012 | 0.543705183 | 0.03 |
| Amiodarone (hydrochloride) | 1.107177033 | 1.081045173 | 0.03 |
| Tolfenamic Acid | 0.998604132 | 0.972500716 | 0.03 |
| Cidofovir | 1.010309278 | 0.984383819 | 0.03 |
| Paeonol | 1.106747066 | 1.080874113 | 0.03 |
| Tinoridine hydrochloride | 1.059381315 | 1.033519553 | 0.03 |
| Tegaserod (maleate) | 0.74734785 | 0.721569751 | 0.03 |
| L-Thyroxine (sodium salt | 0.853225304 | 0.827488487 | 0.03 |
| pentahydrate) | | | |
| Clidinium (bromide) | 1.481615431 | 1.455987718 | 0.03 |
| Trichlormethiazide | 1.045185416 | 1.020191286 | 0.02 |
| L-Carnitine (hydrochloride) | 0.799821866 | 0.775459418 | 0.02 |
| Entrectinib | 0.269588917 | 0.24546222 | 0.02 |
| Cefuroxime axetil | 1.073257626 | 1.049578867 | 0.02 |
| Benzbromarone | 0.981864096 | 0.958221856 | 0.02 |
| Cinoxacin | 0.978159554 | 0.954706363 | 0.02 |
| Colchicine | 0.220528597 | 0.197139652 | 0.02 |
| Carbasalate calcium | 1.128911343 | 1.105528577 | 0.02 |
| Docetaxel | 0.277800744 | 0.254718677 | 0.02 |
| Selamectin | 0.033457249 | 0.010704728 | 0.02 |
| Pamabrom | 0.943325275 | 0.920746551 | 0.02 |
| Ketanserin | 1.461715412 | 1.439374185 | 0.02 |
| Clobetasol propionate | 0.74739922 | 0.725591267 | 0.02 |
| Trovafloxacin | 1.113538563 | 1.091832894 | 0.02 |
| Diethylcarbamazine (citrate) | 0.724733136 | 0.703039257 | 0.02 |
| Galanthamine (hydrobromide) | 1.432288901 | 1.410645257 | 0.02 |
| Riluzole hydrochloride | 0.987663346 | 0.966324771 | 0.02 |
| L-Thyroxine (sodium) | 0.88890492 | 0.867639823 | 0.02 |
| Ezetimibe | 1.268323657 | 1.247467992 | 0.02 |
| DL-Methionine | 1.007036492 | 0.98664158 | 0.02 |
| Emedastine | 0.909320478 | 0.889071825 | 0.02 |
| Cetylpyridinium (chloride) | 0.238055146 | 0.217958365 | 0.02 |
| Suprofen | 0.96366114 | 0.943647108 | 0.02 |
| Reserpine | 0.910241657 | 0.89059728 | 0.02 |
| Acetohydroxamic acid | 0.899204971 | 0.879912862 | 0.02 |
| Migalastat (hydrochloride) | 0.746536452 | 0.727522161 | 0.02 |
| Thiamine (hydrochloride) | 1.11673699 | 1.097748209 | 0.02 |
| Lincomycin (hydrochloride | 0.837800112 | 0.818963048 | 0.02 |
| monohydrate) | | | |
| Ketoprofen | 0.97523531 | 0.956521739 | 0.02 |
| Tenapanor | 1.620053139 | 1.601514302 | 0.02 |
| Rimonabant (Hydrochloride) | 0.838526912 | 0.820368521 | 0.02 |
| Lafutidine | 1.221153846 | 1.203133958 | 0.02 |
| L-Histidine | 1.877915849 | 1.860275513 | 0.02 |
| Visomitin | 0.155400971 | 0.137827265 | 0.02 |
| Fenbufen | 0.892791552 | 0.875900391 | 0.02 |
| Yohimbine (Hydrochloride) | 1.067365603 | 1.050517019 | 0.02 |
| Grazoprevir | 0.938331771 | 0.921512271 | 0.02 |
| Trametinib | 0.258647554 | 0.24229543 | 0.02 |
| Nelarabine | 0.852605329 | 0.83659344 | 0.02 |
| Vilanterol (trifenatate) | 0.936904762 | 0.921323759 | 0.02 |
| Nintedanib | 0.729651163 | 0.714142091 | 0.02 |
| Carbocisteine | 0.949435444 | 0.933960489 | 0.02 |
| Spectinomycin | 1.141626794 | 1.126217892 | 0.02 |
| (dihydrochloride) | | | |
| Vanillin | 1.057186165 | 1.042063635 | 0.02 |
| Omeprazole (sodium) | 0.968848663 | 0.954351643 | 0.01 |
| Bisacodyl | 0.948325359 | 0.934006617 | 0.01 |
| Pidotimod | 1.029614325 | 1.015846882 | 0.01 |
| Vilazodone carboxylic acid | 1.231897106 | 1.218243703 | 0.01 |
| Deferiprone | 1.060714286 | 1.04745551 | 0.01 |
| Bosentan (hydrate) | 1.268617458 | 1.255667258 | 0.01 |
| Temocapril (hydrochloride) | 1.435566134 | 1.422989378 | 0.01 |
| Cytisinicline | 1.015184779 | 1.003101462 | 0.01 |
| Gabapentin (hydrochloride) | 1.102074385 | 1.090496505 | 0.01 |
| Chenodeoxycholic Acid | 0.906141813 | 0.894974936 | 0.01 |
| Dexpramipexole | 1.109857642 | 1.099269678 | 0.01 |
| (dihydrochloride) | | | |
| (−)-Huperzine A | 1.208091437 | 1.197783298 | 0.01 |
| (R)-(−)-Ibuprofen | 1.17845193 | 1.168569582 | 0.01 |
| Oxaprozin | 0.903951701 | 0.894107222 | 0.01 |
| Betahistine (mesylate) | 1.024826216 | 1.015054573 | 0.01 |
| Gemcitabine | 0.090946672 | 0.081430883 | 0.01 |
| Toloxatone | 1.031709957 | 1.022895202 | 0.01 |
| Prednisolone Tebutate | 0.739833684 | 0.731052011 | 0.01 |
| Mildronate | 0.992674617 | 0.983906526 | 0.01 |
| Harringtonine | −0.002433654 | −0.011112826 | 0.01 |
| Belotecan (hydrochloride) | 0.007701194 | −0.000523652 | 0.01 |
| Levocarnitine propionate | 1.114083601 | 1.106058543 | 0.01 |
| (hydrochloride) | | | |
| Batilol | 0.929334583 | 0.921512271 | 0.01 |
| Carbenoxolone (disodium) | 0.982283465 | 0.974478792 | 0.01 |
| Ixabepilone | 0.133575781 | 0.126088038 | 0.01 |
| Halobetasol (propionate) | 0.58495671 | 0.577479446 | 0.01 |
| Indinavir (sulfate) | 0.870752984 | 0.863552171 | 0.01 |
| Chlorquinaldol | 0.007500915 | 0.000886132 | 0.01 |
| Dexchlorpheniramine (maleate) | 0.945891784 | 0.93931853 | 0.01 |
| Pemetrexed (disodium | 0.27458256 | 0.268534239 | 0.01 |
| hemipenta hydrate) | | | |
| Laquinimod | 1.3055288 | 1.299797571 | 0.01 |
| Adapalene | 0.928584765 | 0.923650108 | 0.00 |
| Etidronic acid | 0.901435407 | 0.896811337 | 0.00 |
| 6-Thioguanine | 0.705182529 | 0.701344789 | 0.00 |
| Cevimeline (hydrochloride | 0.948198487 | 0.944369727 | 0.00 |
| hemihydrate) | | | |
| Mebhydrolin (napadisylate) | 0.928969049 | 0.925535988 | 0.00 |
| Ribociclib succinate | 0.609357256 | 0.605951878 | 0.00 |
| Clofarabine | 0.036085826 | 0.033050334 | 0.00 |
| Histamine (dihydrochloride) | 0.923785595 | 0.920939559 | 0.00 |
| Ajmaline | 1.332931485 | 1.330092119 | 0.00 |
| Penciclovir | 1.353211969 | 1.350720318 | 0.00 |
| Quinestrol | 0.021525457 | 0.019149465 | 0.00 |
| Lomustine | 0.760163525 | 0.757914732 | 0.00 |
| Niraparib | 1.193026392 | 1.191295547 | 0.00 |
| Edaravone | 1.020419958 | 1.018825911 | 0.00 |
| Tandospirone (citrate) | 1.040199439 | 1.038611406 | 0.00 |
| Olaparib | 0.834702596 | 0.833116037 | 0.00 |
| L-Eflornithine | 1.267733109 | 1.266365688 | 0.00 |
| (monohydrochloride) | | | |
| Lisinopril (dihydrate) | 0.940926199 | 0.939981185 | 0.00 |
| Triptonide | 0.025217707 | 0.024460157 | 0.00 |
| Ethionamide | 1.611449775 | 1.610922467 | 0.00 |
| Nitazoxanide | 1.209317585 | 1.208842559 | 0.00 |
| Metyrosine | 0.976926853 | 0.976857949 | 0.00 |
| Bosentan | 1.1215311 | 1.121789194 | 0.00 |
| Octenidine (dihydrochloride) | −0.000392503 | −0.000113263 | 0.00 |
| Tetracycline (hydrochloride) | 0.700204406 | 0.700506543 | 0.00 |
| Pimobendan | 1.534852091 | 1.535280728 | 0.00 |
| Topiroxostat | 1.543287327 | 1.543821209 | 0.00 |
| Propranolol (hydrochloride) | 0.87150838 | 0.872229465 | 0.00 |
| Phenytoin (sodium) | 0.888419913 | 0.889270476 | 0.00 |
| Desoximetasone | 0.818396226 | 0.819415249 | 0.00 |
| Alpelisib | 0.550239234 | 0.551372896 | 0.00 |
| Vaborbactam | 0.853246254 | 0.85442575 | 0.00 |
| Gramicidin | 0.214406191 | 0.215686275 | 0.00 |
| Hydrocortisone cypionate | 0.721498662 | 0.72281106 | 0.00 |
| Alarelin (Acetate) | 0.85004697 | 0.85145098 | 0.00 |
| (S)-(−)-Propranolol | 1.663124039 | 1.664890634 | 0.00 |
| (hydrochloride) | | | |
| Fenoterol (hydrobromide) | 0.951235805 | 0.953101072 | 0.00 |
| Enoxacin (hydrate) | 1.164070013 | 1.16609235 | 0.00 |
| Diflunisal | 0.782715337 | 0.784976218 | 0.00 |
| Dihydroergotoxine (mesylate) | 0.90651812 | 0.9088515 | 0.00 |
| Thymopentin (acetate) | 1.151191353 | 1.153892037 | 0.00 |
| Aripiprazole Lauroxil | 0.969690591 | 0.972410334 | 0.00 |
| Ethacridine (lactate monohydrate) | 0.170565524 | 0.173701984 | 0.00 |
| Ulipristal acetate | 0.695044622 | 0.698352941 | 0.00 |
| Captopril | 0.831990794 | 0.835403115 | 0.00 |
| Danoprevir | 0.879905499 | 0.883538971 | 0.00 |
| Histamine (phosphate) | 1.021257523 | 1.0253109 | 0.00 |
| Zofenopril (calcium) | 0.839858583 | 0.844277674 | 0.00 |
| Eprosartan (mesylate) | 0.872414719 | 0.877468217 | −0.01 |
| Canagliflozin | 1.104139505 | 1.109264102 | −0.01 |
| L-Arginine (hydrochloride) | 0.924109075 | 0.929309496 | −0.01 |
| Trifluridine | 0.106575257 | 0.111937655 | −0.01 |
| Galanthamine | 1.046020999 | 1.051421452 | −0.01 |
| Bezafibrate | 1.024288107 | 1.029790891 | −0.01 |
| Lanatoside C | −0.008403361 | −0.002743774 | −0.01 |
| Idarubicin (hydrochloride) | 0.002721335 | 0.008408179 | −0.01 |
| Sulfadoxine | 1.093487011 | 1.099689854 | −0.01 |
| Delamanid | 1.345348529 | 1.351726089 | −0.01 |
| Aclacinomycin A hydrochloride | 0.010234853 | 0.016882999 | −0.01 |
| Asunaprevir | 0.997943723 | 1.004995317 | −0.01 |
| Paroxetine (hydrochloride) | 0.908647594 | 0.916009703 | −0.01 |
| Benzamil (hydrochloride) | 0.811796537 | 0.819336039 | −0.01 |
| Phentolamine (mesylate) | 0.899218314 | 0.907189917 | −0.01 |
| Cabozantinib | 1.160853977 | 1.169032499 | −0.01 |
| Osalmid | 0.89512358 | 0.903330781 | −0.01 |
| Azaperone | 0.998266898 | 1.006957194 | −0.01 |
| Eravacycline (dihydrochloride) | 0.799874529 | 0.808793456 | −0.01 |
| Propafenone (hydrochloride) | 1.100279467 | 1.109796964 | −0.01 |
| Phenazine (methylsulfate) | 0.062092191 | 0.072485578 | −0.01 |
| Etoricoxib | 1.490484316 | 1.501053265 | −0.01 |
| Pazufloxacin (mesylate) | 0.924835989 | 0.935662171 | −0.01 |
| Tandospirone | 0.847929592 | 0.859111038 | −0.01 |
| DL-Arginine | 0.977581411 | 0.988899341 | −0.01 |
| Ouabain (Octahydrate) | −0.011061093 | 0.000544588 | −0.01 |
| Auranofin | 0.000831342 | 0.012507272 | −0.01 |
| Acamprosate (calcium) | 1.327830189 | 1.339957959 | −0.01 |
| Lenalidomide | 0.995755627 | 1.008032675 | −0.01 |
| Chlorpheniramine (maleate) | 1.413648441 | 1.425967471 | −0.01 |
| Olprinone (Hydrochloride) | 1.640110251 | 1.652723443 | −0.01 |
| Ipriflavone | 1.015530741 | 1.028626287 | −0.01 |
| Panobinostat | −0.00472503 | 0.008426121 | −0.01 |
| Lapatinib (ditosylate) | 0.475775194 | 0.488941019 | −0.01 |
| Lumateperone (tosylate) | 1.356558773 | 1.370149912 | −0.01 |
| Dasatinib (hydrochloride) | 0.26802049 | 0.281789987 | −0.01 |
| Propylthiouracil | 0.938976378 | 0.952911574 | −0.01 |
| Daunorubicin (Hydrochloride) | −0.00749489 | 0.006542845 | −0.01 |
| Demeclocycline (hydrochloride) | 1.039935065 | 1.054115933 | −0.01 |
| Telbivudine | 0.86053316 | 0.874772589 | −0.01 |
| Imatinib | 1.199315515 | 1.213858797 | −0.01 |
| Sulfasalazine | 1.155023923 | 1.169619132 | −0.01 |
| Saxagliptin | 0.81601294 | 0.830742049 | −0.01 |
| (R)-Terazosin | 0.922735492 | 0.937906564 | −0.02 |
| Eprazinone (dihydrochloride) | 0.922734358 | 0.938552833 | −0.02 |
| Felbinac | 1.199454173 | 1.215445026 | −0.02 |
| Merbromin | 1.006381934 | 1.022765757 | −0.02 |
| Lenvatinib (mesylate) | 1.153514527 | 1.170019898 | −0.02 |
| Butenafine (Hydrochloride) | 1.060776488 | 1.077775655 | −0.02 |
| Lecithin | 0.87704596 | 0.894308943 | −0.02 |
| Pentoxifylline | 0.926767677 | 0.944227207 | −0.02 |
| Epinastine | 0.965641953 | 0.983111566 | −0.02 |
| Pipemidic acid | 0.989571899 | 1.007532122 | −0.02 |
| Telithromycin | 0.854021847 | 0.872036131 | −0.02 |
| Emetine (dihydrochloride hydrate) | 0.032140751 | 0.050484954 | −0.02 |
| Citric acid | 1.208871235 | 1.22732405 | −0.02 |
| Ibutilide (fumarate) | 1.026775107 | 1.045475175 | −0.02 |
| Chlormadinone acetate | 1.05967285 | 1.078515022 | −0.02 |
| Ensulizole | 0.883253589 | 0.902125775 | −0.02 |
| Pramipexole (dihydrochloride) | 0.865968305 | 0.885041926 | −0.02 |
| Pemetrexed | 0.200412817 | 0.219597115 | −0.02 |
| Labetalol (hydrochloride) | 1.128027049 | 1.147499319 | −0.02 |
| Acetazolamide | 0.939782279 | 0.959374438 | −0.02 |
| Methotrexate | 0.107821751 | 0.127523911 | −0.02 |
| Sacubitril hemicalcium salt | 1.000200924 | 1.019959058 | −0.02 |
| Acefylline | 1.05819497 | 1.078257441 | −0.02 |
| Ganciclovir | 0.859876774 | 0.880021598 | −0.02 |
| Bortezomib | −0.017004128 | 0.003233027 | −0.02 |
| Sulindac | 1.172652242 | 1.193127962 | −0.02 |
| Cinobufotalin | 0.010461678 | 0.031151832 | −0.02 |
| Gemcitabine (hydrochloride) | 0.023060143 | 0.043924903 | −0.02 |
| Proglumide | 0.784190715 | 0.805287759 | −0.02 |
| Phenoxybenzamine | 0.879850047 | 0.901171789 | −0.02 |
| (hydrochloride) | | | |
| Zanubrutinib | 0.703078451 | 0.724501317 | −0.02 |
| Raltitrexed | 0.090699967 | 0.112125163 | −0.02 |
| Lidocaine (hydrochloride) | 0.864637747 | 0.886238919 | −0.02 |
| Ixazomib | −0.009398157 | 0.012230635 | −0.02 |
| Everolimus | 0.372962055 | 0.394620305 | −0.02 |
| Catechin | 1.639060244 | 1.661149564 | −0.02 |
| L-Glutamine | 0.795411917 | 0.817779236 | −0.02 |
| Capsaicin | 1.380283075 | 1.402884855 | −0.02 |
| Dolutegravir | 0.573762893 | 0.596388332 | −0.02 |
| Ambroxol (hydrochloride) | 0.982230074 | 1.004944735 | −0.02 |
| Tacrine hydrochloride hydrate | 1.382760699 | 1.405834186 | −0.02 |
| Sulpiride | 1.053396055 | 1.076529133 | −0.02 |
| Oxaceprol | 0.855224281 | 0.878550176 | −0.02 |
| Sulfamethizole | 1.130316125 | 1.153664921 | −0.02 |
| Acetylleucine | 0.871379898 | 0.89484127 | −0.02 |
| Flutamide | 0.854950937 | 0.878571429 | −0.02 |
| Varenicline (Tartrate) | 1.705076213 | 1.728827818 | −0.02 |
| Betaxolol | 0.917156528 | 0.940971618 | −0.02 |
| Moclobemide | 0.895692277 | 0.919583192 | −0.02 |
| Nebivolol (hydrochloride) | 0.87741642 | 0.901308901 | −0.02 |
| Anamorelin (hydrochloride) | 1.063693685 | 1.087591903 | −0.02 |
| Pericyazine | 1.353211969 | 1.377545951 | −0.02 |
| Anastrozole | 1.04512987 | 1.06951816 | −0.02 |
| Iopromide | 0.795483061 | 0.819894829 | −0.02 |
| Rucaparib (phosphate) | 1.389585947 | 1.414110429 | −0.02 |
| Meclofenoxate (hydrochloride) | 0.97636897 | 1.000933881 | −0.02 |
| Clebopride (malate) | 0.728126086 | 0.753202655 | −0.03 |
| Piperonyl butoxide | 1.180429129 | 1.205882353 | −0.03 |
| Chlorhexidine | 0.591897106 | 0.617562968 | −0.03 |
| Oxytetracycline (hydrochloride) | 0.965243297 | 0.990967256 | −0.03 |
| Fluspirilene | 1.241237113 | 1.267300464 | −0.03 |
| Bucladesine (calcium) | 0.804004329 | 0.830159226 | −0.03 |
| Dabrafenib | 0.703656379 | 0.729917931 | −0.03 |
| Hemin | 1.151196172 | 1.177590788 | −0.03 |
| Cyclosporin A | 1.298619824 | 1.325299445 | −0.03 |
| Glycerol | 1.039334065 | 1.066016837 | −0.03 |
| Homoharringtonine | 0.006300039 | 0.033102618 | −0.03 |
| Imidafenacin | 1.382522951 | 1.409332322 | −0.03 |
| Vecuronium (bromide) | 0.953412073 | 0.98023005 | −0.03 |
| Nimodipine | 1.187106698 | 1.21452608 | −0.03 |
| Etravirine | 0.807105017 | 0.834573149 | −0.03 |
| Racanisodamine | 0.920680165 | 0.948271574 | −0.03 |
| Pirenzepine (dihydrochloride) | 1.26469521 | 1.292566406 | −0.03 |
| Linagliptin | 1.066985646 | 1.094898137 | −0.03 |
| Ganciclovir (sodium) | 1.394684506 | 1.422731357 | −0.03 |
| Teriparatide | 1.019986373 | 1.04833263 | −0.03 |
| Triptolide | −0.021773765 | 0.007136485 | −0.03 |
| Retapamulin | 1.637952734 | 1.667134044 | −0.03 |
| Sulfogaiacol | 0.948828491 | 0.978111873 | −0.03 |
| Tropicamide | 1.135344997 | 1.165133051 | −0.03 |
| Carboxin | 0.927187709 | 0.957695253 | −0.03 |
| Estradiol valerianate | 0.942337567 | 0.973223201 | −0.03 |
| Pirarubicin (Hydrochloride) | 0.524350649 | 0.555416797 | −0.03 |
| L-Cysteine | 1.439369044 | 1.470664928 | −0.03 |
| Apalutamide | 1.081641758 | 1.11299009 | −0.03 |
| Berberine (chloride hydrate) | 0.349455698 | 0.381269099 | −0.03 |
| Tasimelteon | 0.961844197 | 0.994301393 | −0.03 |
| Sivelestat (sodium tetrahydrate) | 1.038565891 | 1.071157283 | −0.03 |
| Digitoxin | −0.022117686 | 0.010532419 | −0.03 |
| Sofosbuvir | 0.962829582 | 0.995507148 | −0.03 |
| Iodixanol | 1.163744943 | 1.196963563 | −0.03 |
| Rilmenidine (phosphate) | 1.214995314 | 1.24872872 | −0.03 |
| Benzyl benzoate | 0.797922811 | 0.831886539 | −0.03 |
| Pinaverium bromide | 0.743766006 | 0.777738516 | −0.03 |
| Sulfameter | 0.947089464 | 0.981573931 | −0.03 |
| Furazolidone | 0.902888333 | 0.937958115 | −0.04 |
| Triamcinolone | 1.165291568 | 1.200504767 | −0.04 |
| Aniracetam | 1.005775895 | 1.041193926 | −0.04 |
| Vigabatrin | 0.774330042 | 0.809878958 | −0.04 |
| Tetramisole (hydrochloride) | 0.9756691 | 1.011265545 | −0.04 |
| Chloroxylenol | 0.951035227 | 0.986634953 | −0.04 |
| Nefopam (hydrochloride) | 1.041396326 | 1.077062721 | −0.04 |
| Nomegestrol acetate | 0.951851011 | 0.987547488 | −0.04 |
| Deflazacort | 0.589926939 | 0.625698324 | −0.04 |
| Dasabuvir | 1.064448629 | 1.10034774 | −0.04 |
| Tanshinone I | 0.864475579 | 0.901188243 | −0.04 |
| Imipenem monohydrate | 0.954795802 | 0.991564863 | −0.04 |
| Elbasvir | 0.937790698 | 0.974642538 | −0.04 |
| Vincristine (sulfate) | 0.111874916 | 0.148763251 | −0.04 |
| Vismodegib | 1.051623377 | 1.088666875 | −0.04 |
| Gemcitabine (elaidate) | 0.096171085 | 0.133430871 | −0.04 |
| Opicapone | 0.897093114 | 0.934557063 | −0.04 |
| Fadrozole hydrochloride | 0.947727273 | 0.985430326 | −0.04 |
| Nepafenac | 1.69948259 | 1.737240606 | −0.04 |
| Neratinib | 0.300707395 | 0.338733833 | −0.04 |
| Azatadine (dimaleate) | 1.047383827 | 1.085511741 | −0.04 |
| Chlorhexidine (digluconate) | 0.96937799 | 1.007528787 | −0.04 |
| Deferasirox (Fe3+ chelate) | 1.516197557 | 1.554564377 | −0.04 |
| Cefoxitin (sodium) | 0.88182721 | 0.920210764 | −0.04 |
| Amsacrine | 0.345973678 | 0.384424817 | −0.04 |
| Lurasidone (Hydrochloride) | 0.804612029 | 0.843074743 | −0.04 |
| Liranaftate | 0.921700695 | 0.960371206 | −0.04 |
| Pramipexole | 1.035039113 | 1.074150168 | −0.04 |
| Succinylsulfathiazole | 1.191114149 | 1.230715654 | −0.04 |
| Fusidic acid (sodium salt) | 0.948373891 | 0.988219895 | −0.04 |
| Eltrombopag (Olamine) | 1.058443007 | 1.098341232 | −0.04 |
| Amifostine | 0.942889871 | 0.982878645 | −0.04 |
| Ferulic acid (sodium) | 0.765978935 | 0.805970149 | −0.04 |
| Pamidronic acid | 1.049469478 | 1.089520043 | −0.04 |
| Tezacaftor | 1.186929596 | 1.227306687 | −0.04 |
| Regorafenib (Hydrochloride) | 0.90259485 | 0.943297687 | −0.04 |
| Methotrexate (disodium) | 0.231784487 | 0.272686433 | −0.04 |
| Imrecoxib | 1.141458107 | 1.182761373 | −0.04 |
| (R)-Lansoprazole | 1.388815266 | 1.430956423 | −0.04 |
| Penfluridol | 1.033138208 | 1.075799447 | −0.04 |
| Tipranavir | 1.176605985 | 1.219572777 | −0.04 |
| Aprotinin | 0.810174991 | 0.853218089 | −0.04 |
| Sulfachloropyridazine | 0.932027257 | 0.975088183 | −0.04 |
| 4-(Aminomethyl)benzoic acid | 1.637235666 | 1.680348608 | −0.04 |
| Cyclic somatostatin | 0.933696022 | 0.976858877 | −0.04 |
| Vorapaxar | 1.004593176 | 1.047807333 | −0.04 |
| Flubendazole | 1.078680529 | 1.122185373 | −0.04 |
| Dichlorphenamide | 1.072996707 | 1.116526362 | −0.04 |
| Succinic acid | 1.685695538 | 1.729331416 | −0.04 |
| Disopyramide | 1.256712627 | 1.300974584 | −0.04 |
| Pitavastatin (Calcium) | 0.44368107 | 0.488319856 | −0.04 |
| Citicoline (sodium) | 1.292287322 | 1.337222431 | −0.04 |
| Paritaprevir | 1.102392344 | 1.147475642 | −0.05 |
| Isocarboxazid | 1.505646173 | 1.550832603 | −0.05 |
| Podofilox | 0.121468927 | 0.166716373 | −0.05 |
| Pheniramine (Maleate) | 1.284258955 | 1.329856584 | −0.05 |
| Granisetron (Hydrochloride) | 0.853608247 | 0.899403051 | −0.05 |
| Paclitaxel | 0.164809835 | 0.210680322 | −0.05 |
| Tazarotene | 0.864565588 | 0.910714286 | −0.05 |
| Levobetaxolol (hydrochloride) | 0.958055378 | 1.004447672 | −0.05 |
| Safinamide | 1.012265725 | 1.059123344 | −0.05 |
| Vesnarinone | 0.90311493 | 0.950642156 | −0.05 |
| Menadione | 1.054810098 | 1.102356021 | −0.05 |
| Adefovir | 0.946183056 | 0.993746091 | −0.05 |
| Dapoxetine (hydrochloride) | 1.031254019 | 1.078829135 | −0.05 |
| Levomefolic acid | 0.85676913 | 0.904413832 | −0.05 |
| Fimasartan | 1.221929969 | 1.269860043 | −0.05 |
| Triamcinolone (acetonide) | 0.587969925 | 0.635904579 | −0.05 |
| Meropenem (trihydrate) | 1.050317401 | 1.098596112 | −0.05 |
| Puromycin (dihydrochloride) | −0.050664612 | −0.002226721 | −0.05 |
| Terazosin (hydrochloride | 0.980570484 | 1.029120978 | −0.05 |
| dihydrate) | | | |
| Leflunomide | 0.949033167 | 0.997805413 | −0.05 |
| Coumarin | 0.794845493 | 0.844112972 | −0.05 |
| Amfenac (Sodium Hydrate) | 0.84619824 | 0.895850418 | −0.05 |
| Nestoron | 0.913157419 | 0.962817771 | −0.05 |
| Adefovir dipivoxil | 0.198643411 | 0.248547811 | −0.05 |
| Bismuth subgallate | 0.983231958 | 1.033313621 | −0.05 |
| Ceritinib | 0.066203244 | 0.116296575 | 0.05 |
| Methacholine (chloride) | 1.194915254 | 1.245153594 | −0.05 |
| Fluvoxamine (maleate) | 0.942592949 | 0.99291498 | −0.05 |
| (+)-Catechin hydrate | 1.059345059 | 1.109923664 | −0.05 |
| Pregnenolone monosulfate | 1.253565988 | 1.304510003 | −0.05 |
| Tenofovir alafenamide fumarate | 0.871174286 | 0.922311631 | −0.05 |
| Gilteritinib | 0.368469294 | 0.419637273 | −0.05 |
| Fipronil | 1.18425492 | 1.235463188 | −0.05 |
| Blonanserin | 0.893867121 | 0.94510582 | −0.05 |
| Vincamine | 0.76857913 | 0.819863851 | −0.05 |
| Icotinib (Hydrochloride) | 0.53432282 | 0.586587436 | −0.05 |
| Pargyline (hydrochloride) | 0.82426405 | 0.876728111 | −0.05 |
| 5-Azacytidine | 0.885848172 | 0.938315277 | −0.05 |
| Zinc Pyrithione | −0.048352919 | 0.004251012 | −0.05 |
| Diclofenac (potassium) | 1.040797658 | 1.09348693 | −0.05 |
| Travoprost | 0.881772268 | 0.935171846 | −0.05 |
| Furosemide (sodium) | 1.020256304 | 1.07370124 | −0.05 |
| Sofalcone | 0.795154583 | 0.848607367 | −0.05 |
| Meglumine | 1.304832714 | 1.358311032 | −0.05 |
| Gadopentetate (dimeglumine) | 1.100280578 | 1.153868528 | −0.05 |
| Dichlorisone acetate | 0.690585308 | 0.744215134 | −0.05 |
| Latanoprost | 0.976940438 | 1.03058104 | −0.05 |
| Doxifluridine | 1.031222123 | 1.085023041 | −0.05 |
| Cytarabine (hydrochloride) | 0.301076772 | 0.355300056 | −0.05 |
| Tolterodine (tartrate) | 1.127325581 | 1.182864164 | −0.06 |
| Stiripentol | 0.875394654 | 0.931276649 | −0.06 |
| Moguisteine | 0.878958841 | 0.935077638 | −0.06 |
| Encorafenib | 0.550089569 | 0.606327521 | −0.06 |
| Ozagrel (sodium) | 1.026082251 | 1.082422729 | −0.06 |
| Cobimetinib | 0.525792189 | 0.582463466 | −0.06 |
| Methenamine (hippurate) | 0.328615716 | 0.385512367 | −0.06 |
| Rotigotine | 1.070356197 | 1.127534262 | −0.06 |
| Cabazitaxel | 0.306524489 | 0.364006989 | −0.06 |
| Furagin | 1.078598764 | 1.136595311 | −0.06 |
| Quinidine hydrochloride | 1.402049427 | 1.460081883 | −0.06 |
| monohydrate | | | |
| Clofazimine | 0.936475127 | 0.994607226 | −0.06 |
| Verapamil (hydrochloride) | 1.012549622 | 1.070958303 | −0.06 |
| Isocorydine | 1.214075166 | 1.272591709 | −0.06 |
| Domiphen (bromide) | −0.042697782 | 0.016138892 | −0.06 |
| Flopropione | 1.044360745 | 1.103328366 | −0.06 |
| Aminophylline | 0.85884807 | 0.918042908 | −0.06 |
| Vigabatrin (hydrochloride) | 1.062636335 | 1.122210414 | −0.06 |
| Palbociclib (isethionate) | 0.691381739 | 0.751426481 | −0.06 |
| Palmatine (chloride) | 0.645862001 | 0.706043273 | −0.06 |
| Ibrutinib (Racemate) | 0.285760728 | 0.345967253 | −0.06 |
| L-Ascorbic acid (sodium salt) | 0.919939369 | 0.980226496 | −0.06 |
| Perhexiline maleate | 0.810999442 | 0.871670009 | −0.06 |
| Ramipril | 0.994092958 | 1.055067837 | −0.06 |
| Rivastigmine (tartrate) | 1.417439703 | 1.478494624 | −0.06 |
| Nitrendipine | 1.042413283 | 1.10348906 | −0.06 |
| i-Inositol | 0.933342952 | 0.994915532 | −0.06 |
| Perospirone | 0.932904184 | 0.994588849 | −0.06 |
| Mitoxantrone | −0.016413916 | 0.045391705 | −0.06 |
| Milnacipran (hydrochloride) | 1.56374238 | 1.625636672 | −0.06 |
| Ponatinib | 0.40635254 | 0.46825158 | −0.06 |
| Floxuridine | 0.029068864 | 0.090981644 | −0.06 |
| Erlotinib | 0.459877101 | 0.521857923 | −0.06 |
| Naftidrofuryl (oxalate) | 1.076025547 | 1.138980018 | −0.06 |
| L-Ornithine (hydrochloride) | 0.813167648 | 0.876300253 | −0.06 |
| Indomethacin (sodium hydrate) | 0.910956637 | 0.974407226 | −0.06 |
| Pomalidomide | 1.029583875 | 1.093086719 | −0.06 |
| Torsemide | 0.990944735 | 1.054535637 | −0.06 |
| Tavaborole | 1.116740512 | 1.180769231 | −0.06 |
| Norepinephrine | 0.884035442 | 0.948124501 | −0.06 |
| Nintedanib esylate | 0.657668968 | 0.721808246 | −0.06 |
| Esaxerenone | 0.911810533 | 0.976266383 | −0.06 |
| Secnidazole | 1.4092827 | 1.474411464 | −0.07 |
| Ranolazine (dihydrochloride) | 0.901738779 | 0.967137809 | −0.07 |
| Levobunolol (hydrochloride) | 0.921015515 | 0.986428659 | −0.07 |
| Salicylamide | 1.140530894 | 1.206484274 | −0.07 |
| Ombitasvir | 0.535771065 | 0.601730688 | −0.07 |
| Sildenafil (citrate) | 0.910569106 | 0.976631314 | −0.07 |
| Methyclothiazide | 1.08076198 | 1.146861429 | −0.07 |
| Mirtazapine | 0.914041995 | 0.98023005 | −0.07 |
| Fludarabine | 1.187054778 | 1.255293767 | −0.07 |
| Diclofenac (diethylamine) | 0.920634921 | 0.989252815 | −0.07 |
| Clopidogrel | 0.968995453 | 1.037749416 | −0.07 |
| Triclocarban | 0.960142507 | 1.028905054 | −0.07 |
| Olsalazine (Disodium) | 1.148919633 | 1.217877095 | −0.07 |
| Naphazoline (hydrochloride) | 0.680218033 | 0.749472351 | −0.07 |
| Flavoxate (hydrochloride) | 0.884894053 | 0.954205119 | −0.07 |
| Degarelix | 1.609329446 | 1.678834182 | −0.07 |
| Revaprazan (hydrochloride) | 0.935679612 | 1.005291577 | −0.07 |
| Dimemorfan (phosphate) | 0.868678517 | 0.93852238 | −0.07 |
| Embelin | 0.829849695 | 0.899764706 | −0.07 |
| Dexmedetomidine (hydrochloride) | 1.1565181 | 1.226527571 | −0.07 |
| Sodium gualenate | 0.809416159 | 0.879631932 | −0.07 |
| Letermovir | 1.456718003 | 1.52720785 | −0.07 |
| Doxorubicin (hydrochloride) | 0.019941836 | 0.090539166 | −0.07 |
| Ondansetron | 0.947958866 | 1.018774354 | −0.07 |
| Triflusal | 1.082981716 | 1.154042989 | −0.07 |
| Rolapitant | 0.930418006 | 1.001497617 | −0.07 |
| Medroxyprogesterone acetate | 0.929065358 | 1.000538561 | −0.07 |
| 6α-Methylprednisolone 21- | 0.68202324 | 0.753613094 | −0.07 |
| hemisuccinate (sodium salt) | | | |
| Nilotinib (monohydrochloride | 0.782122905 | 0.853976532 | −0.07 |
| monohydrate) | | | |
| Dirithromycin | 1.492868024 | 1.564937267 | −0.07 |
| Imipramine (hydrochloride) | 0.869372294 | 0.941721303 | −0.07 |
| 4-Methylumbelliferone | 1.056603774 | 1.129167394 | −0.07 |
| Vancomycin (hydrochloride) | 0.742236333 | 0.815238847 | −0.07 |
| Fosamprenavir | 1.120395706 | 1.194130926 | −0.07 |
| Rizatriptan (benzoate) | 0.979938796 | 1.054248862 | −0.07 |
| Alibendol | 0.877432672 | 0.951753295 | −0.07 |
| Tigecycline (tetramesylate) | 0.333032001 | 0.407457323 | −0.07 |
| Cytarabine | 0.195121951 | 0.269818443 | −0.07 |
| Alprenolol (hydrochloride) | 1.152665193 | 1.227855652 | −0.08 |
| Sulfisoxazole | 1.045059112 | 1.12031452 | −0.08 |
| N-Acetylprocainamide | 0.847929592 | 0.923404953 | −0.08 |
| Diclazuril | 0.976574937 | 1.052094241 | −0.08 |
| Sotalol (hydrochloride) | 0.758346582 | 0.833896159 | −0.08 |
| Milnacipran ((1S-cis) | 0.873126996 | 0.948702654 | −0.08 |
| hydrochloride) | | | |
| Tenofovir alafenamide | 0.650046744 | 0.726886291 | −0.08 |
| Tosufloxacin (tosylate hydrate) | 0.927187709 | 1.004402757 | −0.08 |
| Nitrofurantoin | 0.770764727 | 0.848028356 | −0.08 |
| Dicyclomine (hydrochloride) | 0.918709251 | 0.996134892 | −0.08 |
| Gefitinib (hydrochloride) | 0.845542427 | 0.923444976 | −0.08 |
| Cefsulodin (sodium) | 1.112875188 | 1.190924525 | −0.08 |
| Ruxolitinib | 0.566397746 | 0.645501097 | −0.08 |
| Tadalafil | 0.971059034 | 1.050475494 | −0.08 |
| Troglitazone | 0.89288151 | 0.972513089 | −0.08 |
| Benserazide (hydrochloride) | 1.052444117 | 1.132418729 | −0.08 |
| Mefloquine (hydrochloride) | 1.056782946 | 1.136840929 | −0.08 |
| Boceprevir | 1.413833836 | 1.494432741 | −0.08 |
| Cefathiamidine | 0.832967435 | 0.913602127 | −0.08 |
| 5-Aminolevulinic acid | 1.010260986 | 1.091016803 | −0.08 |
| (hydrochloride) | | | |
| Osimertinib (dimesylate) | 0.361818565 | 0.442688738 | −0.08 |
| Vitamin D2 | 0.968697871 | 1.049609242 | −0.08 |
| Dimethyl fumarate | 0.909466722 | 0.991012044 | −0.08 |
| L-5-Hydroxytryptophan | 1.326725906 | 1.408323176 | −0.08 |
| Bleomycin (hydrochloride) | 0.102294041 | 0.18402493 | −0.08 |
| Rimantadine (hydrochloride) | 0.865590175 | 0.947382199 | −0.08 |
| Nicotinamide | 0.902852418 | 0.985259752 | −0.08 |
| Dexamethasone acetate | 0.872776732 | 0.955290611 | −0.08 |
| DL-alpha-Tocopherol | 1.07209461 | 1.154712042 | −0.08 |
| Novobiocin (Sodium) | 1.056222259 | 1.138971023 | −0.08 |
| (R)-Elagolix | 1.076895629 | 1.159677419 | −0.08 |
| Biotin | 0.976667762 | 1.059973924 | −0.08 |
| Molindone (hydrochloride) | 0.860939176 | 0.944342449 | −0.08 |
| Lornoxicam | 0.988838363 | 1.072263167 | −0.08 |
| Disulfiram | −0.078404932 | 0.005060729 | −0.08 |
| Trioxsalen | 0.97172122 | 1.055704441 | −0.08 |
| Acyclovir | 1.782308657 | 1.866345311 | −0.08 |
| Hydroxyfasudil | 0.85617137 | 0.940440061 | −0.08 |
| (R)-Verapamil (hydrochloride) | 0.920016839 | 1.004514673 | −0.08 |
| Masitinib (mesylate) | 1.00192926 | 1.08645337 | −0.08 |
| Tacrine (hydrochloride) | 1.053955644 | 1.13850207 | −0.08 |
| Phenacetin | 1.04784689 | 1.132418069 | −0.08 |
| Sorafenib (Tosylate) | 1.056865635 | 1.141962422 | −0.09 |
| Phenytoin | 1.230485304 | 1.315688664 | −0.09 |
| Mycophenolate Mofetil | 0.620609187 | 0.705934984 | −0.09 |
| Levetiracetam | 0.917035775 | 1.002425044 | −0.09 |
| Estradiol (cypionate) | 0.894522019 | 0.980861244 | −0.09 |
| Azlocillin (sodium salt) | 0.959228296 | 1.045609258 | −0.09 |
| 4-Aminosalicylic acid | 1.111821705 | 1.198503128 | −0.09 |
| Detomidine (hydrochloride) | 0.931884442 | 1.018958687 | −0.09 |
| Ticarcillin (disodium) | 1.206731863 | 1.293979058 | −0.09 |
| Ingenol | 0.968995453 | 1.056444365 | −0.09 |
| Iproniazid (phosphate) | 1.025915009 | 1.113927826 | −0.09 |
| Monobenzone | 1.137103403 | 1.225704711 | −0.09 |
| Topotecan (Hydrochloride) | 0.046383279 | 0.135529412 | −0.09 |
| Raltegravir | 1.119258168 | 1.208768267 | −0.09 |
| Erythromycin Ethylsuccinate | 0.979737252 | 1.069486983 | −0.09 |
| Simeprevir | 0.755230394 | 0.844999127 | −0.09 |
| Ivacaftor | 0.931028174 | 1.020824103 | −0.09 |
| Gabapentin | 1.036459956 | 1.126461211 | −0.09 |
| Acebutolol (hydrochloride) | 1.104791395 | 1.195181173 | −0.09 |
| L-Carnitine | 0.822118088 | 0.912668583 | −0.09 |
| Natamycin | 1.126916525 | 1.217592593 | −0.09 |
| Almitrine mesylate | 0.670800606 | 0.761639403 | −0.09 |
| Ciclesonide | 0.574150597 | 0.66515744 | −0.09 |
| L-SelenoMethionine | 1.795073032 | 1.886140006 | −0.09 |
| Tafenoquine (Succinate) | 1.045089619 | 1.136177106 | −0.09 |
| Tenofovir alafenamide | 0.419457076 | 0.510695668 | −0.09 |
| hemifumarate | | | |
| Docetaxel (Trihydrate) | 0.279356505 | 0.370823529 | −0.09 |
| Taltirelin (acetate) | 1.062093628 | 1.153729533 | −0.09 |
| Acipimox | 1.260341074 | 1.352188037 | −0.09 |
| Benazepril (hydrochloride) | 0.985370611 | 1.077319588 | −0.09 |
| Zafirlukast | 1.505150913 | 1.597553873 | −0.09 |
| Bacampicillin (hydrochloride) | 0.956036745 | 1.049245147 | −0.09 |
| Cefmenoxime (hydrochloride) | 0.709289121 | 0.80265935 | −0.09 |
| (S)-Glutamic acid | 0.844466888 | 0.937838281 | −0.09 |
| Fulvestrant | 0.95793082 | 1.051363797 | −0.09 |
| Deoxycorticosterone acetate | 0.88870368 | 0.9825162 | −0.09 |
| Belinostat | 0.147101904 | 0.240933929 | −0.09 |
| Avibactam (sodium hydrate) | 1.029392889 | 1.123581777 | −0.09 |
| Cefotaxime | 0.899810566 | 0.994482067 | −0.09 |
| Creatine | 1.830826248 | 1.925499016 | −0.09 |
| Cefuroxime (sodium) | 1.034785436 | 1.129472408 | −0.09 |
| Azaphen (dihydrochloride | 0.925136169 | 1.01991048 | −0.09 |
| monohydrate) | | | |
| Josamycin | 0.824592897 | 0.919427803 | −0.09 |
| Primidone | 0.891401452 | 0.986250358 | −0.09 |
| Rufinamide | 0.899892713 | 0.99497536 | −0.10 |
| Ceftaroline fosamil | 0.931305777 | 1.026509804 | −0.10 |
| (S)-10-Hydroxycamptothecin | 0.13803781 | 0.233657079 | −0.10 |
| Bilastine | 1.071103896 | 1.166926839 | −0.10 |
| Resveratrol | 0.693262156 | 0.78956864 | −0.10 |
| Peramivir (trihydrate) | 0.928275561 | 1.024739949 | −0.10 |
| Avatrombopag (hydrochloride) | 1.082427776 | 1.179683079 | −0.10 |
| Minoxidil sulfate | 1.046659046 | 1.143979058 | −0.10 |
| 18β-Glycyrrhetinic acid | 0.801760208 | 0.89913072 | −0.10 |
| (S)-Terazosin | 0.8794256 | 0.976809354 | −0.10 |
| Davercin | 0.965729688 | 1.063536394 | −0.10 |
| Quinine | 1.045188438 | 1.143110323 | −0.10 |
| Alosetron (Hydrochloride) | 0.825999134 | 0.924061014 | −0.10 |
| Pralatrexate | 0.095700229 | 0.193992933 | −0.10 |
| EC5026 | 1.143126177 | 1.2414307 | −0.10 |
| Acetohexamide | 1.115819209 | 1.214136594 | −0.10 |
| Ethacridine (lactate) | 0.166832175 | 0.265336846 | −0.10 |
| Mebhydrolin | 0.678135275 | 0.776784314 | −0.10 |
| Sulfaphenazole | 1.023966337 | 1.122729287 | −0.10 |
| Gabapentin enacarbil | 0.933157158 | 1.031923384 | −0.10 |
| Trifarotene | 0.835486144 | 0.93427451 | −0.10 |
| Telotristat etiprate | 1.074057551 | 1.17287807 | −0.10 |
| Ursodiol | 1.313839198 | 1.412832629 | −0.10 |
| 17-Hydroxyprogesterone | 0.896238115 | 0.995326263 | −0.10 |
| Sildenafil | 1.131014304 | 1.230139478 | −0.10 |
| Trimetrexate | 0.120318058 | 0.220069548 | −0.10 |
| Mosapride (citrate) | 0.862864078 | 0.962958963 | −0.10 |
| Mianserin (hydrochloride) | 1.020817774 | 1.121058315 | −0.10 |
| Bifendate | 1.168666776 | 1.269071555 | −0.10 |
| Ozenoxacin | 0.762012987 | 0.862628786 | −0.10 |
| Fostamatinib (disodium | 0.874450722 | 0.975161451 | −0.10 |
| hexahydrate) | | | |
| Linaclotide | 0.855432117 | 0.956207971 | −0.10 |
| Bexarotene | 0.986463779 | 1.087365011 | −0.10 |
| Eperisone (Hydrochloride) | 1.222316865 | 1.323412698 | −0.10 |
| Quetiapine sulfoxide | 1.082406734 | 1.18353694 | −0.10 |
| (dihydrochloride) | | | |
| Selumetinib (sulfate) | 0.192833751 | 0.294129555 | −0.10 |
| Baricitinib (phosphate) | 0.561912436 | 0.663240465 | −0.10 |
| Saccharin | 0.776465102 | 0.877893057 | −0.10 |
| Efonidipine (hydrochloride | 1.011717933 | 1.113263785 | −0.10 |
| monoethanolate) | | | |
| Cloperastine (hydrochloride) | 1.546555777 | 1.648247978 | −0.10 |
| Diosmin | 1.15032977 | 1.252094241 | −0.10 |
| Ritodrine (hydrochloride) | 0.961838904 | 1.063935625 | −0.10 |
| Tyloxapol | 1.043329043 | 1.145454545 | −0.10 |
| Dabigatran etexilate (mesylate) | 0.989285714 | 1.091997086 | −0.10 |
| Prasugrel | 0.909104025 | 1.011973102 | −0.10 |
| Avobenzone | 0.967248062 | 1.070263628 | −0.10 |
| Miglustat (hydrochloride) | 0.708196336 | 0.811294118 | −0.10 |
| D-Gluconic acid | 0.999488927 | 1.102954145 | −0.10 |
| Metronidazole Benzoate | 1.000311624 | 1.103790294 | −0.10 |
| Estriol | 0.851040375 | 0.955060219 | −0.10 |
| Choline theophyllinate | 1.170911387 | 1.275395034 | −0.10 |
| Faropenem daloxate | 0.966437525 | 1.071024735 | −0.10 |
| (±)-Darifenacin | 0.872572816 | 0.977213823 | −0.10 |
| Mesna | 1.122488038 | 1.227192205 | −0.10 |
| Xylose | 1.083241859 | 1.188303057 | −0.11 |
| Rofecoxib | 1.146792526 | 1.252024291 | −0.11 |
| Metadoxine | 1.057403591 | 1.163325405 | −0.11 |
| Trametinib (DMSO solvate) | 0.378705281 | 0.48478836 | −0.11 |
| Vinblastine (sulfate) | 0.111948106 | 0.218082464 | −0.11 |
| Hydroxyzine (pamoate) | 1.053009883 | 1.159888288 | −0.11 |
| Gemfibrozil | 1.700503578 | 1.80786644 | −0.11 |
| Binimetinib | 0.380970675 | 0.488661302 | −0.11 |
| Osilodrostat | 1.15213027 | 1.260340371 | −0.11 |
| Salicylic acid | 0.870332093 | 0.978788424 | −0.11 |
| Octisalate | 0.883395961 | 0.992 | −0.11 |
| Olmutinib | 0.448157936 | 0.557063049 | −0.11 |
| Dihydroergocristine (mesylate) | 1.010945064 | 1.119889641 | −0.11 |
| Chloramphenicol succinate | 0.808339029 | 0.917290615 | −0.11 |
| (sodium) | | | |
| Articaine (hydrochloride) | 0.82468295 | 0.933647059 | −0.11 |
| Benzoic acid | 1.168386146 | 1.277363555 | −0.11 |
| Venetoclax | 1.658097005 | 1.767119411 | −0.11 |
| Pemirolast (potassium) | 0.944110443 | 1.053407351 | −0.11 |
| Cisapride | 0.891590453 | 1.00096941 | −0.11 |
| Prednisolone acetate | 0.636670305 | 0.746282406 | −0.11 |
| Danazol | 0.945829428 | 1.055936325 | −0.11 |
| Cangrelor (tetrasodium) | 1.097786553 | 1.208551483 | −0.11 |
| Fostamatinib Disodium | 0.66984019 | 0.781107739 | −0.11 |
| Rasagiline (mesylate) | 1.207574806 | 1.318926975 | −0.11 |
| Sitagliptin | 1.493109332 | 1.60457418 | −0.11 |
| Irinotecan (hydrochloride | 0.516509434 | 0.628320275 | −0.11 |
| trihydrate) | | | |
| Felypressin | 0.979169905 | 1.090981644 | −0.11 |
| Piracetam | 1.066473344 | 1.178479104 | −0.11 |
| Didanosine | 0.86358754 | 0.975824729 | −0.11 |
| Pralidoxime (iodide) | 0.882347169 | 0.994789897 | −0.11 |
| α-Lipoic Acid | 1.003561387 | 1.116073403 | −0.11 |
| L-Lysine hydrochloride | 1.079035809 | 1.192159284 | −0.11 |
| Ixazomib citrate | 0.027868318 | 0.141016063 | −0.11 |
| Polyvinylpyrrolidone | 1.015301015 | 1.128799445 | −0.11 |
| Fesoterodine (L-mandelate) | 0.989229388 | 1.102824306 | −0.11 |
| Roxatidine (Acetate | 1.003576073 | 1.117343845 | −0.11 |
| Hydrochloride) | | | |
| Sotagliflozin | 1.078100775 | 1.19269437 | −0.11 |
| S-Adenosyl-L-methionine | 1.075097529 | 1.190115221 | −0.12 |
| (disulfate tosylate) | | | |
| Thiamphenicol | 0.795762133 | 0.911022114 | −0.12 |
| Lesinurad (sodium) | 0.736545953 | 0.851894374 | −0.12 |
| Methylergometrine (maleate) | 0.900652494 | 1.0165538 | −0.12 |
| Ozagrel (hydrochloride) | 1.13134077 | 1.247303737 | −0.12 |
| Indacaterol (maleate) | 0.915692641 | 1.03246956 | −0.12 |
| Terbinafine hydrochloride | 1.124254473 | 1.24126871 | −0.12 |
| Olopatadine (hydrochloride) | 0.847404473 | 0.964441009 | −0.12 |
| Pirmenol (hydrochloride) | 1.547278973 | 1.664420485 | −0.12 |
| Trifluridine/tipiracil | 0.245930233 | 0.363829312 | −0.12 |
| hydrochloride mixture | | | |
| Terbinafine | 0.94829463 | 1.066309956 | −0.12 |
| Telotristat ethyl | 0.991257806 | 1.109907834 | −0.12 |
| Mexiletine (hydrochloride) | 0.885799404 | 1.004516372 | −0.12 |
| Mefenamic acid | 0.895138563 | 1.013960784 | −0.12 |
| Artesunate | 0.940664733 | 1.059564843 | −0.12 |
| Febuxostat | 1.036127707 | 1.155183585 | −0.12 |
| D-Sorbitol | 1.022684839 | 1.141792657 | −0.12 |
| Solifenacin (Succinate) | 0.765500235 | 0.884705882 | −0.12 |
| Selenomethionine | 1.710486157 | 1.829912848 | −0.12 |
| Levofloxacin (hydrate) | 0.958333333 | 1.077859696 | −0.12 |
| Omadacycline (hydrochloride) | 1.088798821 | 1.208768267 | −0.12 |
| Osimertinib mesylate | 0.26716141 | 0.38737974 | −0.12 |
| Anagliptin | 0.969160105 | 1.089503954 | −0.12 |
| Ethoxzolamide | 1.016110472 | 1.136605559 | −0.12 |
| Levosimendan | 1.04396938 | 1.16511879 | −0.12 |
| Ivabradine (hydrochloride) | 1.21679971 | 1.338429199 | −0.12 |
| Vildagliptin | 1.465658889 | 1.588005215 | −0.12 |
| Gadodiamide (hydrate) | 0.990503876 | 1.113159071 | −0.12 |
| Pixantrone (dimaleate) | 1.211519364 | 1.334211517 | −0.12 |
| Kanamycin (sulfate) | 1.70263789 | 1.825414675 | −0.12 |
| Nifedipine | 1.696262921 | 1.819185059 | −0.12 |
| Idramantone | 1.084764667 | 1.20777682 | −0.12 |
| Dacarbazine | 0.965552651 | 1.089092873 | −0.12 |
| Tricaprilin | 0.94860135 | 1.072263167 | −0.12 |
| Darifenacin (hydrobromide) | 1.073319086 | 1.197319141 | −0.12 |
| Prucalopride (succinate) | 1.048115624 | 1.172352681 | −0.12 |
| Povidone iodine | 0.973544974 | 1.098265094 | −0.12 |
| Azilsartan medoxomil | 1.268932931 | 1.393921156 | −0.12 |
| Antipyrine | 0.853475804 | 0.978526693 | −0.13 |
| Bivalirudin (TFA) | 0.936783933 | 1.06254417 | −0.13 |
| Aceclofenac | 1.328913 | 1.454964176 | −0.13 |
| Tetrahydrozoline (hydrochloride) | 0.933223564 | 1.059813912 | −0.13 |
| Thioridazine (hydrochloride) | 0.945891784 | 1.07254977 | −0.13 |
| Masitinib | 0.806654112 | 0.933432688 | −0.13 |
| Saquinavir (Mesylate) | 1.925345044 | 2.052147239 | −0.13 |
| (R)-Baclofen | 0.858513189 | 0.985380939 | −0.13 |
| Cladribine | 0.027411592 | 0.154495777 | −0.13 |
| Rotigotine (Hydrochloride) | 0.675207089 | 0.802631579 | −0.13 |
| DL-Panthenol | 1.049409664 | 1.176865377 | −0.13 |
| Fluvastatin (sodium) | 0.198980295 | 0.326622326 | −0.13 |
| Nitroxoline | 1.742694918 | 1.870803662 | −0.13 |
| Obeticholic acid | 1.129508407 | 1.257636586 | −0.13 |
| Levamlodipine besylate | 0.937173264 | 1.065766739 | −0.13 |
| Panaxatriol | 0.955980861 | 1.084588131 | −0.13 |
| Cefazolin | 1.068791003 | 1.197435331 | −0.13 |
| Bicalutamide | 1.089784442 | 1.218660181 | −0.13 |
| Clonidine (hydrochloride) | 0.953205601 | 1.082910826 | −0.13 |
| Ceritinib dihydrochloride | 0.036772777 | 0.166592822 | −0.13 |
| Riboflavin (phosphate sodium) | 0.997194222 | 1.127108784 | −0.13 |
| N-Acetyl-L-tyrosine | 1.217514124 | 1.347748285 | −0.13 |
| Cinepazide (Maleate) | 1.001137139 | 1.131675393 | −0.13 |
| Telaprevir | 0.952944843 | 1.083953241 | −0.13 |
| Progesterone | 0.923246521 | 1.054287255 | −0.13 |
| Tropisetron (Hydrochloride) | 0.986600187 | 1.117959617 | −0.13 |
| Tirofiban (hydrochloride | 1.692770242 | 1.824172743 | −0.13 |
| monohydrate) | | | |
| Perindopril (erbumine) | 0.791666667 | 0.9237042 | −0.13 |
| Clofibrate | 1.653856348 | 1.786361064 | −0.13 |
| Biapenem | 1.116746411 | 1.249335695 | −0.13 |
| Atazanavir (sulfate) | 1.127528415 | 1.260121457 | −0.13 |
| Hydroxyprogesterone caproate | 0.895931336 | 1.028601797 | −0.13 |
| Camphor | 0.996889865 | 1.129818343 | −0.13 |
| Pexidartinib (hydrochloride) | 1.001515778 | 1.135538379 | −0.13 |
| Mirabegron | 1.183357396 | 1.317484201 | −0.13 |
| Epirubicin (hydrochloride) | −0.004784689 | 0.129549016 | −0.13 |
| Glycyrrhizic acid | 0.907034632 | 1.041627641 | −0.13 |
| Toltrazuril | 0.846597887 | 0.981509096 | −0.13 |
| Fluticasone (propionate) | 0.607209958 | 0.74227451 | −0.14 |
| Latanoprostene bunod | 1.003866272 | 1.139005236 | −0.14 |
| Fingolimod | 1.097438917 | 1.232642428 | −0.14 |
| Kasugamycin (hydrochloride | 1.152903355 | 1.288126919 | −0.14 |
| hydrate) | | | |
| Silodosin | 1.074057551 | 1.210361913 | −0.14 |
| Acetylspiramycin | 1.100036147 | 1.236516987 | −0.14 |
| Dicloxacillin (Sodium hydrate) | 1.008369952 | 1.145057129 | −0.14 |
| Amitriptyline (hydrochloride) | 0.818438261 | 0.955128989 | −0.14 |
| Nizatidine | 0.98086547 | 1.117567108 | −0.14 |
| Ibrutinib | 0.459449046 | 0.596153846 | −0.14 |
| Macitentan | 1.560309752 | 1.697261511 | −0.14 |
| Cysteamine hydrochloride | 0.860049055 | 0.997539774 | −0.14 |
| Liothyronine | 1.048349688 | 1.18640553 | −0.14 |
| Ethosuximide | 0.995215311 | 1.133303809 | −0.14 |
| Lenampicillin (hydrochloride) | 0.911810533 | 1.049946865 | −0.14 |
| Glutathione oxidized | 0.814845589 | 0.953204476 | −0.14 |
| Fruquintinib | 1.059330144 | 1.197962799 | −0.14 |
| Pyrazinamide | 0.800916566 | 0.939575972 | −0.14 |
| Epalrestat | 0.857877814 | 0.996596324 | −0.14 |
| Caffeic acid | 0.803212851 | 0.94235334 | −0.14 |
| Teprenone | 0.977657695 | 1.117219314 | −0.14 |
| Entacapone | 1.57920987 | 1.718928679 | −0.14 |
| Midostaurin | 0.531208997 | 0.671235905 | −0.14 |
| Trelagliptin (succinate) | 1.616750596 | 1.756932654 | −0.14 |
| Naproxen (sodium) | 1.057996877 | 1.198298147 | −0.14 |
| Florfenicol | 0.997040934 | 1.137394047 | −0.14 |
| 6-Mercaptopurine | 0.980749111 | 1.121212121 | −0.14 |
| Thiamine monochloride | 0.900741276 | 1.041778976 | −0.14 |
| Arotinolol | 1.016710232 | 1.157775378 | −0.14 |
| Talaporfin (sodium) | 0.945949589 | 1.087279152 | −0.14 |
| Rivaroxaban | 1.057334639 | 1.19882221 | −0.14 |
| Isoprenaline (hydrochloride) | 1.354753054 | 1.496283081 | −0.14 |
| Itraconazole | 0.979704072 | 1.121325829 | −0.14 |
| Cefonicid (sodium) | 1.107450444 | 1.249521156 | −0.14 |
| Landiolol (hydrochloride) | 1.168676662 | 1.310982443 | −0.14 |
| (S)-Crizotinib | 0.951788756 | 1.094135802 | −0.14 |
| Aclidinium (Bromide) | 0.735678663 | 0.878091873 | −0.14 |
| Phillyrin | 1.262353706 | 1.404790782 | −0.14 |
| Pentagastrin | 1.149119948 | 1.291557714 | −0.14 |
| Methimazole | 1.001280644 | 1.143996455 | −0.14 |
| Mizoribine | 0.974482944 | 1.11766297 | −0.14 |
| Eltrombopag | 0.867973974 | 1.011285341 | −0.14 |
| Lifitegrast | 0.865247019 | 1.008597884 | −0.14 |
| Benorilate | 1.110006519 | 1.25345536 | −0.14 |
| Oxethazaine | 0.987373737 | 1.131096934 | −0.14 |
| 2-Ethoxybenzamide | 0.975370425 | 1.119379318 | −0.14 |
| Amiloride (hydrochloride) | 0.871653358 | 1.015843137 | −0.14 |
| Almonertinib | 0.285462659 | 0.429803922 | −0.14 |
| Clindamycin palmitate | 0.948198487 | 1.092554102 | −0.14 |
| (hydrochloride) | | | |
| Oclacitinib (maleate) | 0.844521045 | 0.989107586 | −0.14 |
| Darunavir | 0.772398055 | 0.917357547 | −0.14 |
| Cefotaxime (sodium salt) | 1.138983051 | 1.284101382 | −0.15 |
| Conivaptan (hydrochloride) | 1.001716971 | 1.147184604 | −0.15 |
| Roxithromycin | 0.83821882 | 0.983693305 | −0.15 |
| Revefenacin | 1.038170935 | 1.184165817 | −0.15 |
| Fudosteine | 1.16254772 | 1.308597748 | −0.15 |
| Alogliptin (Benzoate) | 0.940166708 | 1.086578413 | −0.15 |
| Trometamol | 0.969422982 | 1.116104869 | −0.15 |
| Lemborexant | 1.038225194 | 1.185198384 | −0.15 |
| Betamethasone | 0.945579461 | 1.092991914 | −0.15 |
| Imatinib (Mesylate) | 0.941096202 | 1.088743455 | −0.15 |
| Quinine (hydrochloride dihydrate) | 1.06016768 | 1.208357974 | −0.15 |
| Levodropropizine | 0.947502698 | 1.095820591 | −0.15 |
| Ranolazine | 1.162373004 | 1.310911523 | −0.15 |
| Efloxate | 0.975370425 | 1.123909843 | −0.15 |
| Bestatin (hydrochloride) | 1.150515464 | 1.29913774 | −0.15 |
| Empagliflozin | 0.826081376 | 0.974864723 | −0.15 |
| Clarithromycin | 1.06375969 | 1.213695264 | −0.15 |
| Terbutaline (sulfate) | 1.039072257 | 1.189170507 | −0.15 |
| Pimavanserin tartrate | 0.941910753 | 1.092016911 | −0.15 |
| Ramatroban | 0.910126269 | 1.060682192 | −0.15 |
| Prazosin (hydrochloride) | 1.078777657 | 1.229503882 | −0.15 |
| Hesperidin | 1.671236671 | 1.82197355 | −0.15 |
| Cortisone | 0.88182721 | 1.03312006 | −0.15 |
| Oxytocin | 0.905064204 | 1.056585206 | −0.15 |
| Hyperoside | 0.814394528 | 0.966480447 | −0.15 |
| Mizolastine | 0.800728041 | 0.953098039 | −0.15 |
| Hydrocortisone acetate | 0.630004044 | 0.782685512 | −0.15 |
| Rifaximin | 0.730397422 | 0.883152858 | −0.15 |
| Ethamsylate | 1.24467589 | 1.397446129 | −0.15 |
| Afatinib (dimaleate) | 0.319342831 | 0.47215919 | −0.15 |
| Laurocapram | 0.883028379 | 1.035961123 | −0.15 |
| Piperacetazine | 0.935209772 | 1.088314005 | −0.15 |
| Tamoxifen (Citrate) | 0.76706455 | 0.920512425 | −0.15 |
| Rebamipide | 1.190366595 | 1.343977922 | −0.15 |
| Polidocanol | 0.891748892 | 1.045523942 | −0.15 |
| Metolazone | 0.975381739 | 1.129295076 | −0.15 |
| Azilsartan | 1.042261251 | 1.196278245 | −0.15 |
| Octocrylene | 1.028980991 | 1.183138505 | −0.15 |
| Benfotiamine | 1.07584207 | 1.230180956 | −0.15 |
| Doripenem (monohydrate) | 0.836281012 | 0.991052789 | −0.15 |
| Ribociclib hydrochloride | 0.830616073 | 0.985730687 | −0.16 |
| Theophylline | 1.026488002 | 1.181721573 | −0.16 |
| Ambroxol | 0.965585541 | 1.120977872 | −0.16 |
| Lopinavir | 1.393361814 | 1.548891786 | −0.16 |
| Glibenclamide | 0.956684325 | 1.112291888 | −0.16 |
| Morinidazole (R enantiomer) | 1.022078304 | 1.177823083 | −0.16 |
| Artemotil | 0.804612354 | 0.960642074 | −0.16 |
| Cefmetazole (sodium) | 1.163975438 | 1.320157068 | −0.16 |
| Gatifloxacin (hydrochloride) | 0.773595004 | 0.930184332 | −0.16 |
| Esmolol (hydrochloride) | 1.053523296 | 1.210158841 | −0.16 |
| Nalfurafine (hydrochloride) | 0.926000809 | 1.083038869 | −0.16 |
| Bendazac L-Lysine | 0.922041287 | 1.079349554 | −0.16 |
| L-Ascorbic acid | 1.002558389 | 1.16001546 | −0.16 |
| Reboxetine (mesylate) | 0.704804502 | 0.86239134 | −0.16 |
| Ceftibuten (dihydrate) | 1.194905617 | 1.352617801 | −0.16 |
| Diltiazem (hydrochloride) | 1.159029086 | 1.316939891 | −0.16 |
| Clioquinol | 1.009369786 | 1.167568511 | −0.16 |
| Plerixafor | 1.011106743 | 1.16960955 | −0.16 |
| Sulfamethazine | 1.05175106 | 1.210361913 | −0.16 |
| Fosfomycin (sodium) | 1.003844955 | 1.163176265 | −0.16 |
| Fenitrothion | 1.425781956 | 1.585354897 | −0.16 |
| Temozolomide | 0.824224806 | 0.984025916 | −0.16 |
| Pamidronate (disodium | 1.008868559 | 1.169006479 | −0.16 |
| pentahydrate) | | | |
| Alfuzosin | 1.024333561 | 1.184746648 | −0.16 |
| Idoxuridine | 0.963585075 | 1.124342829 | −0.16 |
| Sapropterin (dihydrochloride) | 0.984107199 | 1.144881332 | −0.16 |
| Griseofulvin | 1.22379231 | 1.384615385 | −0.16 |
| Lusutrombopag | 0.782692573 | 0.943555635 | −0.16 |
| Delafloxacin (meglumine) | 0.995325647 | 1.156216791 | −0.16 |
| (S)-Verapamil (hydrochloride) | 0.924867505 | 1.085805701 | −0.16 |
| Procyclidine (hydrochloride) | 1.076397919 | 1.237416616 | −0.16 |
| Xylitol | 1.073319086 | 1.23437808 | −0.16 |
| Menadione bisulfite (sodium) | 0.829461838 | 0.99097917 | −0.16 |
| Amiloride hydrochloride dihydrate | 1.069856459 | 1.231620903 | −0.16 |
| Bephenium (hydroxynaphthoate) | 1.472109201 | 1.634065603 | −0.16 |
| Cilazapril (monohydrate) | 1.103567319 | 1.265917603 | −0.16 |
| Bromisoval | 0.92326567 | 1.085846618 | −0.16 |
| Pefloxacin (mesylate) | 0.857724902 | 1.02035861 | −0.16 |
| Imiquimod (hydrochloride) | 1.160136628 | 1.322927329 | −0.16 |
| Monocrotaline | 1.046243375 | 1.209133217 | −0.16 |
| Bekanamycin | 1.293233083 | 1.456308235 | −0.16 |
| Prasugrel (hydrochloride) | 1.049570575 | 1.213066955 | −0.16 |
| Moroxydine (hydrochloride) | 0.856320894 | 1.019909324 | −0.16 |
| Spiramycin | 1.113265971 | 1.277362719 | −0.16 |
| Calcifediol | 1.051619267 | 1.216242854 | −0.16 |
| Danthron | 1.469173748 | 1.633872417 | −0.16 |
| L-Hyoscyamine | 1.026893524 | 1.191847585 | −0.16 |
| Phenprocoumon | 0.906052029 | 1.071024735 | −0.16 |
| Tafluprost | 1.095420369 | 1.260665158 | −0.17 |
| Moxisylyte (hydrochloride) | 1.071277211 | 1.236639676 | −0.17 |
| Aspartame | 0.999817051 | 1.165263624 | −0.17 |
| Sulbenicillin (disodium) | 0.998588114 | 1.1640775 | −0.17 |
| Ozanimod | 0.953054562 | 1.118914605 | −0.17 |
| 6-Mercaptopurine hydrate | 0.745756059 | 0.912334353 | −0.17 |
| Sulfinpyrazone | 0.909286966 | 1.075971731 | −0.17 |
| Bupivacaine (hydrochloride) | 0.892242956 | 1.05892778 | −0.17 |
| Menthol | 0.86417957 | 1.031122746 | −0.17 |
| Paromomycin (sulfate) | 1.216275189 | 1.383312969 | −0.17 |
| Tetrandrine | 0.966086159 | 1.133208255 | −0.17 |
| Deoxycholic acid sodium salt | 0.706273369 | 0.873855544 | −0.17 |
| Lenvatinib | 1.010259599 | 1.177972865 | −0.17 |
| Miglitol | 1.198769651 | 1.366533171 | −0.17 |
| Garenoxacin (Mesylate hydrate) | 1.267597968 | 1.435505446 | −0.17 |
| Cefodizime | 1.538600615 | 1.706648697 | −0.17 |
| Nimustine (hydrochloride) | 1.025653627 | 1.193981597 | −0.17 |
| Carmofur | 0.600466716 | 0.769161945 | −0.17 |
| 2-(Phosphonooxy)benzoic acid | 0.977863154 | 1.146654852 | −0.17 |
| Eliglustat (hemitartrate) | 0.757638238 | 0.926818483 | −0.17 |
| Aminohippurate (sodium) | 1.198845493 | 1.368028631 | −0.17 |
| Upadacitinib | 0.573232323 | 0.742539617 | −0.17 |
| Helicid | 0.923917749 | 1.093245915 | −0.17 |
| Hexylresorcinol | 1.344440666 | 1.514036419 | −0.17 |
| Penicillamine | 1.082523796 | 1.252123683 | −0.17 |
| Lanreotide (acetate) | 1.218496684 | 1.388308977 | −0.17 |
| Diacerein | 0.910456384 | 1.080394241 | −0.17 |
| Metixene hydrochloride hydrate | 1.036268921 | 1.206416314 | −0.17 |
| Omeprazole | 0.688306685 | 0.858502024 | −0.17 |
| Crotamiton | 1.087983833 | 1.258579409 | −0.17 |
| Desfesoterodine | 0.839466598 | 1.010392469 | −0.17 |
| Propyl gallate | 1.135130692 | 1.306130495 | −0.17 |
| Altretamine | 0.963625058 | 1.134876297 | −0.17 |
| Thalidomide | 1.012662338 | 1.183994172 | −0.17 |
| Sulfamethoxazole | 1.051297995 | 1.222665603 | −0.17 |
| Sitagliptin (phosphate | 0.958975548 | 1.130421361 | −0.17 |
| monohydrate) | | | |
| Casanthranol | 1.063349063 | 1.234836919 | −0.17 |
| Butylphthalide | 1.120309302 | 1.291884817 | −0.17 |
| Chlorphenesin | 1.09612387 | 1.268470041 | −0.17 |
| Acetylcholine (iodide) | 1.589709936 | 1.762263015 | −0.17 |
| Phenindione | 1.394604768 | 1.567192521 | −0.17 |
| Foscarnet (sodium) | 1.050815754 | 1.223676556 | −0.17 |
| Cefotetan | 1.026893524 | 1.199822774 | −0.17 |
| Ketorolac (tromethamine salt) | 1.116013629 | 1.289021164 | −0.17 |
| Folic acid | 0.944308181 | 1.117598819 | −0.17 |
| Docusate (Sodium) | 0.906052029 | 1.0795053 | −0.17 |
| Brimonidine | 0.727897306 | 0.901617251 | −0.17 |
| Alcaftadine | 0.990770522 | 1.164610208 | −0.17 |
| Asenapine (maleate) | 1.135430248 | 1.309488233 | −0.17 |
| Azithromycin | 1.162208567 | 1.33630316 | −0.17 |
| Moxidectin | 0.688163037 | 0.862503581 | −0.17 |
| Isosorbide dinitrate | 0.830789103 | 1.005176471 | −0.17 |
| Tranylcypromine (hemisulfate) | 0.702630664 | 0.877295879 | −0.17 |
| Flurbiprofen | 1.041728902 | 1.216954644 | −0.18 |
| Pivmecillinam (hydrochloride) | 1.043888166 | 1.219223772 | −0.18 |
| Fluocinolone (Acetonide) | 0.468940436 | 0.644367073 | −0.18 |
| Ropivacaine | 0.762755897 | 0.938627109 | −0.18 |
| Dihydroergotamine (mesylate) | 0.971276985 | 1.147540984 | −0.18 |
| Ornidazole | 1.208551285 | 1.385078534 | −0.18 |
| Taurochenodeoxycholic acid | 1.002549542 | 1.17919432 | −0.18 |
| Amoxicillin (sodium) | 0.758994757 | 0.935759209 | −0.18 |
| Hexaminolevulinate | 0.897125974 | 1.074384636 | −0.18 |
| (hydrochloride) | | | |
| VAL-083 | 0.701744186 | 0.879021448 | −0.18 |
| Azathioprine | 1.493637254 | 1.671620864 | −0.18 |
| Amantadine (hydrochloride) | 0.961445108 | 1.140064795 | −0.18 |
| Tinidazole | 0.661140132 | 0.839805825 | −0.18 |
| Ceftizoxime | 1.370685077 | 1.54980672 | −0.18 |
| Tolnaftate | 1.239684366 | 1.418884289 | −0.18 |
| L-Serine | 0.946050967 | 1.125326681 | −0.18 |
| Vidarabine | 0.805894786 | 0.98572549 | −0.18 |
| Dalbavancin (hydrochloride) | 0.877939691 | 1.057897327 | −0.18 |
| Tranilast | 1.039782387 | 1.221168437 | −0.18 |
| Estramustine (phosphate sodium) | 1.06967632 | 1.251102293 | −0.18 |
| Pimethixene maleate | 0.915269372 | 1.096854849 | −0.18 |
| L-Leucine | 0.88255663 | 1.06445793 | −0.18 |
| Fluorometholone | 0.560239549 | 0.74227451 | −0.18 |
| Cilnidipine | 0.725105683 | 0.907294118 | −0.18 |
| Proguanil | 1.172316384 | 1.354906054 | −0.18 |
| Chlorpropamide | 0.930754226 | 1.113705276 | −0.18 |
| Tamsulosin | 1.177990431 | 1.36096117 | −0.18 |
| Decamethonium (Bromide) | 0.945675207 | 1.128947368 | −0.18 |
| Acetylcholine (chloride) | 0.802914442 | 0.986386748 | −0.18 |
| Repaglinide | 0.812456938 | 0.996045299 | −0.18 |
| Treosulfan | 1.032364341 | 1.216376229 | −0.18 |
| Meclocycline (Sulfosalicylate Salt) | 0.890442133 | 1.074893875 | −0.18 |
| Streptomycin (sulfate) | 1.585785917 | 1.770312061 | −0.18 |
| Trimetazidine (dihydrochloride) | 1.111532456 | 1.296100819 | −0.18 |
| Ertugliflozin L-pyroglutamic acid | 0.826576252 | 1.011973102 | −0.19 |
| Nilvadipine | 1.393361814 | 1.578878748 | −0.19 |
| Pyrimethamine | 1.019697535 | 1.205291577 | −0.19 |
| Saquinavir | 0.800968992 | 0.986706881 | −0.19 |
| Amlodipine (besylate) | 0.999031008 | 1.18554513 | −0.19 |
| Bendamustine (hydrochloride) | 0.955437831 | 1.142047467 | −0.19 |
| Reldesemtiv | 0.935674945 | 1.122454916 | −0.19 |
| Atropine methyl bromide | 0.956217326 | 1.143088553 | −0.19 |
| Sonidegib (diphosphate) | 1.486924906 | 1.673864274 | −0.19 |
| D-Cycloserine | 1.0052976 | 1.193057032 | −0.19 |
| Zanamivir | 1.106955055 | 1.294775628 | −0.19 |
| Tizanidine (hydrochloride) | 0.98556701 | 1.173557373 | −0.19 |
| Evans Blue | 0.95336839 | 1.141597579 | −0.19 |
| Liothyronine (sodium) | 1.113291704 | 1.301612903 | −0.19 |
| Doxycycline (hyclate) | 0.885170604 | 1.073687994 | −0.19 |
| Pexidartinib | 1.578584681 | 1.767119411 | −0.19 |
| Sodium 4-phenylbutyrate | 0.905059746 | 1.093844492 | −0.19 |
| Chlorothiazide | 0.937173264 | 1.126241901 | −0.19 |
| Olmesartan | 0.941999711 | 1.131376087 | −0.19 |
| Irsogladine | 0.994987222 | 1.184531211 | −0.19 |
| Sulfabenzamide | 1.040918457 | 1.230830219 | −0.19 |
| Cytidine | 1.220741222 | 1.410855064 | −0.19 |
| Antazoline (hydrochloride) | 1.209706083 | 1.400661675 | −0.19 |
| Cyclopentolate (hydrochloride) | 0.872727273 | 1.06421612 | −0.19 |
| Fenspiride (Hydrochloride) | 0.913804867 | 1.105497382 | −0.19 |
| Dipotassium glycyrrhizinate | 0.933065721 | 1.124946004 | −0.19 |
| Ruxolitinib (phosphate) | 0.679465257 | 0.87150838 | −0.19 |
| Linezolid | 1.187486922 | 1.379533035 | −0.19 |
| Teniposide | 0.18832021 | 0.380661395 | −0.19 |
| Iloprost | 0.996317829 | 1.188672922 | −0.19 |
| Levofloxacin | 0.943992248 | 1.136394102 | −0.19 |
| Fenoprofen (Calcium hydrate) | 1.031976744 | 1.224419124 | −0.19 |
| Stavudine | 0.829319977 | 1.022064777 | −0.19 |
| L-Lactic acid | 0.870419543 | 1.063336307 | −0.19 |
| Metoprolol (Succinate) | 0.878612149 | 1.071623974 | −0.19 |
| Dihydroartemisinin | 0.726215206 | 0.919294676 | −0.19 |
| Betaine (hydrochloride) | 1.09897385 | 1.292058713 | −0.19 |
| Setiptiline | 0.854629448 | 1.048246705 | −0.19 |
| Eplerenone | 0.844880225 | 1.039058824 | −0.19 |
| Squalene | 0.793103448 | 0.987698868 | −0.19 |
| 4-Phenylbutyric acid | 1.056403864 | 1.251151258 | −0.19 |
| Penicillin G benzathine | 1.125924708 | 1.321111768 | −0.20 |
| (tetrahydrate) | | | |
| L-Phenylalanine | 0.791790798 | 0.987956342 | −0.20 |
| Rimegepant | 1.114147287 | 1.310656836 | −0.20 |
| Diphylline | 1.050193798 | 1.2467605 | −0.20 |
| Cephalothin (sodium) | 0.790142472 | 0.98673416 | −0.20 |
| Bucladesine (sodium) | 0.897654793 | 1.094461434 | −0.20 |
| Prednisolone | 0.443705858 | 0.641234317 | −0.20 |
| Tolbutamide | 0.925587856 | 1.123174102 | −0.20 |
| Benactyzine hydrochloride | 0.960963935 | 1.159242439 | −0.20 |
| Ethynyl Estradiol | 1.05517177 | 1.253671706 | −0.20 |
| Angiotensin III (TFA) | 0.812456938 | 1.011145066 | −0.20 |
| Entecavir (monohydrate) | 0.971642256 | 1.170386114 | −0.20 |
| Fluralaner | 1.40966123 | 1.608676599 | −0.20 |
| Serotonin hydrochloride | 1.179500797 | 1.378531073 | −0.20 |
| Alectinib (Hydrochloride) | 0.668162585 | 0.867587725 | −0.20 |
| Ropivacaine (hydrochloride) | 0.98990333 | 1.189372954 | −0.20 |
| Valsartan | 0.856831861 | 1.056327725 | −0.20 |
| Methazolamide | 1.24088071 | 1.440677966 | −0.20 |
| Bictegravir | 0.908285403 | 1.108539505 | −0.20 |
| Safinamide (mesylate) | 0.84298036 | 1.043339151 | −0.20 |
| Etoposide phosphate | 0.746129066 | 0.946771379 | −0.20 |
| Piribedil | 0.987806443 | 1.188514575 | −0.20 |
| Bromocriptine (mesylate) | 0.734307359 | 0.935477157 | −0.20 |
| (+)-Penbutolol | 0.951810975 | 1.153232243 | −0.20 |
| Eflornithine (hydrochloride | 1.224010125 | 1.426325247 | −0.20 |
| hydrate) | | | |
| Oxybutynin (chloride) | 1.033841421 | 1.236399327 | −0.20 |
| Cefodizime (sodium) | 1.087535145 | 1.290294053 | −0.20 |
| Bepotastine | 0.763914004 | 0.966734903 | −0.20 |
| L-Cysteine (hydrochloride) | 0.918705452 | 1.121659919 | −0.20 |
| Cobimetinib (hemifumarate) | 0.320257235 | 0.523893805 | −0.20 |
| Betahistine | 1.061623194 | 1.265561373 | −0.20 |
| Diethyltoluamide | 1.098251959 | 1.302456499 | −0.20 |
| Lumacaftor | 1.539309621 | 1.743605176 | −0.20 |
| Daidzin | 0.806131561 | 1.010527701 | −0.20 |
| Bentiromide | 0.711727325 | 0.916127162 | −0.20 |
| Baicalin | 1.093220339 | 1.297643901 | −0.20 |
| Pyridoxal phosphate | 0.905628927 | 1.110160166 | −0.20 |
| Nedaplatin | 0.986009638 | 1.190742219 | −0.20 |
| Thiamine nitrate | 0.701754386 | 0.906661648 | −0.20 |
| Varenicline (Hydrochloride) | 0.987559809 | 1.192648361 | −0.21 |
| Brigatinib | 0.630875998 | 0.836147592 | −0.21 |
| Sulfaguanidine | 0.886642405 | 1.092226148 | −0.21 |
| Glimepiride | 0.784668121 | 0.990435707 | −0.21 |
| Candesartan Cilexetil | 0.973837209 | 1.179736372 | −0.21 |
| Cefetamet pivoxil (hydrochloride) | 0.851994146 | 1.058041648 | −0.21 |
| Magnolol | 1.086649989 | 1.292931937 | −0.21 |
| Losartan (potassium) | 0.963417354 | 1.169862128 | −0.21 |
| Probucol | 1.086714952 | 1.293196473 | −0.21 |
| Sulfalene | 0.953412073 | 1.159956866 | −0.21 |
| Sodium nitroprusside | 1.180450161 | 1.387066031 | −0.21 |
| Methylthiouracil | 0.882024318 | 1.088657106 | −0.21 |
| Fosfomycin (calcium) | 0.88640882 | 1.09349014 | −0.21 |
| Guanidine (hydrochloride) | 0.928584765 | 1.13574514 | −0.21 |
| 5-Aminosalicylic Acid | 1.015899408 | 1.223676556 | −0.21 |
| Fluorescein | 0.846414129 | 1.054498019 | −0.21 |
| Ligustrazine (hydrochloride) | 1.02044902 | 1.228730049 | −0.21 |
| Axitinib | 0.715184187 | 0.923895968 | −0.21 |
| Apronal | 1.47920434 | 1.688331627 | −0.21 |
| Colistin (sulfate) | 0.980115758 | 1.189308855 | −0.21 |
| Aspirin | 0.981226656 | 1.190499876 | −0.21 |
| Pimecrolimus | 0.890930568 | 1.100210653 | −0.21 |
| Erythromycin | 0.992861923 | 1.202175786 | −0.21 |
| Dienogest | 0.708631973 | 0.918172157 | −0.21 |
| Calcium dobesilate | 1.217737653 | 1.427297056 | −0.21 |
| Isoxsuprine hydrochloride | 0.677449791 | 0.887279152 | −0.21 |
| Crocin | 0.925173802 | 1.135135135 | −0.21 |
| Methylprednisolone succinate | 0.705110733 | 0.915123457 | −0.21 |
| Daphnetin | 0.913876257 | 1.124033795 | −0.21 |
| Oritavancin (diphosphate) | 0.810174991 | 1.020527859 | −0.21 |
| Ripasudil | 1.176742395 | 1.387502182 | −0.21 |
| Avanafil | 0.909135925 | 1.120204851 | −0.21 |
| Penbutolol (sulfate) | 0.744833255 | 0.956235294 | −0.21 |
| Chlorzoxazone | 0.939544551 | 1.150985982 | −0.21 |
| S-(+)-Ketoprofen | 0.743709642 | 0.955398162 | −0.21 |
| Uridine triacetate | 0.864138093 | 1.076078431 | −0.21 |
| Regorafenib | 0.858547813 | 1.07054148 | −0.21 |
| Droxidopa | 1.02317664 | 1.235772358 | −0.21 |
| Amrinone | 0.944141847 | 1.156968668 | −0.21 |
| Metaproterenol (hemisulfate) | 0.789055129 | 1.002473498 | −0.21 |
| Ecabet (sodium) | 0.910238751 | 1.123688002 | −0.21 |
| Tolcapone | 0.899421652 | 1.113922547 | −0.21 |
| Quizartinib | 0.807598511 | 1.022342468 | −0.21 |
| Guanabenz (Acetate) | 0.969663172 | 1.184941835 | −0.22 |
| Ofloxacin | 0.938880214 | 1.154351573 | −0.22 |
| Tranexamic acid | 1.67822106 | 1.894011808 | −0.22 |
| Furosemide | 0.893992248 | 1.110031278 | −0.22 |
| Etoposide | 0.501378134 | 0.71802588 | −0.22 |
| Succimer | 1.034638406 | 1.252123683 | −0.22 |
| Isoxicam | 1.064076146 | 1.281572092 | −0.22 |
| Azelastine (hydrochloride) | 0.87408674 | 1.091779729 | −0.22 |
| Asenapine | 0.700211367 | 0.917960784 | −0.22 |
| Edoxaban (tosylate monohydrate) | 1.053315106 | 1.271112659 | −0.22 |
| Trihexyphenidyl (hydrochloride) | 0.691467853 | 0.909893993 | −0.22 |
| Palbociclib (hydrochloride) | 0.818452919 | 1.036944642 | −0.22 |
| 4-Deoxypyridoxine 5′-phosphate | 1.065331793 | 1.284168267 | −0.22 |
| Cefdinir | 0.987015504 | 1.206099196 | −0.22 |
| Zonisamide | 1.065696729 | 1.284984557 | −0.22 |
| Betahistine (dihydrochloride) | 0.800728041 | 1.020235294 | −0.22 |
| Glipizide | 1.200108853 | 1.420217848 | −0.22 |
| Heptaminol (hydrochloride) | 0.848394817 | 1.068798918 | −0.22 |
| Mitotane | 1.124729703 | 1.345187764 | −0.22 |
| Trandolapril | 1.22067324 | 1.441198786 | −0.22 |
| Naratriptan (hydrochloride) | 0.958836443 | 1.179441737 | −0.22 |
| Flufenamic acid | 1.077418684 | 1.298138453 | −0.22 |
| Cyclandelate | 0.966822868 | 1.187707274 | −0.22 |
| Eslicarbazepine | 0.916368724 | 1.137270551 | −0.22 |
| Fabomotizole (hydrochloride) | 0.945932686 | 1.166949824 | −0.22 |
| Treprostinil (sodium) | 0.897964685 | 1.119081272 | −0.22 |
| Sinomenine hydrochloride | 0.784446689 | 1.006068558 | −0.22 |
| Nimorazole | 0.80041073 | 1.022342468 | −0.22 |
| Flavin adenine dinucleotide | 0.88907262 | 1.111091138 | −0.22 |
| (disodium salt) | | | |
| Valproic acid | 0.794818796 | 1.016897358 | −0.22 |
| Edoxaban | 0.809652419 | 1.032156863 | −0.22 |
| Rutin | 1.487519915 | 1.710377639 | −0.22 |
| Larotrectinib sulfate | 1.698818475 | 1.921682196 | −0.22 |
| D-Glucose | 0.599145652 | 0.822038468 | −0.22 |
| Mycophenolic acid | 0.519930676 | 0.742970335 | −0.22 |
| Clopidogrel (hydrogen sulfate) | 0.824597867 | 1.047873604 | −0.22 |
| Phensuximide | 1.332708688 | 1.556216931 | −0.22 |
| Chlorthalidone | 0.990678049 | 1.214520331 | −0.22 |
| Voriconazole | 0.855878959 | 1.079838611 | −0.22 |
| Tilorone (dihydrochloride) | 0.874789166 | 1.09896039 | −0.22 |
| Ampicillin (sodium) | 0.862329506 | 1.086759542 | −0.22 |
| Ketanserin (tartrate) | 1.042878665 | 1.267479549 | −0.22 |
| Tetrahydropalmatine | 0.768570396 | 0.993703968 | −0.23 |
| Santonin | 1.003641178 | 1.228829507 | −0.23 |
| Benfluorex (hydrochloride) | 0.815053231 | 1.040970142 | −0.23 |
| Voglibose | 0.683702088 | 0.909670563 | −0.23 |
| Polymyxin B (Sulfate) | 0.942656943 | 1.168771686 | −0.23 |
| Mirogabalin | 0.854319086 | 1.082387851 | −0.23 |
| Amifampridine | 1.071196359 | 1.299272286 | −0.23 |
| Thymol | 0.956036745 | 1.184399712 | −0.23 |
| Istradefylline | 0.954723674 | 1.183261339 | −0.23 |
| Fertirelin | 1.137525632 | 1.366533171 | −0.23 |
| Finafloxacin | 0.997323221 | 1.226734166 | −0.23 |
| Docosahexaenoic Acid | 0.804068677 | 1.033623093 | −0.23 |
| Icatibant (acetate) | 0.794762548 | 1.024723677 | −0.23 |
| [Sar1, Ile8]-Angiotensin II (TFA) | 1.059810952 | 1.290063504 | −0.23 |
| Dropropizine | 0.958130767 | 1.188445848 | −0.23 |
| Carbamazepine | 0.995539697 | 1.226036866 | −0.23 |
| D-Fructose | 1.053304705 | 1.284341253 | −0.23 |
| Tofacitinib (citrate) | 0.680571046 | 0.912214563 | −0.23 |
| Olanzapine | 1.489423251 | 1.721922731 | −0.23 |
| Candesartan | 0.868412105 | 1.101289833 | −0.23 |
| Lanolin | 0.772546539 | 1.006058446 | −0.23 |
| Cholic acid (sodium) | 1.690429475 | 1.925499016 | −0.24 |
| trans-Tranilast | 0.908475695 | 1.144168519 | −0.24 |
| Aripiprazole | 1.027751196 | 1.263507529 | −0.24 |
| Deserpidine | 0.814110514 | 1.049973036 | −0.24 |
| Pancuronium (dibromide) | 0.885037551 | 1.121364383 | −0.24 |
| Tolazoline (hydrochloride) | 1.126136085 | 1.362660304 | −0.24 |
| Nalidixic acid | 0.96670247 | 1.203475195 | −0.24 |
| Zileuton | 1.065599254 | 1.302474062 | −0.24 |
| Vorinostat | 0.634996582 | 0.872018109 | −0.24 |
| L-Tryptophan | 0.920246581 | 1.157287449 | −0.24 |
| Valproic acid (sodium salt) | 0.723844928 | 0.961046685 | −0.24 |
| D-Cysteine | 0.907825003 | 1.145142525 | −0.24 |
| 5-Hydroxytryptophan | 0.82518107 | 1.062639821 | −0.24 |
| Cabergoline | 0.899835604 | 1.137568627 | −0.24 |
| Cefoperazone (sodium salt) | 0.974887976 | 1.213066955 | −0.24 |
| Carteolol hydrochloride | 0.886475329 | 1.124844566 | −0.24 |
| Ceftazidime | 1.15266916 | 1.391122914 | −0.24 |
| Tetrabenazine | 1.010120052 | 1.248980129 | −0.24 |
| Loperamide (hydrochloride) | 0.957710978 | 1.196652268 | −0.24 |
| Atrasentan (hydrochloride) | 0.853214719 | 1.092226148 | −0.24 |
| Racecadotril | 0.852270493 | 1.091519523 | −0.24 |
| Acetylcysteine | 0.905928068 | 1.145283915 | −0.24 |
| Fenofibrate | 1.401048256 | 1.640652301 | −0.24 |
| Phenolphthalein | 0.786333251 | 1.026282733 | −0.24 |
| Chlorobutanol | 1.522611357 | 1.762898331 | −0.24 |
| Reserpine (hydrochloride) | 0.833963505 | 1.074315001 | −0.24 |
| Guanethidine (sulfate) | 1.673860911 | 1.914253584 | −0.24 |
| Limaprost | 0.972417899 | 1.213712331 | −0.24 |
| Ribavirin | 0.689895813 | 0.931360358 | −0.24 |
| Favipiravir | 1.105263158 | 1.34676705 | −0.24 |
| Guaiazulene | 1.67400637 | 1.915564598 | −0.24 |
| Taltirelin | 1.148260482 | 1.390092166 | −0.24 |
| Argatroban (monohydrate) | 0.936700069 | 1.178970917 | −0.24 |
| Felodipine | 0.905112354 | 1.148374674 | −0.24 |
| Benzethonium chloride | 0.434755332 | 0.678498393 | −0.24 |
| D-Pantothenic acid (sodium) | 0.857968144 | 1.102038844 | −0.24 |
| Sodium Salicylate | 1.045188557 | 1.289569436 | −0.24 |
| Cefixime | 1.418086138 | 1.662597491 | −0.24 |
| ATP | 0.804068677 | 1.048712482 | −0.24 |
| Pentostatin | 1.244804089 | 1.490079365 | −0.25 |
| Pioglitazone (hydrochloride) | 1.001558118 | 1.246900461 | −0.25 |
| Felbamate | 0.923730396 | 1.169438445 | −0.25 |
| Tromantadine (hydrochloride) | 1.00482293 | 1.250584217 | −0.25 |
| Nifurtimox | 1.110800592 | 1.356593732 | −0.25 |
| Miltefosine | 0.991070552 | 1.237172953 | −0.25 |
| Probenecid | 1.265748031 | 1.512221423 | −0.25 |
| Inulin | 0.931788932 | 1.178764747 | −0.25 |
| Meprednisone | 0.976574937 | 1.22382199 | −0.25 |
| Albendazole | 0.988184748 | 1.235708889 | −0.25 |
| Goserelin (acetate) | 1.248587571 | 1.496770989 | −0.25 |
| Indapamide | 0.967635659 | 1.216823056 | −0.25 |
| Nafamostat (mesylate) | 0.950054885 | 1.199822774 | −0.25 |
| Acotiamide (monohydrochloride | 0.910456384 | 1.160401971 | −0.25 |
| trihydrate) | | | |
| D-Panthenol | 0.817033919 | 1.067252875 | −0.25 |
| Parecoxib | 0.724377418 | 0.974733518 | −0.25 |
| Albendazole sulfoxide | 1.125455147 | 1.376364321 | −0.25 |
| Gabexate (mesylate) | 1.18455229 | 1.435486679 | −0.25 |
| Setiptiline (maleate) | 0.770886574 | 1.021885103 | −0.25 |
| Gadobutrol | 1.022009569 | 1.273250664 | −0.25 |
| Iodipamide | 1.134791524 | 1.386731673 | −0.25 |
| Talniflumate | 1.30238394 | 1.5543383 | −0.25 |
| Tocainide (hydrochloride) | 1.005923309 | 1.257998837 | −0.25 |
| Nortriptyline (hydrochloride) | 0.907820573 | 1.160156863 | −0.25 |
| Estradiol benzoate | 1.133987511 | 1.387327189 | −0.25 |
| Talipexole dihydrochloride | 1.079287723 | 1.332743754 | −0.25 |
| Inosine pranobex | 0.979904306 | 1.233392383 | −0.25 |
| Trilostane | 1.504593176 | 1.758087707 | −0.25 |
| Proglumide (sodium) | 0.961594072 | 1.215134236 | −0.25 |
| Pindolol | 0.777732848 | 1.032155477 | −0.25 |
| Lomitapide | 1.070307596 | 1.324888227 | −0.25 |
| Rifabutin | 0.949936743 | 1.205008736 | −0.26 |
| 6-Acetamidohexanoic acid | 0.784741879 | 1.039929329 | −0.26 |
| Medrysone | 1.253705693 | 1.509695291 | −0.26 |
| Oxybenzone | 0.918043004 | 1.174636911 | −0.26 |
| Palonosetron (Hydrochloride) | 0.926962119 | 1.183592618 | −0.26 |
| Fenoldopam (mesylate) | 0.770817859 | 1.028215287 | −0.26 |
| Valemetostat (tosylate) | 0.937890664 | 1.195288854 | −0.26 |
| Prilocaine | 0.972674419 | 1.231121537 | −0.26 |
| Sulfadiazine (sodium) | 0.975579341 | 1.23414776 | −0.26 |
| Darunavir (Ethanolate) | 1.286101572 | 1.545136867 | −0.26 |
| (S)-(+)-Ibuprofen | 0.960455304 | 1.219503908 | −0.26 |
| β-Pinene | 1.464111372 | 1.723719677 | −0.26 |
| Amphotericin B | 0.952109783 | 1.211771058 | −0.26 |
| Baicalein | 1.086833702 | 1.346555324 | −0.26 |
| Aztreonam | 1.44606414 | 1.705998868 | −0.26 |
| Atracurium (besylate) | 1.108178323 | 1.368528214 | −0.26 |
| Deferasirox | 0.959602909 | 1.220343198 | −0.26 |
| Clinofibrate | 1.080787254 | 1.341587442 | −0.26 |
| Drospirenone | 1.139182283 | 1.400132275 | −0.26 |
| Mitiglinide (Calcium) | 1.59376311 | 1.85501963 | −0.26 |
| Lodoxamide | 0.758851675 | 1.02081488 | −0.26 |
| Ruxolitinib (S enantiomer) | 0.700721923 | 0.963420169 | −0.26 |
| Fedratinib (hydrochloride | 0.377429076 | 0.640197789 | −0.26 |
| hydrate) | | | |
| Mezlocillin (sodium) | 0.915886652 | 1.179117251 | −0.26 |
| Levocetirizine (dihydrochloride) | 0.851484175 | 1.115394181 | −0.26 |
| Terpin (hydrate) | 1.209706083 | 1.474490684 | −0.26 |
| Proxyphylline | 0.943069624 | 1.207971635 | −0.26 |
| Droperidol | 0.79938486 | 1.064745361 | −0.27 |
| Rotundine | 0.82773794 | 1.093574897 | −0.27 |
| Glycine | 0.983005842 | 1.248884924 | −0.27 |
| Hyaluronidase | 1.022165022 | 1.288133241 | −0.27 |
| Metronidazole | 1.474787638 | 1.740827024 | −0.27 |
| Sulfamonomethoxine | 0.746461787 | 1.013074205 | −0.27 |
| Relugolix | 1.077325581 | 1.344168901 | −0.27 |
| Mestranol | 0.930296378 | 1.197164377 | −0.27 |
| Diclofenac | 0.820976492 | 1.088536336 | −0.27 |
| Fidaxomicin | 1.226610125 | 1.49434069 | −0.27 |
| Fumagillin | 1.080094016 | 1.348158131 | −0.27 |
| Lofexidine | 0.840141949 | 1.108223017 | −0.27 |
| Alvimopan (dihydrate) | 0.986360514 | 1.254841998 | −0.27 |
| Oxacillin (sodium monohydrate) | 1.034723172 | 1.303848163 | −0.27 |
| Imiquimod | 0.871284536 | 1.140712946 | −0.27 |
| Indirubin | 0.578238648 | 0.848296444 | −0.27 |
| Erythromycin estolate | 1.076113464 | 1.347006458 | −0.27 |
| Trimethoprim | 1.063907209 | 1.334816754 | −0.27 |
| Bestatin | 0.991666667 | 1.262846291 | −0.27 |
| Mephenytoin | 1.003013613 | 1.274287376 | −0.27 |
| Vonoprazan (Fumarate) | 0.783818694 | 1.055372549 | −0.27 |
| Solifenacin | 0.949740035 | 1.221857184 | −0.27 |
| Selumetinib | 0.461839864 | 0.734347443 | −0.27 |
| Hydroquinidine | 1.518351112 | 1.791105121 | −0.27 |
| Anagrelide (hydrochloride) | 0.948313825 | 1.221176107 | −0.27 |
| 5-Fluorouracil | 0.52044293 | 0.793430335 | −0.27 |
| Dapiprazole (hydrochloride) | 1.063166456 | 1.336778332 | −0.27 |
| Sophoridine | 1.063350435 | 1.337031452 | −0.27 |
| Gliclazide | 0.958323017 | 1.233452507 | −0.28 |
| Cimetropium (Bromide) | 1.428674742 | 1.703953279 | −0.28 |
| Clonixin | 0.904090268 | 1.180095366 | −0.28 |
| Atropine | 1.040361126 | 1.316681534 | −0.28 |
| Fumaric acid | 0.840070664 | 1.11663758 | −0.28 |
| Dexrazoxane (hydrochloride) | 1.002145002 | 1.27925052 | −0.28 |
| Nifuratel | 1.12007874 | 1.397196262 | −0.28 |
| Doxofylline | 1.024085638 | 1.301612903 | −0.28 |
| Fesoterodine | 0.826472595 | 1.104450262 | −0.28 |
| Beclometasone dipropionate | 0.854362837 | 1.133134625 | −0.28 |
| Flucytosine | 1.474688577 | 1.753537068 | −0.28 |
| Pinacidil monohydrate | 0.806430868 | 1.085364193 | −0.28 |
| Vortioxetine (hydrobromide) | 1.083081769 | 1.362624135 | −0.28 |
| Medetomidine | 0.81375969 | 1.093945487 | −0.28 |
| Folinic acid | 0.951470435 | 1.231820826 | −0.28 |
| Chloroprocaine (hydrochloride) | 0.902863745 | 1.183592618 | −0.28 |
| Lactose | 0.949704485 | 1.230436796 | −0.28 |
| Triamterene | 1.301129235 | 1.582383874 | −0.28 |
| Ibandronate (Sodium | 0.79604193 | 1.077334284 | −0.28 |
| Monohydrate) | | | |
| Oxymatrine | 0.901401565 | 1.184721703 | −0.28 |
| Rucaparib (Camsylate) | 0.993981823 | 1.277363555 | −0.28 |
| Sodium tauroglycocholate | 0.972882142 | 1.256366723 | −0.28 |
| Mozavaptan | 0.837524626 | 1.121160673 | −0.28 |
| Paliperidone | 0.802294588 | 1.085970057 | −0.28 |
| Frovatriptan (succinate hydrate) | 0.868336278 | 1.153286795 | −0.28 |
| Sodium 4-aminosalicylate | 1.188245315 | 1.473324515 | −0.29 |
| (dihydrate) | | | |
| Tigecycline | 0.892915236 | 1.178545973 | −0.29 |
| Carboprost (tromethamine) | 1.30561987 | 1.591422122 | −0.29 |
| Metaxalone | 1.013691835 | 1.299513094 | −0.29 |
| Choline (bitartrate) | 1.053871063 | 1.340015857 | −0.29 |
| Tripelennamine (hydrochloride) | 0.834344251 | 1.1204947 | −0.29 |
| Quinidine | 0.849211437 | 1.135591148 | −0.29 |
| Hydroxyurea | 1.13118527 | 1.418095801 | −0.29 |
| Urea | 1.641323916 | 1.928450322 | −0.29 |
| Megestrol acetate | 0.964713977 | 1.252176076 | −0.29 |
| Talazoparib tosylate | 0.311989956 | 0.599602583 | −0.29 |
| Bromhexine (hydrochloride) | 0.793212776 | 1.081098039 | −0.29 |
| Uridine | 1.006416049 | 1.294488002 | −0.29 |
| Adenosine | 1.600332364 | 1.888436758 | −0.29 |
| Idelalisib | 1.219087871 | 1.507336483 | −0.29 |
| Rapacuronium bromide | 0.762636474 | 1.051236749 | −0.29 |
| Fondaparinux (sodium) | 0.893692196 | 1.182373473 | −0.29 |
| Oxiracetam | 0.9455843 | 1.234331797 | −0.29 |
| Sarpogrelate (hydrochloride) | 1.089233978 | 1.377990431 | −0.29 |
| Mifepristone | 1.019090098 | 1.308064516 | −0.29 |
| Mivacurium (dichloride) | 1.463388176 | 1.7524708 | −0.29 |
| Rapamycin | 0.870367022 | 1.159478886 | −0.29 |
| Cabozantinib (S-malate) | 0.925507428 | 1.215598609 | −0.29 |
| Maprotiline (hydrochloride) | 0.924000397 | 1.214765101 | −0.29 |
| L-Lysine | 0.729516746 | 1.020527859 | −0.29 |
| Demecarium Bromide | 0.90589736 | 1.197304338 | −0.29 |
| Lobeline (hydrochloride) | 1.116060226 | 1.407683319 | −0.29 |
| Testosterone propionate | 0.766949724 | 1.059010601 | −0.29 |
| Agomelatine | 0.891378999 | 1.184041934 | −0.29 |
| DL-Methionine methylsulfonium | 0.888387999 | 1.181963288 | −0.29 |
| (chloride) | | | |
| Nadolol | 1.014236725 | 1.308027923 | −0.29 |
| Pralidoxime (chloride) | 0.757365111 | 1.051185548 | −0.29 |
| (R)-(+)-Atenolol | 0.871966155 | 1.165964778 | −0.29 |
| Valbenazine | 1.011115127 | 1.305416964 | −0.29 |
| Trometamol (hydrochloride) | 1.378774182 | 1.673405211 | −0.29 |
| Rifamycin (sodium) | 0.750469704 | 1.045333333 | −0.29 |
| Desloratadine | 0.625398144 | 0.92200746 | −0.30 |
| Lenalidomide (hemihydrate) | 1.135093697 | 1.431878307 | −0.30 |
| DHEA | 0.73712307 | 1.034279154 | −0.30 |
| Fexofenadine (hydrochloride) | 1.104330709 | 1.401509705 | −0.30 |
| Flucloxacillin sodium | 0.829658413 | 1.12722737 | −0.30 |
| Amlodipine (maleate) | 0.788515973 | 1.087279152 | −0.30 |
| Prednisone acetate | 0.660566365 | 0.960526316 | −0.30 |
| Camostat (mesylate) | 1.02785265 | 1.328155001 | −0.30 |
| Ledipasvir (D-tartrate) | 0.692007009 | 0.992579505 | −0.30 |
| Norfloxacin | 1.395669291 | 1.696261682 | −0.30 |
| Dorzolamide (hydrochloride) | 0.645925641 | 0.94757085 | −0.30 |
| Adiphenine (hydrochloride) | 0.952737164 | 1.254552352 | −0.30 |
| Benzocaine | 1.006691947 | 1.308595433 | −0.30 |
| Cefazolin (sodium) | 1.076396468 | 1.378470851 | −0.30 |
| Peretinoin | 0.970992228 | 1.273105251 | −0.30 |
| Penicillin G Procaine | 0.949122479 | 1.253671706 | −0.30 |
| Halofantrine hydrochloride | 0.928192871 | 1.232984293 | −0.30 |
| 10-Undecenoic acid | 0.764793099 | 1.069611307 | −0.30 |
| Sumatriptan (succinate) | 0.889729452 | 1.194767086 | −0.31 |
| Chloroquine (phosphate) | 0.980047179 | 1.285501119 | −0.31 |
| Bicyclol | 1.024789629 | 1.330628272 | −0.31 |
| Olmesartan medoxomil | 1.700752823 | 2.007157464 | −0.31 |
| Regadenoson | 1.05521417 | 1.361962461 | −0.31 |
| Uracil | 0.894940992 | 1.202531646 | −0.31 |
| Sulfanilamide | 0.913311501 | 1.221255061 | −0.31 |
| Midodrine (hydrochloride) | 1.751568381 | 2.060327198 | −0.31 |
| Exemestane | 0.905347753 | 1.214765101 | −0.31 |
| Abiraterone acetate | 0.899987178 | 1.209438807 | −0.31 |
| Ritonavir | 0.823273837 | 1.133176471 | −0.31 |
| Lomerizine dihydrochloride | 0.867824551 | 1.177823083 | −0.31 |
| DL-Glutamine | 0.630666012 | 0.940733772 | −0.31 |
| Acrivastine | 0.715610152 | 1.027319031 | −0.31 |
| Tolterodine | 0.856066752 | 1.168698873 | −0.31 |
| Abemaciclib (methanesulfonate) | 0.613139346 | 0.926229508 | −0.31 |
| Ampicillin | 0.756379023 | 1.069568407 | −0.31 |
| Etamivan | 0.914078778 | 1.227398342 | −0.31 |
| β-Carotene | 0.608072225 | 0.92298543 | −0.31 |
| Malotilate | 0.855664585 | 1.171658986 | −0.32 |
| 20(S)-Ginsenoside Rg3 | 1.403362864 | 1.721024259 | −0.32 |
| Baricitinib | 0.967357188 | 1.286140089 | −0.32 |
| Megestrol | 0.739453776 | 1.058320088 | −0.32 |
| Sitafloxacin (hydrate) | 0.723490555 | 1.042753511 | −0.32 |
| Methacycline (hydrochloride) | 0.99503919 | 1.315304645 | −0.32 |
| Menaquinone-4 | 0.7730155 | 1.093647059 | −0.32 |
| Estrone | 1.087983833 | 1.409417398 | −0.32 |
| Phthalylsulfacetamide | 0.709799164 | 1.031448763 | −0.32 |
| Ketotifen (fumarate) | 1.552080572 | 1.875778155 | −0.32 |
| Varenicline | 1.033139535 | 1.359361037 | −0.33 |
| 10-Undecenoic acid (zinc salt) | 1.234897916 | 1.561324304 | −0.33 |
| Treprostinil | 0.850669045 | 1.17718894 | −0.33 |
| Tolmetin | 1.391791719 | 1.718328841 | −0.33 |
| Maltitol | 0.937652103 | 1.264392653 | −0.33 |
| Mebrofenin | 0.872624343 | 1.199646643 | −0.33 |
| Ethynodiol diacetate | 0.932334557 | 1.25950783 | −0.33 |
| Apraclonidine (hydrochloride) | 1.017880364 | 1.345360825 | −0.33 |
| Fluconazole | 0.939161463 | 1.267511521 | −0.33 |
| Pirfenidone | 1.068331847 | 1.397465438 | −0.33 |
| Suramin (sodium salt) | 0.971624169 | 1.303197789 | −0.33 |
| Dapagliflozin | 0.691939273 | 1.024114992 | −0.33 |
| Gliquidone | 1.001785891 | 1.334780892 | −0.33 |
| Gluconate (Calcium) | 0.800440375 | 1.133508257 | −0.33 |
| Permethrin | 0.899394781 | 1.233188577 | −0.33 |
| Pipobroman | 1.050581395 | 1.384383378 | −0.33 |
| Tenofovir (Disoproxil) | 0.907594264 | 1.241748439 | −0.33 |
| Levonorgestrel | 1.542539094 | 1.876910017 | −0.33 |
| Propofol | 1.224470267 | 1.559463695 | −0.33 |
| Chlormezanone | 1.113941324 | 1.44895288 | −0.34 |
| Rucaparib | 0.704407602 | 1.039929329 | −0.34 |
| (−)-Menthol | 0.886625792 | 1.222513089 | −0.34 |
| Pyridoxine (hydrochloride) | 0.993627191 | 1.329765091 | −0.34 |
| Pranoprofen | 1.135774392 | 1.47408377 | −0.34 |
| Argipressin | 1.167053999 | 1.505833188 | −0.34 |
| Midecamycin | 0.91486088 | 1.25492114 | −0.34 |
| Cefoxitin | 1.500271199 | 1.840521114 | −0.34 |
| Doxazosin (mesylate) | 0.983939326 | 1.325398535 | −0.34 |
| Abemaciclib | 0.668587142 | 1.011257036 | −0.34 |
| Lamotrigine | 1.093233083 | 1.43687968 | −0.34 |
| Ferulic acid | 0.711819989 | 1.05547636 | −0.34 |
| Clonidine | 0.915697674 | 1.259718499 | −0.34 |
| Omarigliptin | 1.419560032 | 1.763723826 | −0.34 |
| Lidocaine | 1.08810143 | 1.432686477 | −0.34 |
| Solriamfetol | 0.885167464 | 1.229849424 | −0.34 |
| Gallamine Triethiodide | 1.026983095 | 1.372043663 | −0.35 |
| Taurochenodeoxycholic acid | 1.534984632 | 1.880053908 | −0.35 |
| (sodium salt) | | | |
| Pretomanid | 0.90455464 | 1.250598563 | −0.35 |
| Duvelisib | 0.881709742 | 1.227963887 | −0.35 |
| Aristolochic acid A | 0.576208178 | 0.92298543 | −0.35 |
| Plerixafor (octahydrochloride) | 0.812000939 | 1.158901961 | −0.35 |
| Dantrolene (sodium | 1.217618749 | 1.565325385 | −0.35 |
| hemiheptahydrate) | | | |
| Urapidil (hydrochloride) | 0.869973346 | 1.219941349 | −0.35 |
| Protriptyline (hydrochloride) | 0.792290066 | 1.142402827 | −0.35 |
| Siponimod | 0.770100256 | 1.120865456 | −0.35 |
| Bethanechol (chloride) | 0.988184748 | 1.340468396 | −0.35 |
| Cefozopran (hydrochloride) | 1.537154222 | 1.889937107 | −0.35 |
| Ioxilan | 0.867973974 | 1.221311475 | −0.35 |
| Sulfadimethoxine (sodium) | 1.521243898 | 1.875561545 | −0.35 |
| Tolmetin (sodium dihydrate) | 0.939992307 | 1.294534784 | −0.35 |
| Rifampicin | 0.864073931 | 1.219235469 | −0.36 |
| Ribostamycin (sulfate) | 1.47765424 | 1.833286477 | −0.36 |
| Niraparib (tosylate) | 1.091343601 | 1.447517098 | −0.36 |
| Dehydroandrographolide | 1.432290725 | 1.790206649 | −0.36 |
| sn-Glycero-3-phosphocholine | 0.599916909 | 0.95930824 | −0.36 |
| Sunitinib | 1.120593021 | 1.480233603 | −0.36 |
| Nelfinavir | 0.882783229 | 1.242626728 | −0.36 |
| Agomelatine (hydrochloride) | 0.765588288 | 1.125800815 | −0.36 |
| Cephradine | 0.852288755 | 1.212862208 | −0.36 |
| Sulfisomidin | 0.786183888 | 1.147015517 | −0.36 |
| Cephalexin | 0.979839429 | 1.34124424 | −0.36 |
| Ibuprofen | 1.150085179 | 1.51212522 | −0.36 |
| Azelaic acid | 0.881339402 | 1.244693754 | −0.36 |
| Tetrahydrobiopterin | 1.025491237 | 1.389235801 | −0.36 |
| Fostamatinib | 1.04160651 | 1.405958471 | −0.36 |
| Procainamide (hydrochloride) | 1.164674398 | 1.529262673 | −0.36 |
| Alectinib | 1.019931857 | 1.385141093 | −0.37 |
| Nelfinavir (Mesylate) | 1.199190907 | 1.564574899 | −0.37 |
| Irinotecan | 0.363121715 | 0.728975265 | −0.37 |
| Paliperidone palmitate | 0.826769199 | 1.192844677 | −0.37 |
| Trimethobenzamide hydrochloride | 0.874028556 | 1.241118229 | −0.37 |
| Famotidine | 1.298197892 | 1.665781487 | −0.37 |
| 5-Acetylsalicylic acid | 0.819808422 | 1.187536401 | −0.37 |
| Tafamidis | 0.938761999 | 1.307113286 | −0.37 |
| Quinapril (hydrochloride) | 0.86202028 | 1.230406703 | −0.37 |
| Estradiol dipropionate | 1.521967095 | 1.89083558 | −0.37 |
| Moexipril (hydrochloride) | 1.080656186 | 1.451506181 | −0.37 |
| D-Pantothenic acid (hemicalcium | 0.786040715 | 1.15937606 | −0.37 |
| salt) | | | |
| Voxelotor | 1.474688577 | 1.853140917 | −0.38 |
| Vortioxetine | 0.843024002 | 1.222028277 | −0.38 |
| Xylometazoline (hydrochloride) | 0.780335351 | 1.15949467 | −0.38 |
| Bepotastine (besilate) | 0.863910209 | 1.24466132 | −0.38 |
| Methoxsalen | 0.957464146 | 1.340119537 | −0.38 |
| Epinastine (hydrochloride) | 1.132075472 | 1.514866391 | −0.38 |
| (S)-Rasagiline | 0.899580289 | 1.283320032 | −0.38 |
| Mupirocin | 1.069365476 | 1.454188482 | −0.38 |
| Chlortetracycline (hydrochloride) | 1.105819628 | 1.490734141 | −0.38 |
| Homatropine (methylbromide) | 0.916359617 | 1.301819266 | −0.39 |
| Mepyramine maleate | 0.839888077 | 1.225857941 | −0.39 |
| Fadrozole | 1.037641154 | 1.426380368 | −0.39 |
| Ipratropium (bromide) | 1.146119536 | 1.535714286 | −0.39 |
| Fedratinib | 0.284026775 | 0.675895275 | −0.39 |
| Troxipide | 1.184811471 | 1.578352661 | −0.39 |
| Mequinol | 0.826277131 | 1.21992463 | −0.39 |
| Brimonidine (tartrate) | 1.309078545 | 1.703717754 | −0.39 |
| Oxcarbazepine | 0.909732017 | 1.304804988 | −0.40 |
| Glycerol phenylbutyrate | 1.340360422 | 1.737101669 | −0.40 |
| Levamisole (hydrochloride) | 0.904603154 | 1.301381587 | −0.40 |
| Pyrantel (pamoate) | 0.855955178 | 1.253931275 | −0.40 |
| Triamcinolone hexacetonide | 0.652542373 | 1.053086788 | −0.40 |
| Bretylium (tosylate) | 0.816326531 | 1.217140862 | −0.40 |
| Umeclidinium (bromide) | 0.9344115 | 1.335835224 | −0.40 |
| Vonoprazan | 0.847279957 | 1.249271986 | −0.40 |
| Cimetidine | 0.952002648 | 1.358298833 | −0.41 |
| Ciprofibrate | 1.16082965 | 1.568664643 | −0.41 |
| Cilastatin | 0.849979782 | 1.258303887 | −0.41 |
| Ledipasvir (acetone) | 0.970705168 | 1.382513661 | −0.41 |
| Ethambutol (dihydrochloride) | 1.016311167 | 1.428133216 | −0.41 |
| Nabumetone | 1.111866712 | 1.524658574 | −0.41 |
| L-Glutamic acid monosodium salt | 0.777954162 | 1.191077773 | −0.41 |
| Silibinin | 1.695610026 | 2.112241438 | −0.42 |
| Eletriptan (hydrobromide) | 0.912795952 | 1.329650748 | −0.42 |
| Gluconate (sodium) | 0.987254381 | 1.4058876 | −0.42 |
| Methicillin (sodium salt) | 1.406167979 | 1.828540618 | −0.42 |
| Iopamidol | 1.09213944 | 1.515584189 | −0.42 |
| Piroxicam | 1.008110754 | 1.432697701 | −0.42 |
| Vitamin K1 | 0.635923002 | 1.062394032 | −0.43 |
| Deferoxamine (mesylate) | 1.027067669 | 1.453595682 | −0.43 |
| Carbazochrome (sodium | 1.40700442 | 1.834218513 | −0.43 |
| sulfonate) | | | |
| Dutasteride | 1.205407534 | 1.632861872 | −0.43 |
| Daclatasvir (dihydrochloride) | 0.721489246 | 1.149097263 | −0.43 |
| Flumazenil | 0.839719971 | 1.267819863 | −0.43 |
| Fipexide | 1.225236086 | 1.654554759 | −0.43 |
| Imidapril (hydrochloride) | 0.852288755 | 1.281819293 | −0.43 |
| Micafungin (sodium) | 1.774269492 | 2.204476094 | −0.43 |
| D-Tyrosine | 0.997474216 | 1.429897166 | −0.43 |
| Trospium (chloride) | 1.645542427 | 2.079828758 | −0.43 |
| Abiraterone | 1.472774764 | 1.907369498 | −0.43 |
| Famciclovir | 1.160703076 | 1.597118728 | −0.44 |
| Rilmenidine (hemifumarate) | 1.430844332 | 1.87017071 | −0.44 |
| Norvancomycin (hydrochloride) | 0.895445509 | 1.334877643 | −0.44 |
| Amsacrine (hydrochloride) | 0.387253983 | 0.82776918 | −0.44 |
| Taurodeoxycholic acid (sodium | 1.168483816 | 1.615299824 | −0.45 |
| hydrate) | | | |
| Eliglustat | 0.638442075 | 1.085887384 | −0.45 |
| Deoxycholic acid | 0.930961232 | 1.378531073 | −0.45 |
| Ranitidine (hydrochloride) | 0.821899225 | 1.269548704 | −0.45 |
| Canagliflozin (hemihydrate) | 0.892419729 | 1.340233346 | −0.45 |
| Diclofenac (Sodium) | 0.907500663 | 1.356253537 | −0.45 |
| Teneligliptin (hydrobromide) | 1.115276476 | 1.564448375 | −0.45 |
| Norgestrel | 1.00424854 | 1.455842997 | −0.45 |
| Bevantolol (hydrochloride) | 0.952022251 | 1.403920358 | −0.45 |
| Pyridostigmine (bromide) | 1.089952153 | 1.545533693 | −0.46 |
| Lansoprazole | 0.647131976 | 1.102952913 | −0.46 |
| Methocarbamol | 0.872673053 | 1.329650748 | −0.46 |
| Nevirapine | 0.925356949 | 1.38439138 | −0.46 |
| Letrozole | 1.20960756 | 1.669575685 | −0.46 |
| Ibuprofen piconol | 0.895849526 | 1.356743815 | −0.46 |
| Chromocarb | 1.470620141 | 1.932165319 | −0.46 |
| Iguratimod | 1.252054068 | 1.717317487 | −0.47 |
| Nicardipine (hydrochloride) | 0.825591903 | 1.292370414 | −0.47 |
| Benznidazol | 1.635784517 | 2.104103086 | −0.47 |
| Crisaborole | 0.947858305 | 1.417082442 | −0.47 |
| Nilutamide | 1.003389831 | 1.473963134 | −0.47 |
| Lamivudine | 1.17120954 | 1.643518519 | −0.47 |
| Bimatoprost | 0.956940173 | 1.429530201 | −0.47 |
| Iotalamic acid | 0.985370611 | 1.459369315 | −0.47 |
| Gallic acid | 1.612519042 | 2.089182774 | −0.48 |
| Enzalutamide | 0.311576075 | 0.789267016 | −0.48 |
| Carvedilol | 0.966535433 | 1.44751977 | −0.48 |
| Ilaprazole | 1.084131776 | 1.567258501 | −0.48 |
| Losartan | 0.874118923 | 1.369565217 | −0.50 |
| Ledipasvir | 0.692571842 | 1.188701223 | −0.50 |
| Aliskiren | 0.826830406 | 1.325618516 | −0.50 |
| Rilpivirine | 1.359247876 | 1.859389039 | −0.50 |
| Pyrantel (tartrate) | 1.209248797 | 1.712369598 | −0.50 |
| Allantoin | 0.638692702 | 1.146490336 | −0.51 |
| Spironolactone | 1.272309711 | 1.783968368 | −0.51 |
| Pregnenolone | 0.911842804 | 1.423728814 | −0.51 |
| Atosiban (acetate) | 1.069969264 | 1.587151842 | −0.52 |
| Venlafaxine (hydrochloride) | 1.023239918 | 1.551105694 | −0.53 |
| Gilteritinib hemifumarate | 0.405656671 | 0.939337086 | −0.53 |
| Almotriptan (malate) | 1.105066304 | 1.641502276 | −0.54 |
| Praziquantel | 1.107836457 | 1.644400353 | −0.54 |
| Desvenlafaxine (succinate | 0.886093166 | 1.424506829 | −0.54 |
| hydrate) | | | |
| Gestodene | 1.003076493 | 1.543185642 | −0.54 |
| Chlorphenoxamine | 1.159512652 | 1.703294274 | −0.54 |
| Rabeprazole (sodium) | 0.90653213 | 1.460600654 | −0.55 |
| Zaltoprofen | 0.725603536 | 1.283383915 | −0.56 |
| Meticrane | 0.83164847 | 1.392579505 | −0.56 |
| Amoxapine | 1.017400644 | 1.586250315 | −0.57 |
| Methylbenactyzium Bromide | 1.081203008 | 1.651401706 | −0.57 |
| Fosphenytoin (disodium) | 1.233485939 | 1.806297442 | −0.57 |
| Pioglitazone | 1.521055485 | 2.094933023 | −0.57 |
| Fursultiamine | 1.267832027 | 1.841862845 | −0.57 |
| Mepivacaine | 0.984003368 | 1.558314522 | −0.57 |
| Nicergoline | 0.470775529 | 1.053141361 | −0.58 |
| Salbutamol (hemisulfate) | 1.453061224 | 2.035507429 | −0.58 |
| Regorafenib (monohydrate) | 0.727976564 | 1.310978639 | −0.58 |
| Enalaprilat (dihydrate) | 1.051204282 | 1.635253456 | −0.58 |
| Butamben | 1.080579216 | 1.66468254 | −0.58 |
| α-Vitamin E | 0.907594264 | 1.492714838 | −0.59 |
| Clavulanate (lithium) | 1.386895811 | 1.974061949 | −0.59 |
| Zidovudine | 1.268372703 | 1.855859094 | −0.59 |
| Dinoprost (tromethamine salt) | 1.08330494 | 1.670855379 | −0.59 |
| Capecitabine | 0.647992864 | 1.236175115 | −0.59 |
| Bromfenac (sodium) | 1.029053421 | 1.623701083 | −0.59 |
| Tenofovir (Disoproxil Fumarate) | 0.722117598 | 1.320914059 | −0.60 |
| Desogestrel | 1.02150732 | 1.62085032 | −0.60 |
| Diphenmanil (methylsulfate) | 1.508042653 | 2.108910891 | −0.60 |
| Taurine | 1.362668274 | 1.970829069 | −0.61 |
| Citalopram (hydrobromide) | 0.847553325 | 1.457347356 | −0.61 |
| Budesonide | 0.640504003 | 1.254288294 | −0.61 |
| Triprolidine (hydrochloride | 1.190944882 | 1.805535586 | −0.61 |
| monohydrate) | | | |
| Nedocromil | 1.336197637 | 1.950893981 | −0.61 |
| Ivermectin | 0.455702848 | 1.074330424 | −0.62 |
| Bremelanotide (Acetate) | 1.020854022 | 1.644335717 | −0.62 |
| Cholesterol | 1.069038097 | 1.696764485 | −0.63 |
| Desipramine hydrochloride | 1.494049643 | 2.125569044 | −0.63 |
| Itopride (hydrochloride) | 1.055774278 | 1.710639827 | −0.65 |
| Busulfan | 1.264876477 | 1.935784437 | −0.67 |
| Cephalexin (monohydrate) | 0.916091344 | 1.590246803 | −0.67 |
| Betrixaban | 0.748556026 | 1.423808693 | −0.68 |
| Minoxidil | 1.166010499 | 1.841480949 | −0.68 |
| Ospemifene | 1.222440945 | 1.909058231 | −0.69 |
| Lysipressin | 1.147637795 | 1.850107836 | −0.70 |
| Nitrofurazone | 1.150590763 | 1.854192899 | −0.70 |
| L-Proline | 1.087535145 | 1.794384258 | −0.71 |
| Amodiaquine (dihydrochloride) | 1.058934961 | 1.765989466 | −0.71 |
| Niflumic acid | 0.287923584 | 0.995549738 | −0.71 |
| Roflumilast | 0.89716248 | 1.606872452 | −0.71 |
| Dimetridazole | 0.891533492 | 1.602048558 | −0.71 |
| Lomefloxacin (hydrochloride) | 0.934049409 | 1.656761521 | −0.72 |
| Norethindrone acetate | 0.969058143 | 1.69461305 | −0.73 |
| Acemetacin | 1.508941129 | 2.235926305 | −0.73 |
| Tolvaptan | 1.367070305 | 2.101921957 | −0.73 |
| Carbidopa | 1.220883534 | 1.956256358 | −0.74 |
| Cefamandole (sodium) | 1.429375126 | 2.169396111 | −0.74 |
| Teriflunomide | 0.966535433 | 1.712077642 | −0.75 |
| Nefazodone (hydrochloride) | 0.275187628 | 1.02486911 | −0.75 |
| Artemisinin | 0.860268661 | 1.623262123 | −0.76 |
| Ornipressin | 0.789527372 | 1.553490137 | −0.76 |
| Glucosamine | 1.145653547 | 1.916478555 | −0.77 |
| Oxyphenbutazone | 1.055567249 | 1.844243792 | −0.79 |
| DL-Norepinephrine | 0.176029111 | 0.965183246 | −0.79 |
| (hydrochloride) | | | |
| (S)-Flurbiprofen | 1.038800375 | 1.832412116 | −0.79 |
| Iohexol | 0.831689901 | 1.632397572 | −0.80 |
| Nadifloxacin | 1.054461942 | 1.858734723 | −0.80 |
| Sulfamerazine | 1.355490929 | 2.163106138 | −0.81 |
| Clodronic acid (disodium salt) | 1.075144509 | 1.882776935 | −0.81 |
| Sulfathiazole (sodium) | 1.040026247 | 1.851545651 | −0.81 |
| Guaiacol | 1.473480127 | 2.295354357 | −0.82 |
| Gimeracil | 1.49069221 | 2.326732673 | −0.84 |
| Oxytetracycline | 1.447316103 | 2.283634246 | −0.84 |
| Lacidipine | 0.959973753 | 1.799784328 | −0.84 |
| L-(−)-α-Methyldopa (hydrate) | 1.11707841 | 1.970032737 | −0.85 |
| Anethole (trithione) | 1.115359828 | 1.972047343 | −0.86 |
| Etofenamate | 1.526838966 | 2.387303436 | −0.86 |
| Fructose | 1.164648473 | 2.032032615 | −0.87 |
| Bithionol | 1.352611603 | 2.227722772 | −0.88 |
| Sucrose | 0.977159881 | 1.867143395 | −0.89 |
| Methylcobalamin | 0.858891533 | 1.749241275 | −0.89 |
| Moxalactam (sodium salt) | 1.097314715 | 1.994208008 | −0.90 |
| Acenocoumarol | 1.113641246 | 2.014354067 | −0.90 |
| Metformin | 1.012909633 | 1.921339857 | −0.91 |
| Andrographolide | 0.92064811 | 1.85927433 | −0.94 |
| Suplatast (Tosilate) | 1.500090367 | 2.446709377 | −0.95 |
| Chlorprothixene | 1.06122449 | 2.022412491 | −0.96 |
| Angiotensin II 5-valine | 1.090186156 | 2.070471753 | −0.98 |
| Diatrizoic acid | 0.837130228 | 1.834218513 | −1.00 |
| Anidulafungin | 0.217472408 | 1.251396648 | −1.03 |
| Afoxolaner | 1.371407916 | 2.419918462 | −1.05 |
| Glycopyrrolate | 1.232604374 | 2.313919627 | −1.08 |
| Stearic acid | 0.659188478 | 1.741268227 | −1.08 |
| Piperacillin (sodium) | 1.171028943 | 2.325194981 | −1.15 |
| Parecoxib (Sodium) | 1.250677752 | 2.41875364 | −1.17 |
| Chloroquine | 0.999668984 | 2.192322168 | −1.19 |
| Phenylephrine (hydrochloride) | 1.088381331 | 2.302220549 | −1.21 |
| Cephapirin (sodium) | 1.20947045 | 2.472335469 | −1.26 |
| Tiotropium (Bromide) | 1.196457618 | 2.467676179 | −1.27 |
| L-Isoleucine | 1.020061449 | 2.302271404 | −1.28 |
| Propyphenazone | 1.069221037 | 2.377984857 | −1.31 |

### Example 3: Validation of Identified Drug with Cholesterol-Free Media

A series of experiments were designed to assess the effect of cholesterol depletion and statin treatment on the anti-cancer effect of drugs identified in the FDA drug screens. Cancer cells were grown in media containing cholesterol and cholesterol-free media in the presence or absence of simvastatin. The effect of the drug identified in the FDA drug screens on cancer cell growth was measured.

### Drug Validation in H292 Cells.

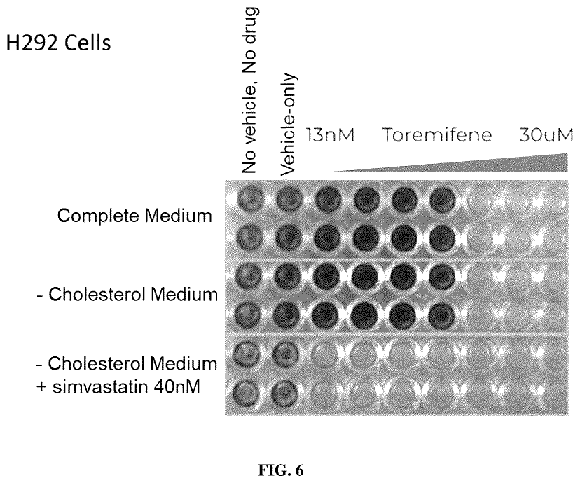

On Day 1, cells were seeded at a density of around 7500 cells/well in a Corning 96-well flat bottom culture plate in complete media containing 10% FBS. On Day 2, the plates were washed with PBS then fed with three different media types, supplemented with increasing concentrations of the validation drug in 3-fold increasing concentration range (e.g., toremifene, from 13 nM to 30 μM. A ‘vehicle-only’ control condition and a ‘no-drug & no-vehicle’ control condition were also used. Medium 1 was ‘complete medium’ containing all essential nutrients, including cholesterol. Medium 2 was cholesterol-free medium. Medium 3 was cholesterol-free medium supplemented with 40 nM of simvastatin. FIG. 6 shows the plate layout of the drug validation study.

On Day 5, spent media was discarded, and wells were stained with a crystal violet staining solution, washed and dried. 100 μL of 10% acetic acid was added to each well to release the crystal violet taken up by the cells. Plates were read using a Biotek Powerwave plate reader to determine the absorbance of the crystal violet released as an indicator of cell growth at a wavelength of 595 nm.

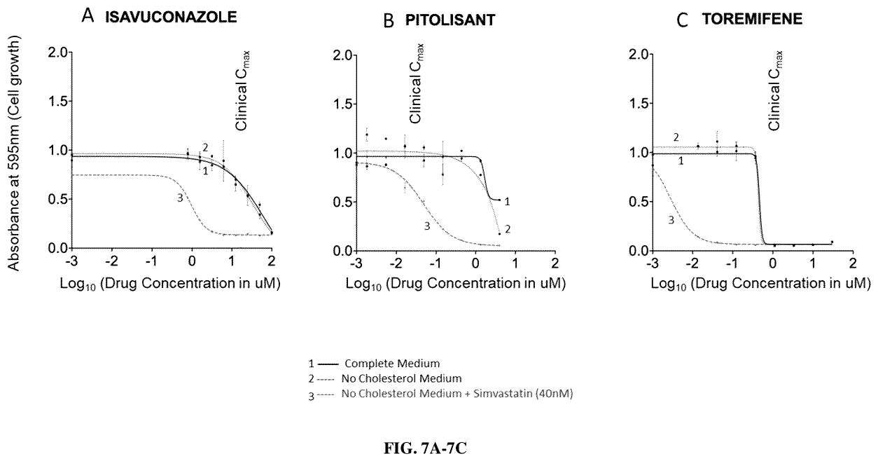

Growth curves showing absorbance of crystal violet at 595 nm were plotted from test wells against the logarithmic concentration of the validation drug. FIG. 7A-7C show growth of cancer cell lines in the three media conditions subjected to increasing doses of isavuconazole (FIG. 7A), pitolisant (FIG. 7B), or toremifene (FIG. 7C) from the FDA screen under validation. For each of the drugs being validated, there is a strong ‘left-shift’ in the dose response curve for cells are grown in the cholesterol-free medium+simvastatin. The data suggest that the anti-proliferative effect of the drug was strongly enhanced by cholesterol restriction and the presence of statin.

### Drug Validation in Multiple Cancer Cell Lines.

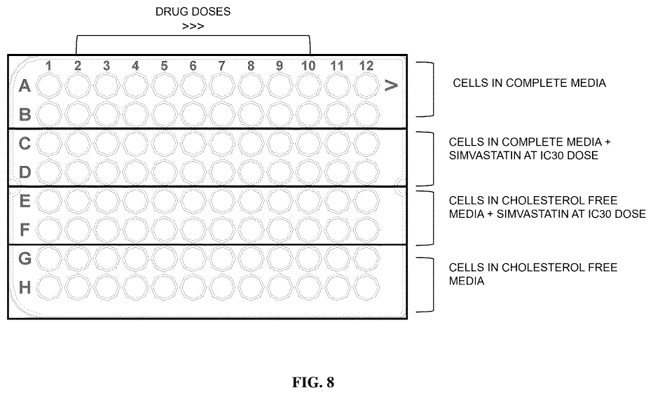

On Day 1, cells were seeded at a density of around 7500 cells/well in a Corning 96-well flat bottom culture plate in complete media containing 10% FBS. On Day 2, the plates were washed with PBS then fed with four different media types, supplemented with increasing concentrations of the validation drug in 3-fold increasing concentration range (e.g. toremifene, from 13 nM to 30 μM. A ‘vehicle-only’ control condition and a ‘no-drug & no-vehicle’ control condition were also used. Medium 1 was ‘complete medium’ containing all essential nutrients, including cholesterol. Medium 2 was cholesterol-free medium. Medium 3 was cholesterol-free medium supplemented with simvastatin 40 nM. Medium 4 was ‘complete medium’ containing all essential nutrients, including cholesterol, supplemented with simvastatin at the IC30 concentration for each cell line. FIG. 8 shows the plate layout of the drug validation study.

On Day 5, cell growth was measured by absorbance or fluorescence. For absorbance growth measurements, spent media was discarded, and wells were stained with a crystal violet staining solution, washed, and dried. 100 μL of 10% acetic acid was added to each well to release the crystal violet taken up by the cells. Plates were read using a Biotek Powerwave plate reader to determine the absorbance of the crystal violet released as an indicator of cell growth at a wavelength of 595 nm. For fluorescence growth measurements, 100 μL of a resazurin solution was added to each well and the plate incubated at 37° C. for four hours. Plates were read using a Biotek Powerwave plate reader to determine the fluorescence of reosurfin as an indicator of cell growth at a wavelength of 590 nm.

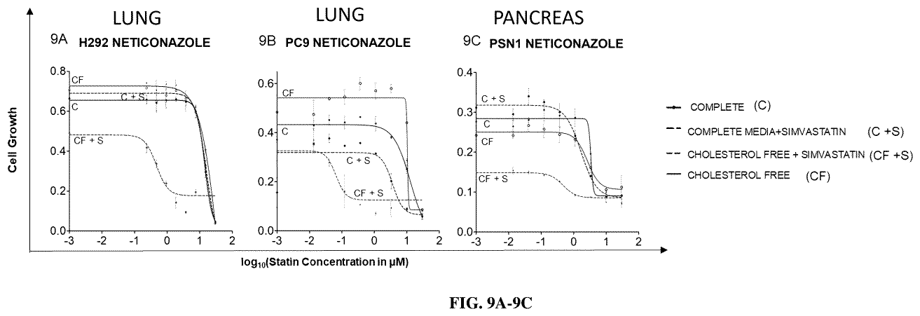

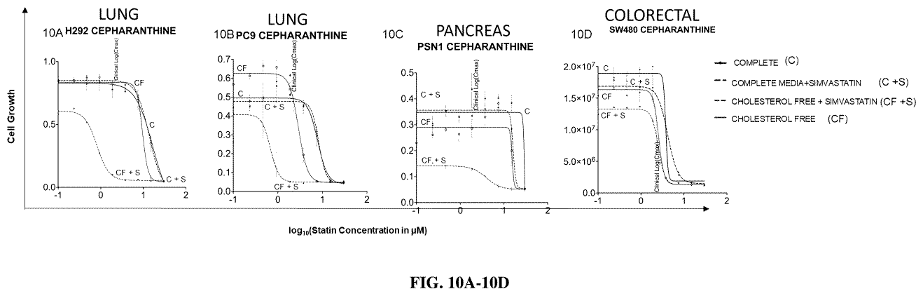

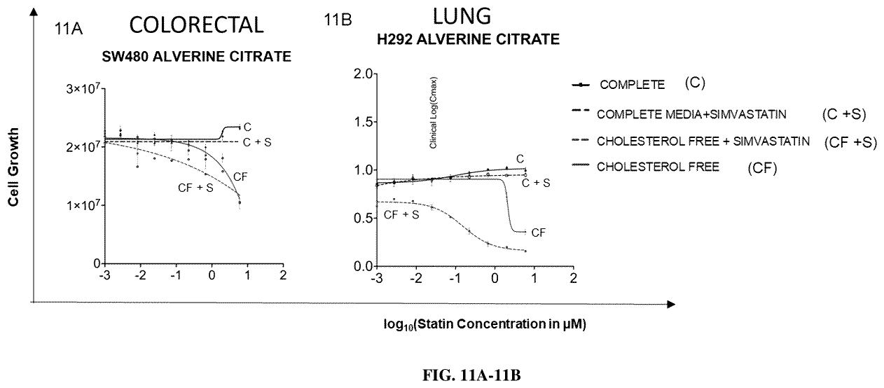

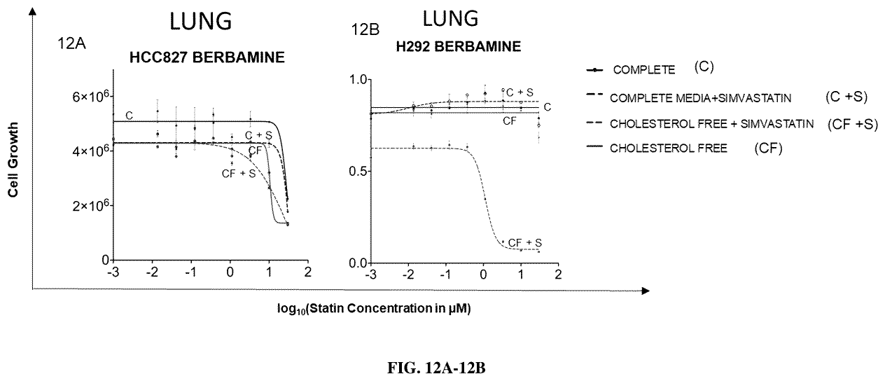

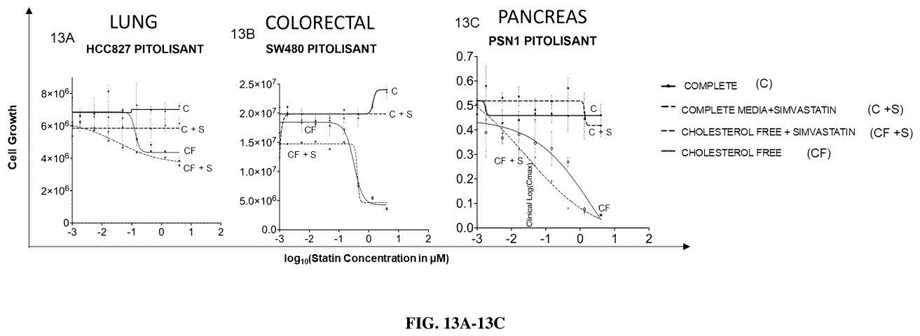

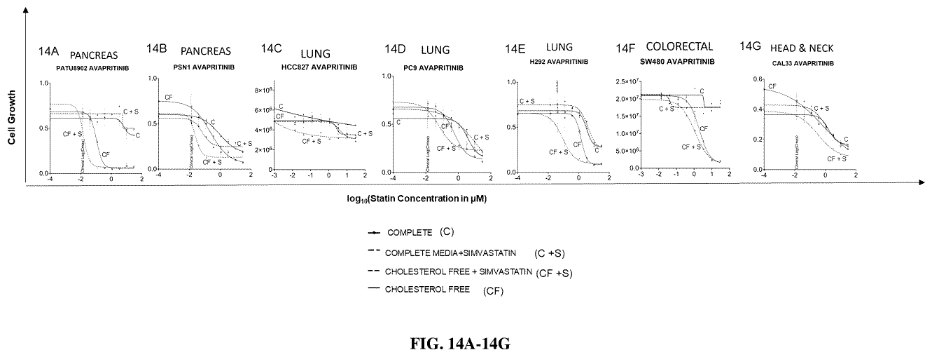

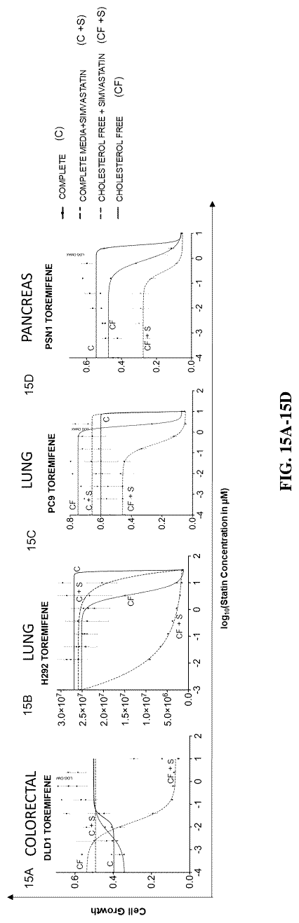

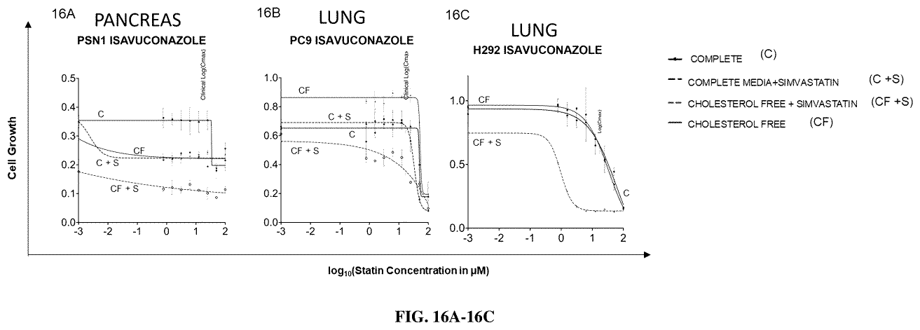

Growth curves showing absorbance of crystal violet at 595 nm or fluorescene of reosurfin at 590 nm were plotted from test wells against the logarithmic concentration of the validation drug. FIG. 9A-9C show growth of cancer cell lines in the four media conditions subjected to increasing doses of neticonazole. FIG. 10A-10D show growth of cancer cell lines in the four media conditions subjected to increasing doses of cepharanthine. FIG. 11A-11B show growth of cancer cell lines in the four media conditions subjected to increasing doses of alverine citrate. FIG. 12A-12B show growth of cancer cell lines in the four media conditions subjected to increasing doses of berbamine. FIG. 13A-13C show growth of cancer cell lines in the four media conditions subjected to increasing doses of pitolisant. FIG. 14A-14G show growth of cancer cell lines in the four media conditions subjected to increasing doses of avapritinib. FIG. 15A-15D show growth of cancer cell lines in the four media conditions subjected to increasing doses of toremifene. FIG. 16A-16C show growth of cancer cell lines in the four media conditions subjected to increasing doses of isavuconazole.

The anti-proliferative effect of neticonazole was strongly enhanced by the lack of cholesterol and presence of statin as indicated by the observed ‘left-shift’ in the dose-response curve when cells are grown in the cholesterol-free medium+simvastatin condition compared to the other media conditions in H292, PC9, and PSN1 cells (FIG. 9A-9C, respectively). Lack of cholesterol and the presence of statin greatly enhanced the anti-proliferative effect of cepharanthine as indicated by the ‘left-shift’ in the dose-response curve when cells are grown in the cholesterol-free mediun+simvastatin condition compared to the other media conditions in H292, PC9, and PSN1 cells. The cholesterol-free medium+simvastatin condition decreased basal cell viability of SW480 cells and enhanced the anti-proliferative effect of cepharanthine (FIG. 10A-10D). The lack of cholesterol- and presence of simvastatin condition enhanced the anti-proliferative effect of alverine citrate as indicated by the left-shift in the dose-response curves for SW480 and H292 cells grown in the cholesterol-free+simvastatin medium condition compared to the other media conditions (FIG. 11A-11B). The anti-proliferaitve effect of berbamine was greatly enhanced in H292 and HCC827 cells grown in cholesterol-free+simvastatin medium when compared to the other three media conditions, indicated by the left-shift in the corresponding dose-response curves (FIG. 12A-12B). Lack of cholesterol enhanced the activity of pitolisant, indicated by the left-shift in dose-response curves for HCC827, SW480, and PSN1 cells grown in cholesterol-free and cholesterol-free+simvastatin media conditions (FIG. 13A-13C). The anti-proliferative effect of avapritinib is enhanced by lack of cholesterol as indicated by the left-shift in the dose-response curves for cells grown in cholesterol-free media compared to complete media. The presence of simvastatin enhanced the anti-proliferative effect of avapritinib in cholesterol-free media, as indicated by the strong left-shift in the dose-response curves for H292, PATU8901, and PSN1 cells (FIG. 14A-14G). There was a strong left-shift in the dose-response curve for cells grown in the cholesterol-free+simvastatin medium treated with toremifene compared to the other three media conditions, indicating that lack of cholesterol and the presence of simvastatin increases the anti-proliferative activity of toremifene (FIG. 15A-15D). The growth of cells in the cholesterol-free medium+simvastatin was greatly decreased relative to the other three media conditions, as indicated by the strong left-shift in the dose-response curves for PC9 and H292 cells and decreased basal viability of PSN1 cells treated with isavuconazole (FIG. 16A-16C). Overall, the data demonstrate that modulating dietary cholesterol and administration of statins can enhance the anti-cancer effects of therapeutics.

### Example 4: Effect of Statins and Cholesterol-Restricted Diet on Anti-Cancer Activity of Therapeutics In Vivo

A series of in vivo experiments were designed to assess the effect of cholesterol depletion on cancer cell growth. The added role of statin treatment and additional therapeutic agents was studied by dosing mice and assessing tumor growth over a defined time period.

### Evaluation of the Anti-Tumour Activity of Atorvastatin and Toremifene in a Low Cholesterol Diet in NCI-H292 Mucoepidermoid Lung Cancer Tumour Bearing BALB/c Nude Mice.

Immunodeficient BALB/c nude mice were subcutaneously injected with human mucoepidermoid lung cancer NCI-H292 cells to initiate tumor development and randomly assigned into one of three experimental groups (n=6 per group): normal cholesterol diet “Normal Chol Diet” containing 0.2% dietary cholesterol without atorvastatin; low cholesterol diet “Low Chol Diet” containing less than 0.05% dietary cholesterol without atorvastatin; or Low Chol Diet treated with atorvastatin at 30 mg/kg by oral gavage. Once the tumor volume reached ˜150 mm³, experimental diets were commenced. Atorvastatin or vehicle treatment was initiated four days after the diet change and continued daily until the tumor volume exceeded 2000 mm³ or the time end-point of three weeks after diet commencement was reached. Throughout the course of the study, tumor volume was measured twice per week.

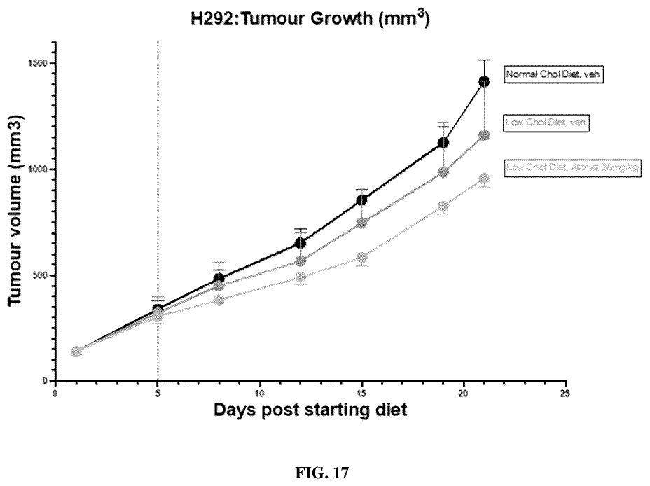

FIG. 17 shows the growth of H292 tumors in the three experimental conditions: Normal Chol Diet+vehicle, Low Chol Diet+vehicle, and Low Chol Diet+atorvastatin 30 mg/kg. A low cholesterol diet decreased tumor growth relative to the normal cholesterol diet. Atorvastatin treatment enhanced the tumor growth suppression observed with a low cholesterol diet. The data indicate that cholesterol modulation and statin treatment can suppress tumor growth in vivo.

### Evaluation of the Anti-Tumour Activity of Atorvastatin and Toremifene in a Low Cholesterol Diet in SW480 Colorectal Tumour Bearing M-NSG Mice.

Immunodeficient M-NSG (NOD-Prkdc^{scid} Il2rgem1Smoc) mice were subcutaneously injected with human colorectal cancer SW480 cells and matrigel to initiate tumor development and randomly assigned into one of four experimental groups (n=6 per group): normal cholesterol diet “Normal Chol Diet” containing 0.2% dietary cholesterol and vehicle, low cholesterol diet “Low Chol Diet” containing less than 0.05% dietary cholesterol and vehicle, Low Chol Diet treated with atorvastatin at 15 mg/kg, or Low Chol Diet treated with toremifene at 2 mg/kg. Once the tumor volume reached ˜150 mm³, experimental diets were commenced. Atorvastatin, vehicle, or toremifene treatment by oral gavage was initiated four days after the diet change and continued daily until the tumor volume exceeded 2000 mm³ or the time end-point of three weeks after treatment initiation was reached. Throughout the course of the study, tumor volume was measured twice per week.

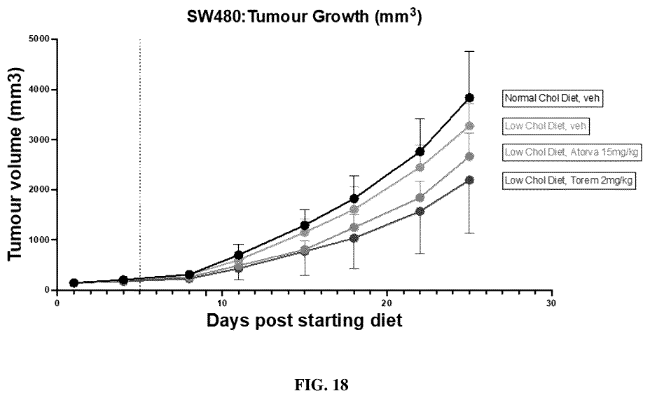

FIG. 18 shows the growth of SW480 tumors in the four experimental conditions: Normal Chol Diet+vehicle, Low Chol Diet+vehicle, Low Chol Diet+atorvastatin 15 mg/kg, and Low Chol Diet+toremifene 2 mg/kg. A low cholesterol diet decreased tumor growth relative to the normal cholesterol diet. The tumor growth suppression observed with the low cholesterol diet was enhanced with atorvastatin and toremifene treatment. Tumor growth suppression was highest in the low cholesterol diet+toremifene condition. The data demonstrate that cholesterol modulation can enhance the anti-tumor effects of therapeutics.

Evaluation of the Anti-Tumour Activity of Combining Simvastatin and Toremifene with a Low Cholesterol Diet in PC9 Bearing C.B-17 SCID Mice.

Immunodeficient C.B-17 SCID (C.B-Igh-1^{b}/IcrTac-Prkdc^{scid}) mice were subcutaneously injected with human lung cancer PC9 cells to initiate tumor development and randomly assigned into one of three experimental groups (n=6 per group): “Low Chol Diet” containing less than 0.05% dietary cholesterol and vehicle, Low Chol Diet and simvastatin 20 mg/kg, and Low Chol Diet and simvastatin 10 mg/kg in combination with toremifene 2 mg/kg. Once the tumor volume reached ˜200 mm³, treatment was commenced with vehicle, simvastatin, and toremifene administered daily by oral gavage. Treatment continued for 28 days or until the tumor volume exceeded 2000 mm³. Throughout the course of the study, tumor volume was measured twice per week.

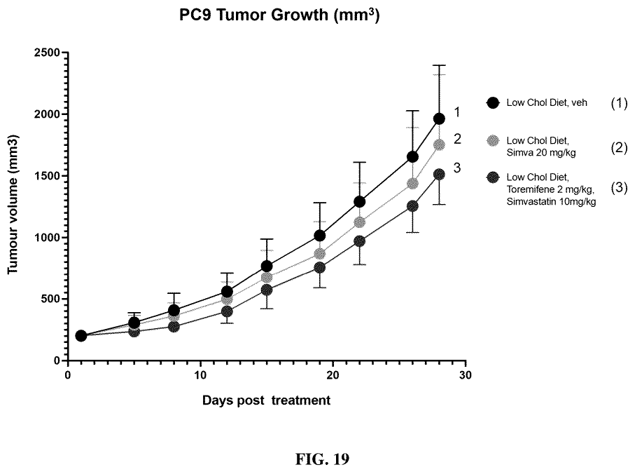

FIG. 19 shows the growth of PC9 tumors in the three experimental conditions: Low Chol Diet+vehicle, Low Chol Diet+simvastatin 20 mg/kg, and Low Chol Diet+simvastatin 10 mg/kg and toremifene 2 mg/kg. The combination of low cholesterol and simvastatin 20 mg/kg decreased tumor growth relative to the low cholesterol and vehicle treatment. Toremifene treatment with a lower simvastatin dose enhanced the tumor growth suppression compared to simvastatin treatment. Overall, the data demonstrate that cholesterol modulation and statin treatment can be used to control cancer cell growth.

### Example 5: Example Diets to be Administered with Compounds or Compositions of the Disclosure

A patient described herein can be administered any of the following diets to reduce dietary fat, dietary cholesterol intake, or both. An example diet can contain little to no animal or animal-derived products. Such diets are expected to contain little to no cholesterol, e.g., no more than 30 g/day total fat, no more than 10 g/day saturated fat, or no more than 200 mg/day cholesterol. Example diets can also be vegan and contain no animal or animal-derived products. Such diets are expected to be devoid of cholesterol.

Diet 1: 1) Breakfast: oatmeal made with non-dairy milk or skim milk, almond nut butter, chia seeds, flax seeds, and blueberries; 2) Lunch: large salad with many different types of plants, crispy chickpeas as a crouton-alternative, and an olive oil-based salad dressing; 3) Dinner: oven-baked salmon over a bed of ½ cup barley, side of green beans steamed with olive oil, lemon and garlic side salad with olive oil-based dressing; 4) Snack: bowl of edamame beans.

Diet 2: 1) Breakfast: oatmeal made with non-dairy milk or skim milk, almond nut butter, chia seeds, flax seeds, and blueberries; 2) Lunch: large salad with many different types of plants, crispy chickpeas as a crouton-alternative, and an olive oil-based salad dressing; 3) Dinner: oven-baked eggplant over a bed of ½ cup barley, side of green beans steamed with olive oil, lemon and garlic side salad with olive oil-based dressing; 4) Snack: bowl of edamame beans.

Diet 3: 1) Breakfast: cinnamon+banana oatmeal bowl, almond milk, flax seeds, and dried blueberries; 2) Lunch: Indian-spiced vegetables, brussel sprouts, green lentils, butternut squash, cremini mushrooms, and tahini sauce; 3) Dinner: baked gigante bean and artichokes, asparagus, cauliflower, basil; 4) Snacks: frozen dessert, cashew nuts, pistachio nuts, hazelnuts, vanilla bean and maple; and raspberry and fig bites, almond, coconut, dried cranberries.

Diet 4: 1) Breakfast: strawberry-almond smoothie, almond milk, lemon; and chia seed pudding, coconut yogurt, strawberry preserves; 2) Lunch: chopped salad, romaine, avocado, walnuts, blueberries, pickled peppers, chickpeas, vegan green-goddess dressing; 3) Dinner: vegan lasagna, whole wheat noodles, almonds, tomato-garlic sauce, butternut squash, kale; 4) Snacks: honey-roasted mixed nuts; and vegan chocolate chip cookies.

### Embodiments

Embodiment 1. A method of treating a condition in a subject in need thereof, the method comprising: a) administering to the subject a therapeutically effective amount of an inhibitor of a mevalonate pathway; and b) administering to the subject a diet, wherein the diet comprises less than about 40 g/day of lipids.

Embodiment 2. A method of treating a condition in a subject in need thereof, the method comprising: a) administering to the subject a therapeutically effective amount of an inhibitor of a mevalonate pathway; and b) administering to the subject a diet, wherein the diet comprises less than about 200 mg/day of cholesterol.

Embodiment 3. A method of treating a condition in a subject in need thereof, the method comprising: a) administering to the subject a therapeutically effective amount of an HMG-CoA reductase inhibitor; and b) administering to the subject a diet, wherein the diet comprises less than about 40 g/day of lipids.

Embodiment 4. A method of treating a condition in a subject in need thereof, the method comprising: a) administering to the subject a therapeutically effective amount of an HMG-CoA reductase inhibitor; and b) administering to the subject a diet, wherein the diet comprises less than about 200 mg/day of cholesterol.

Embodiment 5. The method of any one of embodiments 1-4, wherein the subject is a human.

Embodiment 6. The method of any one of embodiments 1-5, wherein the condition is a cancer.

Embodiment 7. The method of embodiment 6, wherein the cancer is lung cancer.

Embodiment 8. The method of embodiment 6, wherein the cancer is pancreatic cancer.

Embodiment 9. The method of embodiment 6, wherein the cancer is head and neck cancer.

Embodiment 10. The method of embodiment 6, wherein the cancer is colorectal cancer.

Embodiment 11. The method of embodiment 6, wherein the cancer is renal cancer.

Embodiment 12. The method of embodiment 6, wherein the cancer is breast cancer.

Embodiment 13. The method of embodiment 6, wherein the cancer is a blood cancer.

Embodiment 14. The method of embodiment 6, wherein the cancer is liver cancer.

Embodiment 15. The method of embodiment 6, wherein the cancer is a cholesterol-dependent cancer.

Embodiment 16. The method of any one of embodiments 1, 2, and 5-15, wherein the inhibitor of the mevalonate pathway is a statin.

Embodiment 17. The method of any one of embodiments 3-15, wherein the HMG-CoA reductase inhibitor is a statin.

Embodiment 18. The method of embodiment 16 or 17, wherein the statin is atorvastatin or a pharmaceutically acceptable salt thereof.

Embodiment 19. The method of embodiment 18, wherein the statin is atorvastatin calcium.

Embodiment 20. The method of embodiment 18, wherein the therapeutically effective amount of the atorvastatin or the pharmaceutically acceptable salt is from about 10 mg/day to about 80 mg/day.

Embodiment 21. The method of embodiment 16 or 17, wherein the statin is fluvastatin or a pharmaceutically acceptable salt thereof.

Embodiment 22. The method of embodiment 21, wherein the statin is fluvastatin sodium.

Embodiment 23. The method of embodiment 21, wherein the therapeutically effective amount of the fluvastatin or the pharmaceutically acceptable salt is from about 10 mg/day to about 80 mg/day.

Embodiment 24. The method of embodiment 16 or 17, wherein the statin is pravastatin or a pharmaceutically acceptable salt thereof.

Embodiment 25. The method of embodiment 24, wherein the statin is pravastatin sodium.

Embodiment 26. The method of embodiment 24, wherein the therapeutically effective amount of the pravastatin or the pharmaceutically acceptable salt is from about 20 mg/day to about 60 mg/day.

Embodiment 27. The method of embodiment 16 or 17, wherein the statin is rosuvastatin or a pharmaceutically acceptable salt thereof.

Embodiment 28. The method of embodiment 27, wherein the statin is rosuvastatin calcium.

Embodiment 29. The method of embodiment 27, wherein the therapeutically effective amount of the rosuvastatin or the pharmaceutically acceptable salt is from about 5 mg/day to about 40 mg/day.

Embodiment 30. The method of embodiment 16 or 17, wherein the statin is simvastatin or a pharmaceutically acceptable salt thereof.

Embodiment 31. The method of embodiment 30, wherein the statin is simvastatin sodium.

Embodiment 32. The method of embodiment 30, wherein the therapeutically effective amount of the simvastatin or the pharmaceutically acceptable salt is from about 5 mg/day to about 40 mg/day.

Embodiment 33. The method of embodiment 16 or 17, wherein the statin is pitavastatin or a pharmaceutically acceptable salt thereof.

Embodiment 34. The method of embodiment 33, wherein the statin is pitavastatin calcium.

Embodiment 35. The method of embodiment 33, wherein the statin is pitavastatin magnesium.

Embodiment 36. The method of embodiment 33, wherein the therapeutically effective amount of the pitavastatin or the pharmaceutically acceptable salt is from about 1.5 mg/day to about 4.5 mg/day.

Embodiment 37. The method of embodiment 16 or 17, wherein the statin is lovastatin or a pharmaceutically acceptable salt thereof.

Embodiment 38. The method of embodiment 37, wherein the statin is lovastatin sodium.

Embodiment 39. The method of embodiment 37, wherein the therapeutically effective amount of the lovastatin or the pharmaceutically acceptable salt is from about 1 mg/day to about 5 mg/day.

Embodiment 40. The method of any one of embodiments 1-39, further comprising administering a therapeutically-effective amount of a cholesterol absorption inhibitor.

Embodiment 41. The method of embodiment 40, wherein the cholesterol absorption inhibitor is ezetimibe.

Embodiment 42. The method of embodiment 40, wherein the therapeutically effective amount of the cholesterol absorption inhibitor is from about 5 mg/day to about 20 mg/day.

Embodiment 43. The method of any one of embodiments 16-42, wherein the administering the statin is oral.

Embodiment 44. The method of any one of embodiments 1-43, further comprising administering a cancer therapy.

Embodiment 45. The method of embodiment 44, wherein the cancer therapy is a chemotherapeutic agent.

Embodiment 46. The method of embodiment 44, wherein the cancer therapy is radiotherapy.

Embodiment 47. The method of embodiment 44, wherein the cancer therapy is an immunotherapy.

Embodiment 48. The method of any one of embodiments 1-47, wherein the diet is a vegan diet.

Embodiment 49. The method of any one of embodiments 1-47, wherein the diet is a vegetarian diet.

Embodiment 50. The method of any one of embodiments 1-47, wherein the diet is plant-based.

Embodiment 51. The method of any one of embodiments 1-47, wherein the diet is substantially devoid of animal fats.

Embodiment 52. The method of any one of embodiments 1-51, wherein the subject consumes a 2000 kcal/day diet.

Embodiment 53. A method of treating a condition in a subject in need thereof, the method comprising: a) administering to the subject a therapeutically effective amount of a cholesterol lowering agent; and b) administering to the subject a diet, wherein the diet is formulated to reduce a level of fat in the subject.

Embodiment 54. A method of treating a condition in a subject in need thereof, the method comprising: a) administering to the subject a therapeutically effective amount of a cholesterol lowering agent; and b) administering to the subject a diet, wherein the diet is formulated to reduce a level of cholesterol in the subject.

Embodiment 55. The method of embodiment 53 or 54, wherein the subject is a human.

Embodiment 56. The method of any one of embodiments 53-55, wherein the condition is a cancer.

Embodiment 57. The method of embodiment 56, wherein the cancer is lung cancer.

Embodiment 58. The method of embodiment 56, wherein the cancer is pancreatic cancer.

Embodiment 59. The method of embodiment 56, wherein the cancer is head and neck cancer.

Embodiment 60. The method of embodiment 56, wherein the cancer is colorectal cancer.

Embodiment 61. The method of embodiment 56, wherein the cancer is renal cancer.

Embodiment 62. The method of embodiment 56, wherein the cancer is breast cancer.

Embodiment 63. The method of embodiment 56, wherein the cancer is a blood cancer. Embodiment 64. The method of embodiment 56, wherein the cancer is liver cancer.

Embodiment 65. Embodiment 3. The method of embodiment 56, wherein the cancer is a cholesterol-dependent cancer.

Embodiment 66. The method of any one of embodiments 53-65, wherein the cholesterol lowering agent is an inhibitor of the mevalonate pathway.

Embodiment 67. The method of embodiment 66, wherein the inhibitor of the mevalonate pathway inhibits HMG-CoA reductase.

Embodiment 68. The method of embodiment 67, wherein the HMG-CoA reductase inhibitor is a statin.

Embodiment 69. The method of embodiment 68, wherein the statin is atorvastatin or a pharmaceutically acceptable salt thereof.

Embodiment 70. The method of embodiment 69, wherein the statin is atorvastatin calcium.

Embodiment 71. The method of embodiment 69, wherein the therapeutically effective amount of the atorvastatin or the pharmaceutically acceptable salt is from about 10 mg/day to about 80 mg/day.

Embodiment 72. The method of embodiment 68, wherein the statin is fluvastatin or a pharmaceutically acceptable salt thereof.

Embodiment 73. The method of embodiment 72, wherein the statin is fluvastatin sodium. Embodiment 74. The method of embodiment 72, wherein the therapeutically effective amount of the fluvastatin or the pharmaceutically acceptable salt is from about 10 mg/day to about 80 mg/day.

Embodiment 75. The method of embodiment 68, wherein the statin is pravastatin or a pharmaceutically acceptable salt thereof.

Embodiment 76. The method of embodiment 75, wherein the statin is pravastatin sodium.

Embodiment 77. The method of embodiment 75, wherein the therapeutically effective amount of the pravastatin or the pharmaceutically acceptable salt is from about 20 mg/day to about 60 mg/day.

Embodiment 78. The method of embodiment 68, wherein the statin is rosuvastatin or a pharmaceutically acceptable salt thereof.

Embodiment 79. The method of embodiment 78, wherein the statin is rosuvastatin calcium.

Embodiment 80. The method of embodiment 78, wherein the therapeutically effective amount of the rosuvastatin or the pharmaceutically acceptable salt is from about 5 mg/day to about 40 mg/day.

Embodiment 81. The method of embodiment 68, wherein the statin is simvastatin or a pharmaceutically acceptable salt thereof.

Embodiment 82. The method of embodiment 81, wherein the statin is simvastatin sodium.

Embodiment 83. The method of embodiment 81, wherein the therapeutically effective amount of the simvastatin or the pharmaceutically acceptable salt is from about 5 mg/day to about 40 mg/day.

Embodiment 84. The method of embodiment 68, wherein the statin is pitavastatin or a pharmaceutically acceptable salt thereof.

Embodiment 85. The method of embodiment 84, wherein the statin is pitavastatin calcium.

Embodiment 86. The method of embodiment 84, wherein the statin is pitavastatin magnesium.

Embodiment 87. The method of embodiment 84, wherein the therapeutically effective amount of the pitavastatin or the pharmaceutically acceptable salt is from about 1.5 mg/day to about 4.5 mg/day.

Embodiment 88. The method of embodiment 68, wherein the statin is lovastatin or a pharmaceutically acceptable salt thereof.

Embodiment 89. The method of embodiment 88, wherein the statin is lovastatin sodium.

Embodiment 90. The method of embodiment 88, wherein the therapeutically effective amount of the lovastatin or the pharmaceutically acceptable salt is from about 1 mg/day to about 5 mg/day.

Embodiment 91. The method of any one of embodiments 68-90, wherein the administering the statin is oral.

Embodiment 92. The method of any one of embodiments 53-65, wherein the cholesterol lowering agent is a cholesteryl ester transfer protein (CETP) inhibitor.

Embodiment 93. The method of any one of embodiments 53-65, wherein the cholesterol lowering agent is a lecithin-cholesterol acyltransferase (LCAT) inhibitor.

Embodiment 94. The method of any one of embodiments 53-65, wherein the cholesterol lowering agent is a bile acid sequestrant.

Embodiment 95. The method of any one of embodiments 53-65, wherein the cholesterol lowering agent is a cholesterol absorption inhibitor.

Embodiment 96. The method of any one of embodiments 91-95, wherein the administering the cholesterol lowering agent is oral.

Embodiment 97. The method of any one of embodiments 53-96, further comprising administering to the subject a cancer therapy.

Embodiment 98. The method of embodiment 97, wherein the cancer therapy is a chemotherapeutic agent.

Embodiment 99. The method of embodiment 97, wherein the cancer therapy is radiotherapy.

Embodiment 100. The method of embodiment 97, wherein the cancer therapy is an immunotherapy.

Embodiment 101. The method of any one of embodiments 53-100, wherein the diet is a vegan diet.

Embodiment 102. The method of any one of embodiments 53-100, wherein the diet is a vegetarian diet.

Embodiment 103. The method of any one of embodiments 53-100, wherein the diet is plant-based.

Embodiment 104. The method of any one of embodiments 53-100, wherein the diet is devoid of animal fats.

Embodiment 105. The method of any one of embodiments 53-104, wherein the diet is formulated to reduce a blood level of fat in the subject.

Embodiment 106. The method of any one of embodiments 53-105, wherein the diet is formulated to reduce a blood level of cholesterol in the subject.

Embodiment 107. The method of any one of embodiments 53-106, wherein the diet reduces a level of fat in the subject.

Embodiment 108. The method of any one of embodiments 53-107, wherein the diet reduces a level of cholesterol in the subject.

Embodiment 109. The method of any one of embodiments 53-108, wherein the diet reduces a blood level of fat in the subject.

Embodiment 110. The method of any one of embodiments 53-109, wherein the diet reduces a blood level of cholesterol in the subject.

Embodiment 111. The method of any one of embodiments 53-110, wherein the diet comprises less than 70% of a daily recommended cholesterol intake value.

Embodiment 112. The method of embodiment 111, wherein the daily recommended cholesterol intake value is according to a dietary guideline published by the United States Departments of Agriculture and Health and Human Services.

Embodiment 113. The method of embodiment 111, wherein a daily recommended cholesterol intake value is 300 mg/day.

Embodiment 114. The method of any one of embodiments 53-113, wherein the diet comprises less than 1% of the subject's average daily cholesterol intake prior to administering the diet.

Embodiment 115. The method of any one of embodiments 53-114, wherein the diet does not comprise cholesterol.

Embodiment 116. The method of any one of embodiments 53-115, wherein the diet comprises less than 50% of a daily recommended fat intake value.

Embodiment 117. The method of claim 116, wherein a daily recommended fat intake value is according to a dietary guideline published by the United States Departments of Agriculture and Health and Human Services.

Embodiment 118. The method of embodiment 116, wherein a daily recommended fat intake value is about 78 g/day.

Embodiment 119. The method of any one of embodiments 53-118, wherein the diet comprises less than 1% of the subject's average daily fat intake prior to administering the diet.

Embodiment 120. The method of any one of embodiments 53-119, wherein the diet does not comprise fat.

Embodiment 121. The method of any one of embodiments 53-120, wherein the diet comprises less than about 200 mg/day of cholesterol.

Embodiment 122. The method of any one of embodiments 53-121, wherein the diet comprises less than about 40 g/day of lipids.

Embodiment 123. The method of any one of embodiments 53-122, wherein the diet comprises less than 15% of total kcal/day in saturated fats.

Embodiment 124. The method of any one of embodiments 53-123, wherein the diet comprises less than 5% of total kcal/day in trans fats.

Embodiment 125. The method of any one of embodiments 53-124, wherein the diet comprises less than 15% of total kcal/day in polyunsaturated fats.

Embodiment 126. The method of any one of embodiments 53-125, wherein the diet comprises less than 5% of total kcal/day in monounsaturated fats.

Embodiment 127. The method of any one of embodiments 53-126, wherein the diet comprises less than a daily recommended intake value of saturated fat.

Embodiment 128. The method of embodiment 127, wherein a daily recommended saturated fat intake value is 20 g/day.

Embodiment 129. The method of any one of embodiments 53-128, wherein the subject consumes a 2000 kcal/day diet.

Embodiment 130. A composition comprising: a) a cholesterol lowering agent; and b) a dietary product, wherein the dietary product is formulated to reduce a level of fat, a level of cholesterol, or both in a subject.

Embodiment 131. The composition of embodiment 130, wherein the subject is a human.

Embodiment 132. The composition of embodiment 130 or 131, wherein the cholesterol lowering agent is an inhibitor of the mevalonate pathway.

Embodiment 133. The composition of embodiment 132, wherein the inhibitor of the mevalonate pathway inhibits HMG-CoA reductase.

Embodiment 134. The composition of embodiment 133, wherein the HMG-CoA reductase inhibitor is a statin.

Embodiment 135. The composition of embodiment 134, wherein the statin is atorvastatin or a pharmaceutically acceptable salt thereof.

Embodiment 136. The composition of embodiment 134, wherein the statin is fluvastatin or a pharmaceutically acceptable salt thereof.

Embodiment 137. The composition of embodiment 134, wherein the statin is pravastatin or a pharmaceutically acceptable salt thereof.

Embodiment 138. The composition of embodiment 134, wherein the statin is rosuvastatin or a pharmaceutically acceptable salt thereof.

Embodiment 139. The composition of embodiment 134, wherein the statin is simvastatin or a pharmaceutically acceptable salt thereof.

Embodiment 140. The composition of embodiment 134, wherein the statin is pitavastatin or a pharmaceutically acceptable salt thereof.

Embodiment 141. The composition of embodiment 134, wherein the statin is lovastatin or a pharmaceutically acceptable salt thereof.

Embodiment 142. The composition of embodiment 130 or 131, wherein the cholesterol lowering agent is a cholesteryl ester transfer protein (CETP) inhibitor.

Embodiment 143. The composition of embodiment 130 or 131, wherein the cholesterol lowering agent is a lecithin-cholesterol acyltransferase (LCAT) inhibitor.

Embodiment 144. The composition of embodiment 130 or 131, wherein the cholesterol lowering agent is a bile acid sequestrant.

Embodiment 145. The composition of embodiment 130 or 131, wherein the cholesterol lowering agent is a cholesterol absorption inhibitor.

Embodiment 146. The composition of any one of embodiments 130-145, wherein the cholesterol lowering agent is formulated for oral administration.

Embodiment 147. The composition of any one of embodiments 130-146, wherein the dietary product is formulated for oral administration.

Embodiment 148. The composition of any one of embodiments 130-147, wherein the dietary product is formulated as a solid.

Embodiment 149. The composition of any one of embodiments 130-147, wherein the dietary product is formulated as a liquid.

Embodiment 150. The composition of any one of embodiments 130-149, wherein the dietary product is a vegan.

Embodiment 151. The composition of any one of embodiments 130-149, wherein the dietary product is a vegetarian.

Embodiment 152. The composition of any one of embodiments 130-149, wherein the dietary product is plant-based.

Embodiment 153. The composition of any one of embodiments 130-149, wherein the dietary product is devoid of animal fats.

Embodiment 154. The composition of any one of embodiments 130-153, wherein the dietary product is formulated to reduce a blood level of fat in the subject.

Embodiment 155. The composition of any one of embodiments 130-154, wherein the dietary product is formulated to reduce a blood level of cholesterol in the subject.

Embodiment 156. The composition of any one of embodiments 130-155, wherein the dietary product reduces a level of fat in the subject.

Embodiment 157. The composition of any one of embodiments 130-156, wherein the dietary product reduces a level of cholesterol in the subject.

Embodiment 158. The composition of any one of embodiments 130-157, wherein the dietary product reduces a blood level of fat in the subject.

Embodiment 159. The composition of any one of embodiments 130-158, wherein the dietary product reduces a blood level of cholesterol in the subject.

Embodiment 160. The composition of any one of embodiments 130-159, wherein the dietary product comprises less than 70% of a daily recommended cholesterol intake value.

Embodiment 161. The composition of embodiment 160, wherein the daily recommended cholesterol intake value is according to a dietary guideline published by the United States Departments of Agriculture and Health and Human Services.

Embodiment 162. The composition of embodiment 160, wherein a daily recommended cholesterol intake value is 300 mg/day.

Embodiment 163. The composition of any one of embodiments 130-162, wherein the dietary product comprises less than 1% of the subject's average daily cholesterol intake.

Embodiment 164. The composition of any one of embodiments 130-163, wherein the dietary product does not comprise cholesterol.

Embodiment 165. The composition of any one of embodiments 130-164, wherein the dietary product comprises less than 50% of a daily recommended fat intake value.

Embodiment 166. The composition of claim 165, wherein a daily recommended fat intake value is according to a dietary productary guideline published by the United States Departments of Agriculture and Health and Human Services.

Embodiment 167. The composition of embodiment 165, wherein a daily recommended fat intake value is about 78 g/day.

Embodiment 168. The composition of any one of embodiments 130-167, wherein the dietary product comprises less than 1% of the subject's average daily fat intake.

Embodiment 169. The composition of any one of embodiments 130-168, wherein the dietary product does not comprise fat.

Embodiment 170. The composition of any one of embodiments 130-169, wherein the dietary product comprises less than about 200 mg/day of cholesterol.

Embodiment 171. The composition of any one of embodiments 130-170, wherein the dietary product comprises less than about 40 g/day of lipids.

Embodiment 172. The composition of any one of embodiments 130-171, wherein the dietary product comprises less than 15% of total kcal/day in saturated fats.

Embodiment 173. The composition of any one of embodiments 130-172, wherein the dietary product comprises less than 5% of total kcal/day in trans fats.

Embodiment 174. The composition of any one of embodiments 130-173, wherein the dietary product comprises less than 15% of total kcal/day in polyunsaturated fats.

Embodiment 175. The composition of any one of embodiments 130-174, wherein the dietary product comprises less than 5% of total kcal/day in monounsaturated fats.

Embodiment 176. The composition of any one of embodiments 130-175, wherein the dietary product comprises less than a daily recommended intake value of saturated fat.

Embodiment 177. The composition of embodiment 176, wherein a daily recommended saturated fat intake value is 20 g/day.

Embodiment 178. A kit comprising: a) an effective amount of a cholesterol lowering agent; b) an effective amount of a dietary product, wherein the dietary product is formulated to reduce a level of fat, a level of cholesterol, or both in a subject; and optionally, c) written instructions for use of the kit.

Embodiment 179. The kit of embodiment 178, wherein the subject is a human.

Embodiment 180. The kit of embodiment 178 or 179, wherein the cholesterol lowering agent is an inhibitor of the mevalonate pathway.

Embodiment 181. The kit of embodiment 180, wherein the inhibitor of the mevalonate pathway inhibits HMG-CoA reductase.

Embodiment 182. The kit of embodiment 181, wherein the HMG-CoA reductase inhibitor is a statin.

Embodiment 183. The kit of embodiment 182, wherein the statin is atorvastatin or a pharmaceutically acceptable salt thereof.

Embodiment 184. The kit of embodiment 182, wherein the statin is fluvastatin or a pharmaceutically acceptable salt thereof.

Embodiment 185. The kit of embodiment 182, wherein the statin is pravastatin or a pharmaceutically acceptable salt thereof.

Embodiment 186. The kit of embodiment 182, wherein the statin is rosuvastatin or a pharmaceutically acceptable salt thereof.

Embodiment 187. The kit of embodiment 182, wherein the statin is simvastatin or a pharmaceutically acceptable salt thereof.

Embodiment 188. The kit of embodiment 182, wherein the statin is pitavastatin or a pharmaceutically acceptable salt thereof.

Embodiment 189. The kit of embodiment 182, wherein the statin is lovastatin or a pharmaceutically acceptable salt thereof.

Embodiment 190. The kit of embodiment 178 or 179, wherein the cholesterol lowering agent is a cholesteryl ester transfer protein (CETP) inhibitor.

Embodiment 191. The kit of embodiment 178 or 179, wherein the cholesterol lowering agent is a lecithin-cholesterol acyltransferase (LCAT) inhibitor.

Embodiment 192. The kit of embodiment 178 or 179, wherein the cholesterol lowering agent is a bile acid sequestrant.

Embodiment 193. The kit of embodiment 178 or 179, wherein the cholesterol lowering agent is a cholesterol absorption inhibitor.

Embodiment 194. The kit of any one of embodiments 178-193, wherein the cholesterol lowering agent is formulated for oral administration.

Embodiment 195. The kit of any one of embodiments 178-194, wherein the dietary product is formulated for oral administration.

Embodiment 196. The kit of any one of embodiments 178-195, wherein the dietary product is formulated as a solid.

Embodiment 197. The kit of any one of embodiments 178-195, wherein the dietary product is formulated as a liquid.

Embodiment 198. The kit of any one of embodiments 178-197, wherein the dietary product is a vegan.

Embodiment 199. The kit of any one of embodiments 178-197, wherein the dietary product is a vegetarian.

Embodiment 200. The kit of any one of embodiments 178-197, wherein the dietary product is plant-based.

Embodiment 201. The kit of any one of embodiments 178-197, wherein the dietary product is devoid of animal fats.

Embodiment 202. The kit of any one of embodiments 178-201, wherein the dietary product is formulated to reduce a blood level of fat in the subject.

Embodiment 203. The kit of any one of embodiments 178-202, wherein the dietary product is formulated to reduce a blood level of cholesterol in the subject.

Embodiment 204. The kit of any one of embodiments 178-203, wherein the dietary product reduces a level of fat in the subject.

Embodiment 205. The kit of any one of embodiments 178-204, wherein the dietary product reduces a level of cholesterol in the subject.

Embodiment 206. The kit of any one of embodiments 178-205, wherein the dietary product reduces a blood level of fat in the subject.

Embodiment 207. The kit of any one of embodiments 178-206, wherein the dietary product reduces a blood level of cholesterol in the subject.

Embodiment 208. The kit of any one of embodiments 178-207, wherein the dietary product comprises less than 70% of a daily recommended cholesterol intake value.

Embodiment 209. The kit of embodiment 208, wherein the daily recommended cholesterol intake value is according to a dietary guideline published by the United States Departments of Agriculture and Health and Human Services.

Embodiment 210. The kit of embodiment 280, wherein a daily recommended cholesterol intake value is 300 mg/day.

Embodiment 211. The kit of any one of embodiments 178-210, wherein the dietary product comprises less than 1% of the subject's average daily cholesterol intake.

Embodiment 212. The kit of any one of embodiments 178-211, wherein the dietary product does not comprise cholesterol.

Embodiment 213. The kit of any one of embodiments 178-212, wherein the dietary product comprises less than 50% of a daily recommended fat intake value.

Embodiment 214. The kit of claim 213, wherein a daily recommended fat intake value is according to a dietary productary guideline published by the United States Departments of Agriculture and Health and Human Services.

Embodiment 215. The kit of embodiment 213, wherein a daily recommended fat intake value is about 78 g/day.

Embodiment 216. The kit of any one of embodiments 178-215, wherein the dietary product comprises less than 1% of the subject's average daily fat intake.

Embodiment 217. The kit of any one of embodiments 178-216, wherein the dietary product does not comprise fat.

Embodiment 218. The kit of any one of embodiments 178-217, wherein the dietary product comprises less than about 200 mg/day of cholesterol.

Embodiment 219. The kit of any one of embodiments 178-218, wherein the dietary product comprises less than about 40 g/day of lipids.

Embodiment 220. The kit of any one of embodiments 178-219, wherein the dietary product comprises less than 15% of total kcal/day in saturated fats.

Embodiment 221. The kit of any one of embodiments 178-220, wherein the dietary product comprises less than 5% of total kcal/day in trans fats.

Embodiment 222. The kit of any one of embodiments 178-221, wherein the dietary product comprises less than 15% of total kcal/day in polyunsaturated fats.

Embodiment 223. The kit of any one of embodiments 178-222, wherein the dietary product comprises less than 5% of total kcal/day in monounsaturated fats.

Embodiment 224. The kit of any one of embodiments 178-223, wherein the dietary product comprises less than a daily recommended intake value of saturated fat.

Embodiment 225. The kit of embodiment 224, wherein a daily recommended saturated fat intake value is 20 g/day.

1. A method of treating a cancer in a subject in need thereof, the method comprising:
a) administering to the subject a therapeutically effective amount of an inhibitor of a mevalonate pathway; and
b) administering to the subject a diet, wherein the diet comprises less than about 40 g/day of lipids.

2. The method of claim 1, wherein the diet comprises less than about 200 mg/day of cholesterol.

3. The method of claim 1, wherein the inhibitor of the mevalonate pathway is an HMG-CoA reductase inhibitor.

4. The method of claim 3, wherein the diet comprises less than about 200 mg/day of cholesterol.

5. The method of claim 1, wherein the subject is a human.

6. (canceled)

7. The method of claim 1, wherein the cancer is lung cancer, pancreatic cancer, head and neck cancer, colorectal cancer, renal cancer, breast cancer, a blood cancer, or liver cancer.

8-15. (canceled)

16. The method of claim 1, wherein the inhibitor of the mevalonate pathway is a statin.

17. (canceled)

18. The method of claim 16, wherein the statin is atorvastatin or a pharmaceutically acceptable salt thereof, fluvastatin or a pharmaceutically acceptable salt thereof, pravastatin or a pharmaceutically acceptable salt thereof, rosuvastatin or a pharmaceutically acceptable salt thereof, simvastatin or a pharmaceutically acceptable salt thereof, pitavastatin or a pharmaceutically acceptable salt thereof, or lovastatin or a pharmaceutically acceptable salt thereof.

19. The method of claim 18, wherein the statin is atorvastatin calcium, fluvastatin sodium, pravastatin sodium, rosuvastatin calcium, simvastatin sodium, pitavastatin calcium, pitavastatin magnesium, or lovastatin sodium.

20. The method of claim 18, wherein the therapeutically effective amount of the atorvastatin or the pharmaceutically acceptable salt is from about 10 mg/day to about 80 mg/day, the therapeutically effective amount of the fluvastatin or the pharmaceutically acceptable salt is from about 10 mg/day to about 80 mg/day, the therapeutically effective amount of the pravastatin or the pharmaceutically acceptable salt is from about 20 mg/day to about 60 mg/day, the therapeutically effective amount of the rosuvastatin or the pharmaceutically acceptable salt is from about 5 mg/day to about 40 mg/day, the therapeutically effective amount of the simvastatin or the pharmaceutically acceptable salt is from about 5 mg/day to about 40 mg/day, the therapeutically effective amount of the pitavastatin or the pharmaceutically acceptable salt is from about 1.5 mg/day to about 4.5 mg/day, or the therapeutically effective amount of the lovastatin or the pharmaceutically acceptable salt is from about 1 mg/day to about 5 mg/day.

21-39. (canceled)

40. The method of claim 1, further comprising administering a therapeutically-effective amount of a cholesterol lowering agent.

41-43. (canceled)

44. The method of claim 1, further comprising administering a cancer therapy.

45. The method of claim 44, wherein the cancer therapy is a chemotherapeutic agent.

46. The method of claim 44, wherein the cancer therapy is radiotherapy.

47. The method of claim 44, wherein the cancer therapy is an immunotherapy.

48. The method of claim 1, wherein the diet is a vegan diet, a vegetarian diet, a plant based diet, or a diet devoid of animal fats.

49-91. (canceled)

92. The method of claim 40, wherein the cholesterol lowering agent is a cholesteryl ester transfer protein (CETP) inhibitor, a lecithin-cholesterol acyltransferase (LCAT) inhibitor, a bile acid sequestrant, or a cholesterol absorption inhibitor.

93-122.

123. The method of claim 1, wherein the diet comprises less than 15% of total kcal/day in saturated fats.

124. The method of claim 1, wherein the diet comprises less than 5% of total kcal/day in trans fats.

125. The method of claim 1, wherein the diet comprises less than 15% of total kcal/day in polyunsaturated fats.

126. The method of claim 1, wherein the diet comprises less than 5% of total kcal/day in monounsaturated fats.

127. The method of claim 1, wherein the diet comprises less than 20 g/day a daily recommended intake value of saturated fat.

128. (canceled)

129. The method of claim 1, wherein the subject consumes a 2000 kcal/day diet.
